(19) United States
(12) Patent Application Publication
(10) Pub. No.: US 2003/0186308 A1
(43) Pub. Date: Oct. 2, 2003

(54) Prostacyclin-stimulating factor-2

(75) Inventors: Paul E. Young, Gaithersburg, MD (US); Steven M. Ruben, Brookeville, MD (US)

Correspondence Address: HUMAN GENOME SCIENCES INC, 9410 KEY WEST AVENUE, ROCKVILLE, MD, 20850

(73) Assignee: Human Genome Sciences, Inc., Rockville, MD (US)

(21) Appl. No.: 10/418,064

(22) Filed: Apr. 18, 2003

Related U.S. Application Data
(63) Continuation of application No. 09/461,419, filed on Dec. 16, 1999.
(60) Provisional application No. 60/113,009, filed on Dec. 18, 1998.

(51) Int. Cl. C12Q 1/68; C07H 21/04; C12P 21/02; C12N 5/06; C07K 14/705

(52) U.S. Cl. 435/6; 435/69.1; 435/320.1; 435/325; 530/350; 536/23.5

(57) ABSTRACT

The present invention relates to a novel human polypeptide called Prostacyclin-Stimulating Factor-2 (PSF-2), and isolated polynucleotides encoding this polypeptide. Also provided are vectors, host cells, antibodies, and recombinant methods for producing this human polypeptide. The invention further relates to diagnostic and therapeutic methods useful for diagnosing, preventing, and treating disorders related to this novel human polypeptide.

### CROSS-REFERENCE TO RELATED APPLICATIONS

This application is a continuation of U.S. application Ser. No. 09/461,419, filed Dec. 16, 1999, which claims benefit under 35 U.S.C. § 119(e) of the filing date of U.S. Provisional Application Serial No. 60/113,009, filed Dec. 18, 1998, all of which are hereby incorporated by reference in their entirety.

### FIELD OF THE INVENTION

The present invention relates to a novel human gene encoding a polypeptide that is a member of the prostacyclin stimulating factor/MAC25/Insulin-like Growth Factor Binding Polypeptide (IGFBP) family. More specifically, the present invention relates to a polynucleotide encoding a novel human polypeptide named Prostacyclin-Stimulating Factor-2, or “PSF-2.” This invention also relates to PSF-2 polypeptides, as well as vectors, host cells, antibodies directed to PSF-2 polypeptides, and the recombinant methods for producing the same. Also provided are diagnostic methods for detecting disorders related to the vascular and/or immune system, and therapeutic methods for treating such disorders. The invention further relates to screening methods for identifying agonists and antagonists of PSF-2 activity.

### BACKGROUND OF THE INVENTION

Prostacyclin (also termed PGI₂) is a potent vocative polypeptide, which functions at least in vessel wall homeostasis. It is expressed mainly by vascular endothelial cells (Monaca, S. and Vane, J. R., Br. Med. Bull. 34:129-35; 1978). More specifically, synthesis of PGI₂ occurs at the level of the afferent glomerular arteriole in close contact with the renin-producing cells of the juxtaglomerular apparatus allowing PGI₂ to modulate release of renin. Decreases in PGI₂ expression and production have been linked with the vascular complications associated with Diabetes (Inoguchi, T., et al., Diabetes Res. Clin. Pract. 3:243-38 (1987)). A 282 amino acid residue polypeptide designated PGI₂-stimulating factor (PSF) was recently molecularly cloned from cultured human diploid fibroblast cells (Yamauchi, T., et al., Biochem. Mol. Biol. Int. 31:65-71 (1993); Yamauchi, T., et al., Biochem. J. 303:591-98 (1994)). PSF corresponds to a PGI₂-stimulating activity, which is significantly decreased in the plasma-derived serum of patients with Diabetes (Inoguchi, T., et al., Metabolism 38:1561-68 (1989); Umeda, F., et al., Diabetes 45 Suppl. 3:S111-13 (1996); Kawai, C. Circulation 90(2):1033-43 (1994)).

PSF stimulates production of prostacyclin, which, in turn, has been shown to induce elevation of cyclic AMP levels. In this instance, elevation results from stimulation of myosin light chain kinase phosphorylation rather than by reduction of cytosolic free calcium levels. The result is a decrease in calcium sensitivity of the contractile proteins of vascular smooth muscle tissue (Griffith, T. M., et al., J. Am. Coll. Cardiol. 12:797-806 (1988); Adelstein, R. S., et al., Am. J. Cardiol. 44:783-87 (1979)).

Thus, the role of PSF is integrally intertwined with the role(s) of a number of other potentially vasoregulatory factors including, for example, endothelium-derived relaxing factor, renin, angiotensin, adenosine, thrombin, acetylcholine, vocative intestinal peptide, bradykinin, substance P, cholecystokinin, calcitonin-gene-related peptide, noradrenaline, histamine, A23187 (calcium ionophore), norepinephrine, isoproterenol, serotonin, insulin, glucose, histamine, lippopolysacharide, IL-1, leukotriene D₄, mellitin, phospholipase C, phospholipase A₂, IFN-gamma, ergometrine, and others in homeostasis of vessel structures and in the pathophysiology of a number of conditions, disorders, and disease states including, for example, diabetes, diabetic angiopathy, thrombotic thromobocytic purpura (TTP), coronary vasospasm, cerebral vasoconstriction, hypertension, aging, cardiomyopathy, atherogenesis, microvessel disturbances, inflammation, pain, fever, reproduction, gastric secretion, peptic ulcer, ductus arteriosis, congenital heart disease, platelet aggregation, thrombosis, myocardial infarction, ischemia, ischemic heart disease, reperfusion injury, modulation of baroreceptor activity, and the like.

As a result, there is a need for polypeptides that are related to the PSF/MAC25/IGFBP family. IGFBP's have been implicated in a variety of cellular processes including, for example: stimulation of prostacyclin production by endothelial cells; activin binding; tumor suppression; cellular proliferation; and regulation of cellular differentiation. Disturbances of regulation of the aforementioned processes may be involved in disorders relating to vascular and/or immune system. Therefore, there is a need for identification and characterization of such human polypeptides that can play a role in detecting, preventing, ameliorating, or correcting such disorders.

### SUMMARY OF THE INVENTION

The present invention relates to a novel polynucleotide and the encoded polypeptide of PSF-2. Moreover, the present invention relates to vectors, host cells, antibodies, and recombinant methods for producing the polypeptides and polynucleotides. Also provided are diagnostic methods for detecting disorders related to the polypeptides, and therapeutic methods for treating such disorders. The invention further relates to screening methods for identifying binding partners of PSF-2.

### BRIEF DESCRIPTION OF THE DRAWINGS

FIGS. 1A and 1B show the nucleotide sequence (SEQ ID NO:1) and deduced amino acid sequence (SEQ ID NO:2) of PSF-2. The predicted leader sequence of about 30 amino acids is double-underlined (Met-1 to Ala-30). Three potential asparagine-linked glycosylation sites are marked in the amino acid sequence of PSF-2. The potential sites of glycosylation begin at asparagine-159, asparagine-183, and asparagine-277 in FIGS. 1A and 1B. The potential glycosylation sites are marked with a bold pound symbol (#) above the nucleotide sequence coupled with a bolded one letter abbreviation for the asparagine (N) in the amino acid sequence in FIGS. 1A and 1B.

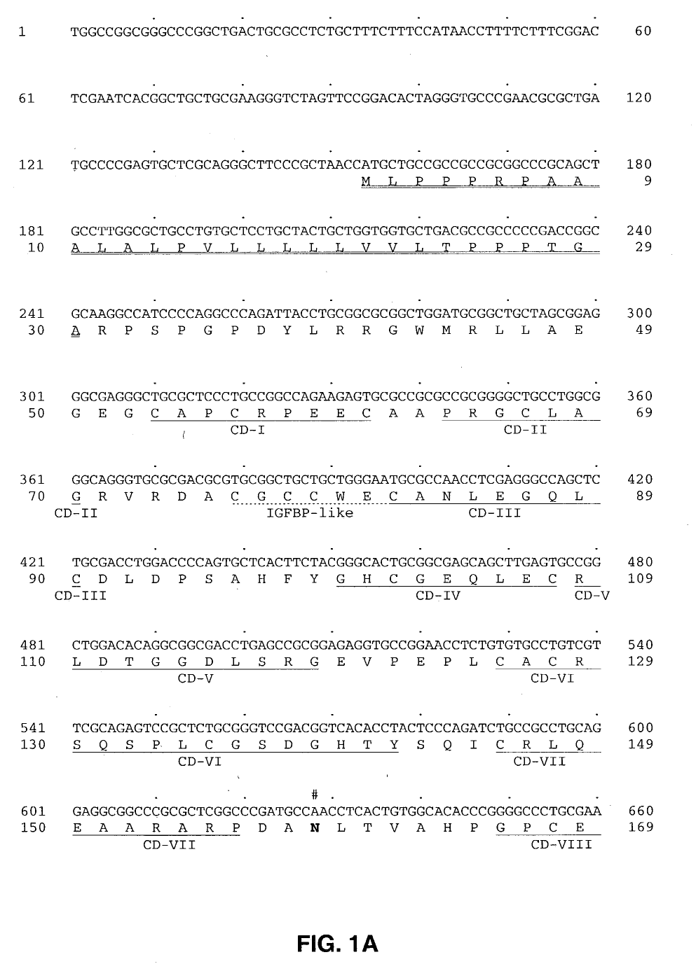

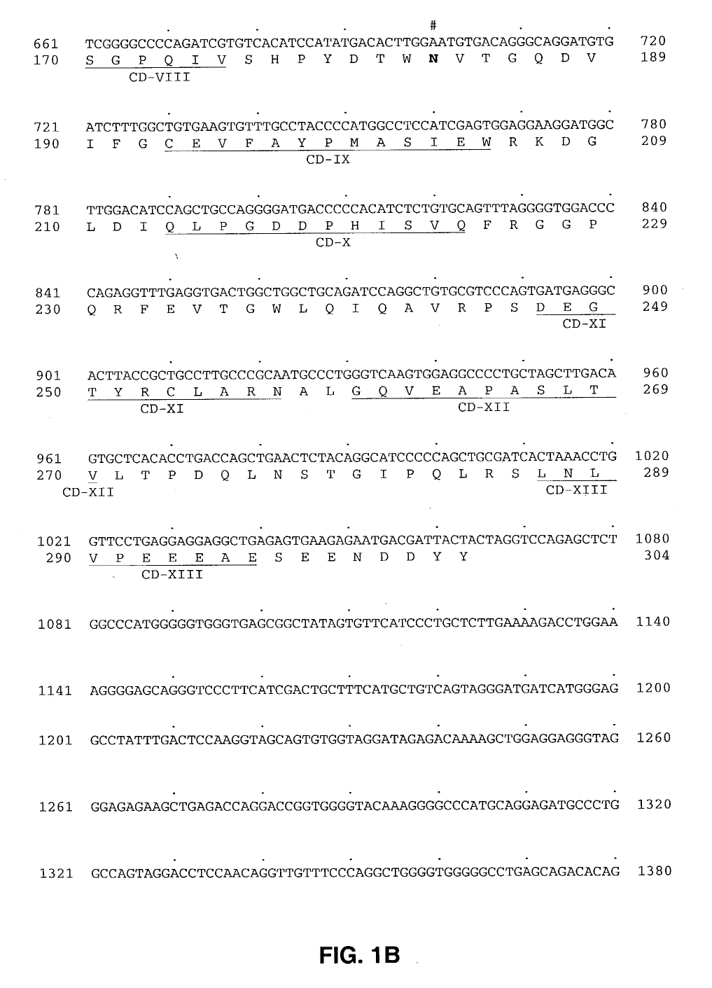

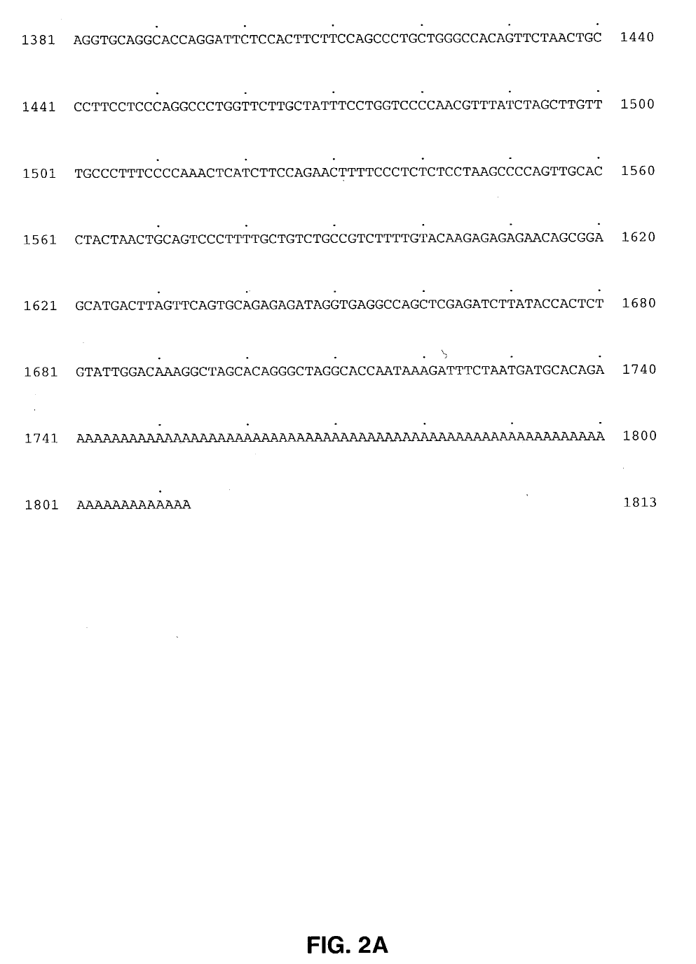

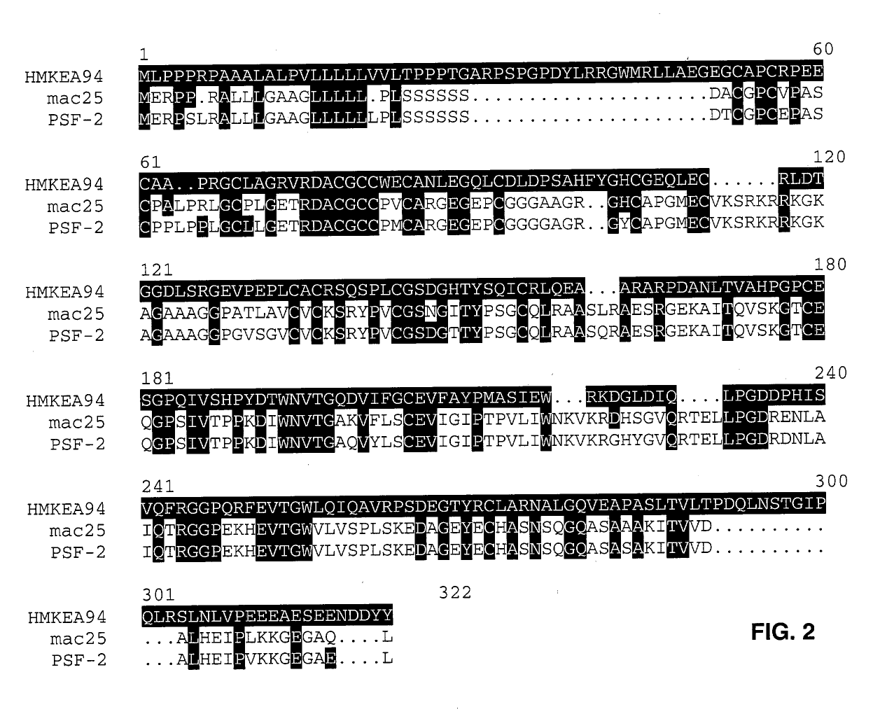

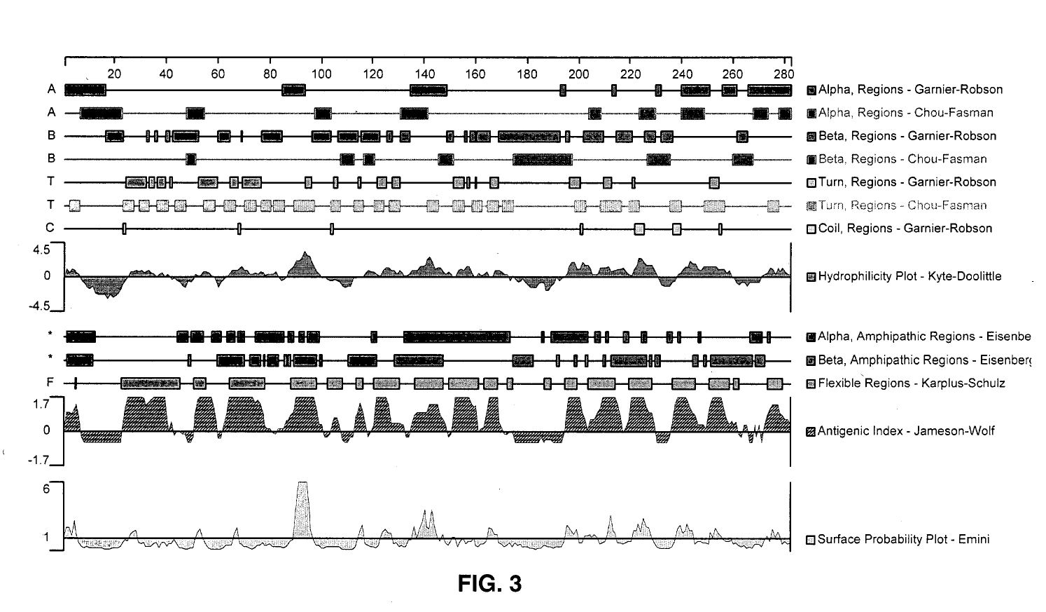

A region of the PSF-2 polypeptide which corresponds to an IGFBP family motif is located at positions Cys-76 through Cys-82 of the PSF-2 polypeptide sequence shown in FIGS. 1A and 1B. This sequence is delineated in FIGS. 1A and 1B with a dotted underline under the sequence between and including amino acid residues Cys-76 through Cys-82. Residues Cys-76, Gly-77, Cys-78, Cys-79, Trp-80, Glu-81, and Cys-82 are underlined with a dotted line in FIGS 1A and 1B.

Regions of high identity between PSF-2, the Mus musculus mac25 gene (SEQ ID NO:3; ATCC Accession No. AB012886), and the closely related human prostacyclin-stimulating factor (PSF) (SEQ ID NO:5; ATCC Accession No. S75725) (an alignment of these sequences is presented in FIGS. 2A and 2B) are underlined in FIGS. 1A and 1B. These regions are not in any way limiting and are labeled as Conserved Domain (CD)-I (Cys-53 through Cys-61), CD-II (Pro-64 through Gly-70), CD-III (Cys-82 through Cys-90), CD-IV (Gly-100 through Cys-108), CD-V (Arg-109 through Gly-119), CD-VI (Cys-126 through Tyr-142), CD-VII (Cys-146 through Arg-155), CD-VIII (Gly-166 through Val-175), CD-IX (Cys-193 through Trp-205), CD-X (Gln-213 through Gln-224), CD-XI (Asp-247 through Asn-257), CD-XII (Gly-260 through Val-270), and CD-XIII (Leu-287 through Glu-296) in FIGS. 1A and 1B. Also preferred are fragments containing 1 or more of conserved domains CD-I through CD-XIII.

FIGS. 2A and 2B show an alignment of the amino acid sequences of the Mus musculus mac25 gene (SEQ ID NO:3; ATCC Accession No. AB012886), human prostacyclin-stimulating factor (PSF) (SEQ ID NO:5; ATCC Accession No. S75725), and PSF-2 (SEQ ID NO:2). The alignment was generated using the “MegAlign” module of the DNA*Star Sequence Analysis computer program (DNASTAR, Inc.) using the default parameters. Amino acid residues of mac25 and PSF which have identity with those of PSF-2 are highlighted in black in the alignment. By examining the regions of amino acids shaded and/or boxed, the skilled artisan can readily identify conserved domains between the two polypeptides. These conserved domains are preferred embodiments of the present invention. Examples of these conserved domains are labeled in FIGS. 1A and 1B as CD-I, CD-II, CD-III, CD-IV, CD-V, CD-VI, CD-VII, CD-VIII, CD-IX, CD-X, CD-XI, CD-XII, and CD-XIII

FIG. 3 shows a protein analysis of the PSF-2 amino acid sequence. Alpha, beta, turn and coil regions; hydrophilicity and hydrophobicity; amphipathic regions; flexible regions; antigenic index and surface probability are shown, and all were generated using the default settings of the “Protean” module of the DNA*Star Sequence Analysis computer program (DNASTAR, Inc.). In the “Antigenic Index or Jameson-Wolf” graph, the positive peaks indicate locations of the highly antigenic regions of the. PSF-2 polypeptide, i.e., regions from which epitope-bearing peptides of the invention can be obtained. The domains defined by these graphs are contemplated by the present invention.

The data presented in FIG. 3 is also represented in tabular form in Table I. The columns in the Table are labeled with the headings “Res”, “Position”, and Roman Numerals I-XIV. The column headings refer to the following features of the amino acid sequence presented in FIG. 3 and Table I: “Res”: amino acid residue of SEQ ID NO:2 and FIGS. 1A and 1B; “Position”: position of the corresponding residue within SEQ ID NO:2 and FIGS. 1A and 1B; I: Alpha, Regions—Garnier-Robson; II: Alpha, Regions—Chou-Fasman; III: Beta, Regions—Garnier-Robson; IV: Beta, Regions—Chou-Fasman; V: Turn, Regions—Garnier-Robson; VI: Turn, Regions—Chou-Fasman; VII: Coil, Regions—Garnier-Robson; VIII: Hydrophilicity Plot—Kyte-Doolittle; IX: Hydrophobicity Plot—Hopp-Woods; X: Alpha, Amphipathic Regions—Eisenberg; XI: Beta, Amphipathic Regions—Eisenberg; XII: Flexible Regions—Karplus-Schulz; XIII: Antigenic Index—Jameson-Wolf; and XIV: Surface Probability Plot—Emini.

### DETAILED DESCRIPTION

Definitions

The following definitions are provided to facilitate understanding of certain terms used throughout this specification.

In the present invention, “isolated” refers to material removed from its original environment (e.g., the natural environment if it is naturally occurring), and thus is altered “by the hand of man” from its natural state. For example, an isolated polynucleotide could be part of a vector or a composition of matter, or could be contained within a cell, and still be “isolated” because that vector, composition of matter, or particular cell is not the original environment of the polynucleotide. In another embodiment, an “isolated” nucleic acid molecule does not encompass a chromosome isolated or removed from a cell or a cell lysate (e.g., a “chromosome spread,” as in a karyotype). In yet another embodiment, an “isolated” nucleic acid molecule does not encompass a cDNA library, a genomic library, a yeast artificial chromosome (YAC), a bacterial artificial chromosome (BAC) or other artificial chromosome type vector which is comprised by a PSF-2 nucleic acid of the invention.

In the present invention, a “secreted” PSF-2 polypeptide refers to a polypeptide capable of being directed to the ER, secretory vesicles, or the extracellular space as a result of a signal sequence, as well as a PSF-2 polypeptide released into the extracellular space without necessarily containing a signal sequence. If the PSF-2 secreted polypeptide is released into the extracellular space, the PSF-2 secreted polypeptide can undergo extracellular processing to produce a “mature” PSF-2 polypeptide. Release into the extracellular space can occur by many mechanisms, including exocytosis and proteolytic cleavage.

As used herein, a PSF-2 “polynucleotide” refers to a molecule having a nucleic acid sequence contained in SEQ ID NO:1 or the cDNA contained within the clone deposited with the American Type Culture Collection (ATCC). For example, the PSF-2 polynucleotide can contain the nucleotide sequence of the full length cDNA sequence, including the 5′ and 3′ untranslated sequences, the coding region, with or without the signal sequence, the secreted polypeptide coding region, as well as fragments, epitopes, domains, and variants of the nucleic acid sequence. Moreover, as used herein, a PSF-2 “polypeptide” refers to a molecule having the translated amino acid sequence generated from the polynucleotide as broadly defined.

In specific embodiments, the polynucleotides of the invention are less than 300 kb, 200 kb, 100 kb, 50 kb, 15 kb, 10 kb, or 7.5 kb in length. In a further embodiment, polynucleotides of the invention comprise at least 15 contiguous nucleotides of PSF-2 coding sequence, but do not comprise all or a portion of any PSF-2 intron. In another embodiment, the nucleic acid comprising PSF-2 coding sequence does not contain coding sequences of a genomic flanking gene (i.e., 5′ or 3′ to the PSF-2 gene in the genome).

In the present invention, the full length PSF-2 sequence identified as SEQ ID NO:1 was generated by overlapping sequences of the deposited clone (contig analysis). A representative clone containing all or most of the sequence for SEQ ID NO:1 was deposited with the ATCC on Dec. 17, 1998, and was given the ATCC Deposit Number 203521. The ATCC is located at 10801 University Boulevard, Manassas, Va. 20110-2209, USA. The ATCC deposit was made pursuant to the terms of the Budapest Treaty on the international recognition of the deposit of microorganisms for purposes of patent procedure.

A PSF-2 “polynucleotide” also includes those polynucleotides capable of hybridizing, under stringent hybridization conditions, to sequences contained in SEQ ID NO:1, the complement thereof, or the cDNA within the deposited clone. “Stringent hybridization conditions” refers to an overnight incubation at 42 C in a solution comprising 50% formamide, 5×SSC (750 mM NaCl, 75 mM sodium citrate), 50 mM sodium phosphate (pH 7.6), 5× Denhardt's solution, 10% dextran sulfate, and 20 μg/ml denatured, sheared salmon sperm DNA, followed by washing the filters in 0.1×SSC at about 65 C.

Also contemplated are nucleic acid molecules that hybridize to the PSF-2 polynucleotides at moderatetly high stringency hybridization conditions. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37 C in a solution comprising 6×SSPE (20×SSPE=3M NaCl; 0.2M NaH₂PO₄; 0.02M EDTA, pH 7.4), 0.5% SDS, 30% formamide, 100 ug/ml salmon sperm blocking DNA; followed by washes at 50 C with 1×SSPE, 0.1% SDS. In addition, to achieve even lower stringency, washes performed following stringent hybridization can be done at higher salt concentrations (e.g., 5×SSC).

Note that variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

Of course, a polynucleotide which hybridizes only to polyA+ sequences (such as any 3′ terminal polyA+ tract of a cDNA shown in the sequence listing), or to a complementary stretch of T (or U) residues, would not be included in the definition of “polynucleotide,” since such a polynucleotide would hybridize to any nucleic acid molecule containing a poly (A) stretch or the complement thereof (e.g., practically any double-stranded cDNA clone).

The PSF-2 polynucleotide can be composed of any polyribonucleotide or polydeoxribonucleotide, which may be unmodified RNA or DNA or modified RNA or DNA. For example, PSF-2 polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, the PSF-2 polynucleotides can be composed of triple-stranded regions comprising RNA or DNA or both RNA and DNA. PSF-2 polynucleotides may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

PSF-2 polypeptides can be composed of amino acids joined to each other by peptide bonds or modified peptide bonds, i.e., peptide isosteres, and may contain amino acids other than the 20 gene-encoded amino acids. The PSF-2 polypeptides may be modified by either natural processes, such as posttranslational processing, or by chemical modification techniques which are well known in the art. Such modifications are well described in basic texts and in more detailed monographs, as well as in a voluminous research literature. Modifications can occur anywhere in the PSF-2 polypeptide, including the peptide backbone, the amino acid side-chains and the amino or carboxyl termini. It will be appreciated that the same type of modification may be present in the same or varying degrees at several sites in a given PSF-2 polypeptide. Also, a given PSF-2 polypeptide may contain many types of modifications. PSF-2 polypeptides may be branched, for example, as a result of ubiquitination, and they may be cyclic, with or without branching. Cyclic, branched, and branched cyclic PSF-2 polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to polypeptides such as arginylation, and ubiquitination. (See, for instance, POLYPEPTIDES—STRUCTURE AND MOLECULAR PROPERTIES, 2nd Ed., T. E. Creighton, W. H. Freeman and Company, New York (1993); POSTTRANSLATIONAL COVALENT MODIFICATION OF POLYPEPTIDES, B. C. Johnson, Ed., Academic Press, New York, pgs. 1-12 (1983); Seifter, et al., Meth Enzymol. 182:626-646 (1990); Rattan, et al., Ann. N.Y. Acad. Sci. 663:48-62 (1992).)

“SEQ ID NO:1” refers to a PSF-2 polynucleotide sequence while “SEQ ID NO:2” refers to a PSF-2 polypeptide sequence.

A PSF-2 polypeptide “having biological activity” refers to polypeptides exhibiting activity similar, but not necessarily identical to, an activity of a PSF-2 polypeptide, including mature forms, as measured in a particular biological assay, with or without dose dependency. In the case where dose dependency does exist, it need not be identical to that of the PSF-2 polypeptide, but rather substantially similar to the dose-dependence in a given activity as compared to the PSF-2 polypeptide (i.e., the candidate polypeptide will exhibit greater activity or not more than about 25-fold less and, preferably, not more than about tenfold less activity, and most preferably, not more than about three-fold less activity relative to the PSF-2 polypeptide.)

PSF-2 Polynucleotides and Polypeptides

Clone HMKEA94 was isolated from a human meningima cDNA library. This clone contains the entire coding region identified as SEQ ID NO:2. The deposited clone contains a cDNA having a total of 1813 nucleotides, which encodes a predicted open reading frame of 304 amino acid residues. (See FIGS. 1A and 1B.) The open reading frame begins at a N-terminal methionine located at nucleotide position 154, and ends at a stop codon beginning at nucleotide position 1066. The predicted molecular weight of the PSF-2 polypeptide should be about 32,962 Daltons.

Subsequent Northern analysis also showed PSF-2 expression. There are two primary transcripts visible on Northern blots (approximately 2 and 3.5 kb in size). The highest levels of expression are clearly seen in spleen, while lower levels of expression are visible in a variety of tissues examined, including prostate, testis, colon, stomach, thyroid, small intestine. There is no obvious expression in peripheral blood cells. PSF-2 is likely to be expressed by endothelial cells.

Using BLAST analysis, SEQ ID NO:2 was found to be homologous to members of the prostacyclin stimulating factor/MAC25/IGFBP family. Particularly, SEQ ID NO:2 contains domains homologous to the translation product of the Mus musculus mRNA mac25 (ATCC Accession No. AB012886; SEQ ID NO:3) (See, FIGS. 2A and 2B).

mac25 is a retinoic acid-inducible gene that is expressed at high levels in senescent epithelial cells. It was initially cloned as a gene that is differentially expressed in meningioma. Although the homology of its product with members of family of insulin-like growth factor-binding polypeptides was suggested, the product also exhibits strong homology to follistatin, an activin-binding polypeptide. However, a domain corresponding to the carboxyl terminus of follistatin is not found in mac25. The carboxyl-terminally truncated form of follistatin, generated by alternative splicing, has stronger activin-binding activity than the complete form. This result suggests that mac25 might act as an “activated follistatin.” Clonal growth of a p53-deficient osteosarcoma cell line was strongly inhibited when the murine mac25 gene, as well as the p53 gene, was introduced. Resembling activins that belong to the transforming growth factor-b (TGF-b) superfamily, mac25 and p53 might associate with similar but distinct targets, namely cyclin-dependent kinase inhibitors. However, there is no evidence for compensation of p53 function by mac25 in the development of p53-deficient mice, as judged from the pattern of expression of mac25 in mice. mac25 might act as a tumor suppressor, modulating signaling of the TGF-beta family, as does alpha-inhibin.

PSF, a human polypeptide which is also related to mac25, is a polypeptide that stimulates the synthesis of prostacyclin (PGI2) by vascular endothelial cells (ECs). Reduced staining for PSF was found in an atherosclerotic versus a normal coronary artery of humans. PSF may be involved in the production of PGI2 in the vessel wall and may participate in the maintenance of vascular homeostasis. PSF abnormalities may be involved in the development of such vascular lesions as atherosclerosis and diabetic angiopathy. Thus, the homology between mac25, PSF, and PSF-2 suggests that PSF-2 may also be involved in the stimulation of prostacyclin production by endothelial cells; activin binding; tumor suppression; cellular proliferation; and the regulation of cellular differentiation.

BLAST analyses indicate that SEQ ID NO:2 is related to members of the prostacyclin stimulating factor/MAC25/IGFBP family. In addition, SEQ ID NO:2 contains a number of domains exhibiting a high level of sequence identity with Mus musculus mac25 mRNA and polypeptide (SEQ ID NO:3; ATCC Accession No. AB012886) and prostacyclin-stimulating factor (PSF) (SEQ ID NO:5; ATCC Accession No. S75725) mRNA and polypeptide (See, FIGS. 2A and 2B), including the following conserved domains: (a) a predicted signal peptide domain located at about amino acids Met-1 to Ala-30; (b) a predicted IGFBP motif located at about amino acids Cys-76 to Cys-82; (c) a predicted mature polypeptide domain located at about amino acids Arg-31 to Tyr-304; (d) a conserved domain (CD)-I located at about amino acids Cys-53 to Cys-61; (e) a conserved domain CD-II located at about amino acids Pro-64 to Gly-70; (f) a conserved domain CD-III located at about amino acids Cys-82 to Cys-90; (g), a conserved domain CD-IV located at about amino acids Gly-100 to Cys-108; (h) a conserved domain CD-V located at about amino acids Arg-109 to Gly-119; (i) a conserved domain CD-VI located at about amino acids Cys-126 to Tyr-142; (j) a conserved domain CD-VII located at about amino acids Cys-146 to Arg-155; (k) a conserved domain CD-VIII located at about amino acids Gly-166 to Val-175; (l) a conserved domain CD-IX located at about amino acids Cys-193 to Trp-205; (m) a conserved domain CD-X located at about amino acids Gln-213 to Gln-224; (n) a conserved domain CD-XI located at about amino acids Asp-247 to Asn-257; (o) a conserved domain CD-XII located at about amino acids Gly-260 to Val-270; and (p) a conserved domain CD-XIII located at about amino acids Leu-287 to Glu-296. These polypeptide fragments of PSF-2 are specifically contemplated in the present invention.

Moreover, the encoded polypeptide has a predicted leader sequence located at about amino acids Met-1 to Ala-30. (See FIGS. 1A and 1B.) Also shown in FIGS. 1A and 1B, the predicted mature polypeptide encompasses about amino acids Arg-31 to Tyr-304, while the predicted secreted form of PSF-2 encompasses about amino acids Arg-31 to Tyr-304. These polypeptide fragments of PSF-2 are specifically contemplated in the present invention.

The PSF-2 nucleotide sequence identified as SEQ ID NO:1 was assembled from partially homologous (“overlapping”) sequences obtained from the deposited clone. The overlapping sequences were assembled into a single contiguous sequence of high redundancy resulting in a final sequence identified as SEQ ID NO:1.

Therefore, SEQ ID NO:1 and the translated SEQ ID NO:2 are sufficiently accurate and otherwise suitable for a variety of uses well known in the art and described further below. For instance, SEQ ID NO:1 is useful for designing nucleic acid hybridization probes that will detect nucleic acid sequences contained in SEQ ID NO:1 or the cDNA contained in the deposited clone. These probes will also hybridize to nucleic acid molecules in biological samples, thereby enabling a variety of forensic and diagnostic methods of the invention. Similarly, polypeptides identified from SEQ ID NO:2 may be used to generate antibodies which bind specifically to PSF-2.

Nevertheless, DNA sequences generated by sequencing reactions can contain sequencing errors. The errors exist as misidentified nucleotides, or as insertions or deletions of nucleotides in the generated DNA sequence. The erroneously inserted or deleted nucleotides cause frame shifts in the reading frames of the predicted amino acid sequence. In these cases, the predicted amino acid sequence diverges from the actual amino acid sequence, even though the generated DNA sequence may be greater than 99.9% identical to the actual DNA sequence (for example, one base insertion or deletion in an open reading frame of over 1000 bases).

Accordingly, for those applications requiring precision in the nucleotide sequence or the amino acid sequence, the present invention provides not only the generated nucleotide sequence identified as SEQ ID NO:1 and the predicted translated amino acid sequence identified as SEQ ID NO:2, but also a sample of plasmid DNA containing a human cDNA of PSF-2 deposited with the ATCC. The nucleotide sequence of the deposited PSF-2 clone can readily be determined by sequencing the deposited clone in accordance with known methods. The predicted PSF-2 amino acid sequence can then be verified from such deposits. Moreover, the amino acid sequence of the polypeptide encoded by the deposited clone can also be directly determined by peptide sequencing or by expressing the polypeptide in a suitable host cell containing the deposited human PSF-2 cDNA, collecting the polypeptide, and determining its sequence.

The present invention also relates to the PSF-2 gene corresponding to SEQ ID NO:1, SEQ ID NO:2, or the deposited clone. The PSF-2 gene can be isolated in accordance with known methods using the sequence information disclosed herein. Such methods include preparing probes or primers from the disclosed sequence and identifying or amplifying the PSF-2 gene from appropriate sources of genomic material.

Also provided in the present invention are species homologs of PSF-2. Species homologs may be isolated and identified by making suitable probes or primers from the sequences provided herein and screening a suitable nucleic acid source for the desired homologue.

The PSF-2 polypeptides can be prepared in any suitable manner. Such polypeptides include isolated naturally occurring polypeptides, recombinantly produced polypeptides, synthetically produced polypeptides, or polypeptides produced by a combination of these methods. Means for preparing such polypeptides are well understood in the art.

The PSF-2 polypeptides may be in the form of the secreted polypeptide, including the mature form, or may be a part of a larger polypeptide, such as a fusion polypeptide (see below). It is often advantageous to include an additional amino acid sequence which contains secretory or leader sequences, pro-sequences, sequences which aid in purification, such as multiple histidine residues, or an additional sequence for stability during recombinant production.

PSF-2 polypeptides are preferably provided in an isolated form, and preferably are substantially purified. A recombinantly produced version of a PSF-2 polypeptide, including the secreted polypeptide, can be substantially purified by the one-step method described by Smith and Johnson (Gene 67:31-40(1988)). PSF-2 polypeptides also can be purified from natural or recombinant sources using antibodies of the invention raised against the PSF-2 polypeptide in methods which are well known in the art.

Polynucleotide and Polypeptide Variants

“Variant” refers to a polynucleotide or polypeptide differing from the PSF-2 polynucleotide or polypeptide, but retaining essential properties thereof. Generally, variants are overall closely similar, and, in many regions, identical to the PSF-2 polynucleotide or polypeptide.

By a polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence encoding the PSF-2 polypeptide. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. The query sequence may be an entire sequence shown of SEQ ID NO:1, the ORF (open reading frame), or any fragement specified as described herein.

As a practical matter, whether any particular nucleic acid molecule or polypeptide is at least 90%, 95%, 96%, 97%, 98% or 99% identical to a nucleotide sequence of the presence invention can be determined conventionally using known computer programs. A preferred method for determing the best overall match between a query sequence (a sequence of the present invention) and a subject sequence, also referred to as a global sequence alignment, can be determined using the FASTDB computer program based on the algorithm of Brutlag, et al. (Comp. App. Biosci. 6:237-245 (1990).) In a sequence alignment the query and subject sequences are both DNA sequences. An RNA sequence can be compared by converting U's to T's. The result of said global sequence alignment is in percent identity. Preferred parameters used in a FASTDB alignment of DNA sequences to calculate percent identiy are: Matrix=Unitary, k-tuple=4, Mismatch Penalty=1, Joining Penalty=30, Randomization Group Length=0, Cutoff Score=1, Gap Penalty=5, Gap Size Penalty 0.05, Window Size=500 or the lenght of the subject nucleotide sequence, whichever is shorter.

If the subject sequence is shorter than the query sequence because of 5′ or 3′ deletions, not because of internal deletions, a manual correction must be made to the results. This is becuase the FASTDB program does not account for 5′ and 3′ truncations of the subject sequence when calculating percent identity. For subject sequences truncated at the 5′ or 3′ ends, relative to the the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by results of the FASTDB sequence alignment. This percentage is then subtracted from the percent identity, calculated by the above FASTDB program using the specified parameters, to arrive at a final percent identity score. This corrected score is what is used for the purposes of the present invention. Only bases outside the 5′ and 3′ bases of the subject sequence, as displayed by the FASTDB alignment, which are not matched/aligned with, the query sequence, are calculated for the purposes of manually adjusting the percent identity score.

For example, a 90 base subject sequence is aligned to a 100 base query sequence to determine percent identity. The deletions occur at the 5′ end of the subject sequence and therefore, the FASTDB alignment does not show a matched/alignement of the first 10 bases at 5′ end. The 10 unpaired bases represent 10% of the sequence (number of bases at the 5′ and 3′ ends not matched/total number of bases in the query sequence) so 10% is subtracted from the percent identity score calculated by the FASTDB program. If the remaining 90 bases were perfectly matched the final percent identity would be 90%. In another example, a 90 base subject sequence is compared with a 100 base query sequence. This time the deletions are internal deletions so that there are no bases on the 5′ or 3′ of the subject sequence which are not matched/aligned with the query. In this case the percent identity calculated by FASTDB is not manually corrected. Once again, only bases 5′ and 3′ of the subject sequence which are not matched/aligned with the query sequnce are manually corrected for. No other manual corrections are to made for the purposes of the present invention.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, (indels) or substituted with another amino acid. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 90%, 95%, 96%, 97%, 98% or 99% identical to, for instance, the amino acid sequences shown in SEQ ID NO:2 or to the amino acid sequence encoded by deposited DNA clone can be determined conventionally using known computer programs. A preferred method for determing the best overall match between a query sequence (a sequence of the present invention) and a subject sequence, also referred to as a global sequence alignment, can be determined using the FASTDB computer program based on the algorithm of Brutlag, et al. (Comp. App. Biosci. 6:237-245 (1990)). In a sequence alignment the query and subject sequences are either both nucleotide sequences or both amino acid sequences. The result of said global sequence alignment is in percent identity. Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter.

If the subject sequence is shorter than the query sequence due to N- or C-terminal deletions, not because of internal deletions, a manual correction must be made to the results. This is becuase the FASTDB program does not account for N- and C-terminal truncations of the subject sequence when calculating global percent identity. For subject sequences truncated at the N- and C-termini, relative to the the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence.

For example, a 90 amino acid residue subject sequence is aligned with a 100 residue query sequence to determine percent identity. The deletion occurs at the N-terminus of the subject sequence and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence which are not matched/aligned with the query. In this case the percent identity calculated by FASTDB is not manually corrected. Once again, only residue positions outside the N- and C-terminal ends of the subject sequence, as displayed in the FASTDB alignment, which are not matched/aligned with the query sequnce are manually corrected for. No other manual corrections are to made for the purposes of the present invention.

The PSF-2 variants may contain alterations in the coding regions, non-coding regions, or both. Especially preferred are polynucleotide variants containing alterations which produce silent substitutions, additions, or deletions, but do not alter the properties or activities of the encoded polypeptide. Nucleotide variants produced by silent substitutions due to the degeneracy of the genetic code are preferred. Moreover, variants in which 5-10, 1-5, or 1-2 amino acids are substituted, deleted, or added in any combination are also preferred. PSF-2 polynucleotide variants can be produced for a variety of reasons, e.g., to optimize codon expression for a particular host (change codons in the human mRNA to those preferred by a bacterial host such as E. coli). In additional embodiments, variants may contain sequence changes in sequence located outside of conserved domains.

Naturally occurring PSF-2 variants are called “allelic variants,” and refer to one of several alternate forms of a gene occupying a given locus on a chromosome of an organism. (Genes II, Lewin, B., ed., John Wiley & Sons, New York (1985).) These allelic variants can vary at either the polynucleotide and/or polypeptide level. Alternatively, non-naturally occurring variants may be produced by mutagenesis techniques or by direct synthesis.

Using known methods of polypeptide engineering and recombinant DNA technology, variants may be generated to improve or alter the characteristics of the PSF-2 polypeptides. For instance, one or more amino acids can be deleted from the N-terminus or C-terminus of the secreted polypeptide without substantial loss of biological function. The authors of Ron, et al., J. Biol. Chem. 268:2984-2988 (1993), reported variant KGF polypeptides having heparin binding activity even after deleting 3, 8, or 27 amino-terminal amino acid residues. Similarly, Interferon gamma exhibited up to ten times higher activity after deleting 8-10 amino acid residues from the carboxy terminus of this polypeptide. (Dobeli, et al., J. Biotechnology 7:199-216 (1988).)

Moreover, ample evidence demonstrates that variants often retain a biological activity similar to that of the naturally occurring polypeptide. For example, Gayle and coworkers (J. Biol. Chem. 268:22105-22111 (1993)) conducted extensive mutational analysis of human cytokine IL-1a. They used random mutagenesis to generate over 3,500 individual IL-1a mutants that averaged 2.5 amino acid changes per variant over the entire length of the molecule. Multiple mutations were examined at every possible amino acid position. The investigators found that “[m]ost of the molecule could be altered with little effect on either [binding or biological activity].” (See, Abstract.) In fact, only 23 unique amino acid sequences, out of more than 3,500 nucleotide sequences examined, produced a polypeptide that significantly differed in activity from wild-type.

Furthermore, even if deleting one or more amino acids from the N-terminus or C-terminus of a polypeptide results in modification or loss of one or more biological functions, other biological activities may still be retained. For example, the ability of a deletion variant to induce and/or to bind antibodies which recognize the secreted form will likely be retained when less than the majority of the residues of the secreted form are removed from the N-terminus or C-terminus. Whether a particular polypeptide lacking N- or C-terminal residues of a polypeptide retains such immunogenic activities can readily be determined by routine methods described herein and otherwise known in the art.

Thus, the invention further includes PSF-2 polypeptide variants which show substantial biological activity. Such variants include deletions, insertions, inversions, repeats, and substitutions selected according to general rules known in the art so as have little effect on activity. For example, guidance concerning how to make phenotypically silent amino acid substitutions is provided in Bowie, J. U., et al., Science 247:1306-1310 (1990), wherein the authors indicate that there are two main strategies for studying the tolerance of an amino acid sequence to change.

The first strategy exploits the tolerance of amino acid substitutions by natural selection during the process of evolution. By comparing amino acid sequences in different species, conserved amino acids can be identified. These conserved amino acids are likely important for polypeptide function. In contrast, the amino acid positions where substitutions have been tolerated by natural selection indicates that these positions are not critical for polypeptide function. Thus, positions tolerating amino acid substitution could be modified while still maintaining biological activity of the polypeptide.

The second strategy uses genetic engineering to introduce amino acid changes at specific positions of a cloned gene to identify regions critical for polypeptide function. For example, site directed mutagenesis or alanine-scanning mutagenesis (introduction of single alanine mutations at every residue in the molecule) can be used. (Cunningham and Wells, Science 244:1081-1085 (1989).) The resulting mutant molecules can then be tested for biological activity.

As the authors state, these two strategies have revealed that polypeptides are surprisingly tolerant of amino acid substitutions. The authors further indicate which amino acid changes are likely to be permissive at certain amino acid positions in the polypeptide. For example, most buried (within the tertiary structure of the polypeptide) amino acid residues require nonpolar side chains, whereas few features of surface side chains are generally conserved. Moreover, tolerated conservative amino acid substitutions involve replacement of the aliphatic or hydrophobic amino acids Ala, Val, Leu and Ile; replacement of the hydroxyl residues Ser and Thr; replacement of the acidic residues Asp and Glu; replacement of the amide residues Asn and Gln, replacement of the basic residues Lys, Arg, and His; replacement of the aromatic residues Phe, Tyr, and Trp, and replacement of the small-sized amino acids Ala, Ser, Thr, Met, and Gly.

For example, site directed changes at the amino acid level of PSF-2 can be made by replacing a particular amino acid with a conservative amino acid. Preferred conservative mutations include: M1 replaced with A, G, I, L, S, T, or V; L2 replaced with A, G, I, S, T, M, or V; R6 replaced with H, or K; A8 replaced with G, I, L, S, T, M, or V; A9 replaced with G, I, L, S, T, M, or V; A10 replaced with G, I, L, S, T, M, or V; L11 replaced with A, G, I, S, T, M, or V; A12 replaced with G, I, L, S, T, M, or V; L13 replaced with A, G, I, S, T, M, or V; V15 replaced with A, G, I, L, S, T, or M; L16 replaced with A, G, I, S, T, M, or V; L17 replaced with A, G, I, S, T, M, or V; L18 replaced with A, G, I, S, T, M, or V; L19 replaced with A, G, I, S, T, M, or V; L20 replaced with A, G, I, S, T, M, or V; V21 replaced with A, G, I, L, S, T, or M; V22 replaced with A, G, I, L, S, T, or M; L23 replaced with A, G, I, S, T, M, or V; T24 replaced with A, G, I, L, S, M, or V; T28 replaced with A, G, I, L, S, M, or V; G29 replaced with A, I, L, S, T, M, or V; A30 replaced with G, T, L, S, T, M, or V; R31 replaced with H, or K; S33 replaced with A, G, I, L, T, M, or V; G35 replaced with A, I, L, S, T, M, or V; D37 replaced with E; Y38 replaced with F, or W; L39 replaced with A, G, I, S, T, M, or V; R40 replaced with H, or K; R41 replaced with H, or K; G42 replaced with A, I, L, S, T, M, or V; W43 replaced with F, or Y; M44 replaced with A, G, I, L, S, T, or V; R45 replaced with H, or K; L46 replaced with A, G, T, S, T, M, or V; L47 replaced with A, G, I, S, T, M, or V; A48 replaced with G, I, L, S, T, M, or V; E49 replaced with D; G50 replaced with A, I, L, S, T, M, or V; E51 replaced with D; G52 replaced with A, I, L, S, T, M, or V; A54 replaced with G, I, L, S, T, M, or V; R57 replaced with H, or K; E59 replaced with D; E60 replaced with D; A62 replaced with G, I, L, S, T, M, or V; A63 replaced with G, I, L, S, T, M, or V; R65 replaced with H, or K; G66 replaced with A, I, L, S, T, M, or V; L68 replaced with A, G, I, S, T, M, or V; A69 replaced with G, I, L, S, T, M, or V; G70 replaced with A, I, L, S, T, M, or V; R71 replaced with H, or K; V72 replaced with A, G, I, L, S, T, or M; R73 replaced with H, or K; D74 replaced with E; A75 replaced with G, I, L, S, T, M, or V; G77 replaced with A, I, L, S, T, M, or V; W80 replaced with F, or Y; E81 replaced with D; A83 replaced with G, I, L, S, T, M, or V; N84 replaced with Q; L85 replaced with A, G, I, S, T, M, or V; E86 replaced with D; G87 replaced with A, I, L, S, T, M, or V; Q88 replaced with N; L89 replaced with A, G, I, S, T, M, or V; D91 replaced with E; L92 replaced with A, G, I, S, T, M, or V; D93 replaced with E; S95 replaced with A, G, I, L, T, M, or V; A96 replaced with G, I, L, S, T, M, or V; H97 replaced with K, or R; F98 replaced with W, or Y; Y99 replaced with F, or W; G100 replaced with A, I, L, S, T, M, or V; H101 replaced with K, or R; G103 replaced with A, I, L, S, T, M, or V; E104 replaced with D; Q105 replaced with N; L106 replaced with A, G, I, S, T, M, or V; E107 replaced with D; R109 replaced with H, or K; L110 replaced with A, G, I, S, T, M, or V; D111 replaced with E; T112 replaced with A, G, I, L, S, M, or V; G113 replaced with A, I, L, S, T, M, or V; G114 replaced with A, I, L, S, T, M, or V; D115 replaced with E; L116 replaced with A, G, I, S, T, M, or V; S117 replaced with A, G, I, L, T, M, or V; R118 replaced with H, or K; G119 replaced with A, I, L, S, T, M, or V; E120 replaced with D; V121 replaced with A, G, I, L, S, T, or M; E123 replaced with D; L125 replaced with A, G, I, S, T, M, or V; A127 replaced with G, I, L, S, T, M, or V; R129 replaced with H, or K; S130 replaced with A, G, I, L, T, M, or V; Q131 replaced with N; S132 replaced with A, G, I, L, T, M, or V; L134 replaced with A, G, I, S, T, M, or V; G136 replaced with A, I, L, S, T, M, or V; S137 replaced with A, G, I, L, T, M, or V; D138 replaced with E; G139 replaced with A, I, L, S, T, M, or V; H140 replaced with K, or R; T141 replaced with A, G, I, L, S, M, or V; Y142 replaced with F, or W; S143 replaced with A, G, I, L, T, M, or V; Q144 replaced with N; I145 replaced with A, G, L, S, T, M, or V; R147 replaced with H, or K; L148 replaced with A, G, I, S, T, M, or V; Q149 replaced with N; E150 replaced with D; A151 replaced with G, I, L, S, T, M, or V; A152 replaced with G, I, L, S, T, M, or V; R153 replaced with H, or K; A154 replaced with G, I, L, S, T, M, or V; R155 replaced with H, or K; D157 replaced with E; A158 replaced with G, I, L, S, T, M, or V; N159 replaced with Q; L160 replaced with A, G, I, S, T, M, or V; T161 replaced with A, G, I, L, S, M, or V; V162 replaced with A, G, I, L, S, T, or M; A163 replaced with G, I, L, S, T, M, or V; H164 replaced with K, or R; G166 replaced with A, I, L, S, T, M, or V; E169 replaced with D; S170 replaced with A, G, I, L, T, M, or V; G171 replaced with A, I, L, S, T, M, or V; Q173 replaced with N; I174 replaced with A, G, L, S, T, M, or V; V175 replaced with A, G, I, L, S, T, or M; S176 replaced with A, G, I, L, T, M, or V; H177 replaced with K, or R; Y179 replaced with F, or W; D180 replaced with E; T181 replaced with A, G, I, L, S, M, or V; W182 replaced with F, or Y; N183 replaced with Q; V184 replaced with A, G, I, L, S, T, or M; T185 replaced with A, G, I, L, S, M, or V; G186 replaced with A, I, L, S, T, M, or V; Q187 replaced with N; D188 replaced with E; V189 replaced with A, G, I, L, S, T, or M; I190 replaced with A, G, L, S, T, M, or V; F191 replaced with W, or Y; G192 replaced with A, I, L, S, T, M, or V; E194 replaced with D; V195 replaced with A, G, I, L, S, T, or M; F196 replaced with W, or Y; A197 replaced with G, I, L, S, T, M, or V; Y198 replaced with F, or W; M200 replaced with A, G, I, L, S, T, or V; A201 replaced with G, I, L, S, T, M, or V; S202 replaced with A, G, I, L, T, M, or V; I203 replaced with A, G, L, S, T, M, or V; E204 replaced with D; W205 replaced with F, or Y; R206 replaced with H, or K; K207 replaced with H, or R; D208 replaced with E; G209 replaced with A, I, L, S, T, M, or V; L210 replaced with A, G, I, S, T, M, or V; D211 replaced with E; I212 replaced with A, G, L, S, T, M, or V; Q213 replaced with N; L214 replaced with A, G, I, S, T, M, or V; G216 replaced with A, I, L, S, T, M, or V; D217 replaced with E; D218 replaced with E; H220 replaced with K, or R; I221 replaced with A, G, L, S, T, M, or V; S222 replaced with A, G, I, L, T, M, or V; V223 replaced with A, G, I, L, S, T, or M; Q224 replaced with N; F225 replaced with W, or Y; R226 replaced with H, or K; G227 replaced with A, I, L, S, T, M, or V; G228 replaced with A, I, L, S, T, M, or V; Q230 replaced with N; R231 replaced with H, or K; F232 replaced with W, or Y; E233 replaced with D; V234 replaced with A, G, I, L, S, T, or M; T235 replaced with A, G, I, L, S, M, or V; G236 replaced with A, I, L, S, T, M, or V; W237 replaced with F, or Y; L238 replaced with A, G, I, S, T, M, or V; Q239 replaced with N; I240 replaced with A, G, L, S, T, M, or V; Q241 replaced with N; A242 replaced with G, I, L, S, T, M, or V; V243 replaced with A, G, I, L, S, T, or M; R244 replaced with H, or K; S246 replaced with A, G, I, L, T, M, or V; D247 replaced with E; E248 replaced with D; G249 replaced with A, I, L, S, T, M, or V; T250 replaced with A, G, I, L, S, M, or V; Y251 replaced with F, or W; R252 replaced with H, or K; L254 replaced with A, G, I, S, T, M, or V; A255 replaced with G, I, L, S, T, M, or V; R256 replaced with H, or K; N257 replaced with Q; A258 replaced with G, I, L, S, T, M, or V; L259 replaced with A, G, I, S, T, M, or V; G260 replaced with A, I, L, S, T, M, or V; Q261 replaced with N; V262 replaced with A, G, I, L, S, T, or M; E263 replaced with D; A264 replaced with G, I, L, S, T, M, or V; A266 replaced with G, I, L, S, T, M, or V; S267 replaced with A, G, I, L, T, M, or V; L268 replaced with A, G, I, S, T, M, or V; T269 replaced with A, G, I, L, S, M, or V; V270 replaced with A, G, I, L, S, T, or M; L271 replaced with A, G, I, S, T, M, or V; T272 replaced with A, G, I, L, S, M, or V; D274 replaced with E; Q275 replaced with N; L276 replaced with A, G, I, S, T, M, or V; N277 replaced with Q; S278 replaced with A, G, I, L, T, M, or V; T279 replaced with A, G, I, L, S, M, or V; G280 replaced with A, I, L, S, T, M, or V; I281 replaced with A, G, L, S, T, M, or V; Q283 replaced with N; L284 replaced with A, G, I, S, T, M, or V; R285 replaced with H, or K; S286 replaced with A, G, I, L, T, M, or V; L287 replaced with A, G, I, S, T, M, or V; N288 replaced with Q; L289 replaced with A, G, I, S, T, M, or V; V290 replaced with A, G, I, L, S, T, or M; E292 replaced with D; E293 replaced with D; E294 replaced with D; A295 replaced with G, I, L, S, T, M, or V; E296 replaced with D; S297 replaced with A, G, I, L, T, M, or V; E298 replaced with D; E299 replaced with D; N300 replaced with Q; D301 replaced with E; D302 replaced with E; Y303 replaced with F, or W; and Y304 replaced with F, or W.

The resulting constructs can be routinely screened for activities or functions described throughout the specification and known in the art. Preferably, the resulting constructs have an increased and/or a decreased PSF-2 activity or function, while the remaining PSF-2 activities or functions are maintained. More preferably, the resulting constructs have more than one increased and/or decreased PSF-2 activity or function, while the remaining PSF-2 activities or functions are maintained.

Besides conservative amino acid substitution, variants of PSF-2 include (i) substitutions with one or more of the non-conserved amino acid residues, where the substituted amino acid residues may or may not be one encoded by the genetic code, or (ii) substitution with one or more of amino acid residues having a substituent group, or (iii) fusion of the mature polypeptide with another compound, such as a compound to increase the stability and/or solubility of the polypeptide (for example, polyethylene glycol), or (iv) fusion of the polypeptide with additional amino acids, such as an IgG Fc fusion region peptide, or leader or secretory sequence, or a sequence facilitating purification. Such variant polypeptides are deemed to be within the scope of those skilled in the art from the teachings herein. In addition, variants containing nucleotide and/or amino acid changes in regions of PSF-2 which do not appear to be conserved are also deemed to be within the scope of those skilled in the art from the teachings herein.

For example, PSF-2 polypeptide variants containing amino acid substitutions of charged amino acids with other charged or neutral amino acids may produce polypeptides with improved characteristics, such as less aggregation. Aggregation of pharmaceutical formulations both reduces activity and increases clearance due to the aggregate's immunogenic activity. (Pinckard, et al., Clin. Exp. Immunol. 2:331-340 (1967); Robbins, et al., Diabetes 36:838-845 (1987); Cleland, et al., Crit. Rev. Therapeutic Drug Carrier Systems 10:307-377 (1993).)

For example, preferred non-conservative substitutions of PSF-2 include: M1 replaced with D, E, H, K, R, N, Q, F, W, Y, P, or C; L2 replaced with D, E, H, K, R, N, Q, F, W, Y, P, or C; P3 replaced with D, E, H, K, R, A, G, I, L, S, T, M, V, N, Q, F, W, Y,or C; P4 replaced with D, E, H, K, R, A, G, I, L, S, T, M, V, N, Q, F, W, Y, or C; P5 replaced with D, E, H, K, R, A, G, I, L, S, T, M, V, N, Q, F, W, Y, or C; R6 replaced with D, E, A, G, I, L, S, T, M, V, N, Q, F, W, Y, P, or C; P7 replaced with D, E, H, K, R, A, G, I, L, S, T, M, V, N, Q, F, W, Y, or C; A8 replaced with D, E, H, K, R, N, Q, F, W, Y, P, or C; A9 replaced with D, E, H, K, R, N, Q, F, W, Y, P, or C; A10 replaced with D, E, H, K, R, N, Q, F, W, Y, P, or C; L11 replaced with D, E, H, K, R, N, Q, F, W, Y, P, or C; A12 replaced with D, E, H, K, R, N, Q, F, W, Y, P, or C; L13 replaced with D, E, H, K, R, N, Q, F, W, Y, P, or C; P14 replaced with D, E, H, K, R, A, G, I, L, S, T, M, V, N, Q, F, W, Y, or C; V15 replaced with D, E, H, K, R, N, Q, F, W, Y, P, or C; L16 replaced with D, E, H, K, R, N, Q, F, W, Y, P, or C; L17 replaced with D, E, H, K, R, N, Q, F, W, Y, P, or C; L18 replaced with D, E, H, K, R, N, Q, F, W, Y, P, or C; L19 replaced with D, E, H, K, R, N, Q, F, W, Y, P, or C; L20 replaced with D, E, H, K, R, N, Q, F, W, Y, P, or C; V21 replaced with D, E, H, K, R, N, Q, F, W, Y, P, or C; V22 replaced with D, E, H, K, R, N, Q, F, W, Y, P, or C; L23 replaced with D, E, H, K, R, N, Q, F, W, Y, P, or C; T24 replaced with D, E, H, K, R, N, Q, F, W, Y, P, or C; P25 replaced with D, E, H, K, R, A, G, I, L, S, T, M, V, N, Q, F, W, Y, or C; P26 replaced with D, E, H, K, R, A, G, I, L, S, T, M, V, N, Q, F, W, Y, or C; P27 replaced with D, E, H, K, R, A, G, I, L, S, T, M, V, N, Q, F, W, Y, or C; T28 replaced with D, E, H, K, R, N, Q, F, W, Y, P, or C; G29 replaced with D, E, H, K, R, N, Q, F, W, Y, P, or C; A30 replaced with D, E, H, K, R, N, Q, F, W, Y, P, or C; R31 replaced with D, E, A, G, I, L, S, T, M, V, N, Q, F, W, Y, P, or C; P32 replaced with D, E, H, K, R, A, G, I, L, S, T, M, V, N, Q, F, W, Y, or C; S33 replaced with D, E, H, K, R, N, Q, F, W, Y, P, or C; P34 replaced with D, E, H, K, R, A, G, I, L, S, T, M, V, N, Q, F, W, Y, or C; G35 replaced with D, E, H, K, R, N, Q, F, W, Y, P, or C; P36 replaced with D, E, H, K, R, A, G, I, L, S, T, M, V, N, Q, F, W, Y, or C; D37 replaced with H, K, R, A, G, I, L, S, T, M, V, N, Q, F, W, Y, P, or C; Y38 replaced with D, E, H, K, R, N, Q, A, G, I, L, S, T, M, V, P, or C; L39 replaced with D, E, H, K, R, N, Q, F, W, Y, P, or C; R40 replaced with D, E, A, G, I, L, S, T, M, V, N, Q, F, W, Y, P, or C; R41 replaced with D, E, A, G, I, L, S, T, M, V, N, Q, F, W, Y, P, or C; G42 replaced with D, E, H, K, R, N, Q, F, W, Y, P, or C; W43 replaced with D, E, H, K, R, N, Q, A, G, I, L, S, T, M, V, P, or C; M44 replaced with D, E, H, K, R, N, Q, F, W, Y, P, or C; R45 replaced with D, E, A, G, I, L, S, T, M, V, N, Q, F, W, Y, P, or C; L46 replaced with D, E, H, K, R, N, Q, F, W, Y, P, or C; L47 replaced with D, E, H, K, R, N, Q, F, W, Y, P, or C; A48 replaced with D, E, H, K, R, N, Q, F, W, Y, P, or C; E49 replaced with H, K, R, A, G, I, L, S, T, M, V, N, Q, F, W, Y, P, or C; G50 replaced with D, E, H, K, R, N, Q, F, W, Y, P, or C; E51 replaced with H, K, R, A, G, I, L, S, T, M, V, N, Q, F, W, Y, P, or C; G52 replaced with D, E, H, K, R, N, Q, F, W, Y, P, or C; C53 replaced with D, E, H, K, R, A, G, I, L, S, T, M, V, N, Q, F, W, Y, or P; A54 replaced with D, E, H, K, R, N, Q, F, W, Y, P, or C; P55 replaced with D, E, H, K, R, A, G, I, L, S, T, M, V, N, Q, F, W, Y, or C; C56 replaced with D, E, H, K, R, A, G, I, L, S, T, M, V, N, Q, F, W, Y, or P; R57 replaced with D, E, A, G, I, L, S, T, M, V, N, Q, F, W, Y, P, or C; P58 replaced with D, E, H, K, R, A, G, I, L, S, T, M, V, N, Q, F, W, Y, or C; E59 replaced with H, K, R, A, G, I, L, S, T, M, V, N, Q, F, W, Y, P, or C; E60 replaced with H, K, R, A, G, I, L, S, T, M, V, N, Q, F, W, Y, P, or C; C61 replaced with D, E, H, K, R, A, G, I, L, S, T, M, V, N, Q, F, W, Y, or P; A62 replaced with D, E, H, K, R, N, Q, F, W, Y, P, or C; A63 replaced with D, E, H, K, R, N, Q, F, W, Y, P, or C; P64 replaced with D, E, H, K, R, A, G, I, L, S, T, M, V, N, Q, F, W, Y, or C; R65 replaced with D, E, A, G, I, L, S, T, M, V, N, Q, F, W, Y, P, or C; G66 replaced with D, E, H, K, R, N, Q, F, W, Y, P, or C; C67 replaced with D, E, H, K, R, A, G, I, L, S, T, M, V, N, Q, F, W, Y, or P; L68 replaced with D, E, H, K, R, N, Q, F, W, Y, P, or C; A69 replaced with D, E, H, K, R, N, Q, F, W, Y, P, or C; G70 replaced with D, E, H, K, R, N, Q, F, W, Y, P, or C; R71 replaced with D, E, A, G, I, L, S, T, M, V, N, Q, F, W, Y, P, or C; V72 replaced with D, E, H, K, R, N, Q, F, W, Y, P, or C; R73 replaced with D, E, A, G, I, L, S, T, M, V, N, Q, F, W, Y, P, or C; D74 replaced with H, K, R, A, G, I, L, S, T, M, V, N, Q, F, W, Y, P, or C; A75 replaced with D, E, H, K, R, N, Q, F, W, Y, P, or C; C76 replaced with D, E, H, K, R, A, G, I, L, S, T, M, V, N, Q, F, W, Y, or P; G77 replaced with D, E, H, K, R, N, Q, F, W, Y, P, or C; C78 replaced with D, E, H, K, R, A, G, I, L, S, T, M, V, N, Q, F, W, Y, or P; C79 replaced with D, E, H, K, R, A, G, I, L, S, T, M, V, N, Q, F, W, Y, or P; W80 replaced with D, E, H, K, R, N, Q, A, G, I, L, S, T, M, V, P, or C; E81 replaced with H, K, R, A, G, I, L, S, T, M, V, N, Q, F, W, Y, P, or C; C82 replaced with D, E, H, K, R, A, G, I, L, S, T, M, V, N, Q, F, W, Y, or P; A83 replaced with D, E, H, K, R, N, Q, F, W, Y, P, or C; N84 replaced with D, E, H, K, R, A, G, I, L, S, T, M, V, F, W, Y, P, or C; L85 replaced with D, E, H, K, R, N, Q, F, W, Y, P, or C; E86 replaced with H, K, R, A, G, I, L, S, T, M, V, N, Q, F, W, Y, P, or C; G87 replaced with D, E, H, K, R, N, Q, F, W, Y, P, or C; Q88 replaced with D, E, H, K, R, A, G, I, L, S, T, M, V, F, W, Y, P, or C; L89 replaced with D, E, H, K, R, N, Q, F, W, Y, P, or C; C90 replaced with D, E, H, K, R, A, G, I, L, S, T, M, V, N, Q, F, W, Y, or P; D91 replaced with H, K, R, A, G, I, L, S, T, M, V, N, Q, F, W, Y, P, or C; L92 replaced with D, E, H, K, R, N, Q, F, W, Y, P, or C; D93 replaced with H, K, R, A, G, I, L, S, T, M, V, N, Q, F, W, Y, P, or C; P94 replaced with D, E, H, K, R, A, G, I, L, S, T, M, V, N, Q, F, W, Y, or C; S95 replaced with D, E, H, K, R, N, Q, F, W, Y, P, or C; A96 replaced with D, E, H, K, R, N, Q, F, W, Y, P, or C; H97 replaced with D, E, A, G, I, L, S, T, M, V, N, Q, F, W, Y, P, or C; F98 replaced with D, E, H, K, R, N, Q, A, G, I, L, S, T, M, V, P, or C; Y99 replaced with D, E, H, K, R, N, Q, A, G, I, L, S, T, M, V, P, or C; G100 replaced with D, E, H, K, R, N, Q, F, W, Y, P, or C; H101 replaced with D, E, A, G, I, L, S, T, M, V, N, Q, F, W, Y, P, or C; C102 replaced with D, E, H, K, R, A, G, I, L, S, T, M, V, N, Q, F, W, Y, or P; G103 replaced with D, E, H, K, R, N, Q, F, W, Y, P, or C; E104 replaced with H, K, R, A, G, I, L, S, T, M, V, N, Q, F, W, Y, P, or C; Q105 replaced with D, E, H, K, R, A, G, I, L, S, T, M, V, F, W, Y, P, or C; L106 replaced with D, E, H, K, R, N, Q, F, W, Y, P, or C; E107 replaced with H, K, R, A, G, I, L, S, T, M, V, N, Q, F, W, Y, P, or C; C108 replaced with D, E, H, K, R, A, G, I, L, S, T, M, V, N, Q, F, W, Y, or P; R109 replaced with D, E, A, G, I, L, S, T, M, V, N, Q, F, W, Y, P, or C; L110 replaced with D, E, H, K, R, N, Q, F, W, Y, P, or C; D111 replaced with H, K, R, A, G, I, L, S, T, M, V, N, Q, F, W, Y, P, or C; T112 replaced with D, E, H, K, R, N, Q, F, W, Y, P, or C; G113 replaced with D, E, H, K, R, N, Q, F, W, Y, P, or C; G114 replaced with D, E, H, K, R, N, Q, F, W, Y, P, or C; D115 replaced with H, K, R, A, G, I, L, S, T, M, V, N, Q, F, W, Y, P, or C; L116 replaced with D, E, H, K, R, N, Q, F, W, Y, P, or C; S117 replaced with D, E, H, K, R, N, Q, F, W, Y, P, or C; R118 replaced with D, E, A, G, I, L, S, T, M, V, N, Q, F, W, Y, P, or C; G119 replaced with D, E, H, K, R, N, Q, F, W, Y, P, or C; E120 replaced with H, K, R, A, G, I, L, S, T, M, V, N, Q, F, W, Y, P, or C; V121 replaced with D, E, H, K, R, N, Q, F, W, Y, P, or C; P122 replaced with D, E, H, K, R, A, G, I, L, S, T, M, V, N, Q, F, W, Y, or C; E123 replaced with H, K, R, A, G, I, L, S, T, M, V, N, Q, F, W, Y, P, or C; P124 replaced with D, E, H, K, R, A, G, I, L, S, T, M, V, N, Q, F, W, Y, or C; L125 replaced with D, E, H, K, R, N, Q, F, W, Y, P, or C; C126 replaced with D, E, H, K, R, A, G, I, L, S, T, M, V, N, Q, F, W, Y, or P; A127 replaced with D, E, H, K, R, N, Q, F, W, Y, P, or C; C128 replaced with D, E, H, K, R, A, G, I, L, S, T, M, V, N, Q, F, W, Y, or P; R129 replaced with D, E, A, G, I, L, S, T, M, V, N, Q, F, W, Y, P, or C; S130 replaced with D, E, H, K, R, N, Q, F, W, Y, P, or C; Q131 replaced with D, E, H, K, R, A, G, I, L, S, T, M, V, F, W, Y, P, or C; S132 replaced with D, E, H, K, R, N, Q, F, W, Y, P, or C; P133 replaced with D, E, H, K, R, A, G, I, L, S, T, M, V, N, Q, F, W, Y, or C; L134 replaced with D, E, H, K, R, N, Q, F, W, Y, P, or C; C135 replaced with D, E, H, K, R, A, G, I, L, S, T, M, V, N, Q, F, W, Y, or P; G136 replaced with D, E, H, K, R, N, Q, F, W, Y, P, or C; S137 replaced with D, E, H, K, R, N, Q, F, W, Y, P, or C; D138 replaced with H, K, R, A, G, I, L, S, T, M, V, N, Q, F, W, Y, P, or C; G139 replaced with D, E, H, K, R, N, Q, F, W, Y, P, or C; H140 replaced with D, E, A, G, I, L, S, T, M, V, N, Q, F, W, Y, P, or C; T141 replaced with D, E, H, K, R, N, Q, F, W, Y, P, or C; Y142 replaced with D, E, H, K, R, N, Q, A, G, I, L, S, T, M, V, P, or C; S143 replaced with D, E, H, K, R, N, Q, F, W, Y, P, or C; Q144 replaced with D, E, H, K, R, A, G, I, L, S, T, M, V, F, W, Y, P, or C; I145 replaced with D, E, H, K, R, N, Q, F, W, Y, P, or C; C146 replaced with D, E, H, K, R, A, G, I, L, S, T, M, V, N, Q, F, W, Y, or P; R147 replaced with D, E, A, G, I, L, S, T, M, V, N, Q, F, W, Y, P, or C; L148 replaced with D, E, H, K, R, N, Q, F, W, Y, P, or C; Q149 replaced with D, E, H, K, R, A, G, I, L, S, T, M, V, F, W, Y, P, or C; E150 replaced with H, K, R, A, G, I, L, S, T, M, V, N, Q, F, W, Y, P, or C; A151 replaced with D, E, H, K, R, N, Q, F, W, Y, P, or C; A152 replaced with D, E, H, K, R, N, Q, F, W, Y, P, or C; R153 replaced with D, E, A, G, I, L, S, T, M, V, N, Q, F, W, Y, P, or C; A154 replaced with D, E, H, K, R, N, Q, F, W, Y, P, or C; R155 replaced with D, E, A, G, I, L, S, T, M, V, N, Q, F, W, Y, P, or C; P156 replaced with D, E, H, K, R, A, G, I, L, S, T, M, V, N, Q, F, W, Y, or C; D157 replaced with H, K, R, A, G, I, L, S, T, M, V, N, Q, F, W, Y, P, or C; A158 replaced with D, E, H, K, R, N, Q, F, W, Y, P, or C; N159 replaced with D, E, H, K, R, A, G, I, L, S, T, M, V, F, W, Y, P, or C; L160 replaced with D, E, H, K, R, N, Q, F, W, Y, P, or C; T161 replaced with D, E, H, K, R, N, Q, F, W, Y, P, or C; V162 replaced with D, E, H, K, R, N, Q, F, W, Y, P, or C; A163 replaced with D, E, H, K, R, N, Q, F, W, Y, P, or C; H164 replaced with D, E, A, G, I, L, S, T, M, V, N, Q, F, W, Y, P, or C; P165 replaced with D, E, H, K, R, A, G, I, L, S, T, M, V, N, Q, F, W, Y, or C; G166 replaced with D, E, H, K, R, N, Q, F, W, Y, P, or C; P167 replaced with D, E, H, K, R, A, G, I, L, S, T, M, V, N, Q, F, W, Y, or C; C168 replaced with D, E, H, K, R, A, G, I, L, S, T, M, V, N, Q, F, W, Y, or P; E169 replaced with H, K, R, A, G, I, L, S, T, M, V, N, Q, F, W, Y, P, or C; S170 replaced with D, E, H, K, R, N, Q, F, W, Y, P, or C; G171 replaced with D, E, H, K, R, N, Q, F, W, Y, P, or C; P172 replaced with D, E, H, K, R, A, G, I, L, S, T, M, V, N, Q, F, W, Y, or C; Q173 replaced with D, E, H, K, R, A, G, I, L, S, T, M, V, F, W, Y, P, or C; I174 replaced with D, E, H, K, R, N, Q, F, W, Y, P, or C; V175 replaced with D, E, H, K, R, N, Q, F, W, Y, P, or C; S176 replaced with D, E, H, K, R, N, Q, F, W, Y, P, or C; H177 replaced with D, E, A, G, I, L, S, T, M, V, N, Q, F, W, Y, P, or C; P178 replaced with D, E, H, K, R, A, G, I, L, S, T, M, V, N, Q, F, W, Y, or C; Y179 replaced with D, E, H, K, R, N, Q, A, G, I, L, S, T, M, V, P, or C; D180 replaced with H, K, R, A, G, I, L, S, T, M, V, N, Q, F, W, Y, P, or C; T181 replaced with D, E, H, K, R, N, Q, F, W, Y, P, or C; W182 replaced with D, E, H, K, R, N, Q, A, G, I, L, S, T, M, V, P, or C; N183 replaced with D, E, H, K, R, A, G, I, L, S, T, M, V, F, W, Y, P, or C; V184 replaced with D, E, H, K, R, N, Q, F, W, Y, P, or C; T185 replaced with D, E, H, K, R, N, Q, F, W, Y, P, or C; G186 replaced with D, E, H, K, R, N, Q, F, W, Y, P, or C; Q187 replaced with D, E, H, K, R, A, G, I, L, S, T, M, V, F, W, Y, P, or C; D188 replaced with H, K, R, A, G, I, L, S, T, M, V, N, Q, F, W, Y, P, or C; V189 replaced with D, E, H, K, R, N, Q, F, W, Y, P, or C; I190 replaced with D, E, H, K, R, N, Q, F, W, Y, P, or C; F191 replaced with D, E, H, K, R, N, Q, A, G, I, L, S, T, M, V, P, or C; G192 replaced with D, E, H, K, R, N, Q, F, W, Y, P, or C; C193 replaced with D, E, H, K, R, A, G, I, L, S, T, M, V, N, Q, F, W, Y, or P; E194 replaced with H, K, R, A, G, I, L, S, T, M, V, N, Q, F, W, Y, P, or C; V195 replaced with D, E, H, K, R, N, Q, F, W, Y, P, or C; F196 replaced with D, E, H, K, R, N, Q, A, G, I, L, S, T, M, V, P, or C; A197 replaced with D, E, H, K, R, N, Q, F, W, Y, P, or C; Y198 replaced with D, E, H, K, R, N, Q, A, G, I, L, S, T, M, V, P, or C; P199 replaced with D, E, H, K, R, A, G, I, L, S, T, M, V, N, Q, F, W, Y, or C; M200 replaced with D, E, H, K, R, N, Q, F, W, Y, P, or C; A201 replaced with D, E, H, K, R, N, Q, F, W, Y, P, or C; S202 replaced with D, E, H, K, R, N, Q, F, W, Y, P, or C; I203 replaced with D, E, H, K, R, N, Q, F, W, Y, P, or C; E204 replaced with H, K, R, A, G, I, L, S, T, M, V, N, Q, F, W, Y, P, or C; W205 replaced with D, E, H, K, R, N, Q, A, G, I, L, S, T, M, V, P, or C; R206 replaced with D, E, A, G, I, L, S, T, M, V, N, Q, F, W, Y, P, or C; K207 replaced with D, E, A, G, I, L, S, T, M, V, N, Q, F, W, Y, P, or C; D208 replaced with H, K, R, A, G, I, L, S, T, M, V, N, Q, F, W, Y, P, or C; G209 replaced with D, E, H, K, R, N, Q, F, W, Y, P, or C; L210 replaced with D, E, H, K, R, N, Q, F, W, Y, P, or C; D211 replaced with H, K, R, A, G, I, L, S, T, M, V, N, Q, F, W, Y, P, or C; I212 replaced with D, E, H, K, R, N, Q, F, W, Y, P, or C; Q213 replaced with D, E, H, K, R, A, G, I, L, S, T, M, V, F, W, Y, P, or C; L214 replaced with D, E, H, K, R, N, Q, F, W, Y, P, or C; P215 replaced with D, E, H, K, R, A, G, I, L, S, T, M, V, N, Q, F, W, Y, or C; G216 replaced with D, E, H, K, R, N, Q, F, W, Y, P, or C; D217 replaced with H, K, R, A, G, I, L, S, T, M, V, N, Q, F, W, Y, P, or C; D218 replaced with H, K, R, A, G, I, L, S, T, M, V, N, Q, F, W, Y, P, or C; P219 replaced with D, E, H, K, R, A, G, I, L, S, T, M, V, N, Q, F, W, Y, or C; H220 replaced with D, E, A, G, I, L, S, T, M, V, N, Q, F, W, Y, P, or C; I221 replaced with D, E, H, K, R, N, Q, F, W, Y, P, or C; S222 replaced with D, E, H, K, R, N, Q, F, W, Y, P, or C; V223 replaced with D, E, H, K, R, N, Q, F, W, Y, P, or C; Q224 replaced with D, E, H, K, R, A, G, I, L, S, T, M, V, F, W, Y, P, or C; F225 replaced with D, E, H, K, R, N, Q, A, G, I, L, S, T, M, V, P, or C; R226 replaced with D, E, A, G, I, L, S, T, M, V, N, Q, F, W, Y, P, or C; G227 replaced with D, E, H, K, R, N, Q, F, W, Y, P, or C; G228 replaced with D, E, H, K, R, N, Q, F, W, Y, P, or C; P229 replaced with D, E, H, K, R, A, G, I, L, S, T, M, V, N, Q, F, W, Y, or C; Q230 replaced with D, E, H, K, R, A, G, I, L, S, T, M, V, F, W, Y, P, or C; R231 replaced with D, E, A, G, I, L, S, T, M, V, N, Q, F, W, Y, P, or C; F232 replaced with D, E, H, K, R, N, Q, A, G, I, L, S, T, M, V, P, or C; E233 replaced with H, K, R, A, G, I, L, S, T, M, V, N, Q, F, W, Y, P, or C; V234 replaced with D, E, H, K, R, N, Q, F, W, Y, P, or C; T235 replaced with D, E, H, K, R, N, Q, F, W, Y, P, or C; G236 replaced with D, E, H, K, R, N, Q, F, W, Y, P, or C; W237 replaced with D, E, H, K, R, N, Q, A, G, I, L, S, T, M, V, P, or C; L238 replaced with D, E, H, K, R, N, Q, F, W, Y, P, or C; Q239 replaced with D, E, H, K, R, A, G, I, L, S, T, M, V, F, W, Y, P, or C; I240 replaced with D, E, H, K, R, N, Q, F, W, Y, P, or C; Q241 replaced with D, E, H, K, R, A, G, I, L, S, T, M, V, F, W, Y, P, or C; A242 replaced with D, E, H, K, R, N, Q, F, W, Y, P, or C; V243 replaced with D, E, H, K, R, N, Q, F, W, Y, P, or C; R244 replaced with D, E, A, G, I, L, S, T, M, V, N, Q, F, W, Y, P, or C; P245 replaced with D, E, H, K, R, A, G, I, L, S, T, M, V, N, Q, F, W, Y, or C; S246 replaced with D, E, H, K, R, N, Q, F, W, Y, P, or C; D247 replaced with H, K, R, A, G, I, L, S, T, M, V, N, Q, F, W, Y, P, or C; E248 replaced with H, K, R, A, G, I, L, S, T, M, V, N, Q, F, W, Y, P, or C; G249 replaced with D, E, H, K, R, N, Q, F, W, Y, P, or C; T250 replaced with D, E, H, K, R, N, Q, F, W, Y, P, or C; Y251 replaced with D, E, H, K, R, N, Q, A, G, I, L, S, T, M, V, P, or C; R252 replaced with D, E, A, G, I, L, S, T, M, V, N, Q, F, W, Y, P, or C; C253 replaced with D, E, H, K, R, A, G, I, L, S, T, M, V, N, Q, F, W, Y, or P; L254 replaced with D, E, H, K, R, N, Q, F, W, Y, P, or C; A255 replaced with D, E, H, K, R, N, Q, F, W, Y, P, or C; R256 replaced with D, E, A, G, I, L, S, T, M, V, N, Q, F, W, Y, P, or C; N257 replaced with D, E, H, K, R, A, G, I, L, S, T, M, V, F, W, Y, P, or C; A258 replaced with D, E, H, K, R, N, Q, F, W, Y, P, or C; L259 replaced with D, E, H, K, R, N, Q, F, W, Y, P, or C; G260 replaced with D, E, H, K, R, N, Q, F, W, Y, P, or C; Q261 replaced with D, E, H, K, R, A, G, I, L, S, T, M, V, F, W, Y, P, or C; V262 replaced with D, E, H, K, R, N, Q, F, W, Y, P, or C; E263 replaced with H, K, R, A, G, I, L, S, T, M, V, N, Q, F, W, Y, P, or C; A264 replaced with D, E, H, K, R, N, Q, F, W, Y, P, or C; P265 replaced with D, E, H, K, R, A, G, I, L, S, T, M, V, N, Q, F, W, Y, or C; A266 replaced with D, E, H, K, R, N, Q, F, W, Y, P, or C; S267 replaced with D, E, H, K, R, N, Q, F, W, Y, P, or C; L268 replaced with D, E, H, K, R, N, Q, F, W, Y, P, or C; T269 replaced with D, E, H, K, R, N, Q, F, W, Y, P, or C; V270 replaced with D, E, H, K, R, N, Q, F, W, Y, P, or C; L271 replaced with D, E, H, K, R, N, Q, F, W, Y, P, or C; T272 replaced with D, E, H, K, R, N, Q, F, W, Y, P, or C; P273 replaced with D, E, H, K, R, A, G, I, L, S, T, M, V, N, Q, F, W, Y, or C; D274 replaced with H, K, R, A, G, I, L, S, T, M, V, N, Q, F, W, Y, P, or C; Q275 replaced with D, E, H, K, R, A, G, I, L, S, T, M, V, F, W, Y, P, or C; L276 replaced with D, E, H, K, R, N, Q, F, W, Y, P, or C; N277 replaced with D, E, H, K, R, A, G, I, L, S, T, M, V, F, W, Y, P, or C; S278 replaced with D, E, H, K, R, N, Q, F, W, Y, P, or C; T279 replaced with D, E, H, K, R, N, Q, F, W, Y, P, or C; G280 replaced with D, E, H, K, R, N, Q, F, W, Y, P, or C; I281 replaced with D, E, H, K, R, N, Q, F, W, Y, P, or C; P282 replaced with D, E, H, K, R, A, G, I, L, S, T, M, V, N, Q, F, W, Y, or C; Q283 replaced with D, E, H, K, R, A, G, I, L, S, T, M, V, F, W, Y, P, or C; L284 replaced with D, E, H, K, R, N, Q, F, W, Y, P, or C; R285 replaced with D, E, A, G, I, L, S, T, M, V, N, Q, F, W, Y, P, or C; S286 replaced with D, E, H, K, R, N, Q, F, W, Y, P, or C; L287 replaced with D, E, H, K, R, N, Q, F, W, Y, P, or C; N288 replaced with D, E, H, K, R, A, G, I, L, S, T, M, V, F, W, Y, P, or C; L289 replaced with D, E, H, K, R, N, Q, F, W, Y, P, or C; V290 replaced with D, E, H, K, R, N, Q, F, W, Y, P, or C; P291 replaced with D, E, H, K, R, A, G, I, L, S, T, M, V, N, Q, F, W, Y, or C; E292 replaced with H, K, R, A, G, I, L, S, T, M, V, N, Q, F, W, Y, P, or C; E293 replaced with H, K, R, A, G, I, L, S, T, M, V, N, Q, F, W, Y, P, or C; E294 replaced with H, K, R, A, G, I, L, S, T, M, V, N, Q, F, W, Y, P, or C; A295 replaced with D, E, H, K, R, N, Q, F, W, Y, P, or C; E296 replaced with H, K, R, A, G, I, L, S, T, M, V, N, Q, F, W, Y, P, or C; S297 replaced with D, E, H, K, R, N, Q, F, W, Y, P, or C; E298 replaced with H, K, R, A, G, I, L, S, T, M, V, N, Q, F, W, Y, P, or C; E299 replaced with H, K, R, A, G, I, L, S, T, M, V, N, Q, F, W, Y, P, or C; N300 replaced with D, E, H, K, R, A, G, I, L, S, T, M, V, F, W, Y, P, or C; D301 replaced with H, K, R, A, G, I, L, S, T, M, V, N, Q, F, W, Y, P, or C; D302 replaced with H, K, R, A, G, I, L, S, T, M, V, N, Q, F, W, Y, P, or C; Y303 replaced with D, E, H, K, R, N, Q, A, G, I, L, S, T, M, V, P, or C; and Y304 replaced with D, E, H, K, R, N, Q, A, G, I, L, S, T, M, V, P, or C.

The resulting constructs can be routinely screened for activities or functions described throughout the specification and known in the art. Preferably, the resulting constructs have an increased and/or decreased PSF-2 activity or function, while the remaining PSF-2 activities or functions are maintained. More preferably, the resulting constructs have more than one increased and/or decreased PSF-2 activity or function, while the remaining PSF-2 activities or functions are maintained.

Additionally, more than one amino acid (e.g., 2, 3, 4, 5, 6, 7, 8, 9 and 10) can be replaced with the substituted amino acids as described above (either conservative or nonconservative). The substituted amino acids can occur in the full length, mature, or proprotein form of PSF-2 protein, as well as the N- and C-terminal deletion mutants, having the general formula m-n, listed below.

A further embodiment of the invention relates to a polypeptide which comprises the amino acid sequence of a PSF-2 polypeptide having an amino acid sequence which contains at least one amino acid substitution, but not more than 50 amino acid substitutions, even more preferably, not more than 40 amino acid substitutions, still more preferably, not more than 30 amino acid substitutions, and still even more preferably, not more than 20 amino acid substitutions. Of course, in order of ever-increasing preference, it is highly preferable for a polypeptide to have an amino acid sequence which comprises the amino acid sequence of a PSF-2 polypeptide, which contains at least one, but not more than 10, 9, 8, 7, 6, 5, 4, 3, 2 or 1 amino acid substitutions. In specific embodiments, the number of additions, substitutions, and/or deletions in the amino acid sequence of FIGS. 1A and 1B or fragments thereof (e.g., the mature form and/or other fragments described herein), is 1-5, 5-10, 5-25, 5-50, 10-50 or 50-150, conservative amino acid substitutions are preferable.

Polynucleotide and Polypeptide Fragments

In the present invention, a “polynucleotide fragment” refers to a short polynucleotide having a nucleic acid sequence contained in the deposited clone or shown in SEQ ID NO:1. The short nucleotide fragments are preferably at least about 15 nt, and more preferably at least about 20 nt, still more preferably at least about 30 nt, and even more preferably, at least about 40 nt in length. A fragment “at least 20 nt in length,” for example, is intended to include 20 or more contiguous bases from the cDNA sequence contained in the deposited clone or the nucleotide sequence shown in SEQ ID NO:1. These nucleotide fragments are useful as diagnostic probes and primers as discussed herein. Of course, larger fragments (e.g., 50, 150, 500, 600, 1800 nucleotides) are preferred. Nucleotide fragments of the PSF-2 can exclude the sequences of the following related cDNA clones, and any subfragments therein: HOABR24R (SEQ ID NO:18); HETEGL74R (SEQ ID NO:19); HETEZ03R (SEQ ID NO:20); HETGM93R (SEQ ID NO:21); AI075710 (SEQ ID NO:22); and R30743 (SEQ ID NO:23).

Moreover, representative examples of PSF-2 polynucleotide fragments include, for example, fragments having a sequence from about nucleotide number 1-50, 51-100, 101-150, 151-200, 201-250, 251-300, 301-350, 351-400, 401-450, 451-500, 501-550, 551-600, 651-700, 701-750, 751-800, 800-850, 851-900, 901-950, 951-1000, 1001-1050, 1051-1100, 1101-1150, 1151-1200, 1201-1250, 1251-1300, 1301-1350, 1351-1400, 1401-1450, 1451-1500, 1501-1550, 1551-1600, 1601-1650, 1651-1700, 1701-1750, 1751-1800 or 1801 to the end of SEQ ID NO:1 or the cDNA contained in the deposited clone. In this context “about” includes the particularly recited ranges, larger or smaller by several (5, 4, 3, 2, or 1) nucleotides, at either terminus or at both termini. Preferably, these fragments encode a polypeptide which has biological activity. More preferably, these polynucleotides can be used as probes or primers as discussed herein.

In the present invention, a “polypeptide fragment” refers to a short amino acid sequence contained in SEQ ID NO:2 or encoded by the cDNA contained in the deposited clone. Polypeptide fragments may be “free-standing,” or comprised within a larger polypeptide of which the fragment forms a part or region, most preferably as a single continuous region. Representative examples of polypeptide fragments of the invention, include, for example, fragments from about amino acid number 1-20, 21-40, 41-60, 61-80, 81-100, 102-120, 121-140, 141-160, 161-180, 181-200, 201-220, 221-240, 241-260, 261-280, 281-300 or 281 to the end of the coding region. Moreover, polypeptide fragments can be about 20, 30, 40, 50, 60, 70, 80, 90, 100, 110, 120, 130, 140, or 150 amino acids in length. In this context “about” includes the particularly recited ranges, larger or smaller by several (5, 4, 3, 2, or 1) amino acids, at either extreme or at both extremes.

Preferred polypeptide fragments include the secreted PSF-2 polypeptide as well as the mature form. Further preferred polypeptide fragments include the secreted PSF-2 polypeptide or the mature form having a continuous series of deleted residues from the amino or the carboxy terminus, or both. For example, any number of amino acids, ranging from 1-60, can be deleted from the amino terminus of either the secreted PSF-2 polypeptide or the mature form. Similarly, any number of amino acids, ranging from 1-30, can be deleted from the carboxy terminus of the secreted PSF-2 polypeptide or mature form. Furthermore, any combination of the above amino and carboxy terminus deletions are preferred. Similarly, polynucleotide fragments encoding these PSF-2 polypeptide fragments are also preferred.

Particularly, N-terminal deletions of the PSF-2 polypeptide can be described by the general formula m¹-304, where m¹ is an integer from 2 to 299, where m¹ corresponds to the position of the amino acid residue identified in SEQ ID NO:2. More in particular, the invention provides polynucleotides encoding polypeptides comprising, or alternatively consisting of, the amino acid sequence of residues of L-2 to Y-304; P-3 to Y-304; P-4 to Y-304; P-5 to Y-304; R-6 to Y-304; P-7 to Y-304; A-8 to Y-304; A-9 to Y-304; A-10 to Y-304; L-11 to Y-304; A-12 to Y-304; L-13 to Y-304; P-14 to Y-304; V-15 to Y-304; L-16 to Y-304; L-17 to Y-304; L-18 to Y-304; L-19 to Y-304; L-20 to Y-304; V-21 to Y-304; V-22 to Y-304; L-23 to Y-304; T-24 to Y-304; P-25 to Y-304; P-26 to Y-304; P-27 to Y-304; T-28 to Y-304; G-29 to Y-304; A-30 to Y-304; R-31 to Y-304; P-32 to Y-304; S-33 to Y-304; P-34 to Y-304; G-35 to Y-304; P-36 to Y-304; D-37 to Y-304; Y-38 to Y-304; L-39 to Y-304; R-40 to Y-304; R-41 to Y-304; G-42 to Y-304; W-43 to Y-304; M-44 to Y-304; R-45 to Y-304; L-46 to Y-304; L-47 to Y-304; A-48 to Y-304; E-49 to Y-304; G-50 to Y-304; E-51 to Y-304; G-52 to Y-304; C-53 to Y-304; A-54 to Y-304; P-55 to Y-304; C-56 to Y-304; R-57 to Y-304; P-58 to Y-304; E-59 to Y-304; E-60 to Y-304; C-61 to Y-304; A-62 to Y-304; A-63 to Y-304; P-64 to Y-304; R-65 to Y-304; G-66 to Y-304; C-67 to Y-304; L-68 to Y-304; A-69 to Y-304; G-70 to Y-304; R-71 to Y-304; V-72 to Y-304; R-73 to Y-304; D-74 to Y-304; A-75 to Y-304; C-76 to Y-304; G-77 to Y-304; C-78 to Y-304; C-79 to Y-304; W-80 to Y-304; E-81 to Y-304; C-82 to Y-304; A-83 to Y-304; N-84 to Y-304; L-85 to Y-304; E-86 to Y-304; G-87 to Y-304; Q-88 to Y-304; L-89 to Y-304; C-90 to Y-304; D-91 to Y-304; L-92 to Y-304; D-93 to Y-304; P-94 to Y-304; S-95 to Y-304; A-96 to Y-304; H-97 to Y-304; F-98 to Y-304; Y-99 to Y-304; G-100 to Y-304; H-101 to Y-304; C-102 to Y-304; G-103 to Y-304; E-104 to Y-304; Q-105 to Y-304; L-106 to Y-304; E-107 to Y-304; C-108 to Y-304; R-109 to Y-304; L-110 to Y-304; D-111 to Y-304; T-112 to Y-304; G-113 to Y-304; G-114 to Y-304; D-115 to Y-304; L-116 to Y-304; S-117 to Y-304; R-118 to Y-304; G-119 to Y-304; E-120 to Y-304; V-121 to Y-304; P-122 to Y-304; E-123 to Y-304; P-124 to Y-304; L-125 to Y-304; C-126 to Y-304; A-127 to Y-304; C-128 to Y-304; R-129 to Y-304; S-130 to Y-304; Q-131 to Y-304; S-132 to Y-304; P-133 to Y-304; L-134 to Y-304; C-135 to Y-304; G-136 to Y-304; S-137 to Y-304; D-138 to Y-304; G-139 to Y-304; H-140 to Y-304; T-141 to Y-304; Y-142 to Y-304; S-143 to Y-304; Q-144 to Y-304; I-145 to Y-304; C-146 to Y-304; R-147 to Y-304; L-148 to Y-304; Q-149 to Y-304; E-150 to Y-304; A-151 to Y-304; A-152 to Y-304; R-153 to Y-304; A-154 to Y-304; R-155 to Y-304; P-156 to Y-304; D-157 to Y-304; A-158 to Y-304; N-159 to Y-304; L-160 to Y-304; T-161 to Y-304; V-162 to Y-304; A-163 to Y-304; H-164 to Y-304; P-165 to Y-304; G-166 to Y-304; P-167 to Y-304; C-168 to Y-304; E-169 to Y-304; S-170 to Y-304; G-171 to Y-304; P-172 to Y-304; Q-173 to Y-304; I-174 to Y-304; V-175 to Y-304; S-176 to Y-304; H-177 to Y-304; P-178 to Y-304; Y-179 to Y-304; D-180 to Y-304; T-181 to Y-304; W-182 to Y-304; N-183 to Y-304; V-184 to Y-304; T-185 to Y-304; G-186 to Y-304; Q-187 to Y-304; D-188 to Y-304; V-189 to Y-304; I-190 to Y-304; F-191 to Y-304; G-192 to Y-304; C-193 to Y-304; E-194 to Y-304; V-195 to Y-304; F-196 to Y-304; A-197 to Y-304; Y-198 to Y-304; P-199 to Y-304; M-200 to Y-304; A-201 to Y-304; S-202 to Y-304; I-203 to Y-304; E-204 to Y-304; W-205 to Y-304; R-206 to Y-304; K-207 to Y-304; D-208 to Y-304; G-209 to Y-304; L-210 to Y-304; D-211 to Y-304; I-212 to Y-304; Q-213 to Y-304; L-214 to Y-304; P-215 to Y-304; G-216 to Y-304; D-217 to Y-304; D-218 to Y-304; P-219 to Y-304; H-220 to Y-304; I-221 to Y-304; S-222 to Y-304; V-223 to Y-304; Q-224 to Y-304; F-225 to Y-304; R-226 to Y-304; G-227 to Y-304; G-228 to Y-304; P-229 to Y-304; Q-230 to Y-304; R-231 to Y-304; F-232 to Y-304; E-233 to Y-304; V-234 to Y-304; T-235 to Y-304; G-236 to Y-304; W-237 to Y-304; L-238 to Y-304; Q-239 to Y-304; I-240 to Y-304; Q-241 to Y-304; A-242 to Y-304; V-243 to Y-304; R-244 to Y-304; P-245 to Y-304; S-246 to Y-304; D-247 to Y-304; E-248 to Y-304; G-249 to Y-304; T-250 to Y-304; Y-251 to Y-304; R-252 to Y-304; C-253 to Y-304; L-254 to Y-304; A-255 to Y-304; R-256 to Y-304; N-257 to Y-304; A-258 to Y-304; L-259 to Y-304; G-260 to Y-304; Q-261 to Y-304; V-262 to Y-304; E-263 to Y-304; A-264 to Y-304; P-265 to Y-304; A-266 to Y-304; S-267 to Y-304; L-268 to Y-304; T-269 to Y-304; V-270 to Y-304; L-271 to Y-304; T-272 to Y-304; P-273 to Y-304; D-274 to Y-304; Q-275 to Y-304; L-276 to Y-304; N-277 to Y-304; S-278 to Y-304; T-279 to Y-304; G-280 to Y-304; I-281 to Y-304; P-282 to Y-304; Q-283 to Y-304; L-284 to Y-304; R-285 to Y-304; S-286 to Y-304; L-287 to Y-304; N-288 to Y-304; L-289 to Y-304; V-290 to Y-304; P-291 to Y-304; E-292 to Y-304; E-293 to Y-304; E-294 to Y-304; A-295 to Y-304; E-296 to Y-304; S-297 to Y-304; E-298 to Y-304; and E-299 to Y-304 of the PSF-2 sequence shown in SEQ ID NO:2 and in FIGS. 1A and 1B. Polynucleotides encoding these polypeptides are also encompassed by the invention. The present application is also directed to nucleic acid molecules comprising, or alternatively, consisting of, a polynucleotide sequence at least 90%, 92%, 95%, 96%, 97%, 98% or 99% identical to the polynucleotide sequences encoding the PSF-2 polypeptides described above. The present invention also encompasses the above polynucleotide sequences fused to a heterologous polynucleotide sequence.

Moreover, C-terminal deletions of the PSF-2 polypeptide can also be described by the general formula 1-n¹, where n¹ is an integer from 6 to 303, where n¹ corresponds to the position of amino acid residue identified in SEQ ID NO:2. More in particular, the invention provides polynucleotides encoding polypeptides comprising, or alternatively consisting of, the amino acid sequence of residues M-1 to Y-303; M-1 to D-302; M-1 to D-301; M-1 to N-300; M-1 to E-299; M-1 to E-298; M-1 to S-297; M-1 to E-296; M-1 to A-295; M-1 to E-294; M-1 to E-293; M-1 to E-292; M-1 to P-291; M-1 to V-290; M-1 to L-289; M-1 to N-288; M-1 to L-287; M-1 to S-286; M-1 to R-285; M-1 to L-284; M-1 to Q-283; M-1 to P-282; M-1 to I-281; M-1 to G-280; M-1 to T-279; M-1 to S-278; M-1 to N-277; M-1 to L-276; M-1 to Q-275; M-1 to D-274; M-1 to P-273; M-1 to T-272; M-1 to L-271; M-1 to V-270; M-1 to T-269; M-1 to L-268; M-1 to S-267; M-1 to A-266; M-1 to P-265; M-1 to A-264; M-1 to E-263; M-1 to V-262; M-1 to Q-261; M-1 to G-260; M-1 to L-259; M-1 to A-258; M-1 to N-257; M-1 to R-256; M-1 to A-255; M-1 to L-254; M-1 to C-253; M-1 to R-252; M-1 to Y-251; M-1 to T-250; M-1 to G-249; M-1 to E-248; M-1 to D-247; M-1 to S-246; M-1 to P-245; M-1 to R-244; M-1 to V-243; M-1 to A-242; M-1 to Q-241; M-1 to I-240; M-1 to Q-239; M-1 to L-238; M-1 to W-237; M-1 to G-236; M-1 to T-235; M-1 to V-234; M-1 to E-233; M-1 to F-232; M-1 to R-231; M-1 to Q-230; M-1 to P-229; M-1 to G-228; M-1 to G-227; M-1 to R-226; M-1 to F-225; M-1 to Q-224; M-1 to V-223; M-1 to S-222; M-1 to I-221; M-1 to H-220; M-1 to P-219; M-1 to D-218; M-1 to D-217; M-1 to G-216; M-1 to P-215; M-1 to L-214; M-1 to Q-213; M-1 to I-212; M-1 to D-211; M-1 to L-210; M-1 to G-209; M-1 to D-208; M-1 to K-207; M-1 to R-206; M-1 to W-205; M-1 to E-204; M-1 to I-203; M-1 to S-202; M-1 to A-201; M-1 to M-200; M-1 to P-199; M-1 to Y-198; M-1 to A-197; M-1 to F-196; M-1 to V-195; M-1 to E-194; M-1 to C-193; M-1 to G-192; M-1 to F-191; M-1 to I-190; M-1 to V-189; M-1 to D-188; M-1 to Q-187; M-1 to G-186; M-1 to T-185; M-1 to V-184; M-1 to N-183; M-1 to W-182; M-1 to T-181; M-1 to D-180; M-1 to Y-179; M-1 to P-178; M-1 to H-177; M-1 to S-176; M-1 to V-175; M-1 to I-174; M-1 to Q-173; M-1 to P-172; M-1 to G-171; M-1 to S-170; M-1 to E-169; M-1 to C-168; M-1 to P-167; M-1 to G-166; M-1 to P-165; M-1 to H-164; M-1 to A-163; M-1 to V-162; M-1 to T-161; M-1 to L-160; M-1 to N-159; M-1 to A-158; M-1 to D-157; M-1 to P-156; M-1 to R-155; M-1 to A-154; M-1 to R-153; M-1 to A-152; M-1 to A-151; M-1 to E-150; M-1 to Q-149; M-1 to L-148; M-1 to R-147; M-1 to C-146; M-1 to I-145; M-1 to Q-144; M-1 to S-143; M-1 to Y-142; M-1 to T-141; M-1 to H-140; M-1 to G-139; M-1 to D-138; M-1 to S-137; M-1 to G-136; M-1 to C-135; M-1 to L-134; M-1 to P-133; M-1 to S-132; M-1 to Q-131; M-1 to S-130; M-1 to R-129; M-1 to C-128; M-1 to A-127; M-1 to C-126; M-1 to L-125; M-1 to P-124; M-1 to E-123; M-1 to P-122; M-1 to V-121; M-1 to E-120; M-1 to G-119; M-1 to R-118; M-1 to S-117; M-1 to L-116; M-1 to D-115; M-1 to G-114; M-1 to G-113; M-1 to T-112; M-1 to D-111; M-1 to L-110; M-1 to R-109; M-1 to C-108; M-1 to E-107; M-1 to L-106; M-1 to Q-105; M-1 to E-104; M-1 to G-103; M-1 to C-102; M-1 to H-101; M-1 to G-100; M-1 to Y-99; M-1 to F-98; M-1 to H-97; M-1 to A-96; M-1 to S-95; M-1 to P-94; M-1 to D-93; M-1 to L-92; M-1 to D-91; M-1 to C-90; M-1 to L-89; M-1 to Q-88; M-1 to G-87; M-1 to E-86; M-1 to L-85; M-1 to N-84; M-1 to A-83; M-1 to C-82; M-1 to E-81; M-1 to W-80; M-1 to C-79; M-1 to C-78; M-1 to G-77; M-1 to C-76; M-1 to A-75; M-1 to D-74; M-1 to R-73; M-1 to V-72; M-1 to R-71; M-1 to G-70; M-1 to A-69; M-1 to L-68; M-1 to C-67; M-1 to G-66; M-1 to R-65; M-1 to P-64; M-1 to A-63; M-1 to A-62; M-1 to C-61; M-1 to E-60; M-1 to E-59; M-1 to P-58; M-1 to R-57; M-1 to C-56; M-1 to P-55; M-1 to A-54; M-1 to C-53; M-1 to G-52; M-1 to E-51; M-1 to G-50; M-1 to E-49; M-1 to A-48; M-1 to L-47; M-1 to L-46; M-1 to R-45; M-1 to M-44; M-1 to W-43; M-1 to G-42; M-1 to R-41; M-1 to R-40; M-1 to L-39; M-1 to Y-38; M-1 to D-37; M-1 to P-36; M-1 to G-35; M-1 to P-34; M-1 to S-33; M-1 to P-32; M-1 to R-31; M-1 to A-30; M-1 to G-29; M-1 to T-28; M-1 to P-27; M-1 to P-26; M-1 to P-25; M-1 to T-24; M-1 to L-23; M-1 to V-22; M-1 to V-21; M-1 to L-20; M-1 to L-19; M-1 to L-18; M-1 to L-17; M-1 to L-16; M-1 to V-15; M-1 to P-14; M-1 to L-13; M-1 to A-12; M-1 to L-1; M-1 to A-10; M-1 to A-9; M-1 to A-8; M-1 to P-7; and M-1 to R-6 of the sequence of the PSF-2 sequence shown in SEQ ID NO:2 and in FIGS. 1A 1B. Polynucleotides encoding these polypeptides are also encompassed by the invention. The present application is also directed to nucleic acid molecules comprising, or alternatively, consisting of, a polynucleotide sequence at least 90%, 92%, 95%, 96%, 97%, 98% or 99% identical to the polynucleotide sequences encoding the PSF-2 polypeptides described above. The present invention also encompasses the above polynucleotide sequences fused to a heterologous polynucleotide sequence.

In certain embodiments, any of the above listed N- or C-terminal deletions can be combined to produce a N- and C-terminally deleted PSF-2 polypeptide.

The invention also provides polypeptides having one or more amino acids deleted from both the amino and the carboxyl termini, which may be described generally as having residues m¹-n¹ of SEQ ID NO:2, where n¹ and m¹ are integers as described above.

Also preferred are PSF-2 polypeptide and polynucleotide fragments characterized by structural or functional domains. Preferred embodiments of the invention include fragments that comprise alpha-helix and alpha-helix forming regions (“alpha-regions”), beta-sheet and beta-sheet-forming regions (“beta-regions”), turn and turn-forming regions (“turn-regions”), coil and coil-forming regions (“coil-regions”), hydrophilic regions, hydrophobic regions, alpha amphipathic regions, beta amphipathic regions, flexible regions, surface-forming regions, substrate binding region, and high antigenic index regions. As set out in the Figures, such preferred regions include Garnier-Robson alpha-regions, beta-regions, turn-regions, and coil-regions, Chou-Fasman alpha-regions, beta-regions, and turn-regions, Kyte-Doolittle hydrophilic regions and hydrophobic regions, Eisenberg alpha and beta amphipathic regions, Karplus-Schulz flexible regions, Emini surface-forming regions, and Jameson-Wolf high antigenic index regions. Polypeptide fragments of SEQ ID NO:2 falling within conserved domains are specifically contemplated by the present invention. (See FIG. 3 and Table I.) Moreover, polynucleotide fragments encoding these domains are also contemplated.

TABLE I

| Res | Position | I | II | III | IV | V | VI | VII | VIII | IX | X | XI | XII | XIII | XIV |
|---|---|---|---|---|---|---|---|---|---|---|---|---|---|---|---|
| Met | 1 | . | . | B | . | . | . | . | −0.10 | 0.44 | . | . | . | −0.40 | 0.73 |
| Leu | 2 | . | . | B | . | . | . | . | 0.40 | 0.44 | . | . | . | −0.16 | 0.88 |
| Pro | 3 | . | . | . | . | . | . | C | 0.58 | 0.01 | * | . | . | 0.73 | 1.35 |
| Pro | 4 | . | . | . | . | . | T | C | 0.38 | 0.01 | * | . | . | 1.17 | 2.12 |
| Pro | 5 | . | . | . | . | . | T | C | 0.18 | −0.10 | . | . | F | 2.16 | 2.59 |
| Arg | 6 | . | . | . | . | . | T | C | 0.19 | −0.29 | * | . | F | 2.40 | 1.69 |
| Pro | 7 | . | . | B | . | . | T | . | 0.19 | −0.21 | * | . | F | 1.96 | 1.11 |
| Ala | 8 | . | A | B | . | . | . | . | −0.19 | 0.04 | . | . | . | 0.42 | 0.59 |
| Ala | 9 | . | A | B | . | . | . | . | −0.79 | 0.11 | * | . | . | 0.18 | 0.30 |
| Ala | 10 | . | A | B | . | . | . | . | −0.79 | 0.80 | * | * | . | −0.36 | 0.16 |
| Leu | 11 | . | A | B | . | . | . | . | −1.76 | 0.80 | * | . | . | −0.60 | 0.25 |
| Ala | 12 | . | . | B | B | . | . | . | −2.36 | 0.94 | . | . | . | −0.60 | 0.18 |
| Leu | 13 | . | . | B | B | . | . | . | −2.58 | 1.13 | . | . | . | −0.60 | 0.15 |
| Pro | 14 | . | . | B | B | . | . | . | −2.80 | 1.31 | . | . | . | −0.60 | 0.15 |
| Val | 15 | . | . | B | B | . | . | . | −3.02 | 1.31 | . | . | . | −0.60 | 0.12 |
| Leu | 16 | . | . | B | B | . | . | . | −3.02 | 1.50 | . | . | . | −0.60 | 0.12 |
| Leu | 17 | . | . | B | B | . | . | . | −3.29 | 1.50 | . | . | . | −0.60 | 0.06 |
| Leu | 18 | . | . | B | B | . | . | . | −3.33 | 1.71 | . | . | . | −0.60 | 0.06 |
| Leu | 19 | . | . | B | B | . | . | . | −3.93 | 1.71 | . | . | . | −0.60 | 0.06 |
| Leu | 20 | . | . | B | B | . | . | . | −3.39 | 1.71 | . | . | . | −0.60 | 0.06 |
| Val | 21 | . | . | B | B | . | . | . | −2.79 | 1.51 | . | . | . | −0.60 | 0.10 |
| Val | 22 | . | . | B | B | . | . | . | −2.19 | 1.26 | . | . | . | −0.60 | 0.19 |
| Leu | 23 | . | . | B | B | . | . | . | −1.59 | 1.00 | . | . | . | −0.60 | 0.36 |
| Thr | 24 | . | . | B | B | . | . | . | −1.09 | 0.74 | . | . | F | −0.45 | 0.75 |
| Pro | 25 | . | . | B | B | . | . | . | −0.62 | 0.59 | . | . | F | −0.30 | 1.46 |
| Pro | 26 | . | . | . | . | . | T | C | −0.36 | 0.37 | * | . | F | 0.60 | 1.75 |
| Pro | 27 | . | . | . | . | T | T | . | 0.61 | 0.19 | * | . | F | 0.80 | 1.22 |
| Thr | 28 | . | . | . | . | T | T | . | 1.21 | −0.30 | * | . | F | 1.40 | 1.55 |
| Gly | 29 | . | . | . | . | T | T | . | 1.22 | −0.30 | * | . | F | 1.40 | 1.55 |
| Ala | 30 | . | . | B | . | . | . | . | 1.22 | −0.34 | * | . | F | 1.10 | 1.34 |
| Arg | 31 | . | . | B | . | . | T | . | 1.09 | −0.34 | * | . | F | 1.60 | 1.44 |
| Pro | 32 | . | . | B | . | . | T | . | 1.09 | −0.40 | . | . | F | 1.90 | 1.44 |
| Ser | 33 | . | . | . | . | . | T | C | 1.40 | −0.40 | . | . | F | 2.40 | 2.20 |
| Pro | 34 | . | . | . | . | . | T | C | 1.50 | −0.90 | . | . | F | 3.00 | 1.88 |
| Gly | 35 | . | . | . | . | . | T | C | 1.28 | −0.14 | . | * | F | 2.40 | 1.90 |
| Pro | 36 | . | . | B | . | . | T | . | 1.28 | 0.11 | * | . | F | 1.30 | 1.17 |
| Asp | 37 | . | . | B | . | . | T | . | 1.60 | −0.27 | * | . | F | 1.60 | 1.48 |
| Tyr | 38 | . | . | B | . | . | T | . | 1.56 | −0.70 | * | . | F | 1.85 | 2.93 |
| Leu | 39 | . | . | B | . | . | . | . | 1.48 | −0.70 | * | . | F | 1.60 | 1.88 |
| Arg | 40 | . | . | B | . | . | T | . | 1.22 | −0.21 | * | . | F | 1.75 | 1.18 |
| Arg | 41 | . | . | B | . | . | T | . | 1.54 | 0.40 | * | * | . | 0.80 | 0.75 |
| Gly | 42 | . | . | . | . | T | T | . | 0.73 | −0.36 | * | . | . | 2.50 | 1.77 |
| Trp | 43 | . | . | B | . | . | T | . | 0.17 | −0.36 | * | . | . | 1.70 | 0.75 |
| Met | 44 | . | A | B | . | . | . | . | 0.39 | 0.33 | * | * | . | 0.45 | 0.31 |
| Arg | 45 | . | A | B | . | . | . | . | 0.28 | 0.83 | * | * | . | −0.10 | 0.32 |
| Leu | 46 | . | A | B | . | . | . | . | −0.18 | 0.40 | * | * | . | −0.35 | 0.53 |
| Leu | 47 | . | A | B | . | . | . | . | 0.17 | −0.09 | * | * | . | 0.61 | 0.53 |
| Ala | 48 | . | A | . | . | . | . | C | 0.11 | −0.70 | * | * | . | 1.42 | 0.47 |
| Glu | 49 | . | A | . | . | T | . | . | 0.04 | −0.27 | * | * | F | 1.78 | 0.56 |
| Gly | 50 | . | . | . | . | T | T | . | −0.66 | −0.39 | * | * | F | 2.49 | 0.36 |
| Glu | 51 | . | . | . | . | T | T | . | −0.06 | −0.57 | . | . | F | 3.10 | 0.36 |
| Gly | 52 | . | . | . | . | T | T | . | 0.09 | −0.64 | . | * | F | 2.79 | 0.33 |
| Cys | 53 | . | . | . | . | T | T | . | 0.79 | −0.07 | . | * | . | 2.29 | 0.18 |
| Ala | 54 | . | . | . | . | . | T | C | 0.58 | −0.50 | . | * | . | 2.04 | 0.20 |
| Pro | 55 | . | . | . | . | T | T | . | 0.92 | −0.07 | . | * | . | 2.19 | 0.31 |
| Cys | 56 | . | . | . | . | T | T | . | 0.92 | −0.50 | . | * | . | 2.29 | 1.01 |
| Arg | 57 | . | . | B | . | . | T | . | 0.60 | −1.07 | * | * | F | 2.60 | 1.73 |
| Pro | 58 | . | A | . | . | T | . | . | 0.68 | −1.00 | * | . | F | 2.19 | 0.60 |
| Glu | 59 | . | A | . | . | T | . | . | 0.68 | −0.93 | * | . | F | 2.28 | 1.13 |
| Glu | 60 | . | A | . | . | T | . | . | 0.68 | −1.00 | * | * | . | 1.92 | 0.58 |
| Cys | 61 | . | A | . | . | T | . | . | 1.46 | −0.57 | * | * | . | 1.86 | 0.58 |
| Ala | 62 | . | A | B | . | T | . | . | 1.00 | −1.00 | * | * | . | 1.80 | 0.66 |
| Ala | 63 | . | . | B | . | . | T | . | 0.54 | −0.57 | . | . | . | 2.00 | 0.38 |
| Pro | 64 | . | . | . | . | T | T | . | −0.27 | 0.00 | . | . | . | 1.30 | 0.38 |
| Arg | 65 | . | . | . | . | T | T | . | −0.86 | 0.11 | . | . | . | 1.10 | 0.31 |
| Gly | 66 | . | . | . | . | T | T | . | −0.53 | 0.11 | * | * | . | 0.90 | 0.31 |
| Cys | 67 | . | . | B | . | . | . | . | 0.17 | 0.04 | . | * | . | 0.10 | 0.20 |
| Leu | 68 | . | . | B | . | . | . | . | −0.10 | −0.39 | . | * | . | 0.50 | 0.20 |
| Ala | 69 | . | . | B | . | . | . | . | 0.22 | 0.26 | . | * | . | −0.10 | 0.15 |
| Gly | 70 | . | . | B | . | . | . | . | 0.11 | −0.17 | . | * | . | 0.78 | 0.54 |
| Arg | 71 | . | . | B | . | . | . | . | −0.13 | −0.74 | * | * | F | 1.66 | 1.09 |
| Val | 72 | . | . | B | . | . | . | . | −0.13 | −0.93 | * | * | F | 1.94 | 1.09 |
| Arg | 73 | . | . | B | . | . | . | . | 0.33 | −0.86 | * | * | F | 2.07 | 0.59 |
| Asp | 74 | . | . | . | . | T | T | . | 0.26 | −0.86 | * | * | . | 2.80 | 0.30 |
| Ala | 75 | . | . | . | . | T | T | . | −0.07 | −0.29 | * | * | . | 2.22 | 0.22 |
| Cys | 76 | . | . | . | . | T | T | . | −0.47 | −0.36 | * | * | . | 1.94 | 0.06 |
| Gly | 77 | . | . | . | . | T | T | . | 0.39 | 0.56 | * | . | . | 0.76 | 0.04 |
| Cys | 78 | . | A | . | . | T | . | . | −0.39 | 0.56 | * | . | . | 0.08 | 0.06 |
| Cys | 79 | . | A | . | . | T | . | . | −0.98 | 0.63 | * | . | . | −0.20 | 0.06 |
| Trp | 80 | . | A | B | . | . | . | . | −0.39 | 0.56 | . | . | . | −0.60 | 0.06 |
| Glu | 81 | . | A | B | . | . | . | . | −0.53 | 0.53 | . | . | . | −0.60 | 0.19 |
| Cys | 82 | . | A | . | . | T | . | . | −0.19 | 0.64 | . | . | . | −0.20 | 0.30 |
| Ala | 83 | . | A | . | . | T | . | . | 0.13 | 0.07 | . | . | . | 0.10 | 0.49 |
| Asn | 84 | . | A | . | . | T | . | . | 0.80 | −0.41 | . | * | . | 0.70 | 0.28 |
| Leu | 85 | . | A | . | . | T | . | . | 0.28 | −0.01 | . | * | . | 0.70 | 0.91 |
| Glu | 86 | . | A | . | . | T | . | . | −0.39 | 0.10 | * | * | F | 0.25 | 0.74 |
| Gly | 87 | . | A | . | . | T | . | . | 0.28 | 0.17 | * | * | F | 0.25 | 0.25 |
| Gln | 88 | . | A | B | . | . | . | . | 0.06 | −0.23 | . | * | F | 0.45 | 0.50 |
| Leu | 89 | . | A | B | . | . | . | . | 0.06 | −0.23 | . | * | . | 0.30 | 0.24 |
| Cys | 90 | . | A | B | . | . | . | . | 0.66 | −0.23 | . | . | . | 0.55 | 0.40 |
| Asp | 91 | . | A | . | . | T | . | . | 0.36 | −0.23 | . | . | . | 1.20 | 0.36 |
| Leu | 92 | . | A | B | . | . | . | . | 0.11 | −0.24 | . | . | . | 1.05 | 0.59 |
| Asp | 93 | . | . | . | . | . | T | C | 0.08 | −0.43 | . | * | F | 2.20 | 1.10 |
| Pro | 94 | . | . | . | . | T | T | . | 0.19 | −0.50 | . | * | F | 2.50 | 0.90 |
| Ser | 95 | . | . | . | . | T | T | . | 0.61 | 0.29 | . | * | F | 1.65 | 0.94 |
| Ala | 96 | . | . | B | . | . | T | . | 0.27 | 0.36 | . | * | . | 0.85 | 0.89 |
| His | 97 | . | . | B | . | . | T | . | 1.04 | 0.79 | . | * | . | 0.30 | 0.57 |
| Phe | 98 | . | . | B | . | . | T | . | 0.38 | 0.86 | . | * | . | 0.05 | 0.58 |
| Tyr | 99 | . | . | . | . | T | T | . | 0.24 | 1.04 | . | . | . | 0.20 | 0.31 |
| Gly | 100 | . | . | . | . | T | T | . | 0.54 | 0.97 | . | . | . | 0.20 | 0.22 |
| His | 101 | . | . | . | . | T | . | . | 1.13 | 0.47 | * | . | . | 0.00 | 0.44 |
| Cys | 102 | . | . | . | . | T | T | . | 0.36 | 0.09 | * | . | . | 0.50 | 0.49 |
| Gly | 103 | . | . | . | . | T | T | . | 1.06 | 0.01 | * | . | . | 0.50 | 0.41 |
| Glu | 104 | . | . | . | . | T | T | . | 0.63 | −0.41 | * | * | F | 1.25 | 0.52 |
| Gln | 105 | . | . | B | . | . | T | . | 1.09 | −0.34 | * | * | F | 0.85 | 0.52 |
| Leu | 106 | . | A | B | . | . | . | . | 0.31 | −0.91 | * | * | . | 0.75 | 1.03 |
| Glu | 107 | . | A | B | . | . | . | . | 0.98 | −0.66 | * | * | . | 0.60 | 0.49 |
| Cys | 108 | . | A | B | . | . | . | . | 1.01 | −0.66 | * | * | . | 0.91 | 0.47 |
| Arg | 109 | . | A | B | . | . | . | . | 0.67 | −0.57 | * | * | . | 1.22 | 0.83 |
| Leu | 110 | . | A | B | . | . | . | . | 0.32 | −0.83 | * | * | . | 1.53 | 0.47 |
| Asp | 111 | . | . | . | . | T | T | . | 1.13 | −0.40 | * | * | F | 2.49 | 0.87 |
| Thr | 112 | . | . | . | . | T | T | . | 0.32 | −0.97 | . | * | F | 3.10 | 0.75 |
| Gly | 113 | . | . | . | . | T | T | . | 0.69 | −0.29 | * | * | F | 2.49 | 0.75 |
| Gly | 114 | . | . | . | . | T | T | . | 0.69 | −0.59 | * | * | F | 2.48 | 0.60 |
| Asp | 115 | . | . | . | . | . | . | C | 1.16 | −0.59 | * | . | F | 1.77 | 0.81 |
| Leu | 116 | . | . | . | . | . | . | C | 1.16 | −0.64 | * | . | F | 1.46 | 0.81 |
| Ser | 117 | . | . | . | . | . | T | C | 0.61 | −1.07 | * | . | F | 1.80 | 1.42 |
| Arg | 118 | . | . | B | . | . | T | . | 0.74 | −0.86 | * | . | F | 1.75 | 0.63 |
| Gly | 119 | . | . | . | . | T | T | . | 1.09 | −0.43 | . | . | F | 2.30 | 1.18 |
| Glu | 120 | . | . | B | . | . | T | . | 0.88 | −1.11 | . | . | F | 2.50 | 1.53 |
| Val | 121 | . | . | . | . | . | T | C | 0.88 | −1.07 | . | . | F | 3.00 | 1.21 |
| Pro | 122 | . | . | . | . | . | T | C | 0.51 | −0.39 | . | * | F | 2.40 | 1.01 |
| Glu | 123 | . | . | B | . | . | T | . | −0.19 | −0.24 | . | * | F | 1.75 | 0.31 |
| Pro | 124 | . | . | B | . | . | T | . | −0.51 | 0.26 | . | . | F | 0.85 | 0.42 |
| Leu | 125 | . | . | . | . | T | . | . | −0.40 | 0.19 | * | . | . | 0.60 | 0.15 |
| Cys | 126 | . | . | B | . | . | . | . | 0.16 | −0.24 | . | . | . | 0.78 | 0.17 |
| Ala | 127 | . | . | B | . | . | . | . | 0.37 | 0.14 | . | . | . | 0.46 | 0.14 |
| Cys | 128 | . | . | . | . | T | T | . | 0.07 | 0.11 | . | . | . | 1.34 | 0.30 |
| Arg | 129 | . | . | . | . | T | T | . | 0.07 | −0.19 | . | . | F | 2.37 | 0.76 |
| Ser | 130 | . | . | . | . | T | T | . | 0.07 | −0.33 | . | . | F | 2.80 | 1.16 |
| Gln | 131 | . | . | B | . | . | T | . | 0.07 | −0.14 | * | * | F | 2.12 | 1.78 |
| Ser | 132 | . | . | B | . | . | T | . | 0.31 | −0.14 | * | * | F | 1.69 | 0.49 |
| Pro | 133 | . | . | . | . | T | T | . | 0.68 | 0.29 | . | * | F | 1.21 | 0.36 |
| Leu | 134 | . | . | . | . | T | T | . | 0.57 | 0.29 | . | * | F | 0.93 | 0.28 |
| Cys | 135 | . | . | . | . | T | T | . | 0.52 | −0.11 | . | . | F | 1.53 | 0.35 |
| Gly | 136 | . | . | . | . | T | T | . | 0.49 | −0.07 | . | . | F | 1.81 | 0.22 |
| Ser | 137 | . | . | . | . | T | T | . | 0.48 | 0.00 | . | . | F | 1.49 | 0.37 |
| Asp | 138 | . | . | . | . | T | T | . | 0.44 | −0.20 | . | . | F | 2.37 | 0.99 |
| Gly | 139 | . | . | . | . | T | T | . | 0.96 | −0.01 | . | . | F | 2.80 | 1.56 |
| His | 140 | . | . | . | . | T | T | . | 1.62 | −0.06 | . | . | F | 2.52 | 1.56 |
| Thr | 141 | . | . | . | . | T | T | . | 1.08 | −0.04 | . | . | . | 2.09 | 1.62 |
| Tyr | 142 | . | . | . | . | T | T | . | 0.71 | 0.64 | * | . | . | 0.91 | 1.15 |
| Ser | 143 | . | . | B | . | . | T | . | 0.82 | 0.79 | * | . | . | 0.08 | 0.45 |
| Gln | 144 | . | A | B | . | . | . | . | 0.36 | 0.29 | * | . | . | −0.30 | 0.61 |
| Ile | 145 | . | A | B | . | . | . | . | 0.39 | 0.49 | * | . | . | −0.60 | 0.32 |
| Cys | 146 | . | A | B | . | . | . | . | 0.70 | 0.13 | * | . | . | −0.30 | 0.42 |
| Arg | 147 | . | A | B | . | . | . | . | 0.36 | −0.26 | * | . | . | 0.30 | 0.42 |
| Leu | 148 | . | A | B | . | . | . | . | 0.07 | −0.16 | * | * | . | 0.30 | 0.60 |
| Gln | 149 | . | A | B | . | . | . | . | 0.18 | −0.34 | * | * | . | 0.45 | 1.13 |
| Glu | 150 | . | A | B | . | . | . | . | 0.48 | −0.91 | * | * | . | 0.75 | 1.13 |
| Ala | 151 | . | A | B | . | . | . | . | 1.26 | −0.41 | . | * | . | 0.45 | 1.39 |
| Ala | 152 | . | A | B | . | . | . | . | 0.93 | −1.10 | * | * | . | 1.05 | 1.57 |
| Arg | 153 | . | A | B | . | . | . | . | 1.74 | −1.07 | * | * | . | 1.35 | 1.40 |
| Ala | 154 | . | A | B | . | . | . | . | 1.16 | −1.07 | * | * | . | 1.65 | 2.32 |
| Arg | 155 | . | . | . | . | . | T | C | 1.16 | −1.07 | . | * | F | 2.70 | 2.32 |
| Pro | 156 | . | . | . | . | . | T | C | 0.93 | −1.17 | . | * | F | 3.00 | 1.90 |
| Asp | 157 | . | . | . | . | T | T | . | 1.21 | −0.49 | . | * | F | 2.60 | 1.55 |
| Ala | 158 | . | . | . | . | . | T | C | 0.24 | −0.50 | . | * | F | 2.10 | 1.14 |
| Asn | 159 | . | . | B | B | . | . | . | 0.24 | 0.14 | . | * | . | 0.30 | 0.55 |
| Leu | 160 | . | . | B | B | . | . | . | 0.10 | 0.21 | . | * | . | 0.00 | 0.33 |
| Thr | 161 | . | . | B | B | . | . | . | 0.10 | 0.71 | . | * | . | −0.60 | 0.45 |
| Val | 162 | . | . | B | B | . | . | . | −0.24 | 0.64 | . | * | . | −0.60 | 0.43 |
| Ala | 163 | . | . | B | B | . | . | . | 0.13 | 0.67 | . | . | . | −0.35 | 0.52 |
| His | 164 | . | . | B | . | . | T | . | −0.53 | 0.41 | . | . | . | 0.30 | 0.55 |
| Pro | 165 | . | . | . | . | . | T | C | 0.28 | 0.50 | . | . | F | 0.90 | 0.40 |
| Gly | 166 | . | . | . | . | . | T | C | 0.29 | −0.14 | . | . | F | 2.05 | 0.69 |
| Pro | 167 | . | . | . | . | T | T | . | 0.80 | −0.26 | . | . | F | 2.50 | 0.67 |
| Cys | 168 | . | . | . | . | T | T | . | 1.18 | −0.33 | . | . | F | 2.25 | 0.43 |
| Glu | 169 | . | . | . | . | T | T | . | 1.21 | −0.33 | . | . | F | 2.00 | 0.67 |
| Ser | 170 | . | . | . | . | T | T | . | 0.53 | −0.36 | * | . | F | 1.75 | 0.76 |
| Gly | 171 | . | . | . | . | . | T | C | 0.02 | −0.10 | * | . | F | 1.30 | 0.99 |
| Pro | 172 | . | . | B | B | . | . | . | −0.07 | −0.03 | * | . | F | 0.45 | 0.42 |
| Gln | 173 | . | . | B | B | . | . | . | 0.57 | 0.36 | * | . | F | −0.15 | 0.42 |
| Ile | 174 | . | . | B | B | . | . | . | 0.36 | 0.47 | * | . | . | −0.60 | 0.58 |
| Val | 175 | . | . | B | B | . | . | . | 0.41 | 0.47 | * | . | . | −0.60 | 0.58 |
| Ser | 176 | . | . | B | . | . | . | . | 0.76 | 0.80 | * | . | . | −0.40 | 0.53 |
| His | 177 | . | . | B | . | . | T | . | 0.66 | 0.40 | * | . | . | 0.25 | 1.26 |
| Pro | 178 | . | . | B | . | . | T | . | 0.37 | 0.20 | * | . | . | 0.25 | 2.44 |
| Tyr | 179 | . | . | . | . | T | T | . | 1.26 | 0.47 | * | . | . | 0.35 | 1.92 |
| Asp | 180 | . | . | . | . | T | T | . | 1.26 | 0.49 | . | . | F | 0.50 | 2.26 |
| Thr | 181 | . | . | . | B | T | . | . | 1.24 | 0.63 | . | . | . | −0.05 | 1.09 |
| Trp | 182 | . | . | B | B | . | . | . | 0.93 | 0.69 | * | . | . | −0.45 | 1.00 |
| Asn | 183 | . | . | B | B | . | . | . | 1.14 | 0.36 | . | . | . | −0.25 | 0.59 |
| Val | 184 | . | . | B | B | . | . | . | 1.39 | 0.76 | * | . | F | −0.35 | 0.71 |
| Thr | 185 | . | . | B | B | . | . | . | 0.53 | 0.27 | * | . | F | 0.15 | 1.13 |
| Gly | 186 | . | . | . | . | T | T | . | −0.04 | −0.00 | . | . | F | 1.45 | 0.52 |
| Gln | 187 | . | . | B | . | . | T | . | −0.46 | 0.29 | . | . | F | 0.50 | 0.49 |
| Asp | 188 | . | . | B | . | . | T | . | −0.80 | 0.43 | . | . | F | 0.15 | 0.30 |
| Val | 189 | . | . | B | . | . | T | . | −0.61 | 0.37 | . | . | . | 0.25 | 0.30 |
| Ile | 190 | . | . | B | B | . | . | . | −0.30 | 0.51 | . | . | . | −0.50 | 0.09 |
| Phe | 191 | . | . | B | B | . | . | . | −0.81 | 0.11 | . | . | . | −0.25 | 0.09 |
| Gly | 192 | . | . | B | B | . | . | . | −1.51 | 0.76 | . | . | . | −0.60 | 0.09 |
| Cys | 193 | . | . | B | B | . | . | . | −2.10 | 0.90 | . | . | . | −0.60 | 0.12 |
| Glu | 194 | . | . | B | B | . | . | . | −1.49 | 0.71 | . | . | . | −0.60 | 0.14 |
| Val | 195 | . | . | B | B | . | . | . | −0.81 | 0.69 | . | . | . | −0.60 | 0.22 |
| Phe | 196 | . | . | B | B | . | . | . | −0.71 | 0.69 | . | . | . | −0.60 | 0.62 |
| Ala | 197 | . | . | B | B | . | . | . | −0.96 | 0.73 | . | . | . | −0.60 | 0.36 |
| Tyr | 198 | . | . | B | B | . | . | . | −0.59 | 1.23 | * | . | . | −0.60 | 0.49 |
| Pro | 199 | . | . | . | B | . | . | C | −1.48 | 0.97 | * | . | . | −0.40 | 0.75 |
| Met | 200 | . | A | . | . | . | . | C | −0.62 | 0.87 | . | . | . | −0.40 | 0.52 |
| Ala | 201 | . | A | B | . | . | . | . | −0.21 | 0.37 | * | * | . | −0.30 | 0.58 |
| Ser | 202 | . | A | B | . | . | . | . | 0.49 | 0.53 | * | * | . | −0.60 | 0.39 |
| Ile | 203 | . | A | B | . | . | . | . | 0.78 | 0.10 | . | * | . | 0.04 | 0.78 |
| Glu | 204 | . | A | B | . | . | . | . | 0.99 | −0.51 | . | * | . | 1.43 | 1.54 |
| Trp | 205 | . | A | B | . | . | . | . | 1.24 | −1.01 | . | * | . | 1.77 | 1.91 |
| Arg | 206 | . | . | . | . | T | T | . | 1.02 | −0.97 | . | * | F | 3.06 | 2.70 |
| Lys | 207 | . | . | . | . | T | T | . | 1.32 | −0.97 | . | * | F | 3.40 | 1.29 |
| Asp | 208 | . | . | . | . | T | T | . | 1.32 | −0.97 | . | * | F | 3.06 | 2.04 |
| Gly | 209 | . | . | . | . | T | T | . | 1.32 | −1.20 | . | * | F | 2.57 | 0.73 |
| Leu | 210 | . | A | . | . | . | . | C | 0.80 | −0.80 | . | * | . | 1.48 | 0.63 |
| Asp | 211 | . | A | B | . | . | . | . | 0.48 | −0.11 | . | * | . | 0.64 | 0.31 |
| Ile | 212 | . | A | B | . | . | . | . | 0.09 | 0.31 | . | * | . | 0.04 | 0.49 |
| Gln | 213 | . | A | B | . | . | . | . | 0.09 | 0.31 | . | * | . | 0.38 | 0.59 |
| Leu | 214 | . | . | B | . | . | T | . | 0.43 | −0.37 | . | * | F | 1.87 | 0.59 |
| Pro | 215 | . | . | . | . | T | T | . | 1.03 | −0.37 | . | * | F | 2.76 | 1.40 |
| Gly | 216 | . | . | . | . | T | T | . | 1.00 | −0.63 | . | * | F | 3.40 | 1.25 |
| Asp | 217 | . | . | . | . | . | T | C | 1.00 | −0.53 | . | * | F | 2.86 | 2.06 |
| Asp | 218 | . | . | . | . | . | T | C | 0.70 | −0.53 | . | * | F | 2.37 | 0.93 |
| Pro | 219 | . | . | . | . | . | T | C | 0.66 | −0.57 | . | * | F | 2.18 | 1.26 |
| His | 220 | . | . | B | . | . | T | . | 0.87 | −0.36 | . | * | . | 1.04 | 0.56 |
| Ile | 221 | . | . | B | . | . | T | . | 0.51 | 0.04 | . | * | . | 0.10 | 0.58 |
| Ser | 222 | . | . | B | B | . | . | . | 0.62 | 0.83 | . | * | . | −0.60 | 0.33 |
| Val | 223 | . | . | B | B | . | . | . | 0.28 | 0.40 | * | * | . | −0.30 | 0.47 |
| Gln | 224 | . | . | B | B | . | . | . | 0.14 | 0.33 | * | * | . | 0.00 | 0.66 |
| Phe | 225 | . | . | B | . | . | T | . | −0.03 | 0.07 | * | * | . | 0.70 | 0.49 |
| Arg | 226 | . | . | . | . | T | T | . | 0.86 | 0.11 | * | * | F | 1.70 | 1.02 |
| Gly | 227 | . | . | . | . | . | T | C | 1.27 | −0.13 | * | * | F | 2.40 | 1.02 |
| Gly | 228 | . | . | . | . | . | T | C | 1.42 | −0.53 | * | * | F | 3.00 | 2.31 |
| Pro | 229 | . | . | . | . | . | . | C | 1.42 | −0.53 | * | * | F | 2.50 | 1.02 |
| Gln | 230 | . | . | . | . | . | . | C | 1.27 | −0.53 | * | * | F | 2.20 | 1.78 |
| Arg | 231 | . | . | B | B | . | . | . | 0.84 | −0.31 | * | * | F | 1.20 | 1.34 |
| Phe | 232 | . | . | B | B | . | . | . | 0.84 | −0.26 | . | * | F | 0.90 | 1.25 |
| Glu | 233 | . | . | B | B | . | . | . | 0.90 | −0.26 | . | * | . | 0.30 | 0.71 |
| Val | 234 | . | . | B | B | . | . | . | 0.30 | 0.26 | . | * | . | −0.30 | 0.38 |
| Thr | 235 | . | . | . | B | T | . | . | 0.30 | 0.94 | * | * | . | −0.20 | 0.36 |
| Gly | 236 | . | . | . | B | T | . | . | −0.70 | 0.56 | * | * | . | −0.20 | 0.36 |
| Trp | 237 | . | . | . | B | T | . | . | 0.00 | 1.24 | . | * | . | −0.20 | 0.34 |
| Leu | 238 | . | . | . | B | . | . | C | −0.59 | 1.00 | . | * | . | −0.40 | 0.41 |
| Gln | 239 | . | . | B | B | . | . | . | −0.59 | 1.01 | . | * | . | −0.60 | 0.42 |
| Ile | 240 | . | . | B | B | . | . | . | −0.17 | 1.23 | . | . | . | −0.60 | 0.30 |
| Gln | 241 | . | . | B | B | . | . | . | −0.03 | 0.31 | . | . | . | −0.30 | 0.71 |
| Ala | 242 | . | . | B | B | . | . | . | −0.04 | 0.06 | . | . | . | 0.04 | 0.63 |
| Val | 243 | . | . | B | B | . | . | . | 0.77 | 0.04 | * | . | . | 0.53 | 1.21 |
| Arg | 244 | . | . | . | . | . | T | C | 0.77 | −0.64 | * | . | F | 2.52 | 1.17 |
| Pro | 245 | . | . | . | . | . | T | C | 1.31 | −1.04 | * | . | F | 2.86 | 2.00 |
| Ser | 246 | . | . | . | . | T | T | . | 1.00 | −1.11 | * | . | F | 3.40 | 2.66 |
| Asp | 247 | . | . | . | . | T | T | . | 1.34 | −1.27 | * | * | F | 3.06 | 1.96 |
| Glu | 248 | . | . | . | . | T | . | . | 2.31 | −0.51 | * | * | F | 2.52 | 1.99 |
| Gly | 249 | . | . | . | . | T | T | . | 1.53 | −0.94 | * | * | F | 2.38 | 2.91 |
| Thr | 250 | . | . | B | . | . | T | . | 0.93 | −0.76 | * | . | F | 1.49 | 0.93 |
| Tyr | 251 | . | . | B | . | . | T | . | 0.64 | −0.07 | * | * | . | 0.70 | 0.44 |
| Arg | 252 | . | . | B | . | . | T | . | 0.76 | 0.43 | * | * | . | −0.20 | 0.45 |
| Cys | 253 | . | A | B | . | . | . | . | 0.76 | −0.00 | * | * | . | 0.30 | 0.62 |
| Leu | 254 | . | A | B | . | . | . | . | 0.51 | −0.09 | * | * | . | 0.30 | 0.63 |
| Ala | 255 | . | A | B | . | . | . | . | 0.01 | −0.34 | * | * | . | 0.30 | 0.33 |
| Arg | 256 | . | A | B | . | . | . | . | −0.09 | 0.34 | * | * | . | −0.30 | 0.50 |
| Asn | 257 | . | A | . | . | T | . | . | −0.20 | 0.20 | * | * | . | 0.10 | 0.60 |
| Ala | 258 | . | A | . | . | . | . | C | −0.39 | −0.09 | * | . | . | 0.65 | 1.03 |
| Leu | 259 | . | A | . | . | . | . | C | 0.42 | 0.06 | * | . | . | −0.10 | 0.39 |
| Gly | 260 | . | A | B | . | . | . | . | 0.42 | 0.06 | * | . | . | −0.30 | 0.42 |
| Gln | 261 | . | A | B | . | . | . | . | 0.10 | 0.16 | * | * | . | −0.30 | 0.42 |
| Val | 262 | . | A | B | . | . | . | . | −0.49 | 0.09 | * | * | . | −0.30 | 0.79 |
| Glu | 263 | . | A | B | . | . | . | . | −0.20 | −0.10 | . | * | . | 0.30 | 0.81 |
| Ala | 264 | . | A | B | . | . | . | . | −0.20 | −0.14 | . | * | . | 0.30 | 0.62 |
| Pro | 265 | . | A | B | . | . | . | . | −0.17 | 0.14 | . | * | . | −0.30 | 0.69 |
| Ala | 266 | . | . | B | B | . | . | . | −1.02 | −0.01 | . | * | . | 0.30 | 0.58 |
| Ser | 267 | . | . | B | B | . | . | . | −0.98 | 0.63 | . | * | . | −0.60 | 0.42 |
| Leu | 268 | . | . | B | B | . | . | . | −1.29 | 0.81 | . | . | . | −0.60 | 0.23 |
| Thr | 269 | . | . | B | B | . | . | . | −0.91 | 0.87 | . | . | . | −0.60 | 0.32 |
| Val | 270 | . | . | B | B | . | . | . | −0.70 | 0.80 | . | . | . | −0.60 | 0.37 |
| Leu | 271 | . | . | B | B | . | . | . | −0.11 | 0.41 | . | . | . | −0.60 | 0.75 |
| Thr | 272 | . | . | B | . | . | T | . | −0.62 | 0.13 | . | . | F | 0.25 | 0.91 |
| Pro | 273 | . | . | B | . | . | T | . | 0.19 | 0.33 | . | . | F | 0.68 | 1.01 |
| Asp | 274 | . | . | . | . | T | T | . | 0.20 | 0.09 | . | . | F | 1.36 | 1.96 |
| Gln | 275 | . | . | B | . | . | T | . | 0.74 | −0.21 | . | . | F | 1.84 | 1.82 |
| Leu | 276 | . | . | B | . | . | . | . | 1.21 | −0.21 | . | . | F | 1.92 | 1.70 |
| Asn | 277 | . | . | . | . | T | T | . | 0.63 | −0.21 | . | . | F | 2.80 | 1.01 |
| Ser | 278 | . | . | . | . | T | T | . | 0.63 | 0.47 | . | . | F | 1.47 | 0.41 |
| Thr | 279 | . | . | . | . | T | T | . | 0.63 | 0.50 | . | . | F | 1.19 | 0.76 |
| Gly | 280 | . | . | B | . | . | T | . | −0.18 | 0.21 | * | * | F | 0.81 | 0.82 |
| Ile | 281 | . | . | B | B | . | . | . | 0.74 | 0.50 | * | * | F | −0.17 | 0.51 |
| Pro | 282 | . | . | B | B | . | . | . | 0.44 | 0.11 | * | . | F | −0.15 | 0.69 |
| Gln | 283 | . | . | B | B | . | . | . | −0.07 | 0.01 | * | . | F | −0.15 | 0.93 |
| Leu | 284 | . | . | B | B | . | . | . | 0.24 | 0.27 | . | . | F | 0.00 | 1.10 |
| Arg | 285 | . | . | B | B | . | . | . | −0.22 | −0.01 | * | . | F | 0.60 | 1.14 |
| Ser | 286 | . | . | B | . | . | . | . | −0.19 | 0.24 | . | . | . | −0.10 | 0.54 |
| Leu | 287 | . | . | B | . | . | . | . | −0.19 | 0.49 | * | . | . | −0.40 | 0.49 |
| Asn | 288 | . | . | . | . | . | . | C | −0.19 | 0.23 | * | . | . | 0.10 | 0.39 |
| Leu | 289 | . | A | . | . | . | . | C | 0.62 | 0.23 | * | . | . | −0.10 | 0.50 |
| Val | 290 | . | A | . | . | . | . | C | 0.51 | −0.16 | * | . | . | 0.65 | 1.05 |
| Pro | 291 | . | A | . | . | . | . | C | 0.22 | −0.84 | . | . | F | 1.10 | 1.13 |
| Glu | 292 | A | A | . | . | . | . | . | 1.03 | −0.74 | . | . | F | 0.90 | 1.38 |
| Glu | 293 | A | A | . | . | . | . | . | 0.73 | −1.43 | . | . | F | 0.90 | 3.22 |
| Glu | 294 | A | A | . | . | . | . | . | 1.54 | −1.69 | . | . | F | 0.90 | 2.79 |
| Ala | 295 | A | A | . | . | . | . | . | 2.40 | −2.11 | . | . | F | 0.90 | 2.79 |
| Glu | 296 | A | A | . | . | . | . | . | 2.61 | −2.11 | . | . | F | 0.90 | 2.79 |
| Ser | 297 | A | A | . | . | . | . | . | 2.61 | −1.71 | . | . | F | 1.24 | 2.59 |
| Glu | 298 | A | A | . | . | . | . | . | 2.61 | −1.71 | . | . | F | 1.58 | 4.29 |
| Glu | 299 | A | A | . | . | . | . | . | 2.37 | −2.21 | . | . | F | 1.92 | 4.13 |
| Asn | 300 | . | . | . | . | T | T | . | 2.71 | −1.46 | . | . | F | 3.06 | 4.83 |
| Asp | 301 | . | . | . | . | T | T | . | 2.32 | −1.09 | . | . | F | 3.40 | 4.37 |
| Asp | 302 | . | . | . | . | T | T | . | 2.23 | −0.66 | . | . | . | 2.91 | 3.23 |
| Tyr | 303 | . | . | . | . | T | T | . | 1.84 | −0.23 | . | . | . | 2.27 | 2.56 |
| Tyr | 304 | . | . | B | . | . | . | . | 1.46 | −0.20 | . | . | . | 1.33 | 1.96 |

Among highly preferred fragments in this regard are those that comprise reigons of PSF-2 that combine several structural features, such as several of the features set out above.

Other preferred fragments are biologically active PSF-2 fragments. Biologically active fragments are those exhibiting activity similar, but not necessarily identical, to an activity of the PSF-2 polypeptide. The biological activity of the fragments may include an improved desired activity, or a decreased undesirable activity.

However, many polynucleotide sequences, such as EST sequences, are publicly available and accessible through sequence databases. Some of these sequences are related to SEQ ID NO:1 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a¹-b¹, where a¹ is any integer between 1 and 1799 of SEQ ID NO:1, b¹ is an integer of 15 to 1813 of SEQ ID NO:1, where both a¹ and b¹ correspond to the positions of nucleotide residues shown in SEQ ID NO:1, and where the b¹ is greater than or equal to a¹+14.

In particular embodiments, polynucleotides comprising a nucleotide sequence disclosed in GenBank ESTs AI075710 (SEQ ID NO:22) and R30743 (SEQ ID NO:23) are preferably excluded from the present invention.

Additional preferred polypeptide fragments comprise, or alternatively consist of, the amino acid sequence of residues: M-1 to A-10; L-2 to L-1 1; P-3 to A-12; P-4 to L-13; P-5 to P-14; R-6 to V-15; P-7 to L-16; A-8 to L-17; A-9 to L-18; A-10 to L-19; L-11 to L-20; A-12 to V-21; L-13 to V-22; P-14 to L-23; V-15 to T-24; L-16 to P-25; L-17 to P-26; L-18 to P-27; L-19 to T-28; L-20 to G-29; V-21 to A-30; V-22 to R-31; L-23 to P-32; T-24 to S-33; P-25 to P-34; P-26 to G-35; P-27 to P-36; T-28 to D-37; G-29 to Y-38; A-30 to L-39; R-31 to R-40; P-32 to R-41; S-33 to G-42; P-34 to W-43; G-35 to M-44; P-36 to R-45; D-37 to L-46; Y-38 to L-47; L-39 to A-48; R-40 to E-49; R-41 to G-50; G-42 to E-51; W-43 to G-52; M-44 to C-53; R-45 to A-54; L-46 to P-55; L-47 to C-56; A-48 to R-57; E-49 to P-58; G-50 to E-59; E-51 to E-60; G-52 to C-61; C-53 to A-62; A-54 to A-63; P-55 to P-64; C-56 to R-65; R-57 to G-66; P-58 to C-67; E-59 to L-68; E-60 to A-69; C-61 to G-70; A-62 to R-71; A-63 to V-72; P-64 to R-73; R-65 to D-74; G-66 to A-75; C-67 to C-76; L-68 to G-77; A-69 to C-78; G-70 to C-79; R-71 to W-80; V-72 to E-81; R-73 to C-82; D-74 to A-83; A-75 to N-84; C-76 to L-85; G-77 to E-86; C-78 to G-87; C-79 to Q-88; W-80 to L-89; E-81 to C-90; C-82 to D-91; A-83 to L-92; N-84 to D-93; L-85 to P-94; E-86 to S-95; G-87 to A-96; Q-88 to H-97; L-89 to F-98; C-90 to Y-99; D-91 to G-100; L-92 to H-101; D-93 to C-102; P-94 to G-103; S-95 to E-104; A-96 to Q-105; H-97 to L-106; F-98 to E-107; Y-99 to C-108; G-100 to R-109; H-101 to L-110; C-102 to D-111; G-103 to T-112; E-104 to G-113; Q-105 to G-114; L-106 to D-115; E-107 to L-116; C-108 to S-117; R-109 to R-118; L-110 to G-119; D-111 to E-120; T-112 to V-121; G-113 to P-122; G-114 to E-123; D-115 to P-124; L-116 to L-125; S-117 to C-126; R-118 to A-127; G-119 to C-128; E-120 to R-129; V-121 to S-130; P-122 to Q-131; E-123 to S-132; P-124 to P-133; L-125 to L-134; C-126 to C-135; A-127 to G-136; C-128 to S-137; R-129 to D-138; S-130 to G-139; Q-131 to H-140; S-132 to T-141; P-133 to Y-142; L-134 to S-143; C-135 to Q-144; G-136 to I-145; S-137 to C-146; D-138 to R-147; G-139; to L-148; H-140 to Q-149; T-141 to E-150; Y-142 to A-151; S-143 to A-152; Q-144 to R-153; I-145 to A-154; C-146 to R-155; R-147 to P-156; L-148 to D-157; Q-149 to A-158; E-150 to N-159; A-151 to L-160; A-152 to T-161; R-153 to V-162; A-154 to A-163; R-155 to H-164; P-156 to P-165; D-157 to G-166; A-158 to P-167; N-159 to C-168; L-160 to E-169; T-161 to S-170; V-162 to G-171; A-163 to P-172; H-164 to Q-173; P-165 to I-174; G-166 to V-175; P-167 to S-176; C-168 to H-177; E-169 to P-178; S-170 to Y-179; G-171 to D-180; P-172 to T-181; Q-173 to W-182; I-174 to N-183; V-175 to V-184; S-176 to T-185; H-177 to G-186; P-178 to Q-187; Y-179 to D-188; D-180 to V-189; T-181 to I-190; W-182 to F-191; N-183 to G-192; V-184 to C-193; T-185 to E-194; G-186 to V-195; Q-187 to F-196; D-188 to A-197; V-189 to Y-198; I-190 to P-199; F-191 to M-200; G-192 to A-201; C-193 to S-202; E-194 to I-203; V-195 to E-204; F-196 to W-205; A-197 to R-206; Y-198 to K-207; P-199 to D-208; M-200 to G-209; A-201 to L-210; S-202 to D-211; I-203 to I-212; E-204 to Q-213; W-205 to L-214; R-206 to P-215; K-207 to G-216; D-208 to D-217; G-209 to D-218; L-210 to P-219; D-211 to H-220; I-212 to I-221; Q-213 to S-222; L-214 to V-223; P-215 to Q-224; G-216 to F-225; D-217 to R-226; D-218 to G-227; P-219 to G-228; H-220 to P-229; I-221 to Q-230; S-222 to R-231; V-223 to F-232; Q-224 to E-233; F-225 to V-234; R-226 to T-235; G-227 to G-236; G-228 to W-237; P-229 to L-238; Q-230 to Q-239; R-231 to I-240; F-232 to Q-241; E-233 to A-242; V-234 to V-243; T-235 to R-244; G-236 to P-245; W-237 to S-246; L-238 to D-247; Q-239 to E-248; I-240 to G-249; Q-241 to T-250; A-242 to Y-251; V-243 to R-252; R-244 to C-253; P-245 to L-254; S-246 to A-255; D-247 to R-256; E-248 to N-257; G-249 to A-258; T-250 to L-259; Y-251 to G-260; R-252 to Q-261; C-253 to V-262; L-254 to E-263; A-255 to A-264; R-256 to P-265; N-257 to A-266; A-258 to S-267; L-259 to L-268; G-260 to T-269; Q-261 to V-270; V-262 to L-271; E-263 to T-272; A-264 to P-273; P-265 to D-274; A-266 to Q-275; S-267 to L-276; L-268 to N-277; T-269 to S-278; V-270 to T-279; L-271 to G-280; T-272 to I-281; P-273 to P-282; D-274 to Q-283; Q-275 to L-284; L-276 to R-285; N-277 to S-286; S-278 to L-287; T-279 to N-288; G-280 to L-289; I-281 to V-290; P-282 to P-291; Q-283 to E-292; L-284 to E-293; R-285 to E-294; S-286 to A-295; L-287 to E-296; N-288 to S-297; L-289 to E-298; V-290 to E-299; P-291 to N-300; E-292 to D-301; E-293 to D-302; E-294 to Y-303; and A-295 to Y-304 of SEQ ID NO:2. These polypeptide fragments may retain, but do not necessarily have to retain, the biological activity of PSF-2 polypeptides of the invention and/or may be useful to generate or screen for antibodies, as described further below. Polynucleotides encoding these polypeptide fragments are also encompassed by the invention. The present application is also directed to nucleic acid molecules comprising, or alternatively, consisting of, a polynucleotide sequence at least 90%, 92%, 95%, 96%, 97%, 98% or 99% identical to the polynucleotide sequences encoding the PSF-2 polypeptides described above. The present invention also encompasses the above polynucleotide sequences fused to a heterologous polynucleotide sequence.

Additional preferred polypeptide fragments comprise, or alternatively consist of, the amino acid sequence of residues: M-1 to V-15; L-2 to L-16; P-3 to L-17; P-4 to L-18; P-5 to L-19; R-6 to L-20; P-7 to V-21; A-8 to V-22; A-9 to L-23; A-10 to T-24; L-11 to P-25; A-12 to P-26; L-13 to P-27; P-14 to T-28; V-15 to G-29; L-16 to A-30; L-17 to R-31; L-18 to P-32; L-19 to S-33; L-20 to P-34; V-21 to G-35; V-22 to P-36; L-23 to D-37; T-24 to Y-38; P-25 to L-39; P-26 to R-40; P-27 to R-41; T-28 to G-42; G-29 to W-43; A-30 to M-44; R-31 to R-45; P-32 to L-46; S-33 to L-47; P-34 to A-48; G-35 to E-49; P-36 to G-50; D-37 to E-51; Y-38 to G-52; L-39 to C-53; R-40 to A-54; R-41 to P-55; G-42 to C-56; W-43 to R-57; M-44 to P-58; R-45 to E-59; L-46 to E-60; L-47 to C-61; A-48 to A-62; E-49 to A-63; G-50 to P-64; E-51 to R-65; G-52 to G-66; C-53 to C-67; A-54 to L-68; P-55 to A-69; C-56 to G-70; R-57 to R-71; P-58 to V-72; E-59 to R-73; E-60 to D-74; C-61 to A-75; A-62 to C-76; A-63 to G-77; P-64 to C-78; R-65 to C-79; G-66 to W-80; C-67 to E-81; L-68 to C-82; A-69 to A-83; G-70 to N-84; R-71 to L-85; V-72 to E-86; R-73 to G-87; D-74 to Q-88; A-75 to L-89; C-76 to C-90; G-77 to D-91; C-78 to L-92; C-79 to D-93; W-80 to P-94; E-81 to S-95; C-82 to A-96; A-83 to H-97; N-84 to F-98; L-85 to Y-99; E-86 to G-100; G-87 to H-101; Q-88 to C-102; L-89 to G-103; C-90 to E-104; D-91 to Q-105; L-92 to L-106; D-93 to E-107; P-94 to C-108; S-95 to R-109; A-96 to L-110; H-97 to D-111; F-98 to T-112; Y-99 to G-113; G-100 to G-114; H-101 to D-115; C-102 to L-116; G-103 to S-117; E-104 to R-118; Q-105 to G-119; L-106 to E-120; E-107 to V-121; C-108 to P-122; R-109 to E-123; L-110 to P-124; D-111 to L-125; T-112 to C-126; G-113 to A-127; G-114 to C-128; D-115 to R-129; L-116 to S-130; S-117 to Q-131; R-118 to S-132; G-119 to P-133; E-120 to L-134; V-121 to C-135; P-122 to G-136; E-123 to S-137; P-124 to D-138; L-125 to G-139; C-126 to H-140; A-127 to T-141; C-128 to Y-142; R-129 to S-143; S-130 to Q-144; Q-131 to 1-145; S-132 to C-146; P-133 to R-147; L-134 to L-148; C-135 to Q-149; G-136 to E-150; S-137 to A-151; D-138 to A-152; G-139 to R-153; H-140 to A-154; T-141 to R-155; Y-142 to P-156; S-143 to D-157; Q-144 to A-158; I-145 to N-159; C-146 to L-160; R-147 to T-161; L-148 to V-162; Q-149 to A-163; E-150 to H-164; A-151 to P-165; A-152 to G-166; R-153 to P-167; A-154 to C-168; R-155 to E-169; P-156 to S-170; D-157 to G-171; A-158 to P-172; N-159 to Q-173; L-160 to I-174; T-161 to V-175; V-162 to S-176; A-163 to H-177; H-164 to P-178; P-165 to Y-179; G-166 to D-180; P-167 to T-181; C-168 to W-182; E-169 to N-183; S-170 to V-184; G-171 to T-185; P-172 to G-186; Q-173 to Q-187; I-174 to D-188; V-175 to V-189; S-176 to I-190; H-177 to F-191; P-178 to G-192; Y-179 to C-193; D-180 to E-194; T-181 to V-195; W-182 to F-196; N-183 to A-197; V-184 to Y-198; T-185 to P-199; G-186 to M-200; Q-187 to A-201; D-188 to S-202; V-189 to 1-203; I-190 to E-204; F-191 to W-205; G-192 to R-206; C-193 to K-207; E-194 to D-208; V-195 to G-209; F-196 to L-210; A-197 to D-211; Y-198 to I-212; P-199 to Q-213; M-200 to L-214; A-201 to P-215; S-202 to G-216; I-203 to D-217; E-204 to D-218; W-205 to P-219; R-206 to H-220; K-207 to I-221; D-208 to S-222; G-209 to V-223; L-210 to Q-224; D-211 to F-225; I-212 to R-226; Q-213 to G-227; L-214 to G-228; P-215 to P-229; G-216 to Q-230; D-217 to R-231; D-218 to F-232; P-219 to E-233; H-220 to V-234; I-221 to T-235; S-222 to G-236; V-223 to W-237; Q-224 to L-238; F-225 to Q-239; R-226 to I-240; G-227 to Q-241; G-228 to A-242; P-229 to V-243; Q-230 to R-244; R-231 to P-245; F-232 to S-246; E-233 to D-247; V-234 to E-248; T-235 to G-249; G-236 to T-250; W-237 to Y-251; L-238 to R-252; Q-239 to C-253; I-240 to L-254; Q-241 to A-255; A-242 to R-256; V-243 to N-257; R-244 to A-258; P-245 to L-259; S-246 to G-260; D-247 to Q-261; E-248 to V-262; G-249 to E-263; T-250 to A-264; Y-251 to P-265; R-252 to A-266; C-253 to S-267; L-254 to L-268; A-255 to T-269; R-256 to V-270; N-257 to L-271; A-258 to T-272; L-259 to P-273; G-260 to D-274; Q-261 to Q-275; V-262 to L-276; E-263 to N-277; A-264 to S-278; P-265 to T-279; A-266 to G-280; S-267 to I-281; L-268 to P-282; T-269 to Q-283; V-270 to L-284; L-271 to R-285; T-272 to S-286; P-273 to L-287; D-274 to N-288; Q-275 to L-289; L-276 to V-290; N-277 to P-291; S-278 to E-292; T-279 to E-293; G-280 to E-294; I-281 to A-295; P-282 to E-296; Q-283 to S-297; L-284 to E-298; R-285 to E-299; S-286 to N-300; L-287 to D-301; N-288 to D-302; L-289 to Y-303; and V-290 to Y-304 of SEQ ID NO:2. These polypeptide fragments may retain, but do not necessarily have to retain, the biological activity of PSF-2 polypeptides of the invention and/or may be useful to generate or screen for antibodies, as described further below. Polynucleotides encoding these polypeptide fragments are also encompassed by the invention. The present application is also directed to nucleic acid molecules comprising, or alternatively, consisting of, a polynucleotide sequence at least 90%, 92%, 95%, 96%, 97%, 98% or 99% identical to the polynucleotide sequences encoding the PSF-2 polypeptides described above. The present invention also encompasses the above polynucleotide sequences fused to a heterologous polynucleotide sequence.

Additional preferred polypeptide fragments comprise, or alternatively consist of, the amino acid sequence of residues: M-1 to A-30; L-2 to R-31; P-3 to P-32; P-4 to S-33; P-5 to P-34; R-6 to G-35; P-7 to P-36; A-8 to D-37; A-9 to Y-38; A-10 to L-39; L-11 to R-40; A-12 to R-41; L-13 to G-42; P-14 to W-43; V-15 to M-44; L-16 to R-45; L-17 to L-46; L-18 to L-47; L-19 to A-48; L-20 to E-49; V-21 to G-50; V-22 to E-51; L-23 to G-52; T-24 to C-53; P-25 to A-54; P-26 to P-55; P-27 to C-56; T-28 to R-57; G-29 to P-58; A-30 to E-59; R-31 to E-60; P-32 to C-61; S-33 to A-62; P-34 to A-63; G-35 to P-64; P-36 to R-65; D-37 to G-66; Y-38 to C-67; L-39 to L-68; R-40 to A-69; R-41 to G-70; G-42 to R-71; W-43 to V-72; M-44 to R-73; R-45 to D-74; L-46 to A-75; L-47 to C-76; A-48 to G-77; E-49 to C-78; G-50 to C-79; E-51 to W-80; G-52 to E-81; C-53 to C-82; A-54 to A-83; P-55 to N-84; C-56 to L-85; R-57 to E-86; P-58 to G-87; E-59 to Q-88; E-60 to L-89; C-61 to C-90; A-62 to D-91; A-63 to L-92; P-64 to D-93; R-65 to P-94; G-66 to S-95; C-67 to A-96; L-68 to H-97; A-69 to F-98; G-70 to Y-99; R-71 to G-100; V-72 to H-101; R-73 to C-102; D-74 to G-103; A-75 to E-104; C-76 to Q-105; G-77 to L-106; C-78 to E-107; C-79 to C-108; W-80 to R-109; E-81 to L-110; C-82 to D-111; A-83 to T-112; N-84 to G-113; L-85 to G-114; E-86 to D-115; G-87 to L-116; Q-88 to S-117; L-89 to R-118; C-90 to G-119; D-91 to E-120; L-92 to V-121; D-93 to P-122; P-94 to E-123; S-95 to P-124; A-96 to L-125; H-97 to C-126; F-98 to A-127; Y-99 to C-128; G-100 to R-129; H-101 to S-130; C-102 to Q-131; G-103 to S-132; E-104 to P-133; Q-105 to L-134; L-106 to C-135; E-107 to G-136; C-108 to S-137; R-109 to D-138; L-110 to G-139; D-111 to H-140; T-112 to T-141; G-113 to Y-142; G-114 to S-143; D-115 to Q-144; L-116 to I-145; S-117 to C-146; R-118 to R-147; G-119 to L-148; E-120 to Q-149; V-121 to E-150; P-122 to A-151; E-123 to A-152; P-124 to R-153; L-125 to A-154; C-126 to R-155; A-127 to P-156; C-128 to D-157; R-129 to A-158; S-130 to N-159; Q-131 to L-160; S-132 to T-161; P-133 to V-162; L-134 to A-163; C-135 to H-164; G-136 to P-165; S-137 to G-166; D-138 to P-167; G-139 to C-168; H-140 to E-169; T-141 to S-170; Y-142 to G-171; S-143 to P-172; Q-144 to Q-173; I-145 to I-174; C-146 to V-175; R-147 to S-176; L-148 to H-177; Q-149 to P-178; E-150 to Y-179; A-151 to D-180; A-152 to T-181; R-153 to W-182; A-154 to N-183; R-155 to V-184; P-156 to T-185; D-157 to G-186; A-158 to Q-187; N-159 to D-188; L-160 to V-189; T-161 to I-190; V-162 to F-191; A-163 to G-192; H-164 to C-193; P-165 to E-194; G-166 to V-195; P-167 to F-196; C-168 to A-197; E-169 to Y-198; S-170 to P-199; G-171 to M-200; P-172 to A-201; Q-173 to S-202; I-174 to I-203; V-175 to E-204; S-176 to W-205; H-177 to R-206; P-178 to K-207; Y-179 to D-208; D-180 to G-209; T-181 to L-210; W-182 to D-211; N-183 to I-212; V-184 to Q-213; T-185 to L-214; G-186 to P-215; Q-187 to G-216; D-188 to D-217; V-189 to D-218; I-190 to P-219; F-191 to H-220; G-192 to I-221; C-193 to S-222; E-194 to V-223; V-195 to Q-224; F-196 to F-225; A-197 to R-226; Y-198 to G-227; P-199 to G-228; M-200 to P-229; A-201 to Q-230; S-202 to R-231; I-203 to F-232; E-204 to E-233; W-205 to V-234; R-206 to T-235; K-207 to G-236; D-208 to W-237; G-209 to L-238; L-210 to Q-239; D-211 to I-240; I-212 to Q-241; Q-213 to A-242; L-214 to V-243; P-215 to R-244; G-216 to P-245; D-217 to S-246; D-218 to D-247; P-219 to E-248; H-220 to G-249; I-221 to T-250; S-222 to Y-251; V-223 to R-252; Q-224 to C-253; F-225 to L-254; R-226 to A-255; G-227 to R-256; G-228 to N-257; P-229 to A-258; Q-230 to L-259; R-231 to G-260; F-232 to Q-261; E-233 to V-262; V-234 to E-263; T-235 to A-264; G-236 to P-265; W-237 to A-266; L-238 to S-267; Q-239 to L-268; I-240 to T-269; Q-241 to V-270; A-242 to L-271; V-243 to T-272; R-244 to P-273; P-245 to D-274; S-246 to Q-275; D-247 to L-276; E-248 to N-277; G-249 to S-278; T-250 to T-279; Y-251 to G-280; R-252 to I-281; C-253 to P-282; L-254 to Q-283; A-255 to L-284; R-256 to R-285; N-257 to S-286; A-258 to L-287; L-259 to N-288; G-260 to L-289; Q-261 to V-290; V-262 to P-291; E-263 to E-292; A-264 to E-293; P-265 to E-294; A-266 to A-295; S-267 to E-296; L-268 to S-297; T-269 to E-298; V-270 to E-299; L-271 to N-300; T-272 to D-301; P-273 to D-302; D-274 to Y-303; and Q-275 to Y-304 of SEQ ID NO:2. These polypeptide fragments may retain, but do not necessarily have to retain, the biological activity of PSF-2 polypeptides of the invention and/or may be useful to generate or screen for antibodies, as described further below. Polynucleotides encoding these polypeptide fragments are also encompassed by the invention. The present application is also directed to nucleic acid molecules comprising, or alternatively, consisting of, a polynucleotide sequence at least 90%, 92%, 95%, 96%, 97%, 98% or 99% identical to the polynucleotide sequences encoding the PSF-2 polypeptides described above. The present invention also encompasses the above polynucleotide sequences fused to a heterologous polynucleotide sequence.

Additional preferred polypeptide fragments comprise, or alternatively consist of, the amino acid sequence of residues: M-1 to G-50; L-2 to E-51; P-3 to G-52; P-4 to C-53; P-5 to A-54; R-6 to P-55; P-7 to C-56; A-8 to R-57; A-9 to P-58; A-10 to E-59; L-11 to E-60; A-12 to C-61; L-13 to A-62; P-14 to A-63; V-15 to P-64; L-16 to R-65; L-17 to G-66; L-18 to C-67; L-19 to L-68; L-20 to A-69; V-21 to G-70; V-22 to R-71; L-23 to V-72; T-24 to R-73; P-25 to D-74; P-26 to A-75; P-27 to C-76; T-28 to G-77; G-29 to C-78; A-30 to C-79; R-31 to W-80; P-32 to E-81; S-33 to C-82; P-34 to A-83; G-35 to N-84; P-36 to L-85; D-37 to E-86; Y-38 to G-87; L-39 to Q-88; R-40 to L-89; R-41 to C-90; G-42 to D-91; W-43 to L-92; M-44 to D-93; R-45 to P-94; L-46 to S-95; L-47 to A-96; A-48 to H-97; E-49 to F-98; G-50 to Y-99; E-51 to G-100; G-52 to H-101; C-53 to C-102; A-54 to G-103; P-55 to E-104; C-56 to Q-105; R-57 to L-106; P-58 to E-107; E-59 to C-108; E-60 to R-109; C-61 to L-110; A-62 to D-111; A-63 to T-112; P-64 to G-113; R-65 to G-114; G-66 to D-115; C-67 to L-116; L-68 to S-117; A-69 to R-118; G-70 to G-119; R-71 to E-120; V-72 to V-121; R-73 to P-122; D-74 to E-123; A-75 to P-124; C-76 to L-125; G-77 to C-126; C-78 to A-127; C-79 to C-128; W-80 to R-129; E-81 to S-130; C-82 to Q-131; A-83 to S-132; N-84 to P-133; L-85 to L-134; E-86 to C-135; G-87 to G-136; Q-88 to S-137; L-89 to D-138; C-90 to G-139; D-91 to H-140; L-92 to T-141; D-93 to Y-142; P-94 to S-143; S-95 to Q-144; A-96 to I-145; H-97 to C-146; F-98 to R-147; Y-99 to L-148; G-100 to Q-149; H-101 to E-150; C-102 to A-151; G-103 to A-152; E-104 to R-153; Q-105 to A-154; L-106 to R-155; E-107 to P-156; C-108 to D-157; R-109 to A-158; L-110 to N-159; D-111 to L-160; T-112 to T-161; G-113 to V-162; G-114 to A-163; D-115 to H-164; L-116 to P-165; S-117 to G-166; R-118 to P-167; G-119 to C-168; E-120 to E-169; V-121 to S-170; P-122 to G-171; E-123 to P-172; P-124 to Q-173; L-125 to I-174; C-126 to V-175; A-127 to S-176; C-128 to H-177; R-129 to P-178; S-130 to Y-179; Q-131 to D-180; S-132 to T-181; P-133 to W-182; L-134 to N-183; C-135 to V-184; G-136 to T-185; S-137 to G-186; D-138 to Q-187; G-139 to D-188; H-140 to V-189; T-141 to I-190; Y-142 to F-191; S-143 to G-192; Q-144 to C-193; I-145 to E-194; C-146 to V-195; R-147 to F-196; L-148 to A-197; Q-149 to Y-198; E-150 to P-199; A-151 to M-200; A-152 to A-201; R-153 to S-202; A-154 to I-203; R-155 to E-204; P-156 to W-205; D-157 to R-206; A-158 to K-207; N-159 to D-208; L-160 to G-209; T-161 to L-210; V-162 to D-211; A-163 to I-212; H-164 to Q-213; P-165 to L-214; G-166 to P-215; P-167 to G-216; C-168 to D-217; E-169 to D-218; S-170 to P-219; G-171 to H-220; P-172 to I-221; Q-173 to S-222; I-174 to V-223; V-175 to Q-224; S-176 to F-225; H-177 to R-226; P-178 to G-227; Y-179 to G-228; D-180 to P-229; T-181 to Q-230; W-182 to R-231; N-183 to F-232; V-184 to E-233; T-185 to V-234; G-186 to T-235; Q-187 to G-236; D-188 to W-237; V-189 to L-238; I-190 to Q-239; F-191 to I-240; G-192 to Q-241; C-193 to A-242; E-194 to V-243; V-195 to R-244; F-196 to P-245; A-197 to S-246; Y-198 to D-247; P-199 to E-248; M-200 to G-249; A-201 to T-250; S-202 to Y-251; I-203 to R-252; E-204 to C-253; W-205 to L-254; R-206 to A-255; K-207 to R-256; D-208 to N-257; G-209 to A-258; L-210 to L-259; D-211 to G-260; I-212 to Q-261; Q-213 to V-262; L-214 to E-263; P-215 to A-264; G-216 to P-265; D-217 to A-266; D-218 to S-267; P-219 to L-268; H-220 to T-269; I-221 to V-270; S-222 to L-271; V-223 to T-272; Q-224 to P-273; F-225 to D-274; R-226 to Q-275; G-227 to L-276; G-228 to N-277; P-229 to S-278; Q-230 to T-279; R-231 to G-280; F-232 to I-281; E-233 to P-282; V-234 to Q-283; T-235 to L-284; G-236 to R-285; W-237 to S-286; L-238 to L-287; Q-239 to N-288; I-240 to L-289; Q-241 to V-290; A-242 to P-291; V-243 to E-292; R-244 to E-293; P-245 to E-294; S-246 to A-295; D-247 to E-296; E-248 to S-297; G-249 to E-298; T-250 to E-299; Y-251 to N-300; R-252 to D-301; C-253 to D-302; L-254 to Y-303; A-255 to Y-304 of SEQ ID NO:2. These polypeptide fragments may retain, but do not necessarily have to retain, the biological activity of PSF-2 polypeptides of the invention and/or may be useful to generate or screen for antibodies, as described further below. Polynucleotides encoding these polypeptide fragments are also encompassed by the invention. The present application is also directed to nucleic acid molecules comprising, or alternatively, consisting of, a polynucleotide sequence at least 90%, 92%, 95%, 96%, 97%, 98% or 99% identical to the polynucleotide sequences encoding the PSF-2 polypeptides described above. The present invention also encompasses the above polynucleotide sequences fused to a heterologous polynucleotide sequence.

In further embodiments, polynucleotides of the invention comprise at least 15, at least 30, at least 50, at least 100, or at least 250, at least 500, or at least 1000 contiguous nucleotides of PSF-2 coding sequence, but consist of less than or equal to 1000 kb, 500 kb, 250 kb, 200 kb, 150 kb, 100 kb, 75 kb, 50 kb, 30 kb, 25 kb, 20 kb, 15 kb, 10 kb, or 5 kb of genomic DNA that flanks the 5′ or 3′ coding nucleotide set forth in FIGS. 1A and 1B (SEQ ID NO:1). In further embodiments, polynucleotides of the invention comprise at least 15, at least 30, at least 50, at least 100, or at least 250, at least 500, or at least 1000 contiguous nucleotides of PSF-2 coding sequence, but do not comprise all or a portion of any PSF-2 intron. In another embodiment, the nucleic acid comprising PSF-2 coding sequence does not contain coding sequences of a genomic flanking gene (i.e., 5′ or 3′ to the PSF-2 gene in the genome). In other embodiments, the polynucleotides of the invention do not contain the coding sequence of more than 1000, 500, 250, 100, 50, 25, 20, 15, 10, 5, 4, 3, 2, or 1 genomic flanking gene(s).

The PSF-2 polypeptides of the invention may be in monomers or multimers (i.e., dimers, trimers, tetramers and higher multimers). Accordingly, the present invention relates to monomers and multimers of the PSF-2 polypeptides of the invention, their preparation, and compositions (preferably, pharmaceutical compositions) containing them. In specific embodiments, the polypeptides of the invention are monomers, dimers, trimers or tetramers. In additional embodiments, the multimers of the invention are at least dimers, at least trimers, or at least tetramers.

Multimers encompassed by the invention may be homomers or heteromers. As used herein, the term homomer, refers to a multimer containing only PSF-2 polypeptides of the invention (including PSF-2 fragments, variants, and fusion proteins, as described herein). These homomers may contain PSF-2 polypeptides having identical or different amino acid sequences. In a specific embodiment, a homomer of the invention is a multimer containing only PSF-2 polypeptides having an identical amino acid sequence. In another specific embodiment, a homomer of the invention is a multimer containing PSF-2 polypeptides having different amino acid sequences. In specific embodiments, the multimer of the invention is a homodimer (e.g., containing PSF-2 polypeptides having identical or different amino acid sequences) or a homotrimer (e.g., containing PSF-2 polypeptides having identical or different amino acid sequences). In a preferred embodiment, the multimer of the invention is a homotrimer. In additional embodiments, the homomeric multimer of the invention is at least a homodimer, at least a homotrimer, or at least a homotetramer.

As used herein, the term heteromer refers to a multimer containing heterologous polypeptides (i.e., polypeptides of a different protein) in addition to the PSF-2 polypeptides of the invention. In a specific embodiment, the multimer of the invention is a heterodimer, a heterotrimer, or a heterotetramer. In additional embodiments, the heteromeric multimer of the invention is at least a heterodimer, at least a heterotrimer, or at least a heterotetramer.

Multimers of the invention may be the result of hydrophobic, hydrophilic, ionic and/or covalent associations and/or may be indirectly linked, by for example, liposome formation. Thus, in one embodiment, multimers of the invention, such as, for example, homodimers or homotrimers, are formed when polypeptides of the invention contact one another in solution. In another embodiment, heteromultimers of the invention, such as, for example, heterotrimers or heterotetramers, are formed when polypeptides of the invention contact antibodies to the polypeptides of the invention (including antibodies to the heterologous polypeptide sequence in a fusion protein of the invention) in solution. In other embodiments, multimers of the invention are formed by covalent associations with and/or between the PSF-2 polypeptides of the invention. Such covalent associations may involve one or more amino acid residues contained in the polypeptide sequence (e.g., that recited in SEQ ID NO:2 or contained in the polypeptide encoded by the clone deposited in connection with this application). In one instance, the covalent associations are cross-linking between cysteine residues located within the polypeptide sequences which interact in the native (i.e., naturally occurring) polypeptide. In another instance, the covalent associations are the consequence of chemical or recombinant manipulation. Alternatively, such covalent associations may involve one or more amino acid residues contained in the heterologous polypeptide sequence in a PSF-2 fusion protein. In one example, covalent associations are between the heterologous sequence contained in a fusion protein of the invention (see, e.g., U.S. Pat. No. 5,478,925). In a specific example, the covalent associations are between the heterologous sequence contained in a PSF-2-Fc fusion protein of the invention (as described herein). In another specific example, covalent associations of fusion proteins of the invention are between heterologous polypeptide sequence from another TNF family ligand/receptor member that is capable of forming covalently associated multimers, such as for example, oseteoprotegerin (see, e.g., International Publication No. WO 98/49305, the contents of which are herein incorporated by reference in its entirety). In another embodiment, two or more PSF-2 polypeptides of the invention are joined through synthetic linkers (e.g., peptide, carbohydrate or soluble polymer linkers). Examples include those peptide linkers described in U.S. Pat. No. 5,073,627 (hereby incorporated by reference). Proteins comprising multiple PSF-2 polypeptides separated by peptide linkers may be produced using conventional recombinant DNA technology.

Another method for preparing multimer PSF-2 polypeptides of the invention involves use of PSF-2 polypeptides fused to a leucine zipper polypeptide sequence. Leucine zipper domains are polypeptides that promote multimerization of the proteins in which they are found. Leucine zippers were originally identified in several DNA-binding proteins (Landschulz et al., Science 240:1759, (1988)), and have since been found in a variety of different proteins. Among the known leucine zippers are naturally occurring peptides and derivatives thereof that dimerize or trimerize. Examples of leucine zipper domains suitable for producing soluble multimeric PSF-2 proteins are those described in PCT application WO 94/10308, hereby incorporated by reference. Recombinant fusion proteins comprising a soluble PSF-2 polypeptide fused to a peptide that dimerizes or trimerizes in solution are expressed in suitable host cells, and the resulting soluble multimeric PSF-2 is recovered from the culture supernatant using techniques known in the art.

Certain members of the TNF family of proteins are believed to exist in trimeric form (Beutler and Huffel, Science 264:667, 1994; Banner et al., Cell 73:431, 1993). Trimeric PSF-2 may offer the advantage of enhanced biological activity. Preferred leucine zipper moieties are those that preferentially form trimers. One example is a leucine zipper derived from lung surfactant protein D (SPD), as described in Hoppe et al. (FEBS Letters 344:191, (1994)) and in U.S. patent application Ser. No. 08/446,922, hereby incorporated by reference. Other peptides derived from naturally occuring trimeric proteins may be employed in preparing trimeric PSF-2.

In another example, proteins of the invention are associated by interactions between the Flag® polypeptide sequence contained in Flag®-PSF-2 fusion proteins of the invention. In a further embodiment, proteins of the invention are associated by interactions between the heterologous polypeptide sequence contained in Flag®PSF-2 fusion proteins of the invention and anti-Flag® antibody.

The multimers of the invention may be generated using chemical techniques known in the art. For example, polypeptides desired to be contained in the multimers of the invention may be chemically cross-linked using linker molecules and linker molecule length optimization techniques known in the art (see, e.g., U.S. Pat. No. 5,478,925, which is herein incorporated by reference in its entirety). Additionally, multimers of the invention may be generated using techniques known in the art to form one or more inter-molecule cross-links between the cysteine residues located within the sequence of the polypeptides desired to be contained in the multimer (see, e.g., U.S. Pat. No. 5,478,925, which is herein incorporated by reference in its entirety). Further, polypeptides of the invention may be routinely modified by the addition of cysteine or biotin to the C terminus or N-terminus of the polypeptide and techniques known in the art may be applied to generate multimers containing one or more of these modified polypeptides (see, e.g., U.S. Pat. No. 5,478,925, which is herein incorporated by reference in its entirety). Additionally, techniques known in the art may be applied to generate liposomes containing the polypeptide components desired to be contained in the multimer of the invention (see, e.g., U.S. Pat. No. 5,478,925, which is herein incorporated by reference in its entirety).

Transgenics and “Knock-Outs”

The polypeptides of the invention can also be expressed in transgenic animals. Animals of any species, including, but not limited to, mice, rats, rabbits, hamsters, guinea pigs, pigs, micro-pigs, goats, sheep, cows and non-human primates, e.g., baboons, monkeys, and chimpanzees may be used to generate transgenic animals. In a specific embodiment, techniques described herein or otherwise known in the art, are used to express polypeptides of the invention in humans, as part of a gene therapy protocol.

Any technique known in the art may be used to introduce the transgene (i.e., polynucleotides of the invention) into animals to produce the founder lines of transgenic animals. Such techniques include, but are not limited to, pronuclear microinjection (Paterson, et al., Appl. Microbiol. Biotechnol. 40:691-698 (1994); Carver et al., Biotechnology (NY) 11:1263-1270 (1993); Wright et al., Biotechnology (NY) 9:830-834 (1991); and Hoppe et al., U.S. Pat. No. 4,873,191 (1989)); retrovirus mediated gene transfer into germ lines (Van der Putten et al., Proc. Natl. Acad. Sci., USA 82:6148-6152 (1985)), blastocysts or embryos; gene targeting in embryonic stem cells (Thompson et al., Cell 56:313-321 (1989)); electroporation of cells or embryos (Lo, 1983, Mol Cell. Biol. 3:1803-1814 (1983)); introduction of the polynucleotides of the invention using a gene gun (see, e.g., Ulmer et al., Science 259:1745 (1993); introducing nucleic acid constructs into embryonic pleuripotent stem cells and transferring the stem cells back into the blastocyst; and sperm-mediated gene transfer (Lavitrano et al., Cell 57:717-723 (1989); etc. For a review of such techniques, see Gordon, “Transgenic Animals,” Intl. Rev. Cytol. 115:171-229 (1989), which is incorporated by reference herein in its entirety. See also, U.S. Pat. No. 5,464,764 (Capecchi, et al., Positive-Negative Selection Methods and Vectors); U.S. Pat. No. 5,631,153 (Capecchi, et al., Cells and Non-Human Organisms Containing Predetermined Genomic Modifications and Positive-Negative Selection Methods and Vectors for Making Same); U.S. Pat. No. 4,736,866 (Leder, et al., Transgenic Non-Human Animals); and U.S. Pat. No. 4,873,191 (Wagner, et al., Genetic Transformation of Zygotes); each of which is hereby incorporated by reference in its entirety.

Any technique known in the art may be used to produce transgenic clones containing polynucleotides of the invention,, for example, nuclear transfer into enucleated oocytes of nuclei from cultured embryonic, fetal, or adult cells induced to quiescence (Campell et al., Nature 380:64-66 (1996); Wilmut et al., Nature 385:810-813 (1997)).

The present invention provides for transgenic animals that carry the transgene in all their cells, as well as animals which carry the transgene in some, but not all their cells, i.e., mosaic or chimeric animals. The transgene may be integrated as a single transgene or as multiple copies such as in concatamers, e.g., head-to-head tandems or head-to-tail tandems. The transgene may also be selectively introduced into and activated in a particular cell type by following, for example, the teaching of Lasko et al. (Lasko et al., Proc. Natl. Acad. Sci. USA 89:6232-6236 (1992)). The regulatory sequences required for such a cell-type specific activation will depend upon the particular cell type of interest, and will be apparent to those of skill in the art. When it is desired that the polynucleotide transgene be integrated into the chromosomal site of the endogenous gene, gene targeting is preferred. Briefly, when such a technique is to be utilized, vectors containing some nucleotide sequences homologous to the endogenous gene are designed for the purpose of integrating, via homologous recombination with chromosomal sequences, into and disrupting the function of the nucleotide sequence of the endogenous gene. The transgene may also be selectively introduced into a particular cell type, thus inactivating the endogenous gene in only that cell type, by following, for example, the teaching of Gu et al. (Gu et al., Science 265:103-106 (1994)). The regulatory sequences required for such a cell-type specific inactivation will depend upon the particular cell type of interest, and will be apparent to those of skill in the art. In addition to expressing the polypeptide of the present invention in a ubiquitous or tissue specific manner in transgenic animals, it would also be routine for one skilled in the art to generate constructs which regulate expression of the polypeptide by a variety of other means (for example, developmentally or chemically regulated expression).

Once transgenic animals have been generated, the expression of the recombinant gene may be assayed utilizing standard techniques. Initial screening may be accomplished by Southern blot analysis or PCR techniques to analyze animal tissues to verify that integration of the transgene has taken place. The level of mRNA expression of the transgene in the tissues of the transgenic animals may also be assessed using techniques which include, but are not limited to, Northern blot analysis of tissue samples obtained from the animal, in situ hybridization analysis, and reverse transcriptase-PCR (rt-PCR). Samples of transgenic gene-expressing tissue may also be evaluated immunocytochemically or immunohistochemically using antibodies specific for the transgene product.

Once the founder animals are produced, they may be bred, inbred, outbred, or crossbred to produce colonies of the particular animal. Examples of such breeding strategies include, but are not limited to: outbreeding of founder animals with more than one integration site in order to establish separate lines; inbreeding of separate lines in order to produce compound transgenics that express the transgene at higher levels because of the effects of additive expression of each transgene; crossing of heterozygous transgenic animals to produce animals homozygous for a given integration site in order to both augment expression and eliminate the need for screening of animals by DNA analysis; crossing of separate homozygous lines to produce compound heterozygous or homozygous lines; and breeding to place the transgene on a distinct background that is appropriate for an experimental model of interest.

Transgenic and “knock-out” animals of the invention have uses which include, but are not limited to, animal model systems useful in elaborating the biological function of PSF-2 polypeptides, studying conditions and/or disorders associated with aberrant PSF-2 expression, and in screening for compounds effective in ameliorating such conditions and/or disorders.

In further embodiments of the invention, cells that are genetically engineered to express the polypeptides of the invention, or alternatively, that are genetically engineered not to express the polypeptides of the invention (e.g., knockouts) are administered to a patient in vivo. Such cells may be obtained from the patient (i.e., animal, including human) or an MHC compatible donor and can include, but are not limited to fibroblasts, bone marrow cells, blood cells (e.g., lymphocytes), adipocytes, muscle cells, endothelial cells etc. The cells are genetically engineered in vitro using recombinant DNA techniques to introduce the coding sequence of polypeptides of the invention into the cells, or alternatively, to disrupt the coding sequence and/or endogenous regulatory sequence associated with the polypeptides of the invention, e.g., by transduction (using viral vectors, and preferably vectors that integrate the transgene into the cell genome) or transfection procedures, including, but not limited to, the use of plasmids, cosmids, YACs, naked DNA, electroporation, liposomes, etc. The coding sequence of the polypeptides of the invention can be placed under the control of a strong constitutive or inducible promoter or promoter/enhancer to achieve expression, and preferably secretion, of the polypeptides of the invention. The engineered cells which express and preferably secrete the polypeptides of the invention can be introduced into the patient systemically, e.g., in the circulation, or intraperitoneally.

Alternatively, the cells can be incorporated into a matrix and implanted in the body, e.g., genetically engineered fibroblasts can be implanted as part of a skin graft; genetically engineered endothelial cells can be implanted as part of a lymphatic or vascular graft. (See, for example, Anderson et al. U.S. Pat. No. 5,399,349; and Mulligan & Wilson, U.S. Pat. No. 5,460,959 each of which is incorporated by reference herein in its entirety).

When the cells to be administered are non-autologous or non-MHC compatible cells, they can be administered using well known techniques which prevent the development of a host immune response against the introduced cells. For example, the cells may be introduced in an encapsulated form which, while allowing for an exchange of components with the immediate extracellular environment, does not allow the introduced cells to be recognized by the host immune system.

Epitopes & Antibodies

In the present invention, “epitopes” refer to PSF-2 polypeptide fragments having antigenic or immunogenic activity in an animal, especially in a human. A preferred embodiment of the present invention relates to a PSF-2 polypeptide fragment comprising an epitope, as well as the polynucleotide encoding this fragment. A region of a polypeptide molecule to which an antibody can bind is defined as an “antigenic epitope.” In contrast, an “immunogenic epitope” is defined as a part of a polypeptide that elicits an antibody response. (See, for instance, Geysen et al., Proc. Natl. Acad. Sci. USA 81:3998-4002 (1983).)

Fragments which function as epitopes may be produced by any conventional means. (See, e.g., Houghten, R. A., Proc. Natl. Acad. Sci. USA 82:5131-5135 (1985) further described in U.S. Pat. No. 4,631,211.)

In the present invention, antigenic epitopes preferably contain a sequence of at least seven, more preferably at least nine, and most preferably between about 15 to about 30 amino acids. Antigenic epitopes are useful to raise antibodies, including monoclonal antibodies, that specifically bind the epitope. (See, for instance, Wilson, et al., Cell 37:767-778 (1984); Sutcliffe, J. G., et al., Science 219:660-666 (1983).)

Similarly, immunogenic epitopes can be used to induce antibodies according to methods well known in the art. (See, for instance, Sutcliffe et al., supra; Wilson et al., supra; Chow, M. et al., Proc. Natl. Acad. Sci. USA 82:910-914; and Bittle, F. J. et al., J. Gen. Virol. 66:2347-2354 (1985).) A preferred immunogenic epitope includes the secreted polypeptide. The immunogenic epitopes may be presented together with a carrier polypeptide, such as an albumin, to an animal system (such as rabbit or mouse) or, if it is long enough (at least about 25 amino acids), without a carrier. However, immunogenic epitopes comprising as few as 8 to 10 amino acids have been shown to be sufficient to raise antibodies capable of binding to, at the very least, linear epitopes in a denatured polypeptide (e.g., in Western blotting.)

Using the Protean module of DNA*STAR (FIG. 3 and Table I), SEQ ID NO:2 was found antigenic at amino acids: Pro-26 to Met-44; Leu-47 to Leu-68; Gly-70 to Cys-78; Ala-83 to Ser-143; Arg-147 to Leu-160; His-164 to Pro-172; Ile-203 to Ile-221; Gln-224 to Glu-233; Ala-242 to Tyr-251; Thr-272 to Gly-280; and Asn-288 to Tyr-304. Thus, these regions could be used as epitopes to produce antibodies against polypeptides of the present invention.

As used herein, the term “antibody” (Ab) or “monoclonal antibody” (Mab) is meant to include intact molecules as well as antibody fragments (such as, for example, Fab and F(ab′)2 fragments) which are capable of specifically binding to protien. Fab and F(ab′)2 fragments lack the Fc fragment of intact antibody, clear more rapidly from the circulation, and may have less non-specific tissue binding than an intact antibody. (Wahl et al., J. Nucl. Med. 24:316-325 (1983).) Thus, these fragments are preferred, as well as the products of a FAB or other immunoglobulin expression library. Moreover, antibodies of the present invention include chimeric, single chain, and humanized antibodies.

The present invention encompasses polypeptides comprising, or alternatively consisting of, an epitope of the polypeptide having an amino acid sequence of SEQ ID NO:2, or an epitope of the polypeptide sequence encoded by a polynucleotide sequence contained in ATCC deposit No. 203521 or encoded by a polynucleotide that hybridizes to the complement of the sequence of SEQ ID NO:1 or contained in ATCC deposit No. 203521 under stringent hybridization conditions or lower stringency hybridization conditions as defined supra. The present invention further encompasses polynucleotide sequences encoding an epitope of a polypeptide sequence of the invention (such as, for example, the sequence disclosed in SEQ ID NO:1), polynucleotide sequences of the complementary strand of a polynucleotide sequence encoding an epitope of the invention, and polynucleotide sequences which hybridize to the complementary strand under stringent hybridization conditions or lower stringency hybridization conditions defined supra.

The term “epitopes,” as used herein, refers to portions of a polypeptide having antigenic or immunogenic activity in an animal, preferably a mammal, and most preferably in a human. In a preferred embodiment, the present invention encompasses a polypeptide comprising an epitope, as well as the polynucleotide encoding this polypeptide. An “immunogenic epitope,” as used herein, is defined as a portion of a protein that elicits an antibody response in an animal, as determined by any method known in the art, for example, by the methods for generating antibodies described infra. (See, for example, Geysen et al., Proc. Natl. Acad. Sci. USA 81:3998-4002 (1983)). The term “antigenic epitope,” as used herein, is defined as a portion of a protein to which an antibody can immunospecifically bind its antigen as determined by any method well known in the art, for example, by the immunoassays described herein. Immunospecific binding excludes non-specific binding but does not necessarily exclude cross-reactivity with other antigens. Antigenic epitopes need not necessarily be immunogenic.

Fragments which function as epitopes may be produced by any conventional means. (See, e.g., Houghten, Proc. Natl. Acad. Sci. USA 82:5131-5135 (1985), further described in U.S. Pat. No. 4,631,211).

In the present invention, antigenic epitopes preferably contain a sequence of at least 4, at least 5, at least 6, at least 7, more preferably at least 8, at least 9, at least 10, at least 11, at least 12, at least 13, at least 14, at least 15, at least 20, at least 25, at least 30, at least 40, at least 50, and, most preferably, between about 15 to about 30 amino acids. Preferred polypeptides comprising immunogenic or antigenic epitopes are at least 10, 15, 20, 25, 30, 35, 40, 45, 50, 55, 60, 65, 70, 75, 80, 85, 90, 95, or 100 amino acid residues in length. Additional non-exclusive preferred antigenic epitopes include the antigenic epitopes disclosed herein, as well as portions thereof. Antigenic epitopes are useful, for example, to raise antibodies, including monoclonal antibodies, that specifically bind the epitope. Preferred antigenic epitopes include the antigenic epitopes disclosed herein, as well as any combination of two, three, four, five or more of these antigenic epitopes. Antigenic epitopes can be used as the target molecules in immunoassays. (See, for instance, Wilson et al., Cell 37:767-778 (1984); Sutcliffe et al., Science 219:660-666 (1983)).

Similarly, immunogenic epitopes can be used, for example, to induce antibodies according to methods well known in the art. (See, for instance, Sutcliffe et al., supra; Wilson et al., supra; Chow et al., Proc. Natl. Acad. Sci. USA 82:910-914; and Bittle et al., J. Gen. Virol. 66:2347-2354 (1985). Preferred immunogenic epitopes include the immunogenic epitopes disclosed herein, as well as any combination of two, three, four, five or more of these immunogenic epitopes. The polypeptides comprising one or more immunogenic epitopes may be presented for eliciting an antibody response together with a carrier protein, such as an albumin, to an animal system (such as rabbit or mouse), or, if the polypeptide is of sufficient length (at least about 25 amino acids), the polypeptide may be presented without a carrier. However, immunogenic epitopes comprising as few as 8 to 10 amino acids have been shown to be sufficient to raise antibodies capable of binding to, at the very least, linear epitopes in a denatured polypeptide (e.g., in Western blotting).

Epitope-bearing polypeptides of the present invention may be used to induce antibodies according to methods well known in the art including, but not limited to, in vivo immunization, in vitro immunization, and phage display methods. See, e.g., Sutcliffe et al., supra; Wilson et al., supra, and Bittle et al., J. Gen. Virol., 66:2347-2354 (1985). If in vivo immunization is used, animals may be immunized with free peptide; however, anti-peptide antibody titer may be boosted by coupling the peptide to a macromolecular carrier, such as keyhole limpet hemacyanin (KLH) or tetanus toxoid. For instance, peptides containing cysteine residues may be coupled to a carrier using a linker such as maleimidobenzoyl-N-hydroxysuccinimide ester (MBS), while other peptides may be coupled to carriers using a more general linking agent such aglutaraldehyde. Animals such as rabbits, rats and mice are immunized with either free or carrier-coupled peptides, for instance, by intraperitoneal and/or intradermal injection of emulsions containing about 100 μg of peptide or carrier protein and Freund's adjuvant or any other adjuvant known for stimulating an immune response. Several booster injections may be needed, for instance, at intervals of about two weeks, to provide a useful titer of anti-peptide antibody which can be detected, for example, by ELISA assay using free peptide adsorbed to a solid surface. The titer of anti-peptide antibodies in serum from an immunized animal may be increased by selection of anti-peptide antibodies, for instance, by adsorption to the peptide on a solid support and elution of the selected antibodies according to methods well known in the art.

As one of skill in the art will appreciate, and as discussed above, the polypeptides of the present invention comprising an immunogenic or antigenic epitope can be fused to other polypeptide sequences. For example, the polypeptides of the present invention may be fused with the constant domain of immunoglobulins (IgA, IgE, IgG, IgM), or portions thereof (CH1, CH2, CH3, or any combination thereof and portions thereof) resulting in chimeric polypeptides. Such fusion proteins may facilitate purification and may increase half-life in vivo. This has been shown for chimeric proteins consisting of the first two domains of the human CD4-polypeptide and various domains of the constant regions of the heavy or light chains of mammalian immunoglobulins. See, e.g., EP 394,827; Traunecker et al., Nature, 331:84-86 (1988). Enhanced delivery of an antigen across the epithelial barrier to the immune system has been demonstrated for antigens (e.g., insulin) conjugated to an FcRn binding partner such as IgG or Fc fragments (see, e.g., PCT Publications WO 96/22024 and WO 99/04813). IgG Fusion proteins that have a disulfide-linked dimeric structure due to the IgG portion desulfide bonds have also been found to be more efficient in binding and neutralizing other molecules than monomeric polypeptides or fragments thereof alone. See, e.g., Fountoulakis et al., J. Biochem., 270:3958-3964 (1995). Nucleic acids encoding the above epitopes can also be recombined with a gene of interest as an epitope tag (e.g., the hemagglutinin (“HA”) tag or flag tag) to aid in detection and purification of the expressed polypeptide. For example, a system described by Janknecht et al. allows for the ready purification of non-denatured fusion proteins expressed in human cell lines (Janknecht et al., 1991, Proc. Natl. Acad. Sci. USA 88:8972-897). In this system, the gene of interest is subcloned into a vaccinia recombination plasmid such that the open reading frame of the gene is translationally fused to an amino-terminal tag consisting of six histidine residues. The tag serves as a matrix binding domain for the fusion protein. Extracts from cells infected with the recombinant vaccinia virus are loaded onto Ni²⁺ nitriloacetic acid-agarose column and histidine-tagged proteins can be selectively eluted with imidazole-containing buffers.

Additional fusion proteins of the invention may be generated through the techniques of gene-shuffling, motif-shuffling, exon-shuffling, and/or codon-shuffling (collectively referred to as “DNA shuffling”). DNA shuffling may be employed to modulate the activities of polypeptides of the invention, such methods can be used to generate polypeptides with altered activity, as well as agonists and antagonists of the polypeptides. See, generally, U.S. Pat. Nos. 5,605,793; 5,811,238; 5,830,721; 5,834,252; and 5,837,458, and Patten et al., Curr. Opinion Biotechnol. 8:724-33 (1997); Harayama, Trends Biotechnol. 16(2):76-82 (1998); Hansson, et al., J. Mol. Biol. 287:265-76 (1999); and Lorenzo and Blasco, Biotechniques 24(2):308-13 (1998) (each of these patents and publications are hereby incorporated by reference in its entirety). In one embodiment, alteration of polynucleotides corresponding to SEQ ID NO:X and the polypeptides encoded by these polynucleotides may be achieved by DNA shuffling. DNA shuffling involves the assembly of two or more DNA segments by homologous or site-specific recombination to generate variation in the polynucleotide sequence. In another embodiment, polynucleotides of the invention, or the encoded polypeptides, may be altered by being subjected to random mutagenesis by error-prone PCR, random nucleotide insertion or other methods prior to recombination. In another embodiment, one or more components, motifs, sections, parts, domains, fragments, etc., of a polynucleotide encoding a polypeptide of the invention may be recombined with one or more components, motifs, sections, parts, domains, fragments, etc. of one or more heterologous molecules.

Antibodies

Further polypeptides of the invention relate to antibodies and T-cell antigen receptors (TCR) which immunospecifically bind a polypeptide, polypeptide fragment, or variant of SEQ ID NO:Y, and/or an epitope, of the present invention (as determined by immunoassays well known in the art for assaying specific antibody-antigen binding). Antibodies of the invention include, but are not limited to, polyclonal, monoclonal, multispecific, human, humanized or chimeric antibodies, single chain antibodies, Fab fragments, F(ab′) fragments, fragments produced by a Fab expression library, anti-idiotypic (anti-Id) antibodies (including, e.g., anti-Id antibodies to antibodies of the invention), and epitope-binding fragments of any of the above. The term “antibody,” as used herein, refers to immunoglobulin molecules and immunologically active portions of immunoglobulin molecules, i.e., molecules that contain an antigen binding site that immunospecifically binds an antigen. The immunoglobulin molecules of the invention can be of any type (e.g., IgG, IgE, IgM, IgD, IgA and IgY), class (e.g., IgG1, IgG2, IgG3, IgG4, IgA1 and IgA2) or subclass of immunoglobulin molecule.

Most preferably the antibodies are human antigen-binding antibody fragments of the present invention and include, but are not limited to, Fab, Fab′ and F(ab′)2, Fd, single-chain Fvs (scFv), single-chain antibodies, disulfide-linked Fvs (sdFv) and fragments comprising either a VL or VH domain. Antigen-binding antibody fragments, including single-chain antibodies, may comprise the variable region(s) alone or in combination with the entirety or a portion of the following: hinge region, CH1, CH2, and CH3 domains. Also included in the invention are antigen-binding fragments also comprising any combination of variable region(s) with a hinge region, CH1, CH2, and CH3 domains. The antibodies of the invention may be from any animal origin including birds and mammals. Preferably, the antibodies are human, murine (e.g., mouse and rat), donkey, ship rabbit, goat, guinea pig, camel, horse, or chicken. As used herein, “human” antibodies include antibodies having the amino acid sequence of a human immunoglobulin and include antibodies isolated from human immunoglobulin libraries or from animals transgenic for one or more human immunoglobulin and that do not express endogenous immunoglobulins, as described infra and, for example in, U.S. Pat. No. 5,939,598 by Kucherlapati et al.

The antibodies of the present invention may be monospecific, bispecific, trispecific or of greater multispecificity. Multispecific antibodies may be specific for different epitopes of a polypeptide of the present invention or may be specific for both a polypeptide of the present invention as well as for a heterologous epitope, such as a heterologous polypeptide or solid support material. See, e.g., PCT publications WO 93/17715; WO 92/08802; WO 91/00360; WO 92/05793; Tutt, et al., J. Immunol. 147:60-69 (1991); U.S. Pat. Nos. 4,474,893; 4,714,681; 4,925,648; 5,573,920; 5,601,819; Kostelny et al., J. Immunol. 148:1547-1553 (1992).

Antibodies of the present invention may be described or specified in terms of the epitope(s) or portion(s) of a polypeptide of the present invention which they recognize or specifically bind. The epitope(s) or polypeptide portion(s) may be specified as described herein, e.g., by N-terminal and C-terminal positions, by size in contiguous amino acid residues, or listed in the Tables and Figures. Antibodies which specifically bind any epitope or polypeptide of the present invention may also be excluded. Therefore, the present invention includes antibodies that specifically bind polypeptides of the present invention, and allows for the exclusion of the same.

Antibodies of the present invention may also be described or specified in terms of their cross-reactivity. Antibodies that do not bind any other analog, ortholog, or homolog of a polypeptide of the present invention are included. Antibodies that bind polypeptides with at least 95%, at least 90%, at least 85%, at least 80%, at least 75%, at least 70%, at least 65%, at least 60%, at least 55%, and at least 50% identity (as calculated using methods known in the art and described herein) to a polypeptide of the present invention are also included in the present invention. In specific embodiments, antibodies of the present invention cross-react with murine, rat and/or rabbit homologs of human proteins and the corresponding epitopes thereof. Antibodies that do not bind polypeptides with less than 95%, less than 90%, less than 85%, less than 80%, less than 75%, less than 70%, less than 65%, less than 60%, less than 55%, and less than 50% identity (as calculated using methods known in the art and described herein) to a polypeptide of the present invention are also included in the present invention. In a specific embodiment, the above-described cross-reactivity is with respect to any single specific antigenic or immunogenic polypeptide, or combination(s) of 2, 3, 4, 5, or more of the specific antigenic and/or immunogenic polypeptides disclosed herein. Further included in the present invention are antibodies which bind polypeptides encoded by polynucleotides which hybridize to a polynucleotide of the present invention under stringent hybridization conditions (as described herein). Antibodies of the present invention may also be described or specified in terms of their binding affinity to a polypeptide of the invention. Preferred binding affinities include those with a dissociation constant or Kd less than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10 ⁻⁶ M, 5×10⁻⁷ M, 10⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10^{31 11} M, 5×10⁻¹² M, ¹⁰⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

The invention also provides antibodies that competitively inhibit binding of an antibody to an epitope of the invention as determined by any method known in the art for determining competitive binding, for example, the immunoassays described herein. In preferred embodiments, the antibody competitively inhibits binding to the epitope by at least 95%, at least 90%, at least 85%, at least 80%, at least 75%, at least 70%, at least 60%, or at least 50%.

Antibodies of the present invention may act as agonists or antagonists of the polypeptides of the present invention. For example, the present invention includes antibodies which disrupt the receptor/ligand interactions with the polypeptides of the invention either partially or fully. Preferrably, antibodies of the present invention bind an antigenic epitope disclosed herein, or a portion thereof. The invention features both receptor-specific antibodies and ligand-specific antibodies. The invention also features receptor-specific antibodies which do not prevent ligand binding but prevent receptor activation. Receptor activation (i.e., signaling) may be determined by techniques described herein or otherwise known in the art. For example, receptor activation can be determined by detecting the phosphorylation (e.g., tyrosine or serine/threonine) of the receptor or its substrate by immunoprecipitation followed by western blot analysis (for example, as described supra). In specific embodiments, antibodies are provided that inhibit ligand activity or receptor activity by at least 95%, at least 90%, at least 85%, at least 80%, at least 75%, at least 70%, at least 60%, or at least 50% of the activity in absence of the antibody.

The invention also features receptor-specific antibodies which both prevent ligand binding and receptor activation as well as antibodies that recognize the receptor-ligand complex, and, preferably, do not specifically recognize the unbound receptor or the unbound ligand. Likewise, included in the invention are neutralizing antibodies which bind the ligand and prevent binding of the ligand to the receptor, as well as antibodies which bind the ligand, thereby preventing receptor activation, but do not prevent the ligand from binding the receptor. Further included in the invention are antibodies which activate the receptor. These antibodies may act as receptor agonists, i.e., potentiate or activate either all or a subset of the biological activities of the ligand-mediated receptor activation, for example, by inducing dimerization of the receptor. The antibodies may be specified as agonists, antagonists or inverse agonists for biological activities comprising the specific biological activities of the peptides of the invention disclosed herein. The above antibody agonists can be made using methods known in the art. See, e.g., PCT publication WO 96/40281; U.S. Pat. No. 5,811,097; Deng et al., Blood 92(6):1981-1988 (1998); Chen et al., Cancer Res. 58(16):3668-3678 (1998); Harrop et al., J. Immunol. 161(4):1786-1794 (1998); Zhu et al., Cancer Res. 58(15):3209-3214 (1998); Yoon et al., J. Immunol. 160(7):3170-3179 (1998); Prat et al., J. Cell. Sci. 111(Pt2):237-247 (1998); Pitard et al., J. Immunol. Methods 205(2):177-190 (1997); Liautard et al., Cytokine 9(4):233-241 (1997); Carlson et al., J. Biol. Chem. 272(17):11295-11301 (1997); Taryman et al., Neuron 14(4):755-762 (1995); Muller et al., Structure 6(9):1153-1167 (1998); Bartunek et al., Cytokine 8(1):14-20 (1996) (which are all incorporated by reference herein in their entireties).

Antibodies of the present invention may be used, for example, but not limited to, to purify, detect, and target the polypeptides of the present invention, including both in vitro and in vivo diagnostic and therapeutic methods. For example, the antibodies have use in immunoassays for qualitatively and quantitatively measuring levels of the polypeptides of the present invention in biological samples. See, e.g., Harlow et al., Antibodies: A Laboratory Manual, (Cold Spring Harbor Laboratory Press, 2nd ed. 1988) (incorporated by reference herein in its entirety).

As discussed in more detail below, the antibodies of the present invention may be used either alone or in combination with other compositions. The antibodies may further be recombinantly fused to a heterologous polypeptide at the N- or C-terminus or chemically conjugated (including covalently and non-covalently conjugations) to polypeptides or other compositions. For example, antibodies of the present invention may be recombinantly fused or conjugated to molecules useful as labels in detection assays and effector molecules such as heterologous polypeptides, drugs, radionuclides, or toxins. See, e.g., PCT publications WO 92/08495; WO 91/14438; WO 89/12624; U.S. Pat. No. 5,314,995; and EP 396,387.

The antibodies of the invention include derivatives that are modified, i.e, by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

The antibodies of the present invention may be generated by any suitable method known in the art. Polyclonal antibodies to an antigen-of-interest can be produced by various procedures well known in the art. For example, a polypeptide of the invention can be administered to various host animals including, but not limited to, rabbits, mice, rats, etc. to induce the production of sera containing polyclonal antibodies specific for the antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art and are discussed in detail in the Examples (e.g., Example 11). In a non-limiting example, mice can be immunized with a polypeptide of the invention or a cell expressing such peptide. Once an immune response is detected, e.g., antibodies specific for the antigen are detected in the mouse serum, the mouse spleen is harvested and splenocytes isolated. The splenocytes are then fused by well known techniques to any suitable myeloma cells, for example cells from cell line SP20 available from the ATCC. Hybridomas are selected and cloned by limited dilution. The hybridoma clones are then assayed by methods known in the art for cells that secrete antibodies capable of binding a polypeptide of the invention. Ascites fluid, which generally contains high levels of antibodies, can be generated by immunizing mice with positive hybridoma clones.

Accordingly, the present invention provides methods of generating monoclonal antibodies as well as antibodies produced by the method comprising culturing a hybridoma cell secreting an antibody of the invention wherein, preferably, the hybridoma is generated by fusing splenocytes isolated from a mouse immunized with an antigen of the invention with myeloma cells and then screening the hybridomas resulting from the fusion for hybridoma clones that secrete an antibody able to bind a polypeptide of the invention.

Antibody fragments which recognize specific epitopes may be generated by known techniques. For example, Fab and F(ab′)2 fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain.

For example, the antibodies of the present invention can also be generated using various phage display methods known in the art. In phage display methods, functional antibody domains are displayed on the surface of phage particles which carry the polynucleotide sequences encoding them. In a particular embodiment, such phage can be utilized to display antigen binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Examples of phage display methods that can be used to make the antibodies of the present invention include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187 9-18 (1997); Burton et al., Advances in Immunology 57:191-280 (1994); PCT application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described in detail below. For example, techniques to recombinantly produce Fab, Fab′ and F(ab′)2 fragments can also be employed using methods known in the art such as those disclosed in PCT publication WO 92/22324; Mullinax et al., BioTechniques 12(6):864-869 (1992); and Sawai et al., AJRI 34:26-34 (1995); and Better et al., Science 240:1041-1043 (1988) (said references incorporated by reference in their entireties).

Examples of techniques which can be used to produce single-chain Fvs and antibodies include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988). For some uses, including in vivo use of antibodies in humans and in vitro detection assays, it may be preferable to use chimeric, humanized, or human antibodies. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See e.g., Morrison, Science 229:1202 (1985); Oi et al., BioTechniques 4:214 (1986); Gillies et al., (1989) J. Immunol. Methods 125:191-202; U.S. Pat. Nos. 5,807,715; 4,816,567; and 4,816397, which are incorporated herein by reference in their entirety. Humanized antibodies are antibody molecules from non-human species antibody that binds the desired antigen having one or more complementarity determining regions (CDRs) from the non-human species and a framework regions from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., Nature 332:323 (1988), which are incorporated herein by reference in their entireties.) Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology 28(4/5):489-498 (1991); Studnicka et al., Protein Engineering 7(6):805-814 (1994); Roguska. et al., PNAS 91:969-973 (1994)), and chain shuffling (U.S. Pat. No. 5,565,332).

Completely human antibodies are particularly desirable for therapeutic treatment of human patients. Human antibodies can be made by a variety of methods known in the art including phage display methods described above using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar, Int. Rev. Immunol. 13:65-93 (1995). For a detailed discussion of this technology for producing human antibodies and human monoclonal antibodies and protocols for producing such antibodies, see, e.g., PCT publications WO 98/24893; WO 92/01047; WO 96/34096; WO 96/33735; European Patent No. 0 598 877; U.S. Pat. Nos. 5,413,923; 5,625,126; 5,633,425; 5,569,825; 5,661,016; 5,545,806; 5,814,318; 5,885,793; 5,916,771; and 5,939,598, which are incorporated by reference herein in their entirety. In addition, companies such as Abgenix, Inc. (Freemont, Calif.) and Genpharm (San Jose, Calif.) can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al., Bio/technology 12:899-903 (1988)).

Further, antibodies to the polypeptides of the invention can, in turn, be utilized to generate anti-idiotype antibodies that “mimic” polypeptides of the invention using techniques well known to those skilled in the art. (See, e.g., Greenspan & Bona, FASEB J. 7(5):437-444; (1989) and Nissinoff, J. Immunol. 147(8):2429-2438 (1991)). For example, antibodies which bind to and competitively inhibit polypeptide multimerization and/or binding of a polypeptide of the invention to a ligand can be used to generate anti-idiotypes that “mimic” the polypeptide multimerization and/or binding domain and, as a consequence, bind to and neutralize polypeptide and/or its ligand. Such neutralizing anti-idiotypes or Fab fragments of such anti-idiotypes can be used in therapeutic regimens to neutralize polypeptide ligand. For example, such anti-idiotypic antibodies can be used to bind a polypeptide of the invention and/or to bind its ligands/receptors, and thereby block its biological activity.

Polynucleotides Encoding Antibodies

The invention further provides polynucleotides comprising a nucleotide sequence encoding an antibody of the invention and fragments thereof. The invention also encompasses polynucleotides that hybridize under stringent or lower stringency hybridization conditions, e.g., as defined supra, to polynucleotides that encode an antibody, preferably, that specifically binds to a polypeptide of the invention, preferably, an antibody that binds to a polypeptide having the amino acid sequence of SEQ ID NO:2.

The polynucleotides may be obtained, and the nucleotide sequence of the polynucleotides determined, by any method known in the art. For example, if the nucleotide sequence of the antibody is known, a polynucleotide encoding the antibody may be assembled from chemically synthesized oligonucleotides (e.g., as described in Kutmeier et al., BioTechniques 17:242 (1994)), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an antibody may be generated from nucleic acid from a suitable source. If a clone containing a nucleic acid encoding a particular antibody is not available, but the sequence of the antibody molecule is known, a nucleic acid encoding the immunoglobulin may be chemically synthesized or obtained from a suitable source (e.g., an antibody cDNA library, or a cDNA library generated from, or nucleic acid, preferably poly A+ RNA, isolated from, any tissue or cells expressing the antibody, such as hybridoma cells selected to express an antibody of the invention) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

Once the nucleotide sequence and corresponding amino acid sequence of the antibody is determined, the nucleotide sequence of the antibody may be manipulated using methods well known in the art for the manipulation of nucleotide sequences, e.g., recombinant DNA techniques, site directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, N.Y. and Ausubel et al., eds., 1998, Current Protocols in Molecular Biology, John Wiley & Sons, NY, which are both incorporated by reference herein in their entireties), to generate antibodies having a different amino acid sequence, for example to create amino acid substitutions, deletions, and/or insertions.

In a specific embodiment, the amino acid sequence of the heavy and/or light chain variable domains may be inspected to identify the sequences of the complementarity determining regions (CDRs) by methods that are well know in the art, e.g., by comparison to known amino acid sequences of other heavy and light chain variable regions to determine the regions of sequence hypervariability. Using routine recombinant DNA techniques, one or more of the CDRs may be inserted within framework regions, e.g., into human framework regions to humanize a non-human antibody, as described supra. The framework regions may be naturally occurring or consensus framework regions, and preferably human framework regions (see, e.g., Chothia et al., J. Mol. Biol. 278: 457-479 (1998) for a listing of human framework regions). Preferably, the polynucleotide generated by the combination of the framework regions and CDRs encodes an antibody that specifically binds a polypeptide of the invention. Preferably, as discussed supra, one or more amino acid substitutions may be made within the framework regions, and, preferably, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present invention and within the skill of the art.

In addition, techniques developed for the production of “chimeric antibodies” (Morrison et al., Proc. Natl. Acad. Sci. 81:851-855 (1984); Neuberger et al., Nature 312:604-608 (1984); Takeda et al., Nature 314:452-454 (1985)) by splicing genes from a mouse antibody molecule of appropriate antigen specificity together with genes from a human antibody molecule of appropriate biological activity can be used. As described supra, a chimeric antibody is a molecule in which different portions are derived from different animal species, such as those having a variable region derived from a murine mAb and a human immunoglobulin constant region, e.g., humanized antibodies.

Alternatively, techniques described for the production of single chain antibodies (U.S. Pat. No. 4,946,778; Bird, Science 242:423-42 (1988); Huston et al., Proc. Natl. Acad. Sci. USA 85:5879-5883 (1988); and Ward et al., Nature 334:544-54 (1989)) can be adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli may also be used (Skerra et al., Science 242:1038-1041 (1988)).

Methods of Producing Antibodies

The antibodies of the invention can be produced by any method known in the art for the synthesis of antibodies, in particular, by chemical synthesis or preferably, by recombinant expression techniques.

Recombinant expression of an antibody of the invention, or fragment, derivative or analog thereof, (e.g., a heavy or light chain of an antibody of the invention or a single chain antibody of the invention), requires construction of an expression vector containing a polynucleotide that encodes the antibody. Once a polynucleotide encoding an antibody molecule or a heavy or light chain of an antibody, or portion thereof (preferably containing the heavy or light chain variable domain), of the invention has been obtained, the vector for the production of the antibody molecule may be produced by recombinant DNA technology using techniques well known in the art. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, or a heavy or light chain thereof, or a heavy or light chain variable domain, operably linked to a promoter. Such vectors may include the nucleotide sequence encoding the constant region of the antibody molecule (see, e.g., PCT Publication WO 86/05807; PCT Publication WO 89/01036; and U.S. Pat. No. 5,122,464) and the variable domain of the antibody may be cloned into such a vector for expression of the entire heavy or light chain.

The expression vector is transferred to a host cell by conventional techniques and the transfected cells are then cultured by conventional techniques to produce an antibody of the invention. Thus, the invention includes host cells containing a polynucleotide encoding an antibody of the invention, or a heavy or light chain thereof, or a single chain antibody of the invention, operably linked to a heterologous promoter. In preferred embodiments for the expression of double-chained antibodies, vectors encoding both the heavy and light chains may be co-expressed in the host cell for expression of the entire immunoglobulin molecule, as detailed below.

A variety of host-expression vector systems may be utilized to express the antibody molecules of the invention. Such host-expression systems represent vehicles by which the coding sequences of interest may be produced and subsequently purified, but also represent cells which may, when transformed or transfected with the appropriate nucleotide coding sequences, express an antibody molecule of the invention in situ. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., Gene 45:101 (1986); Cockett et al., Bio/Technology 8:2 (1990)).

In bacterial systems, a number of expression vectors may be advantageously selected depending upon the use intended for the antibody molecule being expressed. For example, when a large quantity of such a protein is to be produced, for the generation of pharmaceutical compositions of an antibody molecule, vectors which direct the expression of high levels of fusion protein products that are readily purified may be desirable. Such vectors include, but are not limited, to the E. coli expression vector pUR278 (Ruther et al., EMBO J. 2:1791 (1983)), in which the antibody coding sequence may be ligated individually into the vector in frame with the lac Z coding region so that a fusion protein is produced; pIN vectors (Inouye & Inouye, Nucleic Acids Res. 13:3101-3109 (1985); Van Heeke & Schuster, J. Biol. Chem. 24:5503-5509 (1989)); and the like. pGEX vectors may also be used to express foreign polypeptides as fusion proteins with glutathione S-transferase (GST). In general, such fusion proteins are soluble and can easily be purified from lysed cells by adsorption and binding to matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts. (e.g., see Logan & Shenk, Proc. Natl. Acad. Sci. USA 81:355-359 (1984)). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., Methods in Enzymol. 153:51-544 (1987)).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, 293, 3T3, WI38, and in particular, breast cancer cell lines such as, for example, BT483, Hs578T, HTB2, BT20 and T47D, and normal mammary gland cell line such as, for example, CRL7030 and Hs578Bst.

For long-term, high-yield production of recombinant proteins, stable expression is preferred. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the antibody molecule. Such engineered cell lines may be particularly useful in screening and evaluation of compounds that interact directly or indirectly with the antibody molecule.

A number of selection systems may be used, including but not limited to the herpes simplex virus thymidine kinase (Wigler et al., Cell 11:223 (1977)), hypoxanthine-guanine phosphoribosyltransferase (Szybalska & Szybalski, Proc. Natl. Acad. Sci. USA 48:202 (1992)), and adenine phosphoribosyltransferase (Lowy et al., Cell 22:817 (1980)) genes can be employed in tk-, hgprt- or aprt-cells, respectively. Also, antimetabolite resistance can be used as the basis of selection for the following genes: dhfr, which confers resistance to methotrexate (Wigler et al., Natl. Acad. Sci. USA 77:357 (1980); O'Hare et al., Proc. Natl. Acad. Sci. USA 78:1527 (1981)); gpt, which confers resistance to mycophenolic acid (Mulligan & Berg, Proc. Natl. Acad. Sci. USA 78:2072 (1981)); neo, which confers resistance to the aminoglycoside G-418 Clinical Pharmacy 12:488-505; Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); May, 1993, TIB TECH 11(5):155-215); and hygro, which confers resistance to hygromycin (Santerre et al., Gene 30:147 (1984)). Methods commonly known in the art of recombinant DNA technology may be routinely applied to select the desired recombinant clone, and such Methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13; Dracopoli et al. (eds), Current Protocols in Human Genetics, John Wiley & Sons, NY (1994); Colberre-Garapin et al., J. Mol. Biol. 150:1 (1981), which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Vol.3. (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., Mol. Cell. Biol. 3:257 (1983)).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain (Proudfoot, Nature 322:52 (1986); Kohler, Proc. Natl. Acad. Sci. USA 77:2197 (1980)). The coding sequences for the heavy and light chains may comprise cDNA or genomic DNA.

Once an antibody molecule of the invention has been produced by an animal, chemically synthesized, or recombinantly expressed, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. In addition, the antibodies of the present invention or fragments thereof can be fused to heterologous polypeptide sequences described herein or otherwise known in the art, to facilitate purification.

The present invention encompasses antibodies recombinantly fused or chemically conjugated (including both covalently and non-covalently conjugations) to a polypeptide (or portion thereof, preferably at least 10, 20, 30, 40, 50, 60, 70, 80, 90 or 100 amino acids of the polypeptide) of the present invention to generate fusion proteins. The fusion does not necessarily need to be direct, but may occur through linker sequences. The antibodies may be specific for antigens other than polypeptides (or portion thereof, preferably at least 10, 20, 30, 40, 50, 60, 70, 80, 90 or 100 amino acids of the polypeptide) of the present invention. For example, antibodies may be used to target the polypeptides of the present invention to particular cell types, either in vitro or in vivo, by fusing or conjugating the polypeptides of the present invention to antibodies specific for particular cell surface receptors. Antibodies fused or conjugated to the polypeptides of the present invention may also be used in in vitro immunoassays and purification methods using methods known in the art. See e.g., Harbor et al., supra, and PCT publication WO 93/21232; EP 439,095; Naramura et al., Immunol. Lett. 39:91-99 (1994); U.S. Pat. No. 5,474,981; Gillies et al., PNAS 89:1428-1432 (1992); Fell et al., J. Immunol. 146:2446-2452(1991), which are incorporated by reference in their entireties.

The present invention further includes compositions comprising the polypeptides of the present invention fused or conjugated to antibody domains other than the variable regions. For example, the polypeptides of the present invention may be fused or conjugated to an antibody Fc region, or portion thereof. The antibody portion fused to a polypeptide of the present invention may comprise the constant region, hinge region, CH1 domain, CH2 domain, and CH3 domain or any combination of whole domains or portions thereof. The polypeptides may also be fused or conjugated to the above antibody portions to form multimers. For example, Fc portions fused to the polypeptides of the present invention can form dimers through disulfide bonding between the Fc portions. Higher multimeric forms can be made by fusing the polypeptides to portions of IgA and IgM. Methods for fusing or conjugating the polypeptides of the present invention to antibody portions are known in the art. See, e.g., U.S. Pat. Nos. 5,336,603; 5,622,929; 5,359,046; 5,349,053; 5,447,851; 5,112,946; EP 307,434; EP 367,166; PCT publications WO 96/04388; WO 91/06570; Ashkenazi et al., Proc. Natl. Acad. Sci. USA 88:10535-10539 (1991); Zheng et al., J. Immunol. 154:5590-5600 (1995); and Vil et al., Proc. Natl. Acad. Sci. USA 89:11337-11341(1992) (said references incorporated by reference in their entireties).

As discussed, supra, the polypeptides corresponding to a polypeptide, polypeptide fragment, or a variant of SEQ ID NO:Y may be fused or conjugated to the above antibody portions to increase the in vivo half life of the polypeptides or for use in immunoassays using methods known in the art. Further, the polypeptides corresponding to SEQ ID NO:Y may be fused or conjugated to the above antibody portions to facilitate purification. One reported example describes chimeric proteins consisting of the first two domains of the human CD4-polypeptide and various domains of the constant regions of the heavy or light chains of mammalian immunoglobulins. (EP 394,827; Traunecker et al., Nature 331:84-86 (1988). The polypeptides of the present invention fused or conjugated to an antibody having disulfide-linked dimeric structures (due to the IgG) may also be more efficient in binding and neutralizing other molecules, than the monomeric secreted protein or protein fragment alone. (Fountoulakis et al., J. Biochem. 270:3958-3964 (1995)). In many cases, the Fc part in a fusion protein is beneficial in therapy and diagnosis, and thus can result in, for example, improved pharmacokinetic properties. (EP A 232,262). Alternatively, deleting the Fc part after the fusion protein has been expressed, detected, and purified, would be desired. For example, the Fc portion may hinder therapy and diagnosis if the fusion protein is used as an antigen for immunizations. In drug discovery, for example, human proteins, such as hIL-5, have been fused with Fc portions for the purpose of high-throughput screening assays to identify antagonists of hIL-5. (See, Bennett et al., J. Molecular Recognition 8:52-58 (1995); Johanson et al., J. Biol. Chem. 270:9459-9471 (1995).

Moreover, the antibodies or fragments thereof of the present invention can be fused to marker sequences, such as a peptide to facilitate purification. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86:821-824 (1989), for instance, hexa-histidine provides for convenient purification of the fusion protein. Other peptide tags useful for purification include, but are not limited to, the “HA” tag, which corresponds to an epitope derived from the influenza hemagglutinin protein (Wilson et al., Cell 37:767 (1984)) and the “flag” tag.

The present invention further encompasses antibodies or fragments thereof conjugated to a diagnostic or therapeutic agent. The antibodies can be used diagnostically to, for example, monitor the development or progression of a tumor as part of a clinical testing procedure to, e.g., determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, radioactive materials, positron emitting metals using various positron emission tomographies, and nonradioactive paramagnetic metal ions. The detectable substance may be coupled or conjugated either directly to the antibody (or fragment thereof) or indirectly, through an intermediate (such as, for example, a linker known in the art) using techniques known in the art. See, for example, U.S. Pat. No. 4,741,900 for metal ions which can be conjugated to antibodies for use as diagnostics according to the present invention. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ¹¹¹In or ⁹⁹Tc.

Further, an antibody or fragment thereof may be conjugated to a therapeutic moiety such as a cytotoxin, e.g., a cytostatic or cytocidal agent, a therapeutic agent or a radioactive metal ion, e.g., alpha-emitters such as, for example, 213Bi. A cytotoxin or cytotoxic agent includes any agent that is detrimental to cells. Examples include paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the therapeutic agent or drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, a-interferon, β-interferon, nerve growth factor, platelet derived growth factor, tissue plasminogen activator, an apoptotic agent, e.g., TNF-alpha, TNF-beta, AIM I (See, International Publication No. WO 97/33899), AIM II (See, International Publication No. WO 97/34911), Fas Ligand (Takahashi et al., Int. Immunol., 6:1567-1574 (1994)), VEGI (See, International Publication No. WO 99/23105), a thrombotic agent or an anti-angiogenic agent, e.g., angiostatin or endostatin; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Antibodies may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev. 62:119-58 (1982).

Alternatively, an antibody can be conjugated to a second antibody to form an antibody heteroconjugate as described by Segal in U.S. Pat. No. 4,676,980, which is incorporated herein by reference in its entirety.

An antibody, with or without a therapeutic moiety conjugated to it, administered alone or in combination with cytotoxic factor(s) and/or cytokine(s) can be used as a therapeutic.

Immunophenotyping

The antibodies of the invention may be utilized for immunophenotyping of cell lines and biological samples. The translation product of the gene of the present invention may be useful as a cell specific marker, or more specifically as a cellular marker that is differentially expressed at various stages of differentiation and/or maturation of particular cell types. Monoclonal antibodies directed against a specific epitope, or combination of epitopes, will allow for the screening of cellular populations expressing the marker. Various techniques can be utilized using monoclonal antibodies to screen for cellular populations expressing the marker(s), and include magnetic separation using antibody-coated magnetic beads, “panning” with antibody attached to a solid matrix (i.e., plate), and flow cytometry (See, e.g., U.S. Pat. No. 5,985,660; and Morrison et al., Cell, 96:737-49 (1999)).

These techniques allow for the screening of particular populations of cells, such as might be found with hematological malignancies (i.e. minimal residual disease (MRD) in acute leukemic patients) and “non-self” cells in transplantations to prevent Graft-versus-Host Disease (GVHD). Alternatively, these techniques allow for the screening of hematopoietic stem and progenitor cells capable of undergoing proliferation and/or differentiation, as might be found in human umbilical cord blood.

Assays for Antibody Binding

The antibodies of the invention may be assayed for immunospecific binding by any method known in the art. The immunoassays which can be used include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al, eds, 1994, Current Protocols in Molecular Biology, Vol. 1, John Wiley & Sons, Inc., New York, which is incorporated by reference herein in its entirety). Exemplary immunoassays are described briefly below (but are not intended by way of limitation).

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 1-4 hours) at 4° C., adding protein A and/or protein G sepharose beads to the cell lysate, incubating for about an hour or more at 4° C., washing the beads in lysis buffer and resuspending the beads in SDS/sample buffer. The ability of the antibody of interest to immunoprecipitate a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with sepharose beads). For further discussion regarding immunoprecipitation protocols see, e.g., Ausubel et al, eds, 1994, Current Protocols in Molecular Biology, Vol. 1, John Wiley & Sons, Inc., New York at 10.16.1.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody (the antibody of interest) diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., 32P or 125I) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al, eds, 1994, Current Protocols in Molecular Biology, Vol. 1, John Wiley & Sons, Inc., New York at 10.8.1.

ELISAs comprise preparing antigen, coating the well of a 96 well microtiter plate with the antigen, adding-the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art. For further discussion regarding ELISAs see, e.g., Ausubel et al, eds, 1994, Current Protocols in Molecular Biology, Vol. 1, John Wiley & Sons, Inc., New York at 11.2.1.

The binding affinity of an antibody to an antigen and the off-rate of an antibody-antigen interaction can be determined by competitive binding assays. One example of a competitive binding assay is a radioimmunoassay comprising the incubation of labeled antigen (e.g., 3H or 125I) with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest conjugated to a labeled compound (e.g., 3H or 125I) in the presence of increasing amounts of an unlabeled second antibody.

Therapeutic Uses

The present invention is further directed to antibody-based therapies which involve administering antibodies of the invention to an animal, preferably a mammal, and most preferably a human, patient for treating one or more of the disclosed diseases, disorders, or conditions. Therapeutic compounds of the invention include, but are not limited to, antibodies of the invention (including fragments, analogs and derivatives thereof as described herein) and nucleic acids encoding antibodies of the invention (including fragments, analogs and derivatives thereof and anti-idiotypic antibodies as described herein). The antibodies of the invention can be used to treat, inhibit or prevent diseases, disorders or conditions associated with aberrant expression and/or activity of a polypeptide of the invention, including, but not limited to, any one or more of the diseases, disorders, or conditions described herein. The treatment and/or prevention of diseases, disorders, or conditions associated with aberrant expression and/or activity of a polypeptide of the invention includes, but is not limited to, alleviating symptoms associated with those diseases, disorders or conditions. Antibodies of the invention may be provided in pharmaceutically acceptable compositions as known in the art or as described herein.

A summary of the ways in which the antibodies of the present invention may be used therapeutically includes binding polynucleotides or polypeptides of the present invention locally or systemically in the body or by direct cytotoxicity of the antibody, e.g. as mediated by complement (CDC) or by effector cells (ADCC). Some of these approaches are described in more detail below. Armed with the teachings provided herein, one of ordinary skill in the art will know how to use the antibodies of the present invention for diagnostic, monitoring or therapeutic purposes without undue experimentation.

The antibodies of this invention may be advantageously utilized in combination with other monoclonal or chimeric antibodies, or with lymphokines or hematopoietic growth factors (such as, e.g., IL-2, IL-3 and IL-7), for example, which serve to increase the number or activity of effector cells which interact with the antibodies.

The antibodies of the invention may be administered alone or in combination with other types of treatments (e.g., radiation therapy, chemotherapy, hormonal therapy, immunotherapy and anti-tumor agents). Generally, administration of products of a species origin or species reactivity (in the case of antibodies) that is the same species as that of the patient is preferred. Thus, in a preferred embodiment, human antibodies, fragments derivatives, analogs, or nucleic acids, are administered to a human patient for therapy or prophylaxis.

It is preferred to use high affinity and/or potent in vivo inhibiting and/or neutralizing antibodies against polypeptides or polynucleotides of the present invention, fragments or regions thereof, for both immunoassays directed to and therapy of disorders related to polynucleotides or polypeptides, including fragments thereof, of the present invention. Such antibodies, fragments, or regions, will preferably have an affinity for polynucleotides or polypeptides of the invention, including fragments thereof. Preferred binding affinities include those with a dissociation constant or Kd less than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, and 10⁻¹⁵ M.

Gene Therapy

In a specific embodiment, nucleic acids comprising sequences encoding antibodies or functional derivatives thereof, are administered to treat, inhibit or prevent a disease or disorder associated with aberrant expression and/or activity of a polypeptide of the invention, by way of gene therapy. Gene therapy refers to therapy performed by the administration to a subject of an expressed or expressible nucleic acid. In this embodiment of the invention, the nucleic acids produce their encoded protein that mediates a therapeutic effect.

Any of the methods for gene therapy available in the art can be used according to the present invention. Exemplary methods are described below.

For general reviews of the methods of gene therapy, see Goldspiel et al., Clinical Pharmacy 12:488-505 (1993); Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); May, TIBTECH 11(5):155-215 (1993). Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

In a preferred aspect, the compound comprises nucleic acid sequences encoding an antibody, said nucleic acid sequences being part of expression vectors that express the antibody or fragments or chimeric proteins or heavy or light chains thereof in a suitable host. In particular, such nucleic acid sequences have promoters operably linked to the antibody coding region, said promoter being inducible or constitutive, and, optionally, tissue-specific. In another particular embodiment, nucleic acid molecules are used in which the antibody coding sequences and any other desired sequences are flanked by regions that promote homologous recombination at a desired site in the genome, thus providing for intrachromosomal expression of the antibody encoding nucleic acids (Koller and Smithies, Proc. Natl. Acad. Sci. USA 86:8932-8935 (1989); Zijlstra et al., Nature 342:435-438 (1989). In specific embodiments, the expressed antibody molecule is a single chain antibody; alternatively, the nucleic acid sequences include sequences encoding both the heavy and light chains, or fragments thereof, of the antibody.

Delivery of the nucleic acids into a patient may be either direct, in which case the patient is directly exposed to the nucleic acid or nucleic acid-carrying vectors, or indirect, in which case, cells are first transformed with the nucleic acids in vitro, then transplanted into the patient. These two approaches are known, respectively, as in vivo or ex vivo gene therapy.

In a specific embodiment, the nucleic acid sequences are directly administered in vivo, where it is expressed to produce the encoded product. This can be accomplished by any of numerous methods known in the art, e.g., by constructing them as part of an appropriate nucleic acid expression vector and administering it so that they become intracellular, e.g., by infection using defective or attenuated retrovirals or other viral vectors (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, J. Biol. Chem. 262:4429-4432 (1987)) (which can be used to target cell types specifically expressing the receptors), etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180; WO 92/22635; WO92/20316; WO93/14188, WO 93/20221). Alternatively, the nucleic acid can be introduced intracellularly and incorporated within host cell DNA for expression, by homologous recombination (Koller and Smithies, Proc. Natl. Acad. Sci. USA 86:8932-8935 (1989); Zijlstra et al., Nature 342:435-438 (1989)).

In a specific embodiment, viral vectors that contains nucleic acid sequences encoding an antibody of the invention are used. For example, a retroviral vector can be used (see Miller et al., Meth. Enzymol. 217:581-599 (1993)). These retroviral vectors contain the components necessary for the correct packaging of the viral genome and integration into the host cell DNA. The nucleic acid sequences encoding the antibody to be used in gene therapy are cloned into one or more vectors, which facilitates delivery of the gene into a patient. More detail about retroviral vectors can be found in Boesen et al., Biotherapy 6:291-302 (1994), which describes the use of a retroviral vector to deliver the mdr1 gene to hematopoietic stem cells in order to make the stem cells more resistant to chemotherapy. Other references illustrating the use of retroviral vectors in gene therapy are: Clowes et al., J. Clin. Invest. 93:644-651 (1994); Kiem et al., Blood 83:1467-1473 (1994); Salmons and Gunzberg, Human Gene Therapy 4:129-141 (1993); and Grossman and Wilson, Curr. Opin. in Genetics and Devel. 3:110-114 (1993).

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, Current Opinion in Genetics and Development 3:499-503 (1993) present a review of adenovirus-based gene therapy. Bout et al., Human Gene Therapy 5:3-10 (1994) demonstrated the use of adenovirus vectors to transfer genes to the respiratory epithelia of rhesus monkeys. Other instances of the use of adenoviruses in gene therapy can be found in Rosenfeld et al., Science 252:431-434 (1991); Rosenfeld et al., Cell 68:143-155 (1992); Mastrangeli et al., J. Clin. Invest. 91:225-234 (1993); PCT Publication WO94/12649; and Wang, et al., Gene Therapy 2:775-783 (1995). In a preferred embodiment, adenovirus vectors are used.

Adeno-associated virus (AAV) has also been proposed for use in gene therapy (Walsh et al., Proc. Soc. Exp. Biol. Med. 204:289-300 (1993); U.S. Pat. No. 5,436,146).

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler and Behr, Meth. Enzymol. 217:599-618 (1993); Cohen et al., Meth. Enzymol. 217:618-644 (1993); Cline, Pharmac. Ther. 29:69-92m (1985) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a patient by various methods known in the art. Recombinant blood cells (e.g., hematopoietic stem or progenitor cells) are preferably administered intravenously. The amount of cells envisioned for use depends on the desired effect, patient state, etc., and can be determined by one skilled in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as Tlymphocytes, Blymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc.

In a preferred embodiment, the cell used for gene therapy is autologous to the patient.

In an embodiment in which recombinant cells are used in gene therapy, nucleic acid sequences encoding an antibody are introduced into the cells such that they are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the present invention (see e.g. PCT Publication WO 94/08598; Stemple and Anderson, Cell 71:973-985 (1992); Rheinwald, Meth. Cell Bio. 21A:229 (1980); and Pittelkow and Scott, Mayo Clinic Proc. 61:771 (1986)).

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

Demonstration of Therapeutic or Prophylactic Activity

The compounds or pharmaceutical compositions of the invention are preferably tested in vitro, and then in vivo for the desired therapeutic or prophylactic activity, prior to use in humans. For example, in vitro assays to demonstrate the therapeutic or prophylactic utility of a compound or pharmaceutical composition include, the effect of a compound on a cell line or a patient tissue sample. The effect of the compound or composition on the cell line and/or tissue sample can be determined utilizing techniques known to those of skill in the art including, but not limited to, rosette formation assays and cell lysis assays. In accordance with the invention, in vitro assays which can be used to determine whether administration of a specific compound is indicated, include in vitro cell culture assays in which a patient tissue sample is grown in culture, and exposed to or otherwise administered a compound, and the effect of such compound upon the tissue sample is observed.

Therapeutic/Prophylactic Administration and Composition

The invention provides methods of treatment, inhibition and prophylaxis by administration to a subject of an effective amount of a compound or pharmaceutical composition of the invention, preferably an antibody of the invention. In a preferred aspect, the compound is substantially purified (e.g., substantially free from substances that limit its effect or produce undesired side-effects). The subject is preferably an animal, including but not limited to animals such as cows, pigs, horses, chickens, cats, dogs, etc., and is preferably a mammal, and most preferably human.

Formulations and methods of administration that can be employed when the compound comprises a nucleic acid or an immunoglobulin are described above; additional appropriate formulations and routes of administration can be selected from among those described herein below.

Various delivery systems are known and can be used to administer a compound of the invention, e.g., encapsulation in liposomes, microparticles, microcapsules, recombinant cells capable of expressing the compound, receptor-mediated endocytosis (see, e.g., Wu and Wu, J. Biol. Chem. 262:4429-4432 (1987)), construction of a nucleic acid as part of a retroviral or other vector, etc. Methods of introduction include but are not limited to intradermal, intramuscular, intraperitoneal, intravenous, subcutaneous, intranasal, epidural, and oral routes. The compounds or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compounds or compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compounds or compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, including an antibody, of the invention, care must be taken to use materials to which the protein does not absorb.

In another embodiment, the compound or composition can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, the compound or composition can be delivered in a controlled release system. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., Macromol. Sci. Rev. Macromol. Chem. 23:61 (1983); see also Levy et al., Science 228:190 (1985); During et al., Ann. Neurol. 25:351 (1989); Howard et al., J.Neurosurg. 71:105 (1989)). In yet another embodiment, a controlled release system can be placed in proximity of the therapeutic target, i.e., the brain, thus requiring only a fraction of the systemic dose (see, e.g., Goodson, in Medical Applications of Controlled Release, supra, vol. 2, pp. 115-138 (1984)).

Other controlled release systems are discussed in the review by Langer (Science 249:1527-1533 (1990)).

In a specific embodiment where the compound of the invention is a nucleic acid encoding a protein, the nucleic acid can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., Proc. Natl. Acad. Sci. USA 88:1864-1868 (1991)), etc. Alternatively, a nucleic acid can be introduced intracellularly and incorporated within host cell DNA for expression, by homologous recombination.

The present invention also provides pharmaceutical compositions. Such compositions comprise a therapeutically effective amount of a compound, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically scaled container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The amount of the compound of the invention which will be effective in the treatment, inhibition and prevention of a disease or disorder associated with aberrant expression and/or activity of a polypeptide of the invention can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses may be extrapolated from dose-response curves derived from in vitro or animal model test systems.

For antibodies, the dosage administered to a patient is typically 0.1 mg/kg to 100 mg/kg of the patient's body weight. Preferably, the dosage administered to a patient is between 0.1 mg/kg and 20 mg/kg of the patient's body weight, more preferably 1 mg/kg to 10 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the invention may be reduced by enhancing uptake and tissue penetration (e.g., into the brain) of the antibodies by modifications such as, for example, lipidation.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

Diagnosis and Imaging

Labeled antibodies, and derivatives and analogs thereof, which specifically bind to a polypeptide of interest can be used for diagnostic purposes to detect, diagnose, or monitor diseases and/or disorders associated with the aberrant expression and/or activity of a polypeptide of the invention. The invention provides for the detection of aberrant expression of a polypeptide of interest, comprising (a) assaying the expression of the polypeptide of interest in cells or body fluid of an individual using one or more antibodies specific to the polypeptide interest and (b) comparing the level of gene expression with a standard gene expression level, whereby an increase or decrease in the assayed polypeptide gene expression level compared to the standard expression level is indicative of aberrant expression.

The invention provides a diagnostic assay for diagnosing a disorder, comprising (a) assaying the expression of the polypeptide of interest in cells or body fluid of an individual using one or more antibodies specific to the polypeptide interest and (b) comparing the level of gene expression with a standard gene expression level, whereby an increase or decrease in the assayed polypeptide gene expression level compared to the standard expression level is indicative of a particular disorder. With respect to cancer, the presence of a relatively high amount of transcript in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier thereby preventing the development or further progression of the cancer.

Antibodies of the invention can be used to assay protein levels in a biological sample using classical immunohistological methods known to those of skill in the art (e.g., see Jalkanen, et al., J. Cell. Biol. 101:976-985 (1985); Jalkanen, et al., J. Cell. Biol. 105:3087-3096 (1987)). Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase; radioisotopes, such as iodine (125I, 121I), carbon (14C), sulfur (35S), tritium (3H), indium (112In), and technetium (99Tc); luminescent labels, such as luminol; and fluorescent labels, such as fluorescein and rhodamine, and biotin.

One aspect of the invention is the detection and diagnosis of a disease or disorder associated with aberrant expression of a polypeptide of interest in an animal, preferably a mammal and most preferably a human. In one embodiment, diagnosis comprises: a) administering (for example, parenterally, subcutaneously, or intraperitoneally) to a subject an effective amount of a labeled molecule which specifically binds to the polypeptide of interest; b) waiting for a time interval following the administering for permitting the labeled molecule to preferentially concentrate at sites in the subject where the polypeptide is expressed (and for unbound labeled molecule to be cleared to background level); c) determining background level; and d) detecting the labeled molecule in the subject, such that detection of labeled molecule above the background level indicates that the subject has a particular disease or disorder associated with aberrant expression of the polypeptide of interest. Background level can be determined by various methods including, comparing the amount of labeled molecule detected to a standard value previously determined for a particular system.

It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of 99mTc. The labeled antibody or antibody fragment will then preferentially accumulate at the location of cells which contain the specific protein. In vivo tumor imaging is described in S. W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982).

Depending on several variables, including the type of label used and the mode of administration, the time interval following the administration for permitting the labeled molecule to preferentially concentrate at sites in the subject and for unbound labeled molecule to be cleared to background level is 6 to 48 hours or 6 to 24 hours or 6 to 12 hours. In another embodiment the time interval following administration is 5 to 20 days or 5 to 10 days.

In an embodiment, monitoring of the disease or disorder is carried out by repeating the method for diagnosing the disease or disease, for example, one month after initial diagnosis, six months after initial diagnosis, one year after initial diagnosis, etc.

Presence of the labeled molecule can be detected in the patient using methods known in the art for in vivo scanning. These methods depend upon the type of label used. Skilled artisans will be able to determine the appropriate method for detecting a particular label. Methods and devices that may be used in the diagnostic methods of the invention include, but are not limited to, computed tomography (CT), whole body scan such as position emission tomography (PET), magnetic resonance imaging (MRI), and sonography.

In a specific embodiment, the molecule is labeled with a radioisotope and is detected in the patient using a radiation responsive surgical instrument (Thurston et al., U.S. Pat. No. 5,441,050). In another embodiment, the molecule is labeled with a fluorescent compound and is detected in the patient using a fluorescence responsive scanning instrument. In another embodiment, the molecule is labeled with a positron emitting metal and is detected in the patent using positron emission-tomography. In yet another embodiment, the molecule is labeled with a paramagnetic label and is detected in a patient using magnetic resonance imaging (MRI).

Kits

The present invention provides kits that can be used in the above methods. In one embodiment, a kit comprises an antibody of the invention, preferably a purified antibody, in one or more containers. In a specific embodiment, the kits of the present invention contain a substantially isolated polypeptide comprising an epitope which is specifically immunoreactive with an antibody included in the kit. Preferably, the kits of the present invention further comprise a control antibody which does not react with the polypeptide of interest. In another specific embodiment, the kits of the present invention contain a means for detecting the binding of an antibody to a polypeptide of interest (e.g., the antibody may be conjugated to a detectable substrate such as a fluorescent compound, an enzymatic substrate, a radioactive compound or a luminescent compound, or a second antibody which recognizes the first antibody may be conjugated to a detectable substrate).

In another specific embodiment of the present invention, the kit is a diagnostic kit for use in screening serum containing antibodies specific against proliferative and/or cancerous polynucleotides and polypeptides. Such a kit may include a control antibody that does not react with the polypeptide of interest. Such a kit may include a substantially isolated polypeptide antigen comprising an epitope which is specifically immunoreactive with at least one anti-polypeptide antigen antibody. Further, such a kit includes means for detecting the binding of said antibody to the antigen (e.g., the antibody may be conjugated to a fluorescent compound such as fluorescein or rhodamine which can be detected by flow cytometry). In specific embodiments, the kit may include a recombinantly produced or chemically synthesized polypeptide antigen. The polypeptide antigen of the kit may also be attached to a solid support.

In a more specific embodiment the detecting means of the above-described kit includes a solid support to which said polypeptide antigen is attached. Such a kit may also include a non-attached reporter-labeled anti-human antibody. In this embodiment, binding of the antibody to the polypeptide antigen can be detected by binding of the said reporter-labeled antibody.

In an additional embodiment, the invention includes a diagnostic kit for use in screening serum containing antigens of the polypeptide of the invention. The diagnostic kit includes a substantially isolated antibody specifically immunoreactive with polypeptide or polynucleotide antigens, and means for detecting the binding of the polynucleotide or polypeptide antigen to the antibody. In one embodiment, the antibody is attached to a solid support. In a specific embodiment, the antibody may be a monoclonal antibody. The detecting means of the kit may include a second, labeled monoclonal antibody. Alternatively, or in addition, the detecting means may include a labeled, competing antigen.

In one diagnostic configuration, test serum is reacted with a solid phase reagent having a surface-bound antigen obtained by the methods of the present invention. After binding with specific antigen antibody to the reagent and removing unbound serum components by washing, the reagent is reacted with reporter-labeled anti-human antibody to bind reporter to the reagent in proportion to the amount of bound anti-antigen antibody on the solid support. The reagent is again washed to remove unbound labeled antibody, and the amount of reporter associated with the reagent is determined. Typically, the reporter is an enzyme which is detected by incubating the solid phase in the presence of a suitable fluorometric, luminescent or colorimetric substrate (Sigma, St. Louis, Mo.).

The solid surface reagent in the above assay is prepared by known techniques for attaching protein material to solid support material, such as polymeric beads, dip sticks, 96-well plate or filter material. These attachment methods generally include non-specific adsorption of the protein to the support or covalent attachment of the protein, typically through a free amine group, to a chemically reactive group on the solid support, such as an activated carboxyl, hydroxyl, or aldehyde group. Alternatively, streptavidin coated plates can be used in conjunction with biotinylated antigen(s).

Thus, the invention provides an assay system or kit for carrying out this diagnostic method. The kit generally includes a support with surface-bound recombinant antigens, and a reporter-labeled anti-human antibody for detecting surface-bound anti-antigen antibody.

Fusion Polypeptides

Any PSF-2 polypeptide can be used to generate fusion polypeptides. For example, the PSF-2 polypeptide, when fused to a second polypeptide, can be used as an antigenic tag. Antibodies raised against the PSF-2 polypeptide can be used to indirectly detect the second polypeptide by binding to the PSF-2. Moreover, because secreted polypeptides target cellular locations based on trafficking signals, the PSF-2 polypeptides can be used as a targeting molecule once fused to other polypeptides.

Examples of domains that can be fused to PSF-2 polypeptides include not only heterologous signal sequences, but also other heterologous functional regions. The fusion does not necessarily need to be direct, but may occur through linker sequences.

Moreover, fusion polypeptides may also be engineered to improve characteristics of the PSF-2 polypeptide. For instance, a region of additional amino acids, particularly charged amino acids, may be added to the N-terminus of the PSF-2 polypeptide to improve stability and persistence during purification from the host cell or subsequent handling and storage. Also, peptide moieties may be added to the PSF-2 polypeptide to facilitate purification. Such regions may be removed prior to final preparation of the PSF-2 polypeptide. The addition of peptide moieties to facilitate handling of polypeptides are familiar and routine techniques in the art.

Moreover, PSF-2 polypeptides, including fragments, and specifically epitopes, can be combined with parts of the constant domain of immunoglobulins (IgG), resulting in chimeric polypeptides. These fusion polypeptides facilitate purification and show an increased half-life in vivo. One reported example describes chimeric polypeptides consisting of the first two domains of the human CD4-polypeptide and various domains of the constant regions of the heavy or light chains of mammalian immunoglobulins. (EP A 394,827; Traunecker et al., Nature 331:84-86 (1988).) Fusion polypeptides having disulfide-linked dimeric structures (due to the IgG) can also be more efficient in binding and neutralizing other molecules, than the monomeric secreted polypeptide or polypeptide fragment alone. (Fountoulakis et al., J. Biochem. 270:3958-3964 (1995).)

Similarly, EP-A-O 464 533 (Canadian counterpart 2045869) discloses fusion polypeptides comprising various portions of constant region of immunoglobulin molecules together with another human polypeptide or part thereof. In many cases, the Fc part in a fusion polypeptide is beneficial in therapy and diagnosis, and thus can result in, for example, improved pharmacokinetic properties. (EP-A 0232 262.) Alternatively, deleting the Fc part after the fusion polypeptide has been expressed, detected, and purified, would be desired. For example, the Fc portion may hinder therapy and diagnosis if the fusion polypeptide is used as an antigen for immunizations. In drug discovery, for example, human polypeptides, such as hIL-5, have been fused with Fc portions for the purpose of high-throughput screening assays to identify antagonists of hIL-5. (See, D. Bennett et al., J. Molecular Recognition 8:52-58 (1995); K. Johanson et al., J. Biol. Chem. 270:9459-9471 (1995).)

Moreover, the PSF-2 polypeptides can be fused to marker sequences, such as a peptide which facilitates purification of PSF-2. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86:821-824 (1989), for instance, hexa-histidine provides for convenient purification of the fusion polypeptide. Another peptide tag useful for purification, the “HA” tag, corresponds to an epitope derived from the influenza hemagglutinin polypeptide. (Wilson et al., Cell 37:767 (1984).)

Thus, any of these above fusions can be engineered using the PSF-2 polynucleotides or the polypeptides.

Vectors, Host Cells, and Polypeptide Production

The present invention also relates to vectors containing the PSF-2 polynucleotide, host cells, and the production of polypeptides by recombinant techniques. The vector may be, for example, a phage, plasmid, viral, or retroviral vector. Retroviral vectors may be replication competent or replication defective. In the latter case, viral propagation generally will occur only in complementing host cells.

PSF-2 polynucleotides may be joined to a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells.

The PSF-2 polynucleotide insert should be operatively linked to an appropriate promoter, such as the phage lambda PL promoter, the E. coli lac, trp, phoA and tac promoters, the SV40 early and late promoters and promoters of retroviral LTRs, to name a few. Other suitable promoters will be known to the skilled artisan. The expression constructs will further contain sites for transcription initiation, termination, and, in the transcribed region, a ribosome binding site for translation. The coding portion of the transcripts expressed by the constructs will preferably include a translation initiating codon at the beginning and a termination codon (UAA, UGA or UAG) appropriately positioned at the end of the polypeptide to be translated.

As indicated, the expression vectors will preferably include at least one selectable marker. Such markers include dihydrofolate reductase, G418 or neomycin resistance for eukaryotic cell culture and tetracycline, kanamycin or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells (e.g., Saccharomyces cerevisiae or Pichia pastoris (ATCC Accession No. 201178)); insect cells such as Drosophila S2 and Spodoptera Sf9 cells; animal cells such as CHO, COS, 293, and Bowes melanoma cells; and plant cells. Appropriate culture mediums and conditions for the above-described host cells are known in the art.

Among vectors preferred for use in bacteria include pHE4, pQE70, pQE60 and pQE-9, available from QIAGEN, Inc.; pBluescript vectors, Phagescript vectors, pNH8A, pNH16a, pNH18A, pNH46A, available from Stratagene Cloning Systems, Inc.; and ptrc99a, pKK223-3, pKK233-3, pDR540, pRIT5 available from Pharmacia Biotech, Inc. Among preferred eukaryotic vectors are pWLNEO, pSV2CAT, pOG44, pXT1 and pSG available from Stratagene; and pSVK3, pBPV, pMSG and pSVL available from Pharmacia. Preferred expression vectors for use in yeast systems include, but are not limited to, pYES2, pYD1, pTEF1/Zeo, pYES2/GS, pPICZ, pGAPZ, pGAPZalpha, pPIC9, pPIC3.5, pHIL-D2, pHIL-S1, pPIC3.5K, pPIC9K, and PAO815 (all available from Invitrogen, Carlsbad, Calif.). Other suitable vectors will be readily apparent to the skilled artisan.

Introduction of the construct into the host cell can be effected by calcium phosphate transfection, DEAE-dextran mediated transfection, cationic lipid-mediated transfection, electroporation, transduction, infection, or other methods. Such methods are described in many standard laboratory manuals, such as Davis et al., Basic Methods In Molecular Biology (1986). It is specifically contemplated that PSF-2 polypeptides may in fact be expressed by a host cell lacking a recombinant vector.

PSF-2 polypeptides can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification.

PSF-2 polypeptides, and preferably the secreted form, can also be recovered from: products purified from natural sources, including bodily fluids, tissues and cells, whether directly isolated or cultured; products of chemical synthetic procedures; and products produced by recombinant techniques from a prokaryotic or eukaryotic host, including, for example, bacterial, yeast, higher plant, insect, and mammalian cells.

In one embodiment, the yeast Pichia pastoris is used to express PSF-2 protein in a eukaryotic system. Pichia pastoris is a methylotrophic yeast which can metabolize methanol as its sole carbon source. A main step in the methanol metabolization pathway is the oxidation of methanol to formaldehyde using O₂. This reaction is catalyzed by the enzyme alcohol oxidase. In order to metabolize methanol as its sole carbon source, Pichia pastoris must generate high levels of alcohol oxidase due, in part, to the relatively low affinity of alcohol oxidase for O₂. Consequently, in a growth medium depending on methanol as a main carbon source, the promoter region of one of the two alcohol oxidase genes (AOX1) is highly active. In the presence of methanol, alcohol oxidase produced from the AOX1 gene comprises up to approximately 30% of the total soluble protein in Pichia pastoris. See, Ellis, S. B., et al., Mol. Cell. Biol. 5:1111-21 (1985); Koutz, P. J, et al., Yeast 5:167-77 (1989); Tschopp, J. F., et al., Nucl. Acids Res. 15:3859-76 (1987). Thus, a heterologous coding sequence, such as, for example, a PSF-2 polynucleotide of the present invention, under the transcriptional regulation of all or part of the AOX1 regulatory sequence is expressed at exceptionally high levels in Pichia yeast grown in the presence of methanol.

In one example, the plasmid vector pPIC9K is used to express DNA encoding a PSF-2 polypeptide of the invention, as set forth herein, in a Pichea yeast system essentially as described in “Pichia Protocols: Methods in Molecular Biology,” D. R. Higgins and J. Cregg, eds. The Humana Press, Totowa, N.J., 1998. This expression vector allows expression and secretion of a PSF-2 protein of the invention by virtue of the strong AOX1 promoter linked to the Pichia pastoris alkaline phosphatase (PHO) secretory signal peptide (i.e., leader) located upstream of a multiple cloning site.

Many other yeast vectors could be used in place of pPIC9K, such as, pYES2, pYD1, pTEF1/Zeo, pYES2/GS, pPICZ, pGAPZ, pGAPZalpha, pPIC9, pPIC3.5, pHIL-D2, pHIL-S1, pPIC3.5K, and PAO815, as one skilled in the art would readily appreciate, as long as the proposed expression construct provides appropriately located signals for transcription, translation, secretion (if desired), and the like, including an in-frame AUG as required.

In another embodiment, high-level expression of a heterologous coding sequence, such as, for example, a PSF-2 polynucleotide of the present invention, may be achieved by cloning the heterologous polynucleotide of the invention into an expression vector such as, for example, pGAPZ or pGAPZalpha, and growing the yeast culture in the absence of methanol.

Depending upon the host employed in a recombinant production procedure, the PSF-2 polypeptides may be glycosylated or may be non-glycosylated. In addition, PSF-2 polypeptides may also include an initial modified methionine residue, in some cases as a result of host-mediated processes. Thus, it is well known in the art that the N-terminal methionine encoded by the translation initiation codon generally is removed with high efficiency from any polypeptide after translation in all eukaryotic cells. While the N-terminal methionine on most polypeptides also is efficiently removed in most prokaryotes, for some polypeptides, this prokaryotic removal process is inefficient, depending on the nature of the amino acid to which the N-terminal methionine is covalently linked.

In addition to encompassing host cells containing the vector constructs discussed herein, the invention also encompasses primary, secondary, and immortalized host cells of vertebrate origin, particularly mammalian origin, that have been engineered to delete or replace endogenous genetic material (e.g., PSF-2 coding sequence), and/or to include genetic material (e.g., heterologous polynucleotide sequences) that is operably associated with PSF-2 polynucleotides of the invention, and which activates, alters, and/or amplifies endogenous PSF-2 polynucleotides. For example, techniques known in the art may be used to operably associate heterologous control regions (e.g., promoter and/or enhancer) and endogenous PSF-2 polynucleotide sequences via homologous recombination (see, e.g., U.S. Pat. No. 5,641,670, issued Jun. 24, 1997; International Publication No. WO 96/29411, published Sep. 26, 1996; International Publication No. WO 94/12650, published Aug. 4, 1994; Koller et al., Proc. Natl. Acad. Sci. USA 86:8932-8935 (1989); and Zijlstra et al., Nature 342:435-438 (1989), the disclosures of each of which are incorporated by reference in their entireties).

Uses of the PSF-2 Polynucleotides

The PSF-2 polynucleotides identified herein can be used in numerous ways as reagents. The following description should be considered exemplary and utilizes known techniques.

There exists an ongoing need to identify new chromosome markers, since few chromosome marking reagents, based on actual sequence data (repeat polymorphisms), are presently available. Clone HMKEA94 can be chromosomally mapped to a specific human chromosome. Then, PSF-2 polynucleotides can be used in linkage analysis as markers for that specific chromosome.

Briefly, sequences can be mapped to chromosomes by preparing PCR primers (preferably 15-25 bp) from the sequences shown in SEQ ID NO:1. Primers can be selected using computer analysis so that primers do not span more than one predicted exon in the genomic DNA. These primers are then used for PCR screening of somatic cell hybrids containing individual human chromosomes. Only those hybrids containing the human PSF-2 gene corresponding to the SEQ ID NO:1 will yield an amplified fragment.

Similarly, somatic hybrids provide a rapid method of PCR mapping the polynucleotides to particular chromosomes. Three or more clones can be assigned per day using a single thermal cycler. Moreover, sublocalization of the PSF-2 polynucleotides can be achieved with panels of specific chromosome fragments. Other gene mapping strategies that can be used include in situ hybridization, prescreening with labeled flow-sorted chromosomes, and preselection by hybridization to construct chromosome specific-cDNA libraries.

Precise chromosomal location of the PSF-2 polynucleotides can also be achieved using fluorescence in situ hybridization (FISH) of a metaphase chromosomal spread. This technique uses polynucleotides as short as 500 or 600 bases; however, polynucleotides 2,000-4,000 bp are preferred. For a review of this technique, see Verma et al., “Human Chromosomes: a Manual of Basic Techniques,” Pergamon Press, New York (1988).

For chromosome mapping, the PSF-2 polynucleotides can be used individually (to mark a single chromosome or a single site on that chromosome) or in panels (for marking multiple sites and/or multiple chromosomes). Preferred polynucleotides correspond to the noncoding regions of the cDNAs because the coding sequences are more likely conserved within gene families, thus increasing the chance of cross hybridization during chromosomal mapping.

Once a polynucleotide has been mapped to a precise chromosomal location, the physical position of the polynucleotide can be used in linkage analysis. Linkage analysis establishes coinheritance between a chromosomal location and presentation of a particular disease. (Disease mapping data are found, for example, in V. McKusick, Mendelian Inheritance in Man (available on line through Johns Hopkins University Welch Medical Library).) Assuming 1 megabase mapping resolution and one gene per 20 kb, a cDNA precisely localized to a chromosomal region associated with the disease could be one of 50-500 potential causative genes.

Thus, once coinheritance is established, differences in the PSF-2 polynucleotide and the corresponding gene between affected and unaffected individuals can be examined. First, visible structural alterations in the chromosomes, such as deletions or translocations, are examined in chromosome spreads or by PCR. If no structural alterations exist, the presence of point mutations are ascertained. Mutations observed in some or all affected individuals, but not in normal individuals, indicates that the mutation may cause the disease. However, complete sequencing of the PSF-2 polypeptide and the corresponding gene from several normal individuals is required to distinguish the mutation from a polymorphism. If a new polymorphism is identified, this polymorphic polypeptide can be used for further linkage analysis.

Furthermore, increased or decreased expression of the gene in affected individuals as compared to unaffected individuals can be assessed using PSF-2 polynucleotides. Any of these alterations (altered expression, chromosomal rearrangement, or mutation) can be used as a diagnostic or prognostic marker.

In addition to the foregoing, a PSF-2 polynucleotide can be used to control gene expression through triple helix formation or antisense DNA or RNA. Both methods rely on binding of the polynucleotide to DNA or RNA. For these techniques, preferred polynucleotides are usually 20 to 40 bases in length and complementary to either the region of the gene involved in transcription (triple helix—see Lee et al., Nucl. Acids Res. 6:3073 (1979); Cooney et al., Science 241:456 (1988); and Dervan et al., Science 251:1360 (1991) ) or to the mRNA itself (antisense—Okano, J. Neurochem. 56:560 (1991); Oligodeoxy-nucleotides as Antisense Inhibitors of Gene Expression, CRC Press, Boca Raton, Fla. (1988).) Triple helix formation optimally results in a shut-off of RNA transcription from DNA, while antisense RNA hybridization blocks translation of an mRNA molecule into polypeptide. Both techniques are effective in model systems, and the information disclosed herein can be used to design antisense or triple helix polynucleotides in an effort to treat or prevent disease.

PSF-2 polynucleotides are also useful in gene therapy. One goal of gene therapy is to insert a normal gene into an organism having a defective gene, in an effort to correct the genetic defect. PSF-2 offers a means of targeting such genetic defects in a highly accurate manner. Another goal is to insert a new gene that was not present in the host genome, thereby producing a new trait in the host cell.

The PSF-2 polynucleotides are also useful for identifying individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identifying personnel. This method does not suffer from the current limitations of “Dog Tags” which can be lost, switched, or stolen, making positive identification difficult. The PSF-2 polynucleotides can be used as additional DNA markers for RFLP.

The PSF-2 polynucleotides can also be used as an alternative to RFLP, by determining the actual base-by-base DNA sequence of selected portions of an individual's genome. These sequences can be used to prepare PCR primers for amplifying and isolating such selected DNA, which can then be sequenced. Using this technique, individuals can be identified because each individual will have a unique set of DNA sequences. Once an unique ID database is established for an individual, positive identification of that individual, living or dead, can be made from extremely small tissue samples.

Forensic biology also benefits from using DNA-based identification techniques as disclosed herein. DNA sequences taken from very small biological samples such as tissues, e.g., hair or skin, or body fluids, e.g., blood, saliva, semen, etc., can be amplified using PCR. In one prior art technique, gene sequences amplified from polymorphic loci, such as DQa class II HLA gene, are used in forensic biology to identify individuals. (Erlich, H., PCR Technology, Freeman and Co. (1992).) Once these specific polymorphic loci are amplified, they are digested with one or more restriction enzymes, yielding an identifying set of bands on a Southern blot probed with DNA corresponding to the DQa class II HLA gene. Similarly, PSF-2 polynucleotides can be used as polymorphic markers for forensic purposes.

There is also a need for reagents capable of identifying the source of a particular tissue. Such need arises, for example, in forensics when presented with tissue of unknown origin. Appropriate reagents can comprise, for example, DNA probes or primers specific to particular tissue prepared from PSF-2 sequences. Panels of such reagents can identify tissue by species and/or by organ type. In a similar fashion, these reagents can be used to screen tissue cultures for contamination.

There are two primary transcripts visible on Northern blots (approximately 2 and 3.5 kb in size). The highest levels of expression clearly in spleen, while lower levels of expression are visible in a variety of tissues examined, including prostate, testis, colon, stomach, thyroid, small intestine. There is no obvious expression in peripheral blood cells; likely to be expressed by endothelial cells., PSF-2 polynucleotides are useful as hybridization probes for differential identification of the tissue(s) or cell type(s) present in a biological sample. Similarly, polypeptides and antibodies directed to PSF-2 polypeptides are useful to provide immunological probes for differential identification of the tissue(s) or cell type(s). In addition, for a number of disorders of the above tissues or cells, particularly of the vascular and/or immune system, significantly higher or lower levels of PSF-2 gene expression may be detected in certain tissues (e.g., cancerous and wounded tissues) or bodily fluids (e.g., serum, plasma, urine, synovial fluid or spinal fluid) taken from an individual having such a disorder, relative to a “standard” PSF-2 gene expression level, i.e., the PSF-2 expression level in healthy tissue from an individual not having the vascular and/or immune system disorder.

Thus, the invention provides a diagnostic method of a disorder, which involves: (a) assaying PSF-2 gene expression level in cells or body fluid of an individual; (b) comparing the PSF-2 gene expression level with a standard PSF-2 gene expression level, whereby an increase or decrease in the assayed PSF-2 gene expression level compared to the standard expression level is indicative of disorder in the vascular and/or immune system.

In the very least, the PSF-2 polynucleotides can be used as molecular weight markers on Southern gels, as diagnostic probes for the presence of a specific mRNA in a particular cell type, as a probe to “subtract-out” known sequences in the process of discovering novel polynucleotides, for selecting and making oligomers for attachment to a “gene chip” or other support, to raise anti-DNA antibodies using DNA immunization techniques, and as an antigen to elicit an immune response.

Uses of PSF-2 Polypeptides

PSF-2 polypeptides can be used in numerous ways. The following description should be considered exemplary and utilizes known techniques.

PSF-2 polypeptides can be used to assay polypeptide levels in a biological sample using antibody-based techniques. For example, polypeptide expression in tissues can be studied with classical immunohistological methods. (Jalkanen, M., et al., J. Cell. Biol. 101:976-985 (1985); Jalkanen, M., et al., J. Cell. Biol. 105:3087-3096 (1987).) Other antibody-based methods useful for detecting polypeptide gene expression include immunoassays, such as the enzyme linked immunosorbent assay (ELISA) and the radioimmunoassay (RIA). Suitable antibody assay labels are known in the art and include enzyme labels, such as, glucose oxidase, and radioisotopes, such as iodine (¹²⁵I, ¹²¹I), carbon (¹⁴C), sulfur (³⁵S), tritium (³H), indium (¹¹²In), and technetium (⁹⁹mTc), and fluorescent labels, such as fluorescein and rhodamine, and biotin.

In addition to assaying secreted polypeptide levels in a biological sample, polypeptides can also be detected in vivo by imaging. Antibody labels or markers for in vivo imaging of polypeptide include those detectable by X-radiography, NMR or ESR. For X-radiography, suitable labels include radioisotopes such as barium or cesium, which emit detectable radiation but are not overtly harmful to the subject. Suitable markers for NMR and ESR include those with a detectable characteristic spin, such as deuterium, which may be incorporated into the antibody by labeling of nutrients for the relevant hybridoma.

A polypeptide-specific antibody or antibody fragment which has been labeled with an appropriate detectable imaging moiety, such as a radioisotope (for example, ¹³¹I, ¹¹²In, ⁹⁹mTc), a radio-opaque substance, or a material detectable by nuclear magnetic resonance, is introduced (for example, parenterally, subcutaneously, or intraperitoneally) into the mammal. It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ⁹⁹mTc. The labeled antibody or antibody fragment will then preferentially accumulate at the location of cells which contain the specific polypeptide. In vivo tumor imaging is described in S. W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982).)

Thus, the invention provides a diagnostic method of a disorder, which involves (a) assaying the expression of PSF-2 polypeptide in cells or body fluid of an individual; (b) comparing the level of gene expression with a standard gene expression level, whereby an increase or decrease in the assayed PSF-2 polypeptide gene expression level compared to the standard expression level is indicative of a disorder.

Moreover, PSF-2 polypeptides can be used to treat, prevent, and/or diagnose disease. For example, patients can be administered PSF-2 polypeptides in an effort to replace absent or decreased levels of the PSF-2 polypeptide (e.g., insulin), to supplement absent or decreased levels of a different polypeptide (e.g., hemoglobin S for hemoglobin B), to inhibit the activity of a polypeptide (e.g., an oncogene), to activate the activity of a polypeptide (e.g., by binding to a receptor), to reduce the activity of a membrane bound receptor by competing with it for free ligand (e.g., soluble TNF receptors used in reducing inflammation), or to bring about a desired response (e.g., blood vessel growth).

Similarly, antibodies directed to PSF-2 polypeptides can also be used to treat, prevent, and/or diagnose disease. For example, administration of an antibody directed to a PSF-2 polypeptide can bind and reduce overproduction of the polypeptide. Similarly, administration of an antibody can activate the polypeptide, such as by binding to a polypeptide bound to a membrane (receptor).

At the very least, the PSF-2 polypeptides can be used as molecular weight markers on SDS-PAGE gels or on molecular sieve gel filtration columns using methods well known to those of skill in the art. PSF-2 polypeptides can also be used to raise antibodies, which in turn are used to measure polypeptide expression from a recombinant cell, as a way of assessing transformation of the host cell. Moreover, PSF-2 polypeptides can be used to test the following biological activities.

Biological Activities of PSF-2

PSF-2 polynucleotides and polypeptides can be used in assays to test for one or more biological activities. If PSF-2 polynucleotides and polypeptides do exhibit activity in a particular assay, it is likely that PSF-2 may be involved in the diseases associated with the biological activity. Therefore, PSF-2 could be used to treat, prevent, and/or diagnose the associated disease.

Immune Activity

PSF-2 polypeptides or polynucleotides may be useful in treating deficiencies or disorders of the immune system, by activating or inhibiting the proliferation, differentiation, or mobilization (chemotaxis) of immune cells. Immune cells develop through a process called hematopoiesis, producing myeloid (platelets, red blood cells, neutrophils, and macrophages) and lymphoid (B and T lymphocytes) cells from pluripotent stem cells. The etiology of these immune deficiencies or disorders may be genetic, somatic, such as cancer or some autoimmune disorders, acquired (e.g., by chemotherapy or toxins), or infectious. Moreover, PSF-2 polynucleotides or polypeptides can be used as a marker or detector of a particular immune system disease or disorder.

PSF-2 polynucleotides or polypeptides may be useful in treating, preventing, and/or diagnosing deficiencies or disorders of hematopoietic cells. PSF-2 polypeptides or polynucleotides could be used to increase differentiation and proliferation of hematopoietic cells, including the pluripotent stem cells, in an effort to treat or prevent those disorders associated with a decrease in certain (or many) types hematopoietic cells. Examples of immunologic deficiency syndromes include, but are not limited to: blood polypeptide disorders (e.g. agammaglobulinemia, dysgammaglobulinemia), ataxia telangiectasia, common variable immunodeficiency, Digeorge Syndrome, HIV infection, HTLV-BLV infection, leukocyte adhesion deficiency syndrome, lymphopenia, phagocyte bactericidal dysfunction, severe combined immunodeficiency (SCIDs), Wiskott-Aldrich Disorder, anemia, thrombocytopenia, or hemoglobinuria.

Moreover, PSF-2 polypeptides or polynucleotides can also be used to modulate hemostatic (the stopping of bleeding) or thrombolytic activity (clot formation). For example, by increasing hemostatic or thrombolytic activity, PSF-2 polynucleotides or polypeptides could be used to treat or prevent blood coagulation disorders (e.g., afibrinogenemia, factor deficiencies), blood platelet disorders (e.g. thrombocytopenia), or wounds resulting from trauma, surgery, or other causes. Alternatively, PSF-2 polynucleotides or polypeptides that can decrease hemostatic or thrombolytic activity could be used to inhibit or dissolve clotting, important in the treatment or prevention of heart attacks (infarction), strokes, or scarring.

PSF-2 polynucleotides or polypeptides may also be useful in treating, preventing, and/or diagnosing autoimmune disorders. Many autoimmune disorders result from inappropriate recognition of self as foreign material by immune cells. This inappropriate recognition results in an immune response leading to the destruction of the host tissue. Therefore, the administration of PSF-2 polypeptides or polynucleotides that can inhibit an immune response, particularly the proliferation, differentiation, or chemotaxis of T-cells, may be an effective therapy in preventing autoimmune disorders.

Examples of autoimmune disorders that can be treated, prevented, and/or diagnosed or detected by PSF-2 include, but are not limited to: Addison's Disease, hemolytic anemia, antiphospholipid syndrome, rheumatoid arthritis, dermatitis, allergic encephalomyelitis, glomerulonephritis, Goodpasture's Syndrome, Graves' Disease, Multiple Sclerosis, Myasthenia Gravis, Neuritis, Ophthalmia, Bullous Pemphigoid, Pemphigus, Polyendocrinopathies, Purpura, Reiter's Disease, Stiff-Man Syndrome, Autoimmune Thyroiditis, Systemic Lupus Erythematosus, Autoimmune Pulmonary Inflammation, Guillain-Barre Syndrome, insulin dependent diabetes mellitis, and autoimmune inflammatory eye disease.

Similarly, allergic reactions and conditions, such as asthma (particularly allergic asthma) or other respiratory problems, may also be treated, prevented, and/or diagnosed by PSF-2 polypeptides or polynucleotides. Moreover, PSF-2 can be used to treat anaphylaxis, hypersensitivity to an antigenic molecule, or blood group incompatibility.

PSF-2 polynucleotides or polypeptides may also be used to treat, prevent, and/or diagnose organ rejection or graft-versus-host disease (GVHD). Organ rejection occurs by host immune cell destruction of the transplanted tissue through an immune response. Similarly, an immune response is also involved in GVHD, but, in this case, the foreign transplanted immune cells destroy the host tissues. The administration of TPSF-2 polypeptides or polynucleotides that inhibits an immune response, particularly the proliferation, differentiation, or chemotaxis of T-cells, may be an effective therapy in preventing organ rejection or GVHD.

Similarly, PSF-2 polypeptides or polynucleotides may also be used to modulate inflammation. For example, PSF-2 polypeptides or polynucleotides may inhibit the proliferation and differentiation of cells involved in an inflammatory response. These molecules can be used to treat, prevent, and/or diagnose inflammatory conditions, both chronic and acute conditions, including inflammation associated with infection (e.g., septic shock, sepsis, or systemic inflammatory response syndrome (SIRS)), ischemia-reperfusion injury, endotoxin lethality, arthritis, complement-mediated hyperacute rejection, nephritis, cytokine or chemokine induced lung injury, inflammatory bowel disease, Crohn's disease, or resulting from over production of cytokines (e.g., TNF or IL-1.)

Hyperproliferative Disorders

PSF-2 polypeptides or polynucleotides can be used to treat, prevent, and/or diagnose hyperproliferative disorders, including neoplasms. PSF-2 polypeptides or polynucleotides may inhibit the proliferation of the disorder through direct or indirect interactions. Alternatively, PSF-2 polypeptides or polynucleotides may proliferate other cells which can inhibit the hyperproliferative disorder.

For example, by increasing an immune response, particularly increasing antigenic qualities of the hyperproliferative disorder or by proliferating, differentiating, or mobilizing T-cells, hyperproliferative disorders can be treated, prevented, and/or diagnosed. This immune response may be increased by either enhancing an existing immune response, or by initiating a new immune response. Alternatively, decreasing an immune response may also be a method of treating, preventing, and/or diagnosing hyperproliferative disorders, such as a chemotherapeutic agent.

Examples of hyperproliferative disorders that can be treated, prevented, and/or diagnosed by PSF-2 polynucleotides or polypeptides include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, and urogenital.

Similarly, other hyperproliferative disorders can also be treated, prevented, and/or diagnosed by PSF-2 polynucleotides or polypeptides. Examples of such hyperproliferative disorders include, but are not limited to: hypergammaglobulinemia, lymphoproliferative disorders, parapolypeptideemias, purpura, sarcoidosis, Sezary Syndrome, Waldenstron's Macroglobulinemia, Gaucher's Disease, histiocytosis, and any other hyperproliferative disease, besides neoplasia, located in an organ system listed above.

Infectious Disease

PSF-2 polypeptides or polynucleotides can be used to treat, prevent, and/or diagnose infectious agents. For example, by increasing the immune response, particularly increasing the proliferation and differentiation of B and/or T cells, infectious diseases may be treated, prevented, and/or diagnosed. The immune response may be increased by either enhancing an existing immune response, or by initiating a new immune response. Alternatively, PSF-2 polypeptides or polynucleotides may also directly inhibit the infectious agent, without necessarily eliciting an immune response.

Viruses are one example of an infectious agent that can cause disease or symptoms that can be treated, prevented, and/or diagnosed by PSF-2 polynucleotides or polypeptides. Examples of viruses, include, but are not limited to the following DNA and RNA viral families: Arbovirus, Adenoviridae, Arenaviridae, Arterivirus, Birnaviridae, Bunyaviridae, Caliciviridae, Circoviridae, Coronaviridae, Flaviviridae, Hepadnaviridae (Hepatitis), Herpesviridae (such as, Cytomegalovirus, Herpes Simplex, Herpes Zoster), Mononegavirus (e.g., Paramyxoviridae, Morbillivirus, Rhabdoviridae), Orthomyxoviridae (e.g., Influenza), Papovaviridae, Parvoviridae, Picomaviridae, Poxviridae (such as Smallpox or Vaccinia), Reoviridae (e.g., Rotavirus), Retroviridae (HTLV-I, HTLV-II, Lentivirus), and Togaviridae (e.g., Rubivirus). Viruses falling within these families can cause a variety of diseases or symptoms, including, but not limited to: arthritis, bronchiollitis, encephalitis, eye infections (e.g., conjunctivitis, keratitis), chronic fatigue syndrome, hepatitis (A, B, C, E, Chronic Active, Delta), meningitis, opportunistic infections (e.g., AIDS), pneumonia, Burkitt's Lymphoma, chickenpox, hemorrhagic fever, Measles, Mumps, Parainfluenza, Rabies, the common cold, Polio, leukemia, Rubella, sexually transmitted diseases, skin diseases (e.g., Kaposi's, warts), and viremia. PSF-2 polypeptides or polynucleotides can be used to treat, prevent, and/or diagnose any of these symptoms or diseases.

Similarly, bacterial or fungal agents that can cause disease or symptoms and that can be treated, prevented, and/or diagnosed by PSF-2 polynucleotides or polypeptides include, but not limited to, the following Gram-Negative and Gram-positive bacterial families and fungi: Actinomycetales (e.g., Corynebacterium, Mycobacterium, Norcardia), Aspergillosis, Bacillaceae (e.g., Anthrax, Clostridium), Bacteroidaceae, Blastomycosis, Bordetella, Borrelia, Brucellosis, Candidiasis, Campylobacter, Coccidioidomycosis, Cryptococcosis, Dermatocycoses, Enterobacteriaceae (Klebsiella, Salmonella, Serratia, Yersinia), Erysipelothrix, Helicobacter, Legionellosis, Leptospirosis, Listeira, Mycoplasmatales, Neisseriaceae (e.g., Acinetobacter, Gonorrhea, Menigococcal), Pasteurellacea Infections (e.g., Actinobacillus, Heamophilus, Pasteurella), Pseudomonas, Rickettsiaceae, Chlamydiaceae, Syphilis, and Staphylococcal. These bacterial or fungal families can cause the following diseases or symptoms, including, but not limited to: bacteremia, endocarditis, eye infections (conjunctivitis, tuberculosis, uveitis), gingivitis, opportunistic infections (e.g., AIDS related infections), paronychia, prosthesis-related infections, Reiter's Disease, respiratory tract infections, such as Whooping Cough or Empyema, sepsis, Lyme Disease, Cat-Scratch Disease, Dysentery, Paratyphoid Fever, food poisoning, Typhoid, pneumonia, Gonorrhea, meningitis, Chlamydia, Syphilis, Diphtheria, Leprosy, Paratuberculosis, Tuberculosis, Lupus, Botulism, gangrene, tetanus, impetigo, Rheumatic Fever, Scarlet Fever, sexually transmitted diseases, skin diseases (e.g., cellulitis, dermatocycoses), toxemia, urinary tract infections, wound infections. PSF-2 polypeptides or polynucleotides can be used to treat, prevent, and/or diagnose any of these symptoms or diseases.

Moreover, parasitic agents causing disease or symptoms that can be treated, prevented, and/or diagnosed by PSF-2 polynucleotides or polypeptides include, but not limited to, the following families: Amebiasis, Babesiosis, Coccidiosis, Cryptosporidiosis, Dientamoebiasis, Dourine, Ectoparasitic, Giardiasis, Helminthiasis, Leishmaniasis, Theileriasis, Toxoplasmosis, Trypanosomiasis, and Trichomonas. These parasites can cause a variety of diseases or symptoms, including, but not limited to: Scabies, Trombiculiasis, eye infections, intestinal disease (e.g., dysentery, giardiasis), liver disease, lung disease, opportunistic infections (e.g., AIDS related), Malaria, pregnancy complications, and toxoplasmosis. PSF-2 polypeptides or polynucleotides can be used to treat, prevent, and/or diagnose any of these symptoms or diseases.

Preferably, treatment or prevention using PSF-2 polypeptides or polynucleotides could either be by administering an effective amount of PSF-2 polypeptide to the patient, or by removing cells from the patient, supplying the cells with PSF-2 polynucleotide, and returning the engineered cells to the patient (ex vivo therapy). Moreover, the PSF-2 polypeptide or polynucleotide can be used as an antigen in a vaccine to raise an immune response against infectious disease.

Regeneration

PSF-2 polynucleotides or polypeptides can be used to differentiate, proliferate, and attract cells, leading to the regeneration of tissues. (See, Science 276:59-87 (1997).) The regeneration of tissues could be used to repair, replace, or protect tissue damaged by congenital defects, trauma (wounds, burns, incisions, or ulcers), age, disease (e.g. osteoporosis, osteocarthritis, periodontal disease, liver failure), surgery, including cosmetic plastic surgery, fibrosis, reperfusion injury, or systemic cytokine damage.

Tissues that could be regenerated using the present invention include organs (e.g., pancreas, liver, intestine, kidney, skin, endothelium), muscle (smooth, skeletal or cardiac), vascular (including vascular endothelium), nervous, hematopoietic, and skeletal (bone, cartilage, tendon, and ligament) tissue. Preferably, regeneration occurs without or decreased scarring. Regeneration also may include angiogenesis.

Moreover, PSF-2 polynucleotides or polypeptides may increase regeneration of tissues difficult to heal. For example, increased tendon/ligament regeneration would quicken recovery time after damage. PSF-2 polynucleotides or polypeptides of the present invention could also be used prophylactically in an effort to avoid damage. Specific diseases that could be treated, prevented, and/or diagnosed include of tendinitis, carpal tunnel syndrome, and other tendon or ligament defects. A further example of tissue regeneration of non-healing wounds includes pressure ulcers, ulcers associated with vascular insufficiency, surgical, and traumatic wounds.

Similarly, nerve and brain tissue could also be regenerated by using PSF-2 polynucleotides or polypeptides to proliferate and differentiate nerve cells. Diseases that could be treated, prevented, and/or diagnosed using this method include central and peripheral nervous system diseases, neuropathies, or mechanical and traumatic disorders (e.g., spinal cord disorders, head trauma, cerebrovascular disease, and stoke). Specifically, diseases associated with peripheral nerve injuries, peripheral neuropathy (e.g., resulting from chemotherapy or other medical therapies), localized neuropathies, and central nervous system diseases (e.g., Alzheimer's disease, Parkinson's disease, Huntington's disease, amyotrophic lateral sclerosis, and Shy-Drager syndrome), could all be treated, prevented, and/or diagnosed using the PSF-2 polynucleotides or polypeptides.

Clemotaxis

PSF-2 polynucleotides or polypeptides may have chemotaxis activity. A chemotaxic molecule attracts or mobilizes cells (e.g., monocytes, fibroblasts, neutrophils, T-cells, mast cells, eosinophils, epithelial and/or endothelial cells) to a particular site in the body, such as inflammation, infection, or site of hyperproliferation. The mobilized cells can then fight off and/or heal the particular trauma or abnormality.

PSF-2 polynucleotides or polypeptides may increase chemotaxic activity of particular cells. These chemotactic molecules can then be used to treat, prevent, and/or diagnose inflammation, infection, hyperproliferative disorders, or any immune system disorder by increasing the number of cells targeted to a particular location in the body. For example, chemotaxic molecules can be used to treat, prevent, and/or diagnose wounds and other trauma to tissues by attracting immune cells to the injured location. As a chemotactic molecule, PSF-2 could also attract fibroblasts, which can be used to treat, prevent, and/or diagnose wounds.

It is also contemplated that PSF-2 polynucleotides or polypeptides may inhibit chemotactic activity. These molecules could also be used to treat, prevent, and/or diagnose disorders. Thus, PSF-2 polynucleotides or polypeptides could be used as an inhibitor of chemotaxis.

Binding Activity

PSF-2 polypeptides may be used to screen for molecules that bind to PSF-2 or for molecules to which PSF-2 binds. The binding of PSF-2 and the molecule may activate (agonist), increase, inhibit (antagonist), or decrease activity of the PSF-2 or the molecule bound. Examples of such molecules include antibodies, oligonucleotides, polypeptides (e.g., receptors), or small molecules.

Preferably, the molecule is closely related to the natural ligand of PSF-2, e.g., a fragment of the ligand, or a natural substrate, a ligand, a structural or functional mimetic. (See, Coligan et al., Current Protocols in Immunology 1(2): Chapter 5 (1991).) Similarly, the molecule can be closely related to the natural receptor to which PSF-2 binds, or at least, a fragment of the receptor capable of being bound by PSF-2 (e.g., active site). In either case, the molecule can be rationally designed using known techniques.

Preferably, the screening for these molecules involves producing appropriate cells which express PSF-2, either as a secreted polypeptide or on the cell membrane. Preferred cells include cells from mammals, yeast, Drosophila, or E. coli. Cells expressing PSF-2 (or cell membrane containing the expressed polypeptide) are then preferably contacted with a test compound potentially containing the molecule to observe binding, stimulation, or inhibition of activity of either PSF-2 or the molecule.

The assay may simply test binding of a candidate compound to PSF-2, wherein binding is detected by a label, or in an assay involving competition with a labeled competitor. Further, the assay may test whether the candidate compound results in a signal generated by binding to PSF-2.

Alternatively, the assay can be carried out using cell-free preparations, polypeptide/molecule affixed to a solid support, chemical libraries, or natural product mixtures. The assay may also simply comprise the steps of mixing a candidate compound with a solution containing PSF-2, measuring PSF-2/molecule activity or binding, and comparing the PSF-2/molecule activity or binding to a standard.

Preferably, an ELISA assay can measure PSF-2 level or activity in a sample (e.g., biological sample) using a monoclonal or polyclonal antibody. The antibody can measure PSF-2 level or activity by either binding, directly or indirectly, to PSF-2 or by competing with PSF-2 for a substrate.

All of these above assays can be used as diagnostic or prognostic markers. The molecules discovered using these assays can be used to treat, prevent, and/or diagnose disease or to bring about a particular result in a patient (e.g., blood vessel growth) by activating or inhibiting the PSF-2/molecule. Moreover, the assays can discover agents which may inhibit or enhance the production of PSF-2 from suitably manipulated cells or tissues.

Therefore, the invention includes a method of identifying compounds which bind to PSF-2 comprising the steps of: (a) incubating a candidate binding compound with PSF-2; and (b) determining if binding has occurred. Moreover, the invention includes a method of identifying agonists/antagonists comprising the steps of: (a) incubating a candidate compound with PSF-2, (b) assaying a biological activity, and (b) determining if a biological activity of PSF-2 has been altered.

Other Activities

PSF-2 polypeptides or polynucleotides may also increase or decrease the differentiation or proliferation of embryonic stem cells, besides, as discussed above, hematopoietic lineage.

PSF-2 polypeptides or polynucleotides may also be used to modulate mammalian characteristics, such as body height, weight, hair color, eye color, skin, percentage of adipose tissue, pigmentation, size, and shape (e.g., cosmetic surgery). Similarly, PSF-2 polypeptides or polynucleotides may be used to modulate mammalian metabolism affecting catabolism, anabolism, processing, utilization, and storage of energy.

PSF-2 polypeptides or polynucleotides may be used to change a mammal's mental state or physical state by influencing biorhythms, caricadic rhythms, depression (including depressive disorders), tendency for violence, tolerance for pain, reproductive capabilities (preferably by Activin or Inhibin-like activity), hormonal or endocrine levels, appetite, libido, memory, stress, or other cognitive qualities.

PSF-2 polypeptides or polynucleotides may also be used as a food additive or preservative, such as to increase or decrease storage capabilities, fat content, lipid, polypeptide, carbohydrate, vitamins, minerals, cofactors or other nutritional components.

The role of PSF-2 is integrally intertwined with the role(s) of a number of other potentially vasoregulatory factors including, for example, endothelium-derived relaxing factor, renin, angiotensin, adenosine, thrombin, acetylcholine, vocative intestinal peptide, bradykinin, substance P, cholecystokinin, calcitonin-gene-related peptide, noradrenaline, histamine, A23187 (calcium ionophore), norepinephrine, isoproterenol, serotonin, insulin, glucose, histamine, lipopolysaccharide, IL-1, leukotriene D₄, mellitin, phospholipase C, phospholipase A₂, IFN-g, ergometrine, and others in homeostasis of vessel structures and in the pathophysiology of a number of conditions, disorders, and disease states including, for example, diabetes, diabetic agionpathy, thrombotic thromobocytic purpura (TTP), coronary vasospasm, cerebral vasoconstriction, hypertension, aging, cardiomyopathy, atherogenesis, microvessel disturbances, inflammation, pain, fever, reproduction, gastric secretion, peptic ulcer, ductus arteriosis, congenital heart disease, platelet aggregation, thrombosis, myocardial infarction, ischemia, ischemic heart disease, reperfusion injury, modulation of baroreceptor activity, and the like.

Having generally described the invention, the same will be more readily understood by reference to the following examples, which are provided by way of illustration and are not intended as limiting.

### EXAMPLES

### Example 1

Isolation of the PSF-2 cDNA Clone from the Deposited Sample

The cDNA for PSF-2 is inserted into the Sal I and Not I restriction sites in the pCMVSport vector available from Life Technologies, Inc. (Gaithersburg, Md.). pCMVSport contains an ampicillin resistance gene and may be transformed into E. coli strain DH10B, also available from Life Technologies. (See, for instance, Gruber, C. E., et al., Focus 15:59-(1993).)

Two approaches can be used to isolate PSF-2 from the deposited sample. First, the deposited clone is transformed into a suitable host (such as XL-1 Blue (Stratagene)) using techniques known to those of skill in the art, such as those provided by the vector supplier or in related publications or patents. The transformants are plated on 1.5% agar plates (containing the appropriate selection agent, e.g., ampicillin) to a density of about 150 transformants (colonies) per plate. A single colony is then used to generate DNA using nucleic acid isolation techniques well known to those skilled in the art. (e.g., Sambrook et al., Molecular Cloning: A Laboratory Manual, 2nd Edit., (1989), Cold Spring Harbor Laboratory Press.)

Alternatively, two primers of 17-20 nucleotides derived from both ends of the SEQ ID NO:1 (i.e., within the region of SEQ ID NO:1 bounded by the 5′ and 3′ nucleotides of the clone) are synthesized and used to amplify the PSF-2 cDNA using the deposited cDNA plasmid as a template. The polymerase chain reaction is carried out under routine conditions, for instance, in 25 ml of reaction mixture with 0.5 mg of the above cDNA template. A convenient reaction mixture is 1.5-5 mM MgCl₂, 0.01% (w/v) gelatin, 20 uM each of dATP, dCTP, dGTP, dTTP, 25 pmol of each primer and 0.25 Unit of Taq polymerase. Thirty five cycles of PCR (denaturation at 94 C for 1 min; annealing at 55 C for 1 min; elongation at 72 C for 1 min) are performed with a Perkin-Elmer Cetus automated thermal cycler. The amplified product is analyzed by agarose gel electrophoresis and the DNA band with expected molecular weight is excised and purified. The PCR product is verified to be the selected sequence by subcloning and sequencing the DNA product.

Several methods are available for the identification of the 5′ or 3′ non-coding portions of the PSF-2 gene which may not be present in the deposited clone. These methods include but are not limited to, filter probing, clone enrichment using specific probes, and protocols similar or identical to 5′ and 3′ “RACE” protocols which are well known in the art. For instance, a method similar to 5′ RACE is available for generating the missing 5′ end of a desired full-length transcript. (Fromont-Racine et al., Nucleic Acids Res. 21(7):1683-1684 (1993).)

Briefly, a specific RNA oligonucleotide is ligated to the 5′ ends of a population of RNA presumably containing full-length gene RNA transcripts. A primer set containing a primer specific to the ligated RNA oligonucleotide and a primer specific to a known sequence of the PSF-2 gene of interest is used to PCR amplify the 5′ portion of the PSF-2 full-length gene. This amplified product may then be sequenced and used to generate the full length gene.

This above method starts with total RNA isolated from the desired source, although poly-A+ RNA can be used. The RNA preparation can then be treated with phosphatase if necessary to eliminate 5′ phosphate groups on degraded or damaged RNA which may interfere with the later RNA ligase step. The phosphatase should then be inactivated and the RNA treated with tobacco acid pyrophosphatase in order to remove the cap structure present at the 5′ ends of messenger RNAs. This reaction leaves a 5′ phosphate group at the 5′ end of the cap cleaved RNA which can then be ligated to an RNA oligonucleotide using T4 RNA ligase.

This modified RNA preparation is used as a template for first strand cDNA synthesis using a gene specific oligonucleotide. The first strand synthesis reaction is used as a template for PCR amplification of the desired 5′ end using a primer specific to the ligated RNA oligonucleotide and a primer specific to the known sequence of the gene of interest. The resultant product is then sequenced and analyzed to confirm that the 5′ end sequence belongs to the PSF-2 gene.

### Example 2

Isolation of PSF-2 Genomic Clones

A human genomic P1 library (Genomic Systems, Inc.) is screened by PCR using primers selected for the cDNA sequence corresponding to SEQ ID NO:1., according to the method described in Example 1. (See also, Sambrook.)

### Example 3

Tissue Distribution of PSF-2 Polypeptides

Tissue distribution of mRNA expression of PSF-2 is determined using protocols for Northern blot analysis, described by, among others, Sambrook et al. For example, a PSF-2 probe produced by the method described in Example 1 is labeled with ³²P using the rediprime™ DNA labeling system (Amersham Life Science), according to manufacturer's instructions. After labeling, the probe is purified using CHROMA SPIN-100™ column (Clontech Laboratories, Inc.), according to manufacturer's protocol number PT1200-1. The purified labeled probe is then used to examine various human tissues for mRNA expression.

Multiple Tissue Northern (MTN) blots containing various human tissues (H) or human immune system tissues (IM) (Clontech) are examined with the labeled probe using ExpressHyb™ hybridization solution (Clontech) according to manufacturer's protocol number PT1190-1. Following hybridization and washing, the blots are mounted and exposed to film at −70 C overnight, and the films developed according to standard procedures.

### Example 4

Chromosomal Mapping of PSF-2

An oligonucleotide primer set is designed according to the sequence at the 5′ end of SEQ ID NO:1. This primer preferably spans about 100 nucleotides. This primer set is then used in a polymerase chain reaction under the following set of conditions: 30 seconds, 95 C; 1 minute, 56 C; 1 minute, 70 C. This cycle is repeated 32 times followed by one 5 minute cycle at 70 C. Human, mouse, and hamster DNA is used as template in addition to a somatic cell hybrid panel containing individual chromosomes or chromosome fragments (Bios, Inc). The reactions is analyzed on either 8% polyacrylamide gels or 3.5% agarose gels. Chromosome mapping is determined by the presence of an approximately 100 bp PCR fragment in the particular somatic cell hybrid.

### Example 5

Bacterial Expression of PSF-2

PSF-2 polynucleotide encoding a PSF-2 polypeptide invention is amplified using PCR oligonucleotide primers corresponding to the 5′ and 3′ ends of the DNA sequence, as outlined in Example 1, to synthesize insertion fragments. The primers used to amplify the cDNA insert should preferably contain restriction sites, such as Bam HI, Xba I, and Sal I, at the 5′ end of the primers in order to clone the amplified product into the expression vector. DNA can be inserted into the pHE4a vector by restricting the vector with Nde I and Xba I, Bam HI, Xho I, or Asp 718. The pHE4a vector has ATCC Accession Number 209645, and was deposited on Feb. 25, 1998. The vector contains: 1) a neomycinphosphotransferase gene as a selection marker, 2) an E. coli origin of replication, 3) a T5 phage promoter sequence, 4) two lac operator sequences, 5) a Shine-Delgarno sequence, and 6) the lactose operon repressor gene (lacIq). The origin of replication (oriC) is derived from pUC19 (Life Technologies, Inc., Gaithersburg, Md.). The promoter sequence and operator sequences are made synthetically.

Specifically, to clone the mature domain of the PSF-2 polypeptide in the bacterial vector pHE4, the 5′ primer has the sequence 5′-GCA GCA CAT ATG AGG CCA TCC CCA GGC CCA GAT TAC CTG CGG C-3′ (SEQ ID NO:14) containing the underlined Nde I restriction site followed a number of nucleotides of the amino terminal coding sequence of the mature PSF-2 sequence in SEQ ID NO:1. One of ordinary skill in the art would appreciate, of course, that the point in the polypeptide coding sequence where the 5′ primer begins may be varied to amplify a DNA segment encoding any desired portion of the complete PSF-2 polypeptide shorter or longer than the mature domain of the polypeptide. The 3′ primer has the sequence 5′-GCA GCA GGT ACC TTA GTA GTA ATC GTC ATT CTC TTC ACT CTC AGC-3′ (SEQ ID NO:15) containing the underlined Asp 718 restriction site followed by a number nucleotides complementary to the 3′ end of the coding sequence of the PSF-2 DNA sequence of SEQ ID NO:1.

The pHE4 vector is digested with Nde I and Asp 718 and the amplified fragment is ligated into the pHE4 vector maintaining the reading frame initiated at the bacterial RBS. The ligation mixture is then used to transform the E. coli strain M15/rep4 (Qiagen, Inc.) which contains multiple copies of the plasmid pREP4, which expresses the lacI repressor and also confers kanamycin resistance (Kan^{r}). Transformants are identified by their ability to grow on LB plates and ampicillin/kanamycin resistant colonies are selected. Plasmid DNA is isolated and confirmed by restriction analysis.

In a specific embodiment, the cDNA sequence encoding the mature form of the PSF-2 polypeptide in the deposited clone is subcloned into the expression vector pQE70 such that the mature form of a PSF-2 polypeptide will be expressed as a fusion protein with a C-terminal His-tag. PCR amplification of the insert is accomplished using oligonucleotide primers corresponding to the 5′ and 3′ sequences of the gene. The 5′ primer has the sequence 5′-GCA GCA GCA TGC CAT CCC CAG GCC CAG ATT ACC TGC GGC GC-3′ (SEQ ID NO:27) containing the Sph I restriction enzyme site followed by a number of nucleotides of the sequence of the mature PSF-2 polypeptide shown in FIGS. 1A and 1B and SEQ ID NO:1, beginning with the AUG initiation codon. The 3′ primer has the sequence 5′-GCA GCA GGA TCC GTA GTA ATC GTC ATT CTC TTC ACT CTC AGC-3′ (SEQ ID NO:28) containing the Bam HI restriction site followed by a number of nucleotides complementary to the 3′ noncoding sequence in FIGS. 1A and 1B and SEQ ID NO:1.

In a specific embodiment, the cDNA sequence encoding the mature form of the PSF-2 polypeptide in the deposited clone is subcloned into the expression vector pQE9 such that the mature form of a PSF-2 polypeptide will be expressed as a fusion protein with an N-terminal His-tag. PCR amplification of the insert is accomplished using oligonucleotide primers corresponding to the 5′ and 3′ sequences of the gene. The 5′ primer has the sequence 5′-GCA GCA GGA TCC AGG CCA TCC CCA GGC CCA GAT TAC CTG CGG C-3′ (SEQ ID NO:29) containing the Bam HI restriction enzyme site followed by a number of nucleotides of the sequence of the mature PSF-2 polypeptide shown in FIGS. 1A and 1B and SEQ ID NO:1, beginning with the AUG initiation codon. The 3′ primer has the sequence 5′-GCA GCA AAG CTT CTA GTA GTA ATC GTC ATT CTC TTC ACT CTC-3′ (SEQ ID NO:30) containing the Hin dIII restriction site followed by a number of nucleotides complementary to the 3′ noncoding sequence in FIGS. 1A and 1B and SEQ ID NO:1.

Clones containing the desired constructs are grown overnight (O/N) in liquid culture in LB media supplemented with both Amp (100 ug/ml) and Kan (25 ug/ml). The O/N culture is used to inoculate a large culture at a ratio of 1:100 to 1:250. The cells are grown to an optical density 600 (O.D.⁶⁰⁰) of between 0.4 and 0.6. IPTG (Isopropyl-B-D-thiogalacto pyranoside) is then added to a final concentration of 1 mM. IPTG induces by inactivating the lacd repressor, clearing the P/O leading to increased gene expression.

Cells are grown for an extra 3 to 4 hours. Cells are then harvested by centrifugation (20 mins at 6000×g). The cell pellet is solubilized in the chaotropic agent 6 M Guanidine HCl by stirring for 3-4 hours at 4 C. The cell debris is removed by centrifugation, and the supernatant containing the polypeptide is loaded onto a nickel-nitrilo-tri-acetic acid (“Ni-NTA”) affinity resin column (available from QIAGEN, Inc., supra). Polypeptides with a 6× His tag bind to the Ni-NTA resin with high affinity and can be purified in a simple one-step procedure (for details see: The QIAexpressionist (1995) QIAGEN, Inc., supra).

Briefly, the supernatant is loaded onto the column in 6 M guanidine-HCl, pH 8, the column is first washed with 10 volumes of 6 M guanidine-HCl, pH 8, then washed with 10 volumes of 6 M guanidine-HCl pH 6, and finally the polypeptide is eluted with 6 M guanidine-HCl, pH 5.

The purified PSF-2 polypeptide is then renatured by dialyzing it against phosphate-buffered saline (PBS) or 50 mM Na-acetate, pH 6 buffer plus 200 mM NaCl. Alternatively, the PSF-2 polypeptide can be successfully refolded while immobilized on the Ni-NTA column. The recommended conditions are as follows: renature using a linear 6M-1M urea gradient in 500 mM NaCl, 20% glycerol, 20 mM Tris/HCl pH 7.4, containing protease inhibitors. The renaturation should be performed over a period of 1.5 hours or more. After renaturation the polypeptides are eluted by the addition of 250 mM immidazole. Immidazole is removed by a final dialyzing step against PBS or 50 mM sodium acetate pH 6 buffer plus 200 mM NaCl. The purified PSF-2 polypeptide is stored at 4 C or frozen at −80 C.

In addition to the above expression vector, the present invention further includes an expression vector comprising phage operator and promoter elements operatively linked to a PSF-2 polynucleotide, called pQE-9. This plasmid vector encodes antibiotic resistance (Amp^{R}), a bacterial origin of replication (ori), an IPTG-regulatable promoter/operator (P/O), a ribosome binding site (RBS), a 6-histidine tag (6-His), and restriction enzyme cloning sites, for example, Bam HI and Xba I. (Qiagen, Inc., Chatsworth, Calif.). The pQE-9 vector could easily be substituted in the above protocol to express PSF-2 polypeptide in a bacterial system.

### Example 6

Purification of PSF-2 Polypeptide from an Inclusion Body

The following alternative method can be used to purify PSF-2 polypeptide expressed in E coli when it is present in the form of inclusion bodies. Unless otherwise specified, all of the following steps are conducted at 4-10 C.

Upon completion of the production phase of the E. coli fermentation, the cell culture is cooled to 4-10 C and the cells harvested by continuous centrifugation at 15,000 rpm (Heraeus Sepatech). On the basis of the expected yield of polypeptide per unit weight of cell paste and the amount of purified polypeptide required, an appropriate amount of cell paste, by weight, is suspended in a buffer solution containing 100 mM Tris, 50 mM EDTA, pH 7.4. The cells are dispersed to a homogeneous suspension using a high shear mixer.

The cells are then lysed by passing the solution through a microfluidizer (Microfuidics, Corp. or APV Gaulin, Inc.) twice at 4000-6000 psi. The homogenate is then mixed with NaCl solution to a final concentration of 0.5 M NaCl, followed by centrifugation at 7000×g for 15 min. The resultant pellet is washed again using 0.5M NaCl, 100 mM Tris, 50 mM EDTA, pH 7.4.

The resulting washed inclusion bodies are solubilized with 1.5 M guanidine hydrochloride (GuHCl) for 2-4 hours. After 7000×g centrifugation for 15 min., the pellet is discarded and the polypeptide containing supernatant is incubated at 4 C overnight to allow further GuHCl extraction.

Following high speed centrifugation (30,000×g) to remove insoluble particles, the GuHCl solubilized polypeptide is refolded by quickly mixing the GuHCl extract with 20 volumes of buffer containing 50 mM sodium, pH 4.5, 150 mM NaCl, 2 mM EDTA by vigorous stirring. The refolded diluted polypeptide solution is kept at 4 C without mixing for 12 hours prior to further purification steps.

To clarify the refolded polypeptide solution, a previously prepared tangential filtration unit equipped with 0.16 um membrane filter with appropriate surface area (e.g., Filtron), equilibrated with 40 mM sodium acetate, pH 6.0 is employed. The filtered sample is loaded onto a cation exchange resin (e.g., Poros HS-50, Perseptive Biosystems). The column is washed with 40 mM sodium acetate, pH 6.0 and eluted with 250 mM, 500 mM, 1000 mM, and 1500 mM NaCl in the same buffer, in a stepwise manner. The absorbance at 280 nm of the effluent is continuously monitored. Fractions are collected and further analyzed by SDS-PAGE.

Fractions containing the PSF-2 polypeptide are then pooled and mixed with 4 volumes of water. The diluted sample is then loaded onto a previously prepared set of tandem columns of strong anion (Poros HQ-50, Perseptive Biosystems) and weak anion (Poros CM-20, Perseptive Biosystems) exchange resins. The columns are equilibrated with 40 mM sodium acetate, pH 6.0. Both columns are washed with 40 mM sodium acetate, pH 6.0, 200 mM NaCl. The CM-20 column is then eluted using a 10 column volume linear gradient ranging from 0.2 M NaCl, 50 mM sodium acetate, pH 6.0 to 1.0 M NaCl, 50 mM sodium acetate, pH 6.5. Fractions are collected under constant A₂₈₀ monitoring of the effluent. Fractions containing the polypeptide (determined, for instance, by 16% SDS-PAGE) are then pooled.

The resultant PSF-2 polypeptide should exhibit greater than 95% purity after the above refolding and purification steps. No major contaminant bands should be observed from Commassie blue stained 16% SDS-PAGE gel when 5 ug of purified polypeptide is loaded. The purified PSF-2 polypeptide can also be tested for endotoxin/LPS contamination, and typically the LPS content is less than 0.1 ng/ml according to LAL assays.

### Example 7

Cloning and Expression of PSF-2 in a Baculovirus Expression System

In this example, the plasmid shuttle vector pA2 is used to insert PSF-2 polynucleotide into a baculovirus to express PSF-2. This expression vector contains the strong polyhedrin promoter of the Autographa californica nuclear polyhedrosis virus (AcMNPV) followed by convenient restriction sites such as Bam HI, Xba I and Asp 718. The polyadenylation site of the simian virus 40 (“SV40”) is used for efficient polyadenylation. For easy selection of recombinant virus, the plasmid contains the beta-galactosidase gene from E. coli under control of a weak Drosophila promoter in the same orientation, followed by the polyadenylation signal of the polyhedrin gene. The inserted genes are flanked on both sides by viral sequences for cell-mediated homologous recombination with wild-type viral DNA to generate a viable virus that express the cloned PSF-2 polynucleotide.

Many other baculovirus vectors can be used in place of the vector above, such as pAc373, pVL941, and pAcIM1, as one skilled in the art would readily appreciate, as long as the construct provides appropriately located signals for transcription, translation, secretion and the like, including a signal peptide and an in-frame AUG as required. Such vectors are described, for instance, in Luckow et al., Virology 170:31-39 (1989).

Specifically, the PSF-2 cDNA sequence contained in the deposited clone, including the AUG initiation codon and any naturally associated leader sequence, is amplified using the PCR protocol described in Example 1. If the naturally occurring signal sequence is used to produce the secreted polypeptide, the pA2 vector does not need a second signal peptide. Alternatively, the vector can be modified (pA2GP) to include a baculovirus leader sequence, using the standard methods described in Summers et al., “A Manual of Methods for Baculovirus Vectors and Insect Cell Culture Procedures,” Texas Agricultural Experimental Station Bulletin No. 1555 (1987).

More specifically, the cDNA sequence encoding the full length PSF-2 polypeptide in the deposited clone, including the AUG initiation codon and the naturally associated leader sequence shown in SEQ ID NO:1, is amplified using PCR oligonucleotide primers corresponding to the 5′ and 3′ sequences of the gene. The 5′ primer has the sequence 5′-GCA GCA GGA TCC GCC ATC ATG CTG CCG CCG CCG CGG CCC GCA GCT GCC-3′ (SEQ ID NO:16) containing the Bam HI restriction enzyme site, an efficient signal for initiation of translation in eukaryotic cells (Kozak, M., J. Mol. Biol. 196:947-950 (1987)), followed by a number of nucleotides of the sequence of the complete PSF-2 polypeptide shown in FIGS. 1A and 1B, beginning with the AUG initiation codon. The 3′ primer has the sequence 5′-GCA GCA GGT ACC TTA GTA GTA ATC GTC ATT CTC TTC ACT CTC AGC-3′ (SEQ ID NO:17) containing the Asp 718 restriction site followed by a number of nucleotides complementary to the 3′ noncoding sequence in FIGS. 1A and 1B.

In a specific embodiment, the cDNA sequence encoding the full length PSF-2 polypeptide in the deposited clone is subcloned into the expression vector pA2. PCR amplification of the insert is accomplished using oligonucleotide primers corresponding to the 5′ and 3′ sequences of the gene. The 5′ primer has the sequence 5′-GCA GCA GGA TCC GCC ATC ATG CTG CCG CCG CCG CGG CCC GCA GCT GCC TTG-3′ (SEQ ID NO:25) containing the Bam HI restriction enzyme site, an efficient signal for initiation of translation in eukaryotic cells (Kozak, M., J. Mol. Biol. 196:947-950 (1987)), followed by a number of nucleotides of the sequence of the complete PSF-2 polypeptide shown in FIGS. 1A and 1B and in SEQ ID NO:1, beginning with the AUG initiation codon. The 3′ primer has the sequence 5′-GCA GCA TCT AGA TTA GTA GTA ATC GTC ATT CTC TTC ACT CTC AGC CTC-3′ (SEQ ID NO:26) containing the Xba I restriction site followed by a number of nucleotides complementary to the 3′ noncoding sequence in FIGS. 1A and 1B and in SEQ ID NO:1.

The amplified fragment is isolated from a 1% agarose gel using a commercially available kit (“Geneclean,” BIO 101 Inc., La Jolla, Calif.). The fragment then is digested with appropriate restriction enzymes and again purified on a 1% agarose gel.

The plasmid is digested with the corresponding restriction enzymes and optionally, can be dephosphorylated using calf intestinal phosphatase, using routine procedures known in the art. The DNA is then isolated from a 1% agarose gel using a commercially available kit (“Geneclean” BIO 101 Inc., La Jolla, Calif.).

The fragment and the dephosphorylated plasmid are ligated together with T4 DNA ligase. E. coli HB101 or other suitable E. coli hosts such as XL-1 Blue (Stratagene Cloning Systems, La Jolla, Calif.) cells are transformed with the ligation mixture and spread on culture plates. Bacteria containing the plasmid are identified by digesting DNA from individual colonies and analyzing the digestion product by gel electrophoresis. The sequence of the cloned fragment is confirmed by DNA sequencing.

Five micrograms of a plasmid containing the polynucleotide is co-transfected with 1.0 ug of a commercially available linearized baculovirus DNA (“BaculoGold™ baculovirus DNA”, Pharmingen, San Diego, Calif.), using the lipofection method described by Felgner et al., Proc. Natl. Acad. Sci. USA 84:7413-7417 (1987). One mg of BaculoGold™ virus DNA and 5 mg of the plasmid are mixed in a sterile well of a microtiter plate containing 50 ml of serum-free Grace's medium (Life Technologies Inc., Gaithersburg, Md.). Afterwards, 10 ml Lipofectin plus 90 ml Grace's medium are added, mixed and incubated for 15 minutes at room temperature. Then the transfection mixture is added drop-wise to Sf9 insect cells (ATCC CRL 1711) seeded in a 35 mm tissue culture plate with 1 ml Grace's medium without serum. The plate is then incubated for 5 hours at 27 C. The transfection solution is then removed from the plate and 1 ml of Grace's insect medium supplemented with 10% fetal calf serum is added. Cultivation is then continued at 27 C for four days.

After four days the supernatant is collected and a plaque assay is performed, as described by Summers and Smith, supra. An agarose gel with “Blue Gal” (Life Technologies Inc., Gaithersburg) is used to allow easy identification and isolation of gal-expressing clones, which produce blue-stained plaques. (A detailed description of a “plaque assay” of this type can also be found in the user's guide for insect cell culture and baculovirology distributed by Life Technologies Inc., Gaithersburg, page 9-10.) After appropriate incubation, blue stained plaques are picked with the tip of a micropipettor (e.g., Eppendorf). The agar containing the recombinant viruses is then resuspended in a microcentrifuge tube containing 200 ul of Grace's medium and the suspension containing the recombinant baculovirus is used to infect Sf9 cells seeded in 35 mm dishes. Four days later the supernatants of these culture dishes are harvested and then they are stored at 4 C.

To verify the expression of the polypeptide, Sf9 cells are grown in Grace's medium supplemented with 10% heat-inactivated FBS. The cells are infected with the recombinant baculovirus containing the polynucleotide at a multiplicity of infection (“MOI”) of about 2. If radiolabeled polypeptides are desired, 6 hours later the medium is removed and is replaced with SF900 II medium minus methionine and cysteine (available from Life Technologies Inc., Rockville, Md.). After 42 hours, 5 mCi of ³⁵S-methionine and 5 mCi ³⁵S-cysteine (available from Amersham) are added. The cells are further incubated for 16 hours and then are harvested by centrifugation. The polypeptides in the supernatant as well as the intracellular polypeptides are analyzed by SDS-PAGE followed by autoradiography (if radiolabeled).

Microsequencing of the amino acid sequence of the amino terminus of purified polypeptide may be used to determine the amino terminal sequence of the produced PSF-2 polypeptide.

### Example 8

Expression of PSF-2 in Mammalian Cells

PSF-2 polypeptide can be expressed in a mammalian cell. A typical mammalian expression vector contains a promoter element, which mediates the initiation of transcription of mRNA, a polypeptide coding sequence, and signals required for the termination of transcription and polyadenylation of the transcript. Additional elements include enhancers, Kozak sequences and intervening sequences flanked by donor and acceptor sites for RNA splicing. Highly efficient transcription is achieved with the early and late promoters from SV40, the long terminal repeats (LTRs) from Retroviruses, e.g., RSV, HTLV-I, HIV-1 and the early promoter of the cytomegalovirus (CMV). However, cellular elements can also be used (e.g., the human actin promoter).

Suitable expression vectors for use in practicing the present invention include, for example, vectors such as pSVL and pMSG (Pharmacia, Uppsala, Sweden), pRSVcat (ATCC 37152), pSV2DHFR (ATCC 37146), pBC12MI (ATCC 67109), pCMVSport 2.0, and pCMVSport 3.0. Mammalian host cells that could be used include, human Hela, 293, H9 and Jurkat cells, mouse NIH3T3 and C127 cells, COS 1, Cos 7 and CV1, quail QC1-3 cells, mouse L cells and Chinese hamster ovary (CHO) cells.

Alternatively, PSF-2 polypeptide can be expressed in stable cell lines containing the PSF-2 polynucleotide integrated into a chromosome. The co-transfection with a selectable marker such as DHFR, gpt, neomycin, hygromycin allows the identification and isolation of the transfected cells.

The transfected PSF-2 gene can also be amplified to express large amounts of the encoded polypeptide. The DHFR (dihydrofolate reductase) marker is useful in developing cell lines that carry several hundred or even several thousand copies of the gene of interest. (See, e.g., Alt, F. W., et al., J. Biol. Chem. 253:1357-1370 (1978); Hamlin, J. L. and Ma, C., Biochem. et Biophys. Acta, 1097:107-143 (1990); Page, M. J. and Sydenham, M. A., Biotechnology 9:64-68 (1991).) Another useful selection marker is the enzyme glutamine synthase (GS) (Murphy et al., Biochem J. 227:277-279 (1991); Bebbington et al., Bio/Technology 10:169-175 (1992). Using these markers, the mammalian cells are grown in selective medium and the cells with the highest resistance are selected. These cell lines contain the amplified gene(s) integrated into a chromosome. Chinese hamster ovary (CHO) and NSO cells are often used for the production of polypeptides.

Derivatives of the plasmid pSV2-DHFR (ATCC Accession No. 37146), the expression vectors pC4 (ATCC Accession No. 209646) and pC6 (ATCC Accession No.209647) contain the strong promoter (LTR) of the Rous Sarcoma Virus (Cullen et al., Molecular and Cellular Biology, 438-447 (March, 1985)) plus a fragment of the CMV-enhancer (Boshart et al., Cell 41:521-530 (1985).) Multiple cloning sites, e.g., with the restriction enzyme cleavage sites BamHI, XbaI and Asp718, facilitate the cloning of PSF-2. The vectors also contain the 3′ intron, the polyadenylation and termination signal of the rat preproinsulin gene, and the mouse DHFR gene under control of the SV40 early promoter.

Specifically, the plasmid pC4 is digested with Bam HI and Asp 718 and then dephosphorylated using calf intestinal phosphates by procedures known in the art. The vector is then isolated from a 1% agarose gel.

The cDNA sequence encoding the full length PSF-2 polypeptide in the deposited clone is amplified using PCR oligonucleotide primers corresponding to the 5′ and 3′ sequences of the gene. The 5′ primer has the sequence 5′-GCA GCA GGA TCC GCC ATC ATG CTG CCG CCG CCG CGG CCC GCA GCT GCC-3′ (SEQ ID NO:16) containing the Bam HI restriction enzyme site, an efficient signal for initiation of translation in eukaryotic cells (Kozak, M., J. Mol. Biol. 196:947-950 (1987)), followed by a number of nucleotides of the sequence of the complete PSF-2 polypeptide shown in FIGS. 1A and 1B, beginning with the AUG initiation codon. The 3′ primer has the sequence 5′-GCA GCA GGT ACC TTA GTA GTA ATC GTC ATT CTC TTC ACT CTC AGC-3′ (SEQ ID NO:17) containing the Asp 718 restriction site followed by a number of nucleotides complementary to the 3′ noncoding sequence in FIGS. 1A and 1B.

In a specific embodiment, the cDNA sequence encoding the full length PSF-2 polypeptide in the deposited clone is subcloned into the expression vector pC4. PCR amplification of the insert is accomplished using oligonucleotide primers corresponding to the 5′ and 3′ sequences of the gene. The 5′ primer has the sequence 5′-GCA GCA GGA TCC GCC ATC ATG CTG CCG CCG CCG CGG CCC GCA GCT GCC TTG-3′ (SEQ ID NO:25) containing the Bam HI restriction enzyme site, an efficient signal for initiation of translation in eukaryotic cells (Kozak, M., J. Mol. Biol. 196:947-950 (1987)), followed by a number of nucleotides of the sequence of the complete PSF-2 polypeptide shown in FIGS. 1A and 1B, beginning with the AUG initiation codon. The 3′ primer has the sequence 5′-GCA GCA TCT AGA TTA GTA GTA ATC GTC ATT CTC TTC ACT CTC AGC CTC-3′ (SEQ ID NO:26) containing the Xba I restriction site followed by a number of nucleotides complementary to the 3′ noncoding sequence in FIGS. 1A and 1B.

If a naturally occurring signal sequence is used to produce a secreted polypeptide, the vector does not need a second signal peptide. Alternatively, if a naturally occurring signal sequence is not used, the vector can be modified to include a heterologous signal sequence in an effort to secrete the polypeptide from the cell. (See, e.g., WO 96/34891.)

The amplified fragment is then digested with the Bam HI and Asp 718 and purified on a 1% agarose gel using a commercially available kit (“Geneclean,” BIO 101 Inc., La Jolla, Calif.). The isolated fragment and the dephosphorylated vector are then ligated with T4 DNA ligase. E. coli HB101 or XL-1 Blue cells are then transformed and bacteria are identified that contain the fragment inserted into plasmid pC4 using, for instance, restriction enzyme analysis.

Chinese hamster ovary cells lacking an active DHFR gene is used for transfection. Five mg of the expression plasmid pC4 is cotransfected with 0.5 mg of the plasmid pSVneo using lipofectin (Felgner et al., supra). The plasmid pSV2-neo contains a dominant selectable marker, the neo gene from Tn5 encoding an enzyme that confers resistance to a group of antibiotics including G418. The cells are seeded in alpha minus MEM supplemented with 1 mg/ml G418. After 2 days, the cells are trypsinized and seeded in hybridoma cloning plates (Greiner, Germany) in alpha minus MEM supplemented with 10, 25, or 50 ng/ml of metothrexate plus 1 mg/ml G418. After about 10-14 days single clones are trypsinized and then seeded in 6-well petri dishes or 10 ml flasks using different concentrations of methotrexate (50 nM, 100 nM, 200 nM, 400 nM, 800 nM). Clones growing at the highest concentrations of methotrexate are then transferred to new 6-well plates containing even higher concentrations of methotrexate (1 mM, 2 mM, 5 mM, 10 mM, 20 mM). The same procedure is repeated until clones are obtained which grow at a concentration of 100-200 mM. Expression of PSF-2 is analyzed, for instance, by SDS-PAGE and Western blot or by reversed phase HPLC analysis.

### Example 9

Construction of N-Terminal and/or C-Terminal Deletion Mutants

The following general approach may be used to clone a N-terminal or C-terminal deletion PSF-2 deletion mutant. Generally, two oligonucleotide primers of about 15-25 nucleotides are derived from the desired 5′ and 3′ positions of a polynucleotide of SEQ ID NO:1. The 5′ and 3′ positions of the primers are determined based on the desired PSF-2 polynucleotide fragment. An initiation and stop codon are added to the 5′ and 3′ primers respectively, if necessary, to express the PSF-2 polypeptide fragment encoded by the polynucleotide fragment. Preferred PSF-2 polynucleotide fragments are those encoding the N-terminal and C-terminal deletion mutants disclosed above in the “Polynucleotide and Polypeptide Fragments” section of the Specification.

Additional nucleotides containing restriction sites to facilitate cloning of the PSF-2 polynucleotide fragment in a desired vector may also be added to the 5′ and 3′ primer sequences. The PSF-2 polynucleotide fragment is amplified from genomic DNA or from the deposited cDNA clone using the appropriate PCR oligonucleotide primers and conditions discussed herein or known in the art. The PSF-2 polypeptide fragments encoded by the PSF-2 polynucleotide fragments of the present invention may be expressed and purified in the same general manner as the full length polypeptides, although routine modifications may be necessary due to the differences in chemical and physical properties between a particular fragment and full length polypeptide.

As a means of exemplifying but not limiting the present invention, the polynucleotide encoding the PSF-2 polypeptide fragment Cys-53 to Asp-247 is amplified and cloned as follows: A 5′ primer is generated comprising a restriction enzyme site followed by an initiation codon in frame with the polynucleotide sequence encoding the N-terminal portion of the polypeptide fragment beginning with Cys-53. A complementary 3′ primer is generated comprising a restriction enzyme site followed by a stop codon in frame with the polynucleotide sequence encoding C-terminal portion of the PSF-2 polypeptide fragment ending with Asp-247.

The amplified polynucleotide fragment and the expression vector are digested with restriction enzymes which recognize the sites in the primers. The digested polynucleotides are then ligated together. The PSF-2 polynucleotide fragment is inserted into the restricted expression vector, preferably in a manner which places the PSF-2 polypeptide fragment coding region downstream from the promoter. The ligation mixture is transformed into competent E. coli cells using standard procedures and as described in the Examples herein. Plasmid DNA is isolated from resistant colonies and the identity of the cloned DNA confirmed by restriction analysis, PCR and DNA sequencing.

### Example 10

Polypeptide Fusions of PSF-2

PSF-2 polypeptides are preferably fused to other polypeptides. These fusion polypeptides can be used for a variety of applications. For example, fusion of PSF-2 polypeptides to His-tag, HA-tag, polypeptide A, IgG domains, and maltose binding polypeptide facilitates purification. (See Example 5; see also EP A 394,827; Traunecker, et al., Nature 331:84-86 (1988).) Similarly, fusion to IgG-1, IgG-3, and albumin increases the halflife time in vivo. Nuclear localization signals fused to PSF-2 polypeptides can target the polypeptide to a specific subcellular localization, while covalent heterodimer or homodimers can increase or decrease the activity of a fusion polypeptide. Fusion polypeptides can also create chimeric molecules having more than one function. Finally, fusion polypeptides can increase solubility and/or stability of the fused polypeptide compared to the non-fused polypeptide. All of the types of fusion polypeptides described above can be made by modifying the following protocol, which outlines the fusion of a polypeptide to an IgG molecule, or the protocol described in Example 5.

Briefly, the human Fc portion of the IgG molecule can be PCR amplified, using primers that span the 5′ and 3′ ends of the sequence described below. These primers also should have convenient restriction enzyme sites that will facilitate cloning into an expression vector, preferably a mammalian expression vector.

For example, if pC4 (Accession No. 209646) is used, the human Fc portion can be ligated into the BamHI cloning site. Note that the 3′ BamHI site should be destroyed. Next, the vector containing the human Fc portion is re-restricted with BamHI, linearizing the vector, and PSF-2 polynucleotide, isolated by the PCR protocol described in Example 1, is ligated into this BamHI site. Note that the polynucleotide is cloned without a stop codon, otherwise a fusion polypeptide will not be produced.

If the naturally occurring signal sequence is used to produce the secreted polypeptide, pC4 does not need a second signal peptide. Alternatively, if the naturally occurring signal sequence is not used, the vector can be modified to include a heterologous signal sequence. (See, e.g., WO 96/34891.)

| | |
|---|---|
| Human IgG Fc region: | |
| GGG ATC CGG AGC CCA AAT CTT CTG ACA AAA CTC ACA CAT GCC CAC | (SEQ ID NO:4) |
| CGT GCC CAG CAC CTG AAT TCG AGG GTG CAC CGT CAG TCT TCC TCT TCC | |
| CCC CAA AAC CCA AGG ACA CCC TCA TGA TCT CCC GGA CTC CTG AGG | |
| TCA CAT GCG TGG TGG TGG ACG TAA GCC ACG AAG ACC CTG AGG TCA | |
| AGT TCA ACT GGT ACG TGG ACG GCG TGG AGG TGC ATA ATG CCA AGA | |
| CAA AGC CGC GGG AGG AGC AGT ACA ACA GCA CGT ACC GTG TGG TCA | |
| GCG TCC TCA CCG TCC TGC ACC AGG ACT GGC TGA ATG GCA AGG AGT | |
| ACA AGT GCA AGG TCT CCA ACA AAG CCC TCC CAA CCC CCA TCG AGA | |
| AAA CCA TCT CCA AAG CCA AAG GGC AGC CCC GAG AAC CAC AGG TGT | |
| ACA CCC TGC CCC CAT CCC GGG ATG AGC TGA CCA AGA ACC AGG TCA | |
| GCC TGA CCT GCC TGG TCA AAG GCT TCT ATC CAA GCG ACA TCG CCG | |
| TGG AGT GGG AGA GCA ATG GGC AGC CGG AGA ACA ACT ACA AGA CCA | |
| CGC CTC CCG TGC TGG ACT CCG ACG GCT CCT TCT TCC TCT ACA GCA AGC | |
| TCA CCG TGG ACA AGA GCA GGT GGC AGC AGG GGA ACG TCT TCT CAT | |
| GCT CCG TGA TGC ATG AGG CTC TGC ACA ACC ACT ACA CGC AGA AGA | |
| GCC TCT CCC TGT CTC CGG GTA AAT GAG TGC GAC GGC CGC GAC TCT | |
| AGA GGA T | |

### Example 11

Production of an Antibody

(a)Hybridoma Technology

The antibodies of the present invention can be prepared by a variety of methods. (See, Current Protocols, Chapter 2.) As one example of such methods, cells expressing polypeptide(s) of the invention are administered to an animal to induce the production of sera containing polyclonal antibodies. In a preferred method, a preparation of polypeptide(s) of the invention is prepared and purified to render it substantially free of natural contaminants. Such a preparation is then introduced into an animal in order to produce polyclonal antisera of greater specific activity.

Monoclonal antibodies specific for polypeptide(s) of the invention are prepared using hybridoma technology. (Kohler et al., Nature 256:495 (1975); Kohler et al., Eur. J. Immunol. 6:511 (1976); Kohler et al., Eur. J. Immunol. 6:292 (1976); Hammerling et al., in: Monoclonal Antibodies and T-Cell Hybridomas, Elsevier, N.Y., pp. 563-681 (1981)). In general, an animal (preferably a mouse) is immunized with polypeptide(s) of the invention or, more preferably, with a secreted polypeptide-expressing cell. Such polypeptide-expressing cells are cultured in any suitable tissue culture medium, preferably in Earle's modified Eagle's medium supplemented with 10% fetal bovine serum (inactivated at about 56° C.), and supplemented with about 10 g/l of nonessential amino acids, about 1,000 U/ml of penicillin, and about 100 μg/ml of streptomycin.

The splenocytes of such mice are extracted and fused with a suitable myeloma cell line. Any suitable myeloma cell line may be employed in accordance with the present invention; however, it is preferable to employ the parent myeloma cell line (SP2O), available from the ATCC. After fusion, the resulting hybridoma cells are selectively maintained in HAT medium, and then cloned by limiting dilution as described by Wands et al. (Gastroenterology 80:225-232 (1981)). The hybridoma cells obtained through such a selection are then assayed to identify clones which secrete antibodies capable of binding the polypeptide(s) of the invention.

Alternatively, additional antibodies capable of binding to polypeptide(s) of the invention can be produced in a two-step procedure using anti-idiotypic antibodies. Such a method makes use of the fact that antibodies are themselves antigens, and therefore, it is possible to obtain an antibody which binds to a second antibody. In accordance with this method, protein specific antibodies are used to immunize an animal, preferably a mouse. The splenocytes of such an animal are then used to produce hybridoma cells, and the hybridoma cells are screened to identify clones which produce an antibody whose ability to bind to the protein-specific antibody can be blocked by polypeptide(s) of the invention. Such antibodies comprise anti-idiotypic antibodies to the protein-specific antibody and are used to immunize an animal to induce formation of further protein-specific antibodies.

For in vivo use of antibodies in humans, an antibody is “humanized”. Such antibodies can be produced using genetic constructs derived from hybridoma cells producing the monoclonal antibodies described above. Methods for producing chimeric and humanized antibodies are known in the art and are discussed herein. (See, for review, Morrison, Science 229:1202 (1985); Oi et al., BioTechniques 4:214 (1986); Cabilly et al., U.S. Pat. No. 4,816,567; Taniguchi et al., EP 171496; Morrison et al., EP 173494; Neuberger et al., WO 8601533; Robinson et al., WO 8702671; Boulianne et al., Nature 312:643 (1984); Neuberger et al., Nature 314:268 (1985).)

(b)Isolation of Antibody Fragments Directed Against Polypeptide(s) from a Library of scFvs

Naturally occurring V-genes isolated from human PBLs are constructed into a library of antibody fragments which contain reactivities against polypeptide(s) of the invention to which the donor may or may not have been exposed (see e.g., U.S. Pat. No. 5,885,793 incorporated herein by reference in its entirety).

Rescue of the Library.

A library of scFvs is constructed from the RNA of human PBLs as described in PCT publication WO 92/01047. To rescue phage displaying antibody fragments, approximately 109 E. coli harboring the phagemid are used to inoculate 50 ml of 2×TY containing 1% glucose and 100 μg/ml of ampicillin (2×TY-AMP-GLU) and grown to an O.D. of 0.8 with shaking. Five ml of this culture is used to innoculate 50 ml of 2×TY-AMP-GLU, 2×108 TU of delta gene 3 helper (M13 delta gene III, see PCT publication WO 92/01047) are added and the culture incubated at 37° C. for 45 minutes without shaking and then at 37° C. for 45 minutes with shaking. The culture is centrifuged at 4000 r.p.m. for 10 min. and the pellet resuspended in 2 liters of 2×TY containing 100 μg/ml ampicillin and 50 ug/ml kanamycin and grown overnight. Phage are prepared as described in PCT publication WO 92/01047.

M13 delta gene III is prepared as follows: M13 delta gene III helper phage does not encode gene III protein, hence the phage(mid) displaying antibody fragments have a greater avidity of binding to antigen. Infectious M13 delta gene III particles are made by growing the helper phage in cells harboring a pUC19 derivative supplying the wild type gene III protein during phage morphogenesis. The culture is incubated for 1 hour at 37° C. without shaking and then for a further hour at 37° C. with shaking. Cells are spun down (IEC-Centra 8,400 r.p.m. for 10 min), resuspended in 300 ml 2×TY broth containing 100 μg ampicillin/ml and 25 μg kanamycin/ml (2×TY-AMP-KAN) and grown overnight, shaking at 37° C. Phage particles are purified and concentrated from the culture medium by two PEG-precipitations (Sambrook et al., 1990), resuspended in 2 ml PBS and passed through a 0.45 μm filter (Minisart NML; Sartorius) to give a final concentration of approximately 1013 transducing units/ml (ampicillin-resistant clones).

Panning of the Library.

Immunotubes (Nunc) are coated overnight in PBS with 4 ml of either 100 μg/ml or 10 μg/ml of a polypeptide of the present invention. Tubes are blocked with 2% Marvel-PBS for 2 hours at 37° C. and then washed 3 times in PBS. Approximately 1013 TU of phage is applied to the tube and incubated for 30 minutes at room temperature tumbling on an over and under turntable and then left to stand for another 1.5 hours. Tubes are washed 10 times with PBS 0.1% Tween-20 and 10 times with PBS. Phage are eluted by adding 1 ml of 100 mM triethylamine and rotating 15 minutes on an under and over turntable after which the solution is immediately neutralized with 0.5 ml of 1.0M Tris-HCl, pH 7.4. Phage are then used to infect 10 ml of mid-log E. coli TG1 by incubating eluted phage with bacteria for 30 minutes at 37° C. The E. coli are then plated on TYE plates containing 1% glucose and 100 μg/ml ampicillin. The resulting bacterial library is then rescued with delta gene 3 helper phage as described above to prepare phage for a subsequent round of selection. This process is then repeated for a total of 4 rounds of affinity purification with tube-washing increased to 20 times with PBS, 0.1% Tween-20 and 20 times with PBS for rounds 3 and 4.

Characterization of Binders.

Eluted phage from the 3rd and 4th rounds of selection are used to infect E. coli HB 2151 and soluble scFv is produced (Marks, et al., 1991) from single colonies for assay. ELISAs are performed with microtitre plates coated with either 10 pg/ml of the polypeptide of the present invention in 50 mM bicarbonate pH 9.6. Clones positive in ELISA are further characterized by PCR fingerprinting (see, e.g., PCT publication WO 92/01047) and then by sequencing. These ELISA positive clones may also be further characterized by techniques known in the art, such as, for example, epitope mapping, binding affinity, receptor signal transduction, ability to block or competitively inhibit antibody/antigen binding, and competitive agonistic or antagonistic activity.

### Example 12

Production of PSF-2 Polypeptide for High-Throughput Screening Assays

The following protocol produces a supernatant containing PSF-2 polypeptide to be tested. This supernatant can then be used in the Screening Assays described in Examples 14-21.

First, dilute Poly-D-Lysine (644 587 Boehringer-Mannheim) stock solution (1 mg/ml in PBS) 1:20 in PBS (w/o calcium or magnesium 17-516F Biowhittaker) for a working solution of 50 ug/ml. Add 200 ul of this solution to each well (24 well plates) and incubate at RT for 20 minutes. Be sure to distribute the solution over each well (note: a 12-channel pipetter may be used with tips on every other channel). Aspirate off the Poly-D-Lysine solution and rinse with 1 ml PBS (Phosphate Buffered Saline). The PBS should remain in the well until just prior to plating the cells and plates may be poly-lysine coated in advance for up to two weeks.

Plate 293T cells (do not carry cells past P+20) at 2×10⁵ cells/well in 0.5 ml DMEM(Dulbecco's Modified Eagle Medium)(with 4.5 G/L glucose and L-glutamine (12-604F Biowhittaker))/10% heat inactivated FBS(14-503F Biowhittaker)/1× Penstrep(17-602E Biowhittaker). Let the cells grow overnight.

The next day, mix together in a sterile solution basin: 300 ul Lipofectamine (18324-012 Gibco/BRL) and 5 ml Optimem I (31985070 Gibco/BRL)/96-well plate. With a small volume multi-channel pipetter, aliquot approximately 2 ug of an expression vector containing a polynucleotide insert, produced by the methods described in Examples 8-10, into an appropriately labeled 96-well round bottom plate. With a multi-channel pipetter, add 50 ul of the Lipofectamine/Optimem I mixture to each well. Pipette up and down gently to mix. Incubate at RT 15-45 minutes. After about 20 minutes, use a multi-channel pipetter to add 150 ul Optimem I to each well. As a control, one plate of vector DNA lacking an insert should be transfected with each set of transfections.

Preferably, the transfection should be performed by tag-teaming the following tasks. By tag-teaming, hands on time is cut in half, and the cells do not spend too much time on PBS. First, person A aspirates off the media from four 24-well plates of cells, and then person B rinses each well with 0.5-1 ml PBS. Person A then aspirates off PBS rinse, and person B, using a12-channel pipetter with tips on every other channel, adds the 200 ul of DNA/Lipofectamine/Optimem I complex to the odd wells first, then to the even wells, to each row on the 24-well plates. Incubate at 37 C for 6 hours.

While cells are incubating, prepare appropriate media, either 1%BSA in DMEM with 1× penstrep, or HGS CHO-5 media (116.6 mg/L of CaCl2 (anhyd); 0.00130 mg/L CuSO₄-5H₂O; 0.050 mg/L of Fe(NO₃)₃-9H₂O; 0.417 mg/L of FeSO₄-7H₂O; 311.80 mg/L of Kcl; 28.64 mg/L of MgCl₂; 48.84 mg/L of MgSO₄; 6995.50 mg/L of NaCl; 2400.0 mg/L of NaHCO₃; 62.50 mg/L of NaH₂PO₄-H₂O; 71.02 mg/L of Na₂HPO4; 0.4320 mg/L of ZnSO₄-7H₂O; 0.002 mg/L of Arachidonic Acid; 1.022 mg/L of Cholesterol; 0.070 mg/L of DL-alpha-Tocopherol-Acetate; 0.0520 mg/L of Linoleic Acid; 0.010 mg/L of Linolenic Acid; 0.010 mg/L of Myristic Acid; 0.010 mg/L of Oleic Acid; 0.010 mg/L of Palmitric Acid; 0.010 mg/L of Palmitic Acid; 100 mg/L of Pluronic F-68; 0.010 mg/L of Stearic Acid; 2.20 mg/L of Tween 80; 4551 mg/L of D-Glucose; 130.85 mg/ml of L-Alanine; 147.50 mg/ml of L-Arginine-HCL; 7.50 mg/ml of L-Asparagine-H₂O; 6.65 mg/ml of L-Aspartic Acid; 29.56 mg/ml of L-Cystine-2HCL-H₂O; 31.29 mg/ml of L-Cystine-2HCL; 7.35 mg/ml of L-Glutamic Acid; 365.0 mg/ml of L-Glutamine; 18.75 mg/ml of Glycine; 52.48 mg/ml of L-Histidine-HCL-H₂O; 106.97 mg/ml of L-Isoleucine; 111.45 mg/ml of L-Leucine; 163.75 mg/ml of L-Lysine HCL; 32.34 mg/ml of L-Methionine; 68.48 mg/ml of L-Phenylalainine; 40.0 mg/ml of L-Proline; 26.25 mg/ml of L-Serine; 101.05 mg/ml of L-Threonine; 19.22 mg/ml of L-Tryptophan; 91.79 mg/ml of L-Tryrosine-2Na-2H₂O; and 99.65 mg/ml of L-Valine; 0.0035 mg/L of Biotin; 3.24 mg/L of D-Ca Pantothenate; 11.78 mg/L of Choline Chloride; 4.65 mg/L of Folic Acid; 15.60 mg/L of i-Inositol; 3.02 mg/L of Niacinamide; 3.00 mg/L of Pyridoxal HCL; 0.031 mg/L of Pyridoxine HCL; 0.319 mg/L of Riboflavin; 3.17 mg/L of Thiamine HCL; 0.365 mg/L of Thymidine; 0.680 mg/L of Vitamin B₁₂; 25 mM of HEPES Buffer; 2.39 mg/L of Na Hypoxanthine; 0.105 mg/L of Lipoic Acid; 0.081 mg/L of Sodium Putrescine-2HCL; 55.0 mg/L of Sodium Pyruvate; 0.0067 mg/L of Sodium Selenite; 20 uM of Ethanolamine; 0.122 mg/L of Ferric Citrate; 41.70 mg/L of Methyl-B-Cyclodextrin complexed with Linoleic Acid; 33.33 mg/L of Methyl-B-Cyclodextrin complexed with Oleic Acid; 10 mg/L of Methyl-B-Cyclodextrin complexed with Retinal Acetate. Adjust osmolarity to 327 mOsm) with 2 mm glutamine and 1× penstrep. (BSA (81-068-3 Bayer) 100 gm dissolved in 1 L DMEM for a 10% BSA stock solution). Filter the media and collect 50 ul for endotoxin assay in 15 ml polystyrene conical.

The transfection reaction is terminated, preferably by tag-teaming, at the end of the incubation period. Person A aspirates off the transfection media, while person B adds 1.5 ml appropriate media to each well. Incubate at 37 C for 45 or 72 hours depending on the media used: 1%BSA for 45 hours or CHO-5 for 72 hours.

On day four, using a 300 ul multichannel pipetter, aliquot 600 ul in one 1 ml deep well plate and the remaining supernatant into a 2 ml deep well. The supernatants from each well can then be used in the assays described in Examples 14-21.

It is specifically understood that when activity is obtained in any of the assays described below using a supernatant, the activity originates from either the PSF-2 polypeptide directly (e.g., as a secreted polypeptide) or by PSF-2 inducing expression of other polypeptides, which are then secreted into the supernatant. Thus, the invention further provides a method of identifying the polypeptide in the supernatant characterized by an activity in a particular assay.

### Example 13

Construction of GAS Reporter Construct

One signal transduction pathway involved in the differentiation and proliferation of cells is called the Jaks-STATs pathway. Activated polypeptides in the Jaks-STATs pathway bind to gamma activation site “GAS” elements or interferon-sensitive responsive element (“ISRE”), located in the promoter of many genes. The binding of a polypeptide to these elements alter the expression of the associated gene.

GAS and ISRE elements are recognized by a class of transcription factors called Signal Transducers and Activators of Transcription, or “STATs.” There are six members of the STATs family. Stat1 and Stat3 are present in many cell types, as is Stat2 (as response to IFN-alpha is widespread). Stat4 is more restricted and is not in many cell types though it has been found in T helper class I, cells after treatment with IL-12. Stat5 was originally called mammary growth factor, but has been found at higher concentrations in other cells including myeloid cells. It can be activated in tissue culture cells by many cytokines.

The STATs are activated to translocate from the cytoplasm to the nucleus upon tyrosine phosphorylation by a set of kinases known as the Janus Kinase (“Jaks”) family. Jaks represent a distinct family of soluble tyrosine kinases and include Tyk2, Jak1, Jak2, and Jak3. These kinases display significant sequence similarity and are generally catalytically inactive in resting cells.

The Jaks are activated by a wide range of receptors summarized in the Table below. (Adapted from review by Schidler and Darnell, Ann. Rev. Biochem. 64:621-51 (1995).) A cytokine receptor family, capable of activating Jaks, is divided into two groups: (a) Class 1 includes receptors for IL-2, IL-3, IL-4, IL-6, IL-7, IL-9, IL-11, IL-12, IL-15, Epo, PRL, GH, G-CSF, GM-CSF, LIF, CNTF, and thrombopoietin; and (b) Class 2 includes IFN-alpha, IFN-gamma, and IL-10. The Class 1 receptors share a conserved cysteine motif (a set of four conserved cysteines and one tryptophan) and a WSXWS motif (a membrane proxial region encoding Trp-Ser-Xxx-Trp-Ser (SEQ ID NO:5)).

Thus, on binding of a ligand to a receptor, Jaks are activated, which in turn activate STATs, which then translocate and bind to GAS elements. This entire process is encompassed in the Jaks-STATs signal transduction pathway.

Therefore, activation of the Jaks-STATs pathway, reflected by the binding of the GAS or the ISRE element, can be used to indicate polypeptides involved in the proliferation and differentiation of cells. For example, growth factors and cytokines are known to activate the Jaks-STATs pathway. (See Table below.) Thus, by using GAS elements linked to reporter molecules, activators of the Jaks-STATs pathway can be identified.

| | JAKs | | | | | |
|---|---|---|---|---|---|---|
| Ligand | tyk2 | Jak1 | Jak2 | Jak3 | STATS | GAS (elements) or ISRE |
| IFN family | | | | | | |
| IFN-a/B | + | + | − | − | 1,2,3 | ISRE |
| IFN-g | | + | + | − | 1 | GAS (IRF1 > Lys6 > IFP) |
| I1-10 | + | ? | ? | − | 1,3 | |
| gp130 family | | | | | | |
| IL-6 (Pleiotrohic) | + | + | + | ? | 1,3 | GAS (IRF1 > Lys6 > IFP) |
| I1-11 (Pleiotrohic) | ? | + | ? | ? | 1,3 | |
| OnM (Pleiotrohic) | ? | + | + | ? | 1,3 | |
| LIF (Pleiotrohic) | ? | + | + | ? | 1,3 | |
| CNTF (Pleiotrohic) | −/+ | + | + | ? | 1,3 | |
| G-CSF (Pleiotrohic) | ? | + | ? | ? | 1,3 | |
| IL-12 (Pleiotrohic) | + | − | + | + | 1,3 | |
| g-C family | | | | | | |
| IL-2 (lymphocytes) | − | + | − | + | 1,3,5 | GAS |
| IL-4 (lymph/myeloid) | − | + | − | + | 6 | GAS (IRF1 = IFP >> Ly6)(IgH) |
| IL-7 (lymphocytes) | − | + | − | + | 5 | GAS |
| IL-9 (lymphocytes) | − | + | − | + | 5 | GAS |
| IL-13 (lymphocyte) | − | + | ? | ? | 6 | GAS |
| IL-15 | ? | + | ? | + | 5 | GAS |
| gp140 family | | | | | | |
| IL-3 (myeloid) | − | − | + | − | 5 | GAS (IRF1 > IFP >> Ly6) |
| IL-5 (myeloid) | − | − | + | − | 5 | GAS |
| GM-CSF (myeloid) | − | − | + | − | 5 | GAS |
| Growth hormone family | | | | | | |
| GH | ? | − | + | − | 5 | |
| PRL | ? | +/− | + | − | 1,3,5 | |
| EPO | ? | − | + | − | 5 | GAS (B − CAS > IRF1 = IFP >> Ly6) |
| Receptor Tyrosine Kinases | | | | | | |
| EGF | ? | + | + | − | 1,3 | GAS (IRF1) |
| PDGF | ? | + | + | − | 1,3 | |
| CSF-1 | ? | + | + | − | 1,3 | GAS (not IRF1) |

To construct a synthetic GAS containing promoter element, which is used in the Biological Assays described in Examples 14-15, a PCR based strategy is employed to generate a GAS-SV40 promoter sequence. The 5′ primer contains four tandem copies of the GAS binding site found in the IRF1 promoter and previously demonstrated to bind STATs upon induction with a range of cytokines (Rothman et al., Immunity 1:457-468 (1994).), although other GAS or ISRE elements can be used instead. The 5′ primer also contains 18 bp of sequence complementary to the SV40 early promoter sequence and is flanked with an Xho I site. The sequence of the 5′ primer is: 5′-GCG CCT CGA GAT TTC CCC GAA ATC TAG ATT TCC CCG AAA TGA TTT CCC CGA AAT GAT TTC CCC GAA ATA TCT GCC ATC TCA ATT AG-3′ (SEQ ID NO:6). The downstream primer is complementary to the SV40 promoter and is flanked with a Hin dIII site: 5′-GCG GCA AGC TTT TTG CAA AGC CTA GGC-3′ (SEQ ID NO:7).

PCR amplification is performed using the SV40 promoter template present in the beta-gal:promoter plasmid obtained from Clontech. The resulting PCR fragment is digested with Xho I and Hin dIII and subcloned into BLSK2-. (Stratagene.) Sequencing with forward and reverse primers confirms that the insert contains the following sequence: 5′-CTC GAG ATT TCC CCG AAA TCT AGA TTT CCC CGA AAT GAT TTC CCC GAA ATG ATT TCC CCG AAA TAT CTG CCA TCT CAA TTA GTC AGC AAC CAT AGT CCC GCC CCT AAC TCC GCC CAT CCC GCC CCT AAC TCC GCC CAG TTC CGC CCA TTC TCC GCC CCA TGG CTG ACT AAT TTT TTT TAT TTA TGC AGA GGC CGA GGC CGC CTC GGC CTC TGA GCT ATT CCA GAA GTA GTG AGG AGG CTT TTT TGG AGG CCT AGG CTT TTG CAA AAA GCT T-3′ (SEQ ID NO:8)

With this GAS promoter element linked to the SV40 promoter, a GAS:SEAP2 reporter construct is next engineered. Here, the reporter molecule is a secreted alkaline phosphatase, or “SEAP.” Clearly, however, any reporter molecule can be instead of SEAP, in this or in any of the other Examples. Well known reporter molecules that can be used instead of SEAP include chloramphenicol acetyltransferase (CAT), luciferase, alkaline phosphatase, B-galactosidase, green fluorescent polypeptide (GFP), or any polypeptide detectable by an antibody.

The above sequence confirmed synthetic GAS-SV40 promoter element is subcloned into the pSEAP-Promoter vector obtained from Clontech using HindIII and XhoI, effectively replacing the SV40 promoter with the amplified GAS:SV40 promoter element, to create the GAS-SEAP vector. However, this vector does not contain a neomycin resistance gene, and therefore, is not preferred for mammalian expression systems.

Thus, in order to generate mammalian stable cell lines expressing the GAS-SEAP reporter, the GAS-SEAP cassette is removed from the GAS-SEAP vector using SalI and NotI, and inserted into a backbone vector containing the neomycin resistance gene, such as pGFP-1 (Clontech), using these restriction sites in the multiple cloning site, to create the GAS-SEAP/Neo vector. Once this vector is transfected into mammalian cells, this vector can then be used as a reporter molecule for GAS binding as described in Examples 14-15.

Other constructs can be made using the above description and replacing GAS with a different promoter sequence. For example, construction of reporter molecules containing NF-kappaB and EGR promoter sequences are described in Examples 16 and 17. However, many other promoters can be substituted using the protocols described in these Examples. For instance, SRE, IL-2, NFAT, or Osteocalcin promoters can be substituted, alone or in combination (e.g., GAS/NF-kappaB/EGR, GAS/NF-kappaB, Il-2/NFAT, or NF-kappaB/GAS). Similarly, other cell lines can be used to test reporter construct activity, such as HELA (epithelial), HUVEC (endothelial), Reh (B-cell), Saos-2 (osteoblast), HUVAC (aortic), or Cardiomyocyte.

### Example 14

High-Throughput Screening Assay for T-Cell Activity

The following protocol is used to assess T-cell activity of PSF-2 by determining whether PSF-2 supernatant proliferates and/or differentiates T-cells. T-cell activity is assessed using the GAS/SEAP/Neo construct produced in Example 13. Thus, factors that increase SEAP activity indicate the ability to activate the Jaks-STATS signal transduction pathway. The T-cell used in this assay is Jurkat T-cells (ATCC Accession No. TIB-152), although Molt-3 cells (ATCC Accession No. CRL-1552) and Molt-4 cells (ATCC Accession No. CRL-1582) cells can also be used.

Jurkat T-cells are lymphoblastic CD4+ Th1 helper cells. In order to generate stable cell lines, approximately 2 million Jurkat cells are transfected with the GAS-SEAP/neo vector using DMRIE-C (Life Technologies)(transfection procedure described below). The transfected cells are seeded to a density of approximately 20,000 cells per well and transfectants resistant to 1 mg/ml genticin selected. Resistant colonies are expanded and then tested for their response to increasing concentrations of interferon gamma. The dose response of a selected clone is demonstrated.

Specifically, the following protocol will yield sufficient cells for 75 wells containing 200 ul of cells. Thus, it is either scaled up, or performed in multiple to generate sufficient cells for multiple 96 well plates. Jurkat cells are maintained in RPMI+10% serum with 1%Pen-Strep. Combine 2.5 mls of OPTI-MEM (Life Technologies) with 10 ug of plasmid DNA in a T25 flask. Add 2.5 ml OPTI-MEM containing 50 ul of DMRIE-C and incubate at room temperature for 15-45 mins.

During the incubation period, count cell concentration, spin down the required number of cells (10⁷ per transfection), and resuspend in OPTI-MEM to a final concentration of 10⁷ cells/ml. Then add 1 ml of 1×10⁷ cells in OPTI-MEM to T25 flask and incubate at 37 C for 6 hrs. After the incubation, add 10 ml of RPMI+15% serum.

The Jurkat:GAS-SEAP stable reporter lines are maintained in RPMI+10% serum, 1 mg/ml Genticin, and 1% Pen-Strep. These cells are treated with supernatants containing PSF-2 polypeptides or PSF-2 induced polypeptides as produced by the protocol described in Example 12.

On the day of treatment with the supernatant, the cells should be washed and resuspended in fresh RPMI+10% serum to a density of 500,000 cells per ml. The exact number of cells required will depend on the number of supernatants being screened. For one 96 well plate, approximately 10 million cells (for 10 plates, 100 million cells) are required.

Transfer the cells to a triangular reservoir boat, in order to dispense the cells into a 96 well dish, using a 12 channel pipette. Using a 12 channel pipette, transfer 200 ul of cells into each well (therefore adding 100,000 cells per well).

After all the plates have been seeded, 50 ul of the supernatants are transferred directly from the 96 well plate containing the supernatants into each well using a 12 channel pipette. In addition, a dose of exogenous interferon gamma (0.1, 1.0, 10 ng) is added to wells H9, H10, and H11 to serve as additional positive controls for the assay.

The 96 well dishes containing Jurkat cells treated with supernatants are placed in an incubator for 48 hrs (note: this time is variable between 48-72 hrs). 35 ul samples from each well are then transferred to an opaque 96 well plate using a 12 channel pipette. The opaque plates should be covered (using sellophene covers) and stored at −20 C until SEAP assays are performed according to Example 18. The plates containing the remaining treated cells are placed at 4 C and serve as a source of material for repeating the assay on a specific well if desired.

As a positive control, 100 Unit/ml interferon gamma can be used which is known to activate Jurkat T cells. Over 30 fold induction is typically observed in the positive control wells.

### Example 15

High-Throughput Screening Assay Identifying Myeloid Activity

The following protocol is used to assess myeloid activity of PSF-2 by determining whether PSF-2 proliferates and/or differentiates myeloid cells. Myeloid cell activity is assessed using the GAS/SEAP/Neo construct produced in Example 13. Thus, factors that increase SEAP activity indicate the ability to activate the Jaks-STATS signal transduction pathway. The myeloid cell used in this assay is U937, a pre-monocyte cell line, although TF-1, HL60, or KG1 can be used.

To transiently transfect U937 cells with the GAS/SEAP/Neo construct produced in Example 13, a DEAE-Dextran method (Kharbanda et. al., 1994, Cell Growth & Differentiation, 5:259-265) is used. First, harvest 2×10e⁷ U937 cells and wash with PBS. The U937 cells are usually grown in RPMI 1640 medium containing 10% heat-inactivated fetal bovine serum (FBS) supplemented with 100 units/ml penicillin and 100 mg/ml streptomycin.

Next, suspend the cells in 1 ml of 20 mM Tris-HCl (pH 7.4) buffer containing 0.5 mg/ml DEAE-Dextran, 8 ug GAS-SEAP2 plasmid DNA, 140 mM NaCl, 5 mM KCl, 375 uM Na₂HPO₄.7H₂O, 1 mM MgCl₂, and 675 uM CaCl₂. Incubate at 37 C for 45 min.

Wash the cells with RPMI 1640 medium containing 10% FBS and then resuspend in 10 ml complete medium and incubate at 37 C for 36 hr.

The GAS-SEAP/U937 stable cells are obtained by growing the cells in 400 ug/ml G418. The G418-free medium is used for routine growth but every one to two months, the cells should be re-grown in 400 ug/ml G418 for couple of passages.

These cells are tested by harvesting 1×10⁸ cells (this is enough for ten 96-well plates assay) and wash with PBS. Suspend the cells in 200 ml above described growth medium, with a final density of 5×10⁵ cells/ml. Plate 200 ul cells per well in the 96-well plate (or 1×10⁵ cells/well).

Add 50 ul of the supernatant prepared by the protocol described in Example 12. Incubate at 37 degee C. for 48 to 72 hr. As a positive control, 100 Unit/ml interferon gamma can be used which is known to activate U937 cells. Over 30 fold induction is typically observed in the positive control wells. SEAP assay the supernatant according to the protocol described in Example 18.

### Example 16

High-Throughput Screening Assay Identifying Neuronal Activity

When cells undergo differentiation and proliferation, a group of genes are activated through many different signal transduction pathways. One of these genes, EGR1 (early growth response gene 1), is induced in various tissues and cell types upon activation. The promoter of EGR1 is responsible for such induction. Using the EGR1 promoter linked to reporter molecules, activation of cells can be assessed by PSF-2.

Particularly, the following protocol is used to assess neuronal activity in PC12 cell lines. PC12 cells (rat phenochromocytoma cells) are known to proliferate and/or differentiate by activation with a number of mitogens, such as TPA (tetradecanoyl phorbol acetate), NGF (nerve growth factor), and EGF (epidermal growth factor). The EGR1 gene expression is activated during this treatment. Thus, by stably transfecting PC12 cells with a construct containing an EGR promoter linked to SEAP reporter, activation of PC12 cells by PSF-2 can be assessed.

The EGR/SEAP reporter construct can be assembled by the following protocol. The EGR-1 promoter sequence (−633 to +1)(Sakamoto K et al., Oncogene 6:867-871 (1991)) can be PCR amplified from human genomic DNA using the following primers: 5′ primer: 5′-GCG CTC GAG GGA TGA CAG CGA TAG AAC CCC GG-3′ (SEQ ID NO:9) and 3′ primer: 5′-GCG AAG CTT CGC GAC TCC CCG GAT CCG CCT C-3′ (SEQ ID NO:10).

Using the GAS:SEAP/Neo vector produced in Example 13, EGR1 amplified product can then be inserted into this vector. Linearize the GAS:SEAP/Neo vector using restriction enzymes Xho I and Hin dIII, removing the GAS/SV40 stuffer. Restrict the EGR1 amplified product with these same enzymes. Ligate the vector and the EGR1 promoter.

To prepare 96 well-plates for cell culture, two mls of a coating solution (1:30 dilution of collagen type I (Upstate Biotech Inc. Cat#08-115) in 30% ethanol (filter sterilized)) is added per one 10 cm plate or 50 ml per well of the 96-well plate, and allowed to air dry for 2 hr.

PC12 cells are routinely grown in RPMI-1640 medium (Bio Whittaker) containing 10% horse serum (JRH BIOSCIENCES, Cat. #12449-78P), 5% heat-inactivated fetal bovine serum (FBS) supplemented with 100 units/ml penicillin and 100 ug/ml streptomycin on a precoated 10 cm tissue culture dish. One to four split is done every three to four days. Cells are removed from the plates by scraping and resuspended with pipetting up and down for more than 15 times.

Transfect the EGR/SEAP/Neo construct into PC12 using the Lipofectamine protocol described in Example 12. EGR-SEAP/PC12 stable cells are obtained by growing the cells in 300 ug/ml G418. The G418-free medium is used for routine growth but every one to two months, the cells should be re-grown in 300 ug/ml G418 for couple of passages.

To assay for neuronal activity, a 10 cm plate with cells around 70 to 80% confluent is screened by removing the old medium. Wash the cells once with PBS (Phosphate buffered saline). Then starve the cells in low serum medium (RPMI-1640 containing 1% horse serum and 0.5% FBS with antibiotics) overnight.

The next morning, remove the medium and wash the cells with PBS. Scrape off the cells from the plate, suspend the cells well in 2 ml low serum medium. Count the cell number and add more low serum medium to reach final cell density as 5×10⁵ cells/ml.

Add 200 ul of the cell suspension to each well of 96-well plate (equivalent to 1×10⁵ cells/well). Add 50 ul supernatant produced by Example 12, 37 C for 48 to 72 hr. As a positive control, a growth factor known to activate PC12 cells through EGR can be used, such as 50 ng/ul of Neuronal Growth Factor (NGF). Over fifty-fold induction of SEAP is typically seen in the positive control wells. SEAP assay the supernatant according to Example 18.

### Example 17

High-Throughput Screening Assay for T-cell Activity

NF-kappaB (Nuclear Factor-kappaB) is a transcription factor activated by a wide variety of agents including the inflammatory cytokines IL-1 and TNF, CD30 and CD40, lymphotoxin-alpha and lymphotoxin-beta, by exposure to LPS or thrombin, and by expression of certain viral gene products. As a transcription factor, NF-kappaB regulates the expression of genes involved in immune cell activation, control of apoptosis (NF-kappaB appears to shield cells from apoptosis), B and T-cell development, anti-viral and antimicrobial responses, and multiple stress responses.

In non-stimulated conditions, NF-kappaB is retained in the cytoplasm with I-kappaB (Inhibitor-kappaB). However, upon stimulation, I-kappaB is phosphorylated and degraded, causing NF-kappaB to shuttle to the nucleus, thereby activating transcription of target genes. Target genes activated by NF-kappaB include IL-2, IL-6, GM-CSF, ICAM-1 and class 1 MHC.

Due to its central role and ability to respond to a range of stimuli, reporter constructs utilizing the NF-kappaB promoter element are used to screen the supernatants produced in Example 12. Activators or inhibitors of NF-kappaB would be useful in treating diseases. For example, inhibitors of NF-kappaB could be used to treat those diseases related to the acute or chronic activation of NF-kappaB, such as rheumatoid arthritis.

To construct a vector containing the NF-kappaB promoter element, a PCR based strategy is employed. The upstream primer contains four tandem copies of the NF-kappaB binding site (GGGGACTTTCCC) (SEQ ID NO:1), 18 bp of sequence complementary to the 5′ end of the SV40 early promoter sequence, and is flanked with an Xho I site: 5′-GCG GCC TCG AGG GGA CTT TCC CGG GGA CTT TCC GGG GAC TTT CCG GGA CTT TCC ATC CTG CCA TCT CAA TTA G-3′ (SEQ ID NO:12). The downstream primer is complementary to the 3′ end of the SV40 promoter and is flanked with a Hin dIII site: 5′-GCG GCA AGC TTT TTG CAA AGC CTA GGC-3′ (SEQ ID NO:7).

PCR amplification is performed using the SV40 promoter template present in the pb-gal:promoter plasmid obtained from Clontech. The resulting PCR fragment is digested with Xho I and Hin dIII and subcloned into BLSK2-. (Stratagene) Sequencing with the T7 and T3 primers confirms the insert contains the following sequence: 5′-CTC GAG GGG ACT TTC CCG GGG ACT TTC CGG GGA CTT TCC GGG ACT TTC CAT CTG CCA TCT CAA TTA GTC AGC AAC CAT AGT CCC GCC CCT AAC TCC GCC CAT CCC GCC CCT AAC TCC GCC CAG TTC CGC CCA TTC TCC GCC CCA TGG CTG ACT AAT TTT TTT TAT TTA TGC AGA GGC CGA GGC CGC CTC GGC CTC TGA GCT ATT CCA GAA GTA GTG AGG AGG CTT TTT TGG AGG CCT AGG CTT TTG CAA AAA GCT T-3′ (SEQ ID NO:13).

Next, replace the SV40 minimal promoter element present in the pSEAP2-promoter plasmid (Clontech) with this NF-kB/SV40 fragment using Xho I and Hin dIII. However, this vector does not contain a neomycin resistance gene, and therefore, is not preferred for mammalian expression systems.

In order to generate stable mammalian cell lines, the NF-kappaB/SV40/SEAP cassette is removed from the above NF-kappaB/SEAP vector using restriction enzymes Sal I and Not I, and inserted into a vector containing neomycin resistance. Particularly, the NF-kappaB/SV40/SEAP cassette was inserted into pGFP-1 (Clontech), replacing the GFP gene, after restricting pGFP-1 with Sal I and Not I.

Once NF-kappaB/SV40/SEAP/Neo vector is created, stable Jurkat T-cells are created and maintained according to the protocol described in Example 14. Similarly, the method for assaying supernatants with these stable Jurkat T-cells is also described in Example 14. As a positive control, exogenous TNF alpha (0.1, 1, 10 ng) is added to wells H9, H10, and H11, with a 5-10 fold activation typically observed.

### Example 18

Assay for SEAP Activity

As a reporter molecule for the assays described in Examples 14-17, SEAP activity is assayed using the Tropix Phospho-light Kit (Cat. BP-400) according to the following general procedure. The Tropix Phospho-light Kit supplies the Dilution, Assay, and Reaction Buffers used below.

Prime a dispenser with the 2.5× Dilution Buffer and dispense 15 ul of 2.5× dilution buffer into Optiplates containing 35 ul of a supernatant. Seal the plates with a plastic sealer and incubate at 65 C for 30 min. Separate the Optiplates to avoid uneven heating.

Cool the samples to room temperature for 15 minutes. Empty the dispenser and prime with the Assay Buffer. Add 50 ml Assay Buffer and incubate at room temperature 5 min. Empty the dispenser and prime with the Reaction Buffer (see the table below). Add 50 ul Reaction Buffer and incubate at room temperature for 20 minutes. Since the intensity of the chemiluminescent signal is time dependent, and it takes about 10 minutes to read 5 plates on luminometer, one should treat 5 plates at each time and start the second set 10 minutes later.

Read the relative light unit in the luminometer. Set H12 as blank, and print the results. An increase in chemiluminescence indicates reporter activity.

Reaction Buffer Formulation:

| # of plates | Rxn buffer diluent (ml) | CSPD (ml) |
|---|---|---|
| 10 | 60 | 3 |
| 11 | 65 | 3.25 |
| 12 | 70 | 3.5 |
| 13 | 75 | 3.75 |
| 14 | 80 | 4 |
| 15 | 85 | 4.25 |
| 16 | 90 | 4.5 |
| 17 | 95 | 4.75 |
| 18 | 100 | 5 |
| 19 | 105 | 5.25 |
| 20 | 110 | 5.5 |
| 21 | 115 | 5.75 |
| 22 | 120 | 6 |
| 23 | 125 | 6.25 |
| 24 | 130 | 6.5 |
| 25 | 135 | 6.75 |
| 26 | 140 | 7 |
| 27 | 145 | 7.25 |
| 28 | 150 | 7.5 |
| 29 | 155 | 7.75 |
| 30 | 160 | 8 |
| 31 | 165 | 8.25 |
| 32 | 170 | 8.5 |
| 33 | 175 | 8.75 |
| 34 | 180 | 9 |
| 35 | 185 | 9.25 |
| 36 | 190 | 9.5 |
| 37 | 195 | 9.75 |
| 38 | 200 | 10 |
| 39 | 205 | 10.25 |
| 40 | 210 | 10.5 |
| 41 | 215 | 10.75 |
| 42 | 220 | 11 |
| 43 | 225 | 11.25 |
| 44 | 230 | 11.5 |
| 45 | 235 | 11.75 |
| 46 | 240 | 12 |
| 47 | 245 | 12.25 |
| 48 | 250 | 12.5 |
| 49 | 255 | 12.75 |
| 50 | 260 | 13 |

### Example 19

High-Throughput Screening Assay Identifying Changes in Small Molecule Concentration and Membrane Permeability

Binding of a ligand to a receptor is known to alter intracellular levels of small molecules, such as calcium, potassium, sodium, and pH, as well as alter membrane potential. These alterations can be measured in an assay to identify supernatants which bind to receptors of a particular cell. Although the following protocol describes an assay for calcium, this protocol can easily be modified to detect changes in potassium, sodium, pH, membrane potential, or any other small molecule which is detectable by a fluorescent probe.

The following assay uses Fluorometric Imaging Plate Reader (“FLIPR”) to measure changes in fluorescent molecules (Molecular Probes) that bind small molecules. Clearly, any fluorescent molecule detecting a small molecule can be used instead of the calcium fluorescent molecule, fluo-3, used here.

For adherent cells, seed the cells at 10,000-20,000 cells/well in a Co-star black 96-well plate with clear bottom. The plate is incubated in a CO₂ incubator for 20 hours. The adherent cells are washed two times in Biotek washer with 200 ul of HBSS (Hank's Balanced Salt Solution) leaving 100 ul of buffer after the final wash.

A stock solution of 1 mg/ml fluo-3 is made in 10% pluronic acid DMSO. To load the cells with fluo-3, 50 ul of 12 ug/ml fluo-3 is added to each well. The plate is incubated at 37 C in a CO₂ incubator for 60 min. The plate is washed four times in the Biotek washer with HBSS leaving 100 ul of buffer.

For non-adherent cells, the cells are spun down from culture media. Cells are re-suspended to 2-5×10⁶ cells/ml with HBSS in a 50-ml conical tube. 4 ul of 1 mg/ml fluo-3 solution in 10% pluronic acid DMSO is added to each ml of cell suspension. The tube is then placed in a 37 C water bath for 30-60 min. The cells are washed twice with HBSS, resuspended to 1×10⁶ cells/ml, and dispensed into a microplate, 100 ul/well. The plate is centrifuged at 1000 rpm for 5 min. The plate is then washed once in Denley CellWash with 200 ul, followed by an aspiration step to 100 ul final volume.

For a non-cell based assay, each well contains a fluorescent molecule, such as fluo-3. The supernatant is added to the well, and a change in fluorescence is detected.

To measure the fluorescence of intracellular calcium, the FLIPR is set for the following parameters: (1) System gain is 300-800 mW; (2) Exposure time is 0.4 second; (3) Camera F/stop is F/2; (4) Excitation is 488 nm; (5) Emission is 530 nm; and (6) Sample addition is 50 ul. Increased emission at 530 nm indicates an extracellular signaling event caused by the a molecule, either PSF-2 or a molecule induced by PSF-2, which has resulted in an increase in the intracellular Ca²⁺ concentration.

### Example 20

High-Throughput Screening Assay Identifying Tyrosine Kinase Activity

The Polypeptide Tyrosine Kinases (PTK) represent a diverse group of transmembrane and cytoplasmic kinases. Within the Receptor Polypeptide Tyrosine Kinase RPTK) group are receptors for a range of mitogenic and metabolic growth factors including the PDGF, FGF, EGF, NGF, HGF and Insulin receptor subfamilies. In addition there are a large family of RPTKs for which the corresponding ligand is unknown. Ligands for RPTKs include mainly secreted small polypeptides, but also membrane-bound and extracellular matrix polypeptides.

Activation of RPTK by ligands involves ligand-mediated receptor dimerization, resulting in transphosphorylation of the receptor subunits and activation of the cytoplasmic tyrosine kinases. The cytoplasmic tyrosine kinases include receptor associated tyrosine kinases of the src-family (e.g., src, yes, lck, lyn, fyn) and non-receptor linked and cytosolic polypeptide tyrosine kinases, such as the Jak family, members of which mediate signal transduction triggered by the cytokine superfamily of receptors (e.g., the Interleukins, Interferons, GM-CSF, and Leptin).

Because of the wide range of known factors capable of stimulating tyrosine kinase activity, identifying whether PSF-2 or a molecule induced by PSF-2 is capable of activating tyrosine kinase signal transduction pathways is of interest. Therefore, the following protocol is designed to identify such molecules capable of activating the tyrosine kinase signal transduction pathways.

Seed target cells (e.g., primary keratinocytes) at a density of approximately 25,000 cells per well in a 96 well Loprodyne Silent Screen Plates purchased from Nalge Nunc (Naperville, Ill.). The plates are sterilized with two 30 minute rinses with 100% ethanol, rinsed with water and dried overnight. Some plates are coated for 2 hr with 100 ml of cell culture grade type I collagen (50 mg/ml), gelatin (2%) or polylysine (50 mg/ml), all of which can be purchased from Sigma Chemicals (St. Louis, Mo.) or 10% Matrigel purchased from Becton Dickinson (Bedford, Mass.), or calf serum, rinsed with PBS and stored at 4 C. Cell growth on these plates is assayed by seeding 5,000 cells/well in growth medium and indirect quantitation of cell number through use of alamarBlue as described by the manufacturer Alamar Biosciences, Inc. (Sacramento, Calif.) after 48 hr. Falcon plate covers #3071 from Becton Dickinson (Bedford, Mass.) are used to cover the Loprodyne Silent Screen Plates. Falcon Microtest III cell culture plates can also be used in some proliferation experiments.

To prepare extracts, A431 cells are seeded onto the nylon membranes of Loprodyne plates (20,000/200 ml/well) and cultured overnight in complete medium. Cells are quiesced by incubation in serum-free basal medium for 24 hr. After 5-20 minutes treatment with EGF (60ng/ml) or 50 ul of the supernatant produced in Example 12, the medium was removed and 100 ml of extraction buffer ((20 mM HEPES pH 7.5, 0.15 M NaCl, 1% Triton X-100, 0.1% SDS, 2 mM Na3VO4, 2 mM Na4P2O7 and a cocktail of protease inhibitors (#1836170) obtained from Boeheringer Mannheim (Indianapolis, Ind.) is added to each well and the plate is shaken on a rotating shaker for 5 minutes at 4° C. The plate is then placed in a vacuum transfer manifold and the extract filtered through the 0.45 mm membrane bottoms of each well using house vacuum. Extracts are collected in a 96-well catch/assay plate in the bottom of the vacuum manifold and immediately placed on ice. To obtain extracts clarified by centrifugation, the content of each well, after detergent solubilization for 5 minutes, is removed and centrifuged for 15 minutes at 4 C at 16,000×g.

Test the filtered extracts for levels of tyrosine kinase activity. Although many methods of detecting tyrosine kinase activity are known, one method is described here.

Generally, the tyrosine kinase activity of a supernatant is evaluated by determining its ability to phosphorylate a tyrosine residue on a specific substrate (a biotinylated peptide). Biotinylated peptides that can be used for this purpose include PSK1 (corresponding to amino acids 6-20 of the cell division kinase cdc2-p34) and PSK2 (corresponding to amino acids 1-17 of gastrin). Both peptides are substrates for a range of tyrosine kinases and are available from Boehringer Mannheim.

The tyrosine kinase reaction is set up by adding the following components in order. First, add 10 ul of 5 uM Biotinylated Peptide, then 10 ul ATP/Mg₂₊ (5 mM ATP/50 mM MgCl₂), then 10 ul of 5× Assay Buffer (40 mM imidazole hydrochloride, pH7.3, 40 mM beta-glycerophosphate, 1 mM EGTA, 100 mM MgCl₂, 5 mM MnCl₂, 0.5 mg/ml BSA), then 5 ul of Sodium Vanadate(1 mM), and then 5 ul of water. Mix the components gently and preincubate the reaction mix at 30 C for 2 min. Initial the reaction by adding 10 ul of the control enzyme or the filtered supernatant.

The tyrosine kinase assay reaction is then terminated by adding 10 ul of 120 mm EDTA and place the reactions on ice.

Tyrosine kinase activity is determined by transferring 50 ul aliquot of reaction mixture to a microtiter plate (MTP) module and incubating at 37 C for 20 min. This allows the streptavadin coated 96 well plate to associate with the biotinylated peptide. Wash the MTP module with 300 ul/well of PBS four times. Next add 75 ul of anti-phospotyrosine antibody conjugated to horse radish peroxidase (anti-P-Tyr-POD(0.5 u/ml)) to each well and incubate at 37 C for one hour. Wash the well as above.

Next add 100 ul of peroxidase substrate solution (Boehringer Mannheim) and incubate at room temperature for at least 5 mins (up to 30 min). Measure the absorbance of the sample at 405 nm by using ELISA reader. The level of bound peroxidase activity is quantitated using an ELISA reader and reflects the level of tyrosine kinase activity.

### Example 21

High-Throughput Screening Assay Identifying Phosphorylation Activity

As a potential alternative and/or compliment to the assay of polypeptide tyrosine kinase activity described in Example 20, an assay which detects activation (phosphorylation) of major intracellular signal transduction intermediates can also be used. For example, as described below one particular assay can detect tyrosine phosphorylation of the Erk-1 and Erk-2 kinases. However, phosphorylation of other molecules, such as Raf, JNK, p38 MAP, Map kinase kinase (MEK), MEK kinase, Src, Muscle specific kinase (MuSK), IRAK, Tec, and Janus, as well as any other phosphoserine, phosphotyrosine, or phosphothreonine molecule, can be detected by substituting these molecules for Erk-1 or Erk-2 in the following assay.

Specifically, assay plates are made by coating the wells of a 96-well ELISA plate with 0.1 ml of polypeptide G (1 ug/ml) for 2 hr at room temp, (RT). The plates are then rinsed with PBS and blocked with 3% BSA/PBS for 1 hr at RT. The polypeptide G plates are then treated with 2 commercial monoclonal antibodies (10 ng/well) against Erk-1 and Erk-2 (1 hr at RT) (Santa Cruz Biotechnology). (To detect other molecules, this step can easily be modified by substituting a monoclonal antibody detecting any of the above described molecules.) After 3-5 rinses with PBS, the plates are stored at 4 C until use.

A431 cells are seeded at 20,000/well in a 96-well Loprodyne filterplate and cultured overnight in growth medium. The cells are then starved for 48 hr in basal medium (DMEM) and then treated with EGF (6 ng/well) or 50 ul of the supernatants obtained in Example 12 for 5-20 minutes. The cells are then solubilized and extracts filtered directly into the assay plate.

After incubation with the extract for 1 hr at RT, the wells are again rinsed. As a positive control, a commercial preparation of MAP kinase (10 ng/well) is used in place of A431 extract. Plates are then treated with a commercial polyclonal (rabbit) antibody (1 ug/ml) which specifically recognizes the phosphorylated epitope of the Erk-1 and Erk-2 kinases (1 hr at RT). This antibody is biotinylated by standard procedures. The bound polyclonal antibody is then quantitated by successive incubations with Europium-streptavidin and Europium fluorescence enhancing reagent in the Wallac DELFIA instrument (time-resolved fluorescence). An increased fluorescent signal over background indicates a phosphorylation by PSF-2 or a molecule induced by PSF-2.

### Example 22

Method of Determining Alterations in the PSF-2 Gene

RNA isolated from entire families or individual patients presenting with a phenotype of interest (such as a disease) is be isolated. cDNA is then generated from these RNA samples using protocols known in the art. (See, Sambrook.) The cDNA is then used as a template for PCR, employing primers surrounding regions of interest in SEQ ID NO:1. Suggested PCR conditions consist of 35 cycles at 95 C for 30 seconds; 60-120 seconds at 52-58 C; and 60-120 seconds at 70 C, using buffer solutions described in Sidransky, D., et al., Science 252:706 (1991).

PCR products are then sequenced using primers labeled at their 5′ end with T4 polynucleotide kinase, employing SequiTherm Polymerase. (Epicentre Technologies). The intron-exon borders of selected exons of PSF-2 is also determined and genomic PCR products analyzed to confirm the results. PCR products harboring suspected mutations in PSF-2 is then cloned and sequenced to validate the results of the direct sequencing.

PCR products of PSF-2 are cloned into T-tailed vectors as described in Holton, T. A. and Graham, M. W., Nucleic Acids Research, 19:1156 (1991) and sequenced with T7 polymerase (United States Biochemical). Affected individuals are identified by mutations in PSF-2 not present in unaffected individuals.

Genomic rearrangements are also observed as a method of determining alterations in the PSF-2 gene. Genomic clones isolated according to Example 2 are nick-translated with digoxigenindeoxy-uridine 5′-triphosphate (Boehringer Manheim), and FISH performed as described in Johnson, Cg. et al., Methods Cell Biol. 35:73-99 (1991). Hybridization with the labeled probe is carried out using a vast excess of human cot-1 DNA for specific hybridization to the PSF-2 genomic locus.

Chromosomes are counterstained with 4,6-diamino-2-phenylidole and propidium iodide, producing a combination of C- and R-bands. Aligned images for precise mapping are obtained using a triple-band filter set (Chroma Technology, Brattleboro, Vt.) in combination with a cooled charge-coupled device camera (Photometrics, Tucson, Ariz.) and variable excitation wavelength filters. (Johnson, Cv. et al., Genet. Anal. Tech. Appl., 8:75 (1991).) Image collection, analysis and chromosomal fractional length measurements are performed using the ISee Graphical Program System. (Inovision Corporation, Durham, N.C.) Chromosome alterations of the genomic region of PSF-2 (hybridized by the probe) are identified as insertions, deletions, and translocations. These PSF-2 alterations are used as a diagnostic marker for an associated disease.

### Example 23

Method of Detecting Abnormal Levels of PSF-2 in a Biological Sample

PSF-2 polypeptides can be detected in a biological sample, and if an increased or decreased level of PSF-2 is detected, this polypeptide is a marker for a particular phenotype. Methods of detection are numerous, and thus, it is understood that one skilled in the art can modify the following assay to fit their particular needs.

For example, antibody-sandwich ELISAs are used to detect PSF-2 in a sample, preferably a biological sample. Wells of a microtiter plate are coated with specific antibodies to PSF-2, at a final concentration of 0.2 to 10 ug/ml. The antibodies are either monoclonal or polyclonal and are produced by the method described in Example 11. The wells are blocked so that non-specific binding of PSF-2 to the well is reduced.

The coated wells are then incubated for >2 hours at RT with a sample containing PSF-2. Preferably, serial dilutions of the sample should be used to validate results. The plates are then washed three times with deionized or distilled water to remove unbounded PSF-2.

Next, 50 ul of specific antibody-alkaline phosphatase conjugate, at a concentration of 25-400 ng, is added and incubated for 2 hours at room temperature. The plates are again washed three times with deionized or distilled water to remove unbounded conjugate.

Add 75 ul of 4-methylumbelliferyl phosphate (MUP) or p-nitrophenyl phosphate (NPP) substrate solution to each well and incubate 1 hour at room temperature. Measure the reaction by a microtiter plate reader. Prepare a standard curve, using serial dilutions of a control sample, and plot PSF-2 polypeptide concentration on the X-axis (log scale) and fluorescence or absorbance of the Y-axis (linear scale). Interpolate the concentration of the PSF-2 in the sample using the standard curve.

### Example 24

Formulating a Polypeptide

The invention also provides methods of treatment and/or prevention of diseases or disorders (such as, for example, any one or more of the diseases or disorders disclosed herein) by administration to a subject of an effective amount of a Therapeutic. By “therapeutic” is meant a polynucleotide or polypeptide of the invention (including fragments and variants thereof), agonists or antagonists thereof, and/or antibodies thereto, in combination with a pharmaceutically acceptable carrier type (e.g., a sterile carrier).

The therapeutic will be formulated and dosed in a fashion consistent with good medical practice, taking into account the clinical condition of the individual patient (especially the side effects of treatment with the therapeutic alone), the site of delivery, the method of administration, the scheduling of administration, and other factors known to practitioners. The “effective amount” for purposes herein is thus determined by such considerations.

As a general proposition, the total pharmaceutically effective amount of the therapeutic of the invention administered parenterally per dose will be in the range of about 1 ug/kg/day to 10 mg/kg/day of patient body weight, although, as noted above, this will be subject to therapeutic discretion. More preferably, this dose is at least 0.01 mg/kg/day, and most preferably for humans between about 0.01 and 1 mg/kg/day for the hormone. If given continuously, the therapeutic of the invention is typically administered at a dose rate of about 1 ug/kg/hour to about 50 ug/kg/hour, either by 1-4 injections per day or by continuous subcutaneous infusions, for example, using a mini-pump. An intravenous bag solution may also be employed. The length of treatment needed to observe changes and the interval following treatment for responses to occur appears to vary depending on the desired effect.

Pharmaceutical compositions containing PSF-2 are administered orally, rectally, parenterally, intracistemally, intravaginally, intraperitoneally, topically (as by powders, ointments, gels, drops or transdermal patch), bucally, or as an oral or nasal spray. “Pharmaceutically acceptable carrier” refers to a non-toxic solid, semisolid or liquid filler, diluent, encapsulating material or formulation auxiliary of any type. The term “parenteral” as used herein refers to modes of administration which include intravenous, intramuscular, intraperitoneal, intrasternal, subcutaneous and intraarticular injection and infusion.

Therapeutics of the invention are also suitably administered by sustained-release systems. Suitable examples of sustained-release compositions include semi-permeable polymer matrices in the form of shaped articles, e.g., films, or mirocapsules. Sustained-release matrices include polylactides (U.S. Pat. No. 3,773,919, EP 58,481), copolymers of L-glutamic acid and gamma-ethyl-L-glutamate (Sidman, U. et al., Biopolymers 22:547-556 (1983)), poly (2-hydroxyethyl methacrylate) (R. Langer et al., J. Biomed. Mater. Res. 15:167-277 (1981), and R. Langer, Chem. Tech. 12:98-105 (1982)), ethylene vinyl acetate (R. Langer et al.) or poly-D-(−)-3-hydroxybutyric acid (EP 133,988). Sustained-release compositions also include liposomally entrapped therapeutics of the invention. Liposomes containing the therapeutics of the invention are prepared by methods known per se: DE 3,218,121; Epstein et al., Proc. Natl. Acad. Sci. USA 82:3688-3692 (1985); Hwang et al., Proc. Natl. Acad. Sci. USA 77:4030-4034 (1980); EP 52,322; EP 36,676; EP 88,046; EP 143,949; EP 142,641; Japanese Pat. Appl. 83-118008; U.S. Pat. Nos. 4,485,045 and 4,544,545; and EP 102,324. Ordinarily, the liposomes are of the small (about 200-800 Angstroms) unilamellar type in which the lipid content is greater than about 30 mol. percent cholesterol, the selected proportion being adjusted for the optimal secreted polypeptide therapy.

For parenteral administration, in one embodiment, PSF-2 is formulated generally by mixing it at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation. For example, the formulation preferably does not include oxidizing agents and other compounds that are known to be deleterious to polypeptides.

Generally, the formulations are prepared by contacting the therapeutics of the invention uniformly and intimately with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringer's solution, and dextrose solution. Non-aqueous vehicles such as fixed oils and ethyl oleate are also useful herein, as well as liposomes.

The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; polypeptides, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, manose, or dextrins; chelating agents such as EDTA; sugar alcohols such as mannitol or sorbitol; counterions such as sodium; and/or nonionic surfactants such as polysorbates, poloxamers, or PEG.

Therapeutics of the invention are typically formulated in such vehicles at a concentration of about 0.1 mg/ml to 100 mg/ml, preferably 1-10 mg/ml, at a pH of about 3 to 8. It will be understood that the use of certain of the foregoing excipients; carriers, or stabilizers will result in the formation of polypeptide salts.

Therapeutics of the invention used for therapeutic administration can be sterile. Sterility is readily accomplished by filtration through sterile filtration membranes (e.g., 0.2 micron membranes). Therapeutic polypeptide compositions generally are placed into a container having a sterile access port, for example, an intravenous solution bag or vial having a stopper pierceable by a hypodermic injection needle.

Therapeutics of the invention ordinarily will be stored in unit or multi-dose containers, for example, sealed ampoules or vials, as an aqueous solution or as a lyophilized formulation for reconstitution. As an example of a lyophilized formulation, 10-ml vials are filled with 5 ml of sterile-filtered 1% (w/v) aqueous PSF-2 polypeptide solution, and the resulting mixture is lyophilized. The infusion solution is prepared by reconstituting the lyophilized therapeutic using bacteriostatic Water-for-Injection.

The invention also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration. In addition, PSF-2 may be employed in conjunction with other therapeutic compounds.

Therapeutics of the invention may be administered alone or in combination with adjuvants. Adjuvants that may be administered with the therapeutics of the invention include, but are not limited to, alum, alum plus deoxycholate (ImmunoAg), MTP-PE (Biocine Corp.), QS21 (Genentech, Inc.), BCG, and MPL. In a specific embodiment, therapeutics of the invention are administered in combination with alum. In another specific embodiment, therapeutics of the invention are administered in combination with QS-21. Further adjuvants that may be administered with the therapeutics of the invention include, but are not limited to, Monophosphoryl lipid immunomodulator, AdjuVax 100a, QS-21, QS-18, CRL1005, Aluminum salts, MF-59, and Virosomal adjuvant technology. Vaccines that may be administered with the therapeutics of the invention include, but are not limited to, vaccines directed toward protection against MMR (measles, mumps, rubella), polio, varicella, tetanus/diptheria, hepatitis A, hepatitis B, haemophilus influenzac B, whooping cough, pneumonia, influenza, Lyme's Disease, rotavirus, cholera, yellow fever, Japanese encephalitis, poliomyelitis, rabies, typhoid fever, and pertussis. Combinations may be administered either concomitantly, e.g., as an admixture, separately but simultaneously or concurrently; or sequentially. This includes presentations in which the combined agents are administered together as a therapeutic mixture, and also procedures in which the combined agents are administered separately but simultaneously, e.g., as through separate intravenous lines into the same individual. Administration “in combination” further includes the separate administration of one of the compounds or agents given first, followed by the second.

The therapeutics of the invention may be administered alone or in combination with other therapeutic agents. Therapeutic agents that may be administered in combination with the therapeutics of the invention, include but not limited to, members of the TNF family, chemotherapeutic agents, antibiotics, steroidal and non-steroidal anti-inflammatories, conventional immunotherapeutic agents, cytokines and/or growth factors. Combinations may be administered either concomitantly, e.g., as an admixture, separately but simultaneously or concurrently; or sequentially. This includes presentations in which the combined agents are administered together as a therapeutic mixture, and also procedures in which the combined agents are administered separately but simultaneously, e.g., as through separate intravenous lines into the same individual. Administration “in combination” further includes the separate administration of one of the compounds or agents given first, followed by the second.

In one embodiment, the therapeutics of the invention are administered in combination with members of the TNF family. TNF, TNF-related or TNF-like molecules that may be administered with the therapeutics of the invention include, but are not limited to, soluble forms of TNF-alpha, lymphotoxin-alpha (LT-alpha, also known as TNF-beta), LT-beta (found in complex heterotrimer LT-alpha2-beta), OPGL, FasL, CD27L, CD30L, CD40L, 4-1BBL, DcR3, OX40L, TNF-gamma (International Publication No. WO 96/14328), AIM-I (International Publication No. WO 97/33899), endokine-alpha (International Publication No. WO 98/07880), TR6 (International Publication No. WO 98/30694), OPG, and neutrokine-alpha (International Publication No. WO 98/18921, OX40, and nerve growth factor (NGF), and soluble forms of Fas, CD30, CD27, CD40 and 4-IBB, TR2 (International Publication No. WO 96/34095), DR3 (International Publication No. WO 97/33904), DR4 (International Publication No. WO 98/32856), TR5 (International Publication No. WO 98/30693), TR6 (International Publication No. WO 98/30694), TR7 (International Publication No. WO 98/41629), TRANK, TR9 (International Publication No. WO 98/56892),TR10 (International Publication No. WO 98/54202), 312C2 (International Publication No. WO 98/06842), and TR12, and soluble forms CD154, CD70, and CD153.

In certain embodiments, Therapeutics of the invention are administered in combination with antiretroviral agents, nucleoside reverse transcriptase inhibitors, non-nucleoside reverse transcriptase inhibitors, and/or protease inhibitors. Nucleoside reverse transcriptase inhibitors that may be administered in combination with the Therapeutics of the invention, include, but are not limited to, RETROVIR™ (zidovudine/AZT), VIDEX™ (didanosine/ddI), HIVID™ (zalcitabine/ddC), ZERIT™ (stavudine/d4T), EPIVIR™ (lamivudine/3TC), and COMBIVIR™ (zidovudine/lamivudine). Non-nucleoside reverse transcriptase inhibitors that may be administered in combination with the therapeutics of the invention, include, but are not limited to, VIRAMUNE™ (nevirapine), RESCRIPTOR™ (delavirdine), and SUSTIVA™ (efavirenz). Protease inhibitors that may be administered in combination with the therapeutics of the invention, include, but are not limited to, CRIXIVAN™ (indinavir), NORVIR™ (ritonavir), INVIRASE™ (saquinavir), and VIRACEPT™ (nelfinavir). In a specific embodiment, antiretroviral agents, nucleoside reverse transcriptase inhibitors, non-nucleoside reverse transcriptase inhibitors, and/or protease inhibitors may be used in any combination with therapeutics of the invention to treat AIDS and/or to prevent or treat HIV infection.

In other embodiments, therapeutics of the invention may be administered in combination with anti-opportunistic infection agents. Anti-opportunistic agents that may be administered in combination with the therapeutics of the invention, include, but are not limited to, TRIMETHOPRIM-SULFAMETHOXAZOLE™, DAPSONE™, PENTAMIDINE™, ATOVAQUONE™, ISONIAZID™, RIFAMPIN™, PYRAZINAMIDE™, ETHAMBUTOL™, RIFABUTIN™, CLARITHROMYCIN™, AZITHROMYCIN™, GANCICLOVIR™, FOSCARNET™, CIDOFOVIR™, FLUCONAZOLE™, ITRACONAZOLE™, KETOCONAZOLE™, ACYCLOVIR™, FAMCICOLVIR™, PYRIMETHAMINE™, LEUCOVORIN™, NEUPOGEN™ (filgrastim/G-CSF), and LEUKINE™ (sargramostim/GM-CSF). In a specific embodiment, therapeutics of the invention are used in any combination with TREMETHOPRMI-SULFAMETHOXAZOLE™, DAPSONE™, PENTAMIDINE™, and/or ATOVAQUONE™ to prophylactically treat or prevent an opportunistic Pneumocystis carinii pneumonia infection. In another specific embodiment, therapeutics of the invention are used in any combination with ISONIAZID™, RIFAMPIN™, PYRAZINAMIDE™, and/or ETHAMBUTOL™ to prophylactically treat or prevent an opportunistic Mycobacterium avium complex infection. In another specific embodiment, therapeutics of the invention are used in any combination with RIFABUTIN™, CLARITHROMYCIN™, and/or AZITHROMYCIN™ to prophylactically treat or prevent an opportunistic Mycobacterium tuberculosis infection. In another specific embodiment, therapeutics of the invention are used in any combination with GANCICLOVIR™, FOSCARNET™, and/or CIDOFOVIR™ to prophylactically treat or prevent an opportunistic cytomegalovirus infection. In another specific embodiment, therapeutics of the invention are used in any combination with FLUCONAZOLE™, ITRACONAZOLE™, and/or KETOCONAZOLE™ to prophylactically treat or prevent an opportunistic fungal infection. In another specific embodiment, therapeutics of the invention are used in any combination with ACYCLOVIR™ and/or FAMCICOLVIR™ to prophylactically treat or prevent an opportunistic herpes simplex virus type I and/or type II infection. In another specific embodiment, therapeutics of the invention are used in any combination with PYRIMETHAMINE™ and/or LEUCOVORIN™ to prophylactically treat or prevent an opportunistic Toxoplasma gondii infection. In another specific embodiment, therapeutics of the invention are used in any combination with LEUCOVORIN™ and/or NEUPOGEN™ to prophylactically treat or prevent an opportunistic bacterial infection.

In a further embodiment, the therapeutics of the invention are administered in combination with an antiviral agent. Antiviral agents that may be administered with the therapeutics of the invention include, but are not limited to, acyclovir, ribavirin, amantadine, and remantidine.

In a further embodiment, the therapeutics of the invention are administered in combination with an antibiotic agent. Antibiotic agents that may be administered with the therapeutics of the invention include, but are not limited to, amoxicillin, beta-lactamases, aminoglycosides, beta-lactam (glycopeptide), beta-lactamases, Clindamycin, chloramphenicol, cephalosporins, ciprofloxacin, ciprofloxacin, erythromycin, fluoroquinolones, macrolides, metronidazole, penicillins, quinolones, rifampin, streptomycin, sulfonamide, tetracyclines, trimethoprim, trimethoprim-sulfamthoxazole, and vancomycin.

Conventional nonspecific immunosuppressive agents, that may be administered in combination with the therapeutics of the invention include, but are not limited to, steroids, cyclosporine, cyclosporine analogs, cyclophosphamide methylprednisone, prednisone, azathioprine, FK-506, 15-deoxyspergualin, and other immunosuppressive agents that act by suppressing the function of responding T cells.

In specific embodiments, therapeutics of the invention are administered in combination with immunosuppressants. Immunosuppressants preparations that may be administered with the therapeutics of the invention include, but are not limited to, ORTHOCLONE™ (OKT3), SANDIMMUNE™/NEORAL™/SANGDYA™ (cyclosporin), PROGRAF™ (tacrolimus), CELLCEPT™ (mycophenolate), Azathioprine, glucorticosteroids, and RAPAMUNE™ (sirolimus). In a specific embodiment, immunosuppressants may be used to prevent rejection of organ or bone marrow transplantation.

In an additional embodiment, therapeutics of the invention are administered alone or in combination with one or more intravenous immune globulin preparations. Intravenous immune globulin preparations that may be administered with the therapeutics of the invention include, but not limited to, GAMMAR™, IVEEGAM™, SANDOGLOBULIN™, GAMMAGARD S/D™, and GAMIMUNE™. In a specific embodiment, therapeutics of the invention are administered in combination with intravenous immune globulin preparations in transplantation therapy (e.g., bone marrow transplant).

In an additional embodiment, the therapeutics of the invention are administered alone or in combination with an anti-inflammatory agent. Anti-inflammatory agents that may be administered with the therapeutics of the invention include, but are not limited to, glucocorticoids and the nonsteroidal anti-inflammatories, aminoarylcarboxylic acid derivatives, arylacetic acid derivatives, arylbutyric acid derivatives, arylcarboxylic acids, arylpropionic acid derivatives, pyrazoles, pyrazolones, salicylic acid derivatives, thiazinecarboxamides, e-acetamidocaproic acid, S-adenosylmethionine, 3-amino-4-hydroxybutyric acid, amixetrine, bendazac, benzydamine, bucolome, difenpiramide, ditazol, emorfazone, guaiazulene, nabumetone, nimesulide, orgotein, oxaceprol, paranyline, perisoxal, pifoxime, proquazone, proxazole, and tenidap.

In another embodiment, compostions and/or therapeutics of the invention are administered in combination with a chemotherapeutic agent. Chemotherapeutic agents that may be administered with the therapeutics of the invention include, but are not limited to, antibiotic derivatives (e.g., doxorubicin, bleomycin, daunorubicin, and dactinomycin); antiestrogens (e.g., tamoxifen); antimetabolites (e.g., fluorouracil, 5-FU, methotrexate, floxuridine, interferon alpha-2b, glutamic acid, plicamycin, mercaptopurine, and 6-thioguanine); cytotoxic agents (e.g., carmustine, BCNU, lomustine, CCNU, cytosine arabinoside, cyclophosphamide, estramustine, hydroxyurea, procarbazine, mitomycin, busulfan, cis-platin, and vincristine sulfate); hormones (e.g., medroxyprogesterone, estramustine phosphate sodium, ethinyl estradiol, estradiol, megestrol acetate, methyltestosterone, diethylstilbestrol diphosphate, chlorotrianisene, and testolactone); nitrogen mustard derivatives (e.g., mephalen, chorambucil, mechlorethamine (nitrogen mustard) and thiotepa); steroids and combinations (e.g., bethamethasone sodium phosphate); and others (e.g., dicarbazine, asparaginase, mitotane, vincristine sulfate, vinblastine sulfate, and etoposide).

In a specific embodiment, therapeutics of the invention are administered in combination with CHOP (cyclophosphamide, doxorubicin, vincristine, and prednisone) or any combination of the components of CHOP. In another embodiment, therapeutics of the invention are administered in combination with Rituximab. In a further embodiment, therapeutics of the invention are administered with Rituxmab and CHOP, or Rituxmab and any combination of the components of CHOP.

In an additional embodiment, the therapeutics of the invention are administered in combination with cytokines. Cytokines that may be administered with the therapeutics of the invention include, but are not limited to, IL2, IL3, IL4, IL5, IL6, IL7, IL10, IL12, IL13, IL15, anti-CD40, CD40L, IFN-gamma and TNF-alpha. In another embodiment, therapeutics of the invention may be administered with any interleukin, including, but not limited to, IL-1 alpha, IL-1beta, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, and IL-22.

In an additional embodiment, the therapeutics of the invention are administered in combination with angiogenic proteins. Angiogenic proteins that may be administered with the therapeutics of the invention include, but are not limited to, Glioma Derived Growth Factor (GDGF), as disclosed in European Patent Number EP-399816; Platelet Derived Growth Factor-A (PDGF-A), as disclosed in European Patent Number EP-682110; Platelet Derived Growth Factor-B (PDGF-B), as disclosed in European Patent Number EP-282317; Placental Growth Factor (PIGF), as disclosed in International Publication Number WO 92/06194; Placental Growth Factor-2 (PIGF-2), as disclosed in Hauser et al., Gorwth Factors, 4:259-268 (1993); Vascular Endothelial Growth Factor (VEGF), as disclosed in International Publication Number WO 90/13649; Vascular Endothelial Growth Factor-A (VEGF-A), as disclosed in European Patent Number EP-506477; Vascular Endothelial Growth Factor-2 (VEGF-2), as disclosed in International Publication Number WO 96/39515; Vascular Endothelial Growth Factor B (VEGF-3); Vascular Endothelial Growth Factor B-186 (VEGF-B186), as disclosed in International Publication Number WO 96/26736; Vascular Endothelial Growth Factor-D (VEGF-D), as disclosed in International Publication Number WO 98/02543; Vascular Endothelial Growth Factor-D (VEGF-D), as disclosed in International Publication Number WO 98/07832; and Vascular Endothelial Growth Factor-E (VEGF-E), as disclosed in German Patent Number DE19639601. The above mentioned references are incorporated herein by reference herein.

In an additional embodiment, the therapeutics of the invention are administered in combination with hematopoietic growth factors. Hematopoietic growth factors that may be administered with the Therapeutics of the invention include, but are not limited to, LEUKINE™ (SARGRAMOSTIM™) and NEUPOGEN™ (FILGRASTIM™).

In an additional embodiment, the therapeutics of the invention are administered in combination with Fibroblast Growth Factors. Fibroblast Growth Factors that may be administered with the therapeutics of the invention include, but are not limited to, FGF-1, FGF-2, FGF-3, FGF-4, FGF-5, FGF-6, FGF-7, FGF-8, FGF-9, FGF-10, FGF-11, FGF-12, FGF-13, FGF-14, and FGF-15.

In an additional embodiment, the compositions of the invention are administered with a chemokine. In another embodiment, the compositions of the invention are administered with chemokine beta-8, chemokine beta-1, and/or macrophage inflammatory protein-4. In a preferred embodiment, the compositions of the invention are administered with chemokine beta-8.

In additional embodiments, the therapeutics of the invention are administered in combination with other therapeutic or prophylactic regimens, such as, for example, radiation therapy.

### Example 25

Method of Treating Decreased Levels of PSF-2

The present invention relates to a method for treating an individual in need of a decreased level of PSF-2 activity in the body comprising, administering to such an individual a composition comprising a therapeutically effective amount of PSF-2 antagonist. Preferred antagonists for use in the present invention are PSF-2-specific antibodies.

Moreover, it will be appreciated that conditions caused by a decrease in the standard or normal expression level of PSF-2 in an individual can be treated by administering PSF-2, preferably in the secreted form. Thus, the invention also provides a method of treatment of an individual in need of an increased level of PSF-2 polypeptide comprising administering to such an individual a pharmaceutical composition comprising an amount of PSF-2 to increase the activity level of PSF-2 in such an individual.

For example, a patient with decreased levels of PSF-2 polypeptide receives a daily dose 0.1-100 ug/kg of the polypeptide for six consecutive days. Preferably, the polypeptide is in the secreted form. The exact details of the dosing scheme, based on administration and formulation, are provided in Example 24.

### Example 26

Method of Treating Increased Levels of PSF-2

The present invention also relates to a method for treating an individual in need of an increased level of PSF-2 activity in the body comprising administering to such an individual a composition comprising a therapeutically effective amount of PSF-2 or an agonist thereof.

Antisense technology is used to inhibit production of PSF-2. This technology is one example of a method of decreasing levels of PSF-2 polypeptide, preferably a secreted form, due to a variety of etiologies, such as cancer.

For example, a patient diagnosed with abnormally increased levels of PSF-2 is administered intravenously antisense polynucleotides at 0.5, 1.0, 1.5, 2.0 and 3.0 mg/kg day for 21 days. This treatment is repeated after a 7-day rest period if the treatment was well tolerated. The formulation of the antisense polynucleotide is provided in Example 24.

### Example 27

### Method of Treatment Using Gene Therapy—Ex Vivo

One method of gene therapy transplants fibroblasts, which are capable of expressing PSF-2 polypeptides, onto a patient. Generally, fibroblasts are obtained from a subject by skin biopsy. The resulting tissue is placed in tissue-culture medium and separated into small pieces. Small chunks of the tissue are placed on a wet surface of a tissue culture flask, approximately ten pieces are placed in each flask. The flask is turned upside down, closed tight and left at room temperature over night. After 24 hours at room temperature, the flask is inverted and the chunks of tissue remain fixed to the bottom of the flask and fresh media (e.g., Ham's F12 media, with 10% FBS, penicillin and streptomycin) is added. The flasks are then incubated at 37 C. for approximately one week.

At this time, fresh media is added and subsequently changed every several days. After an additional two weeks in culture, a monolayer of fibroblasts emerge. The monolayer is trypsinized and scaled into larger flasks.

pMV-7 (Kirschmeier, P. T. et al., DNA, 7:219-25 (1988)), flanked by the long terminal repeats of the Moloney murine sarcoma virus, is digested with EcoRI and HindIII and subsequently treated with calf intestinal phosphatase. The linear vector is fractionated on agarose gel and purified, using glass beads.

The cDNA encoding PSF-2 can be amplified using PCR primers which correspond to the 5′ and 3′ end sequences respectively as set forth in Example 1. Preferably, the 5′ primer contains an EcoRI site and the 3′ primer includes a HindIII site. Equal quantities of the Moloney murine sarcoma virus linear backbone and the amplified EcoRI and HindIII fragment are added together, in the presence of T4 DNA ligase. The resulting mixture is maintained under conditions appropriate for ligation of the two fragments. The ligation mixture is then used to transform bacteria HB101, which are then plated onto agar containing kanamycin for the purpose of confirming that the vector contains properly inserted PSF-2.

The amphotropic pA317 or GP+am12 packaging cells are grown in tissue culture to confluent density in Dulbecco's Modified Eagles Medium (DMEM) with 10% calf serum (CS), penicillin and streptomycin. The MSV vector containing the PSF-2 gene is then added to the media and the packaging cells transduced with the vector. The packaging cells now produce infectious viral particles containing the PSF-2 gene(the packaging cells are now referred to as producer cells).

Fresh media is added to the transduced producer cells, and subsequently, the media is harvested from a 10 cm plate of confluent producer cells. The spent media, containing the infectious viral particles, is filtered through a millipore filter to remove detached producer cells and this media is then used to infect fibroblast cells. Media is removed from a sub-confluent plate of fibroblasts and quickly replaced with the media from the producer cells. This media is removed and replaced with fresh media. If the titer of virus is high, then virtually all fibroblasts will be infected and no selection is required. If the titer is very low, then it is necessary to use a retroviral vector that has a selectable marker, such as neo or his. Once the fibroblasts have been efficiently infected, the fibroblasts are analyzed to determine whether PSF-2 polypeptide is produced.

The engineered fibroblasts are then transplanted onto the host, either alone or after having been grown to confluence on cytodex 3 microcarrier beads.

### Example 28

Method of Treatment Using Gene Therapy—In Vivo

Another aspect of the present invention is using in vivo gene therapy methods to treat disorders, diseases and conditions. The gene therapy method relates to the introduction of naked nucleic acid (DNA, RNA, and antisense DNA or RNA) PSF-2 sequences into an animal to increase or decrease the expression of the PSF-2 polypeptide. The PSF-2 polynucleotide may be operatively linked to a promoter or any other genetic elements necessary for the expression of the PSF-2 polypeptide by the target tissue. Such gene therapy and delivery techniques and methods are known in the art, see, for example, WO90/11092, WO98/11779; U.S. Pat. Nos. 5693622, 5705151, 5580859; Tabata H. et al. (1997) Cardiovasc. Res. 35(3):470-479, Chao J et al. (1997) Pharmacol. Res. 35(6):517-522, Wolff J. A. (1997) Neuromuscul. Disord. 7(5):314-318, Schwartz B. et al. (1996) Gene Ther. 3(5):405-411, Tsurumi Y. et al. (1996) Circulation 94(12):3281-3290 (incorporated herein by reference).

The PSF-2 polynucleotide constructs may be delivered by any method that delivers injectable materials to the cells of an animal, such as, injection into the interstitial space of tissues (heart, muscle, skin, lung, liver, intestine and the like). The PSF-2 polynucleotide constructs can be delivered in a pharmaceutically acceptable liquid or aqueous carrier.

The term “naked” polynucleotide, DNA or RNA, refers to sequences that are free from any delivery vehicle that acts to assist, promote, or facilitate entry into the cell, including viral sequences, viral particles, liposome formulations, lipofectin or precipitating agents and the like. However, the PSF-2 polynucleotides may also be delivered in liposome formulations (such as those taught in Felgner P. L. et al. (1995) Ann. NY Acad. Sci. 772:126-139 and Abdallah B. et al. (1995) Biol. Cell 85(1):1-7) which can be prepared by methods well known to those skilled in the art.

The PSF-2 polynucleotide vector constructs used in the gene therapy method are preferably constructs that will not integrate into the host genome nor will they contain sequences that allow for replication. Any strong promoter known to those skilled in the art can be used for driving the expression of DNA. Unlike other gene therapies techniques, one major advantage of introducing naked nucleic acid sequences into target cells is the transitory nature of the polynucleotide synthesis in the cells. Studies have shown that non-replicating DNA sequences can be introduced into cells to provide production of the desired polypeptide for periods of up to six months.

The PSF-2 polynucleotide construct can be delivered to the interstitial space of tissues within the an animal, including of muscle, skin, brain, lung, liver, spleen, bone marrow, thymus, heart, lymph, blood, bone, cartilage, pancreas, kidney, gall bladder, stomach, intestine, testis, ovary, uterus, rectum, nervous system, eye, gland, and connective tissue. Interstitial space of the tissues comprises the intercellular fluid, mucopolysaccharide matrix among the reticular fibers of organ tissues, elastic fibers in the walls of vessels or chambers, collagen fibers of fibrous tissues, or that same matrix within connective tissue ensheathing muscle cells or in the lacunae of bone. It is similarly the space occupied by the plasma of the circulation and the lymph fluid of the lymphatic channels. Delivery to the interstitial space of muscle tissue is preferred for the reasons discussed below. They may be conveniently delivered by injection into the tissues comprising these cells. They are preferably delivered to and expressed in persistent, non-dividing cells which are differentiated, although delivery and expression may be achieved in non-differentiated or less completely differentiated cells, such as, for example, stem cells of blood or skin fibroblasts. In vivo muscle cells are particularly competent in their ability to take up and express polynucleotides.

For the naked PSF-2 polynucleotide injection, an effective dosage amount of DNA or RNA will be in the range of from about 0.05 g/kg body weight to about 50 mg/kg body weight. Preferably the dosage will be from about 0.005 mg/kg to about 20 mg/kg and more preferably from about 0.05 mg/kg to about 5 mg/kg. Of course, as the artisan of ordinary skill will appreciate, this dosage will vary according to the tissue site of injection. The appropriate and effective dosage of nucleic acid sequence can readily be determined by those of ordinary skill in the art and may depend on the condition being treated and the route of administration. The preferred route of administration is by the parenteral route of injection into the interstitial space of tissues. However, other parenteral routes may also be used, such as, inhalation of an aerosol formulation particularly for delivery to lungs or bronchial tissues, throat or mucous membranes of the nose. In addition, naked PSF-2 polynucleotide constructs can be delivered to arteries during angioplasty by the catheter used in the procedure.

The dose response effects of injected PSF-2 polynucleotide in muscle in vivo is determined as follows. Suitable PSF-2 template DNA for production of mRNA coding for PSF-2 polypeptide is prepared in accordance with a standard recombinant DNA methodology. The template DNA, which may be either circular or linear, is either used as naked DNA or complexed with liposomes. The quadriceps muscles of mice are then injected with various amounts of the template DNA.

Five to six week old female and male Balb/C mice are anesthetized by intraperitoneal injection with 0.3 ml of 2.5% Avertin. A 1.5 cm incision is made on the anterior thigh, and the quadriceps muscle is directly visualized. The PSF-2 template DNA is injected in 0.1 ml of carrier in a 1 cc syringe through a 27 gauge needle over one minute, approximately 0.5 cm from the distal insertion site of the muscle into the knee and about 0.2 cm deep. A suture is placed over the injection site for future localization, and the skin is closed with stainless steel clips.

After an appropriate incubation time (e.g., 7 days) muscle extracts are prepared by excising the entire quadriceps. Every fifth 15 um cross-section of the individual quadriceps muscles is histochemically stained for PSF-2 polypeptide expression. A time course for PSF-2 polypeptide expression may be done in a similar fashion except that quadriceps from different mice are harvested at different times. Persistence of PSF-2 DNA in muscle following injection may be determined by Southern blot analysis after preparing total cellular DNA and HIRT supernatants from injected and control mice. The results of the above experimentation in mice can be use to extrapolate proper dosages and other treatment parameters in humans and other animals using PSF-2 naked DNA.

### Example 29

Suppression of TNF Alpha-Induced Adhesion Molecule Expression by PSF-2

The recruitment of lymphocytes to areas of inflammation and angiogenesis involves specific receptor-ligand interactions between cell surface adhesion molecules (CAMs) on lymphocytes and the vascular endothelium. The adhesion process, in both normal and pathological settings, follows a multi-step cascade that involves intercellular adhesion molecule-1 (ICAM-1), vascular cell adhesion molecule-1 (VCAM-1), and endothelial leukocyte adhesion molecule-1 (E-selectin) expression on endothelial cells (EC). The expression of these molecules and others on the vascular endothelium determines the efficiency with which leukocytes may adhere to the local vasculature and extravasate into the local tissue during the development of an inflammatory response. The local concentration of cytokines and growth factor participate in the modulation of the expression of these CAMs.

Tumor necrosis factor alpha (TNF-a), a potent proinflammatory cytokine, is a stimulator of all three CAMs on endothelial cells and may be involved in a wide variety of inflammatory responses, often resulting in a pathological outcome.

The potential of PSF-2 to mediate a suppression of TNF-a induced CAM expression can be examined. A modified ELISA assay which uses ECs as a solid phase absorbent is employed to measure the amount of CAM expression on TNF-a treated ECs when co-stimulated with a member of the FGF family of proteins.

To perform the experiment, human umbilical vein endothelial cell (HUVEC) cultures are obtained from pooled cord harvests and maintained in growth medium (EGM-2; Clonetics, San Diego, Calif.) supplemented with 10% FCS and 1% penicillin/streptomycin in a 37 C humidified incubator containing 5% CO₂. HUVECs are seeded in 96-well plates at concentrations of 1×10⁴ cells/well in EGM medium at 37 C for 18-24 hrs or until confluent. The monolayers are subsequently washed 3 times with a serum-free solution of RPMI-1640 supplemented with 100 U/ml penicillin and 100 mg/ml streptomycin, and treated with a given cytokine and/or growth factor(s) for 24 h at 37 C. Following incubation, the cells are then evaluated for CAM expression.

Human Umbilical Vein Endothelial cells (VECs) are grown in a standard 96 well plate to confluence. Growth medium is removed from the cells and replaced with 90 ul of 199 Medium (10% FBS). Samples for testing and positive or negative controls are added to the plate in triplicate (in 10 ul volumes). Plates are incubated at 37 C for either 5 h (selectin and integrin expression) or 24 h (integrin expression only). Plates are aspirated to remove medium and 100 μl of 0.1% paraformaldehyde-PBS (with Ca²⁺ and Mg²⁺) is added to each well. Plates are held at 4° C. for 30 min.

Fixative is then removed from the wells and wells are washed 1× with PBS (including Ca²⁺ and Mg²⁺)+0.5% BSA and drained. Do not allow the wells to dry. Add 10 μl of diluted primary antibody to the test and control wells. Anti-ICAM-1-Biotin, Anti-VCAM-1-Biotin and Anti-E-selectin-Biotin are used at a concentration of 10 μg/ml (1:10 dilution of 0.1 mg/ml stock antibody). Cells are incubated at 37° C. for 30 min. in a humidified environment. Wells are washed ×3 with PBS(+Ca,Mg)+0.5% BSA.

Then add 20 μl of diluted ExtrAvidin-Alkaline Phosphotase (1:5,000 dilution) to each well and incubated at 37° C. for 30 min. Wells are washed ×3 with PBS(+Ca,Mg)+0.5% BSA. 1 tablet of p-Nitrophenol Phosphate pNPP is dissolved in 5 ml of glycine buffer (pH 10.4). 100 μl of pNPP substrate in glycine buffer is added to each test well. Standard wells in triplicate are prepared from the working dilution of the ExtrAvidin-Alkaline Phosphotase in glycine buffer: 1:5,000 (10⁰)>10^{-0.5}>10⁻¹>10^{-1.5}0.5 μl of each dilution is added to triplicate wells and the resulting AP content in each well is 5.50 ng, 1.74 ng, 0.55 ng, 0.18 ng. 100 μl of pNNP reagent must then be added to each of the standard wells. The plate must be incubated at 37° C. for 4 h. A volume of 50 μl of 3M NaOH is added to all wells. The results are quantified on a plate reader at 405 nm. The background subtraction option is used on blank wells filled with glycine buffer only. The template is set up to indicate the concentration of AP-conjugate in each standard well [5.50 ng; 1.74 ng; 0.55 ng; 0.18 ng]. Results are indicated as amount of bound AP-conjugate in each sample.

The studies described in this example test activity in PSF-2 protein. However, one skilled in the art could easily modify the exemplified studies to test the activity of PSF-2 polynucleotides (e.g., gene therapy), agonists, and/or antagonists of PSF-2.

### Example 30

PSF-2 Transgenic Animals

The PSF-2 polypeptides can also be expressed in transgenic animals. Animals of any species, including, but not limited to, mice, rats, rabbits, hamsters, guinea pigs, pigs, micro-pigs, goats, sheep, cows and non-human primates, e.g., baboons, monkeys, and chimpanzees may be used to generate transgenic animals. In a specific embodiment, techniques described herein or otherwise known in the art, are used to express polypeptides of the invention in humans, as part of a gene therapy protocol.

Any technique known in the art may be used to introduce the transgene (i.e., polynucleotides of the invention) into animals to produce the founder lines of transgenic animals. Such techniques include, but are not limited to, pronuclear microinjection (Paterson et al., Appl. Microbiol. Biotechnol. 40:691-698 (1994); Carver et al., Biotechnology (NY) 11:1263-1270 (1993); Wright et al., Biotechnology (NY) 9:830-834 (1991); and Hoppe et al., U.S. Pat. No. 4,873,191 (1989)); retrovirus mediated gene transfer into germ lines (Van der Putten et al., Proc. Natl. Acad. Sci., USA 82:6148-6152 (1985)), blastocysts or embryos; gene targeting in embryonic stem cells (Thompson et al., Cell 56:313-321 (1989)); electroporation of cells or embryos (Lo, 1983, Mol Cell. Biol. 3:1803-1814 (1983)); introduction of the polynucleotides of the invention using a gene gun (see, e.g., Ulmer et al., Science 259:1745 (1993); introducing nucleic acid constructs into embryonic pleuripotent stem cells and transferring the stem cells back into the blastocyst; and sperm-mediated gene transfer (Lavitrano et al., Cell 57:717-723 (1989); etc. For a review of such techniques, see Gordon, “Transgenic Animals,” Intl. Rev. Cytol. 115:171-229 (1989), which is incorporated by reference herein in its entirety.

Any technique known in the art may be used to produce transgenic clones containing polynucleotides of the invention, for example, nuclear transfer into enucleated oocytes of nuclei from cultured embryonic, fetal, or adult cells induced to quiescence (Campell et al., Nature 380:64-66 (1996); Wilmut et al., Nature 385:810-813 (1997)).

The present invention provides for transgenic animals that carry the transgene in all their cells, as well as animals which carry the transgene in some, but not all their cells, i.e., mosaic animals or chimeric. The transgene may be integrated as a single transgene or as multiple copies such as in concatamers, e.g., head-to-head tandems or head-to-tail tandems. The transgene may also be selectively introduced into and activated in a particular cell type by following, for example, the teaching of Lasko et al. (Lasko et al., Proc. Natl. Acad. Sci. USA 89:6232-6236 (1992)). The regulatory sequences required for such a cell-type specific activation will depend upon the particular cell type of interest, and will be apparent to those of skill in the art. When it is desired that the polynucleotide transgene be integrated into the chromosomal site of the endogenous gene, gene targeting is preferred.

Briefly, when such a technique is to be utilized, vectors containing some nucleotide sequences homologous to the endogenous gene are designed for the purpose of integrating, via homologous recombination with chromosomal sequences, into and disrupting the function of the nucleotide sequence of the endogenous gene. The transgene may also be selectively introduced into a particular cell type, thus inactivating the endogenous gene in only that cell type, by following, for example, the teaching of Gu et al. (Gu et al., Science 265:103-106 (1994)). The regulatory sequences required for such a cell-type specific inactivation will depend upon the particular cell type of interest, and will be apparent to those of skill in the art. The contents of each of the documents recited in this paragraph is herein incorporated by reference in its entirety.

Any of the PSF-2 polypeptides disclosed throughout this application may be used to generate transgenic animals. For example, DNA encoding amino acids Met-(−1) to Tyr-274 of SEQ ID NO:2 can be inserted into a vector containing a promoter, such as the actin promoter, which will ubiquitously express the inserted fragment. Primers that can be used to generate such fragments include a 5′ primer containing an underlined Bam HI restriction site: 5′-GCA GCA GGA TCC ATG CTG CCG CCG CCG CGG CCC GCA GCT GCC-3′ (SEQ ID NO:24) and a 3′ primer, containing an underlined Xba I restriction site: 5′-GCA GCA TCT AGA GTA GTA ATC GTC ATT CTC TTC ACT CTC AGC CTC CTC CTC AGG-3′ (SEQ ID NO:25). This construct will express a full-length PSF-2 under the control of the actin promoter for ubiquitous expression.

In a specific embodiment, to generate transgenic animals, the cDNA sequence encoding the full length PSF-2 polypeptide in the deposited clone is subcloned into the expression vector pAC. PCR amplification of the insert is accomplished using oligonucleotide primers corresponding to the 5′ and 3′ sequences of the gene. The 5′ primer has the sequence 5′-GCA GCA GGA TCC GCC ATC ATG CTG CCG CCG CCG CGG CCC GCA GCT GCC TTG-3′ (SEQ ID NO:25) containing the Bam HI restriction enzyme site, an efficient signal for initiation of translation in eukaryotic cells (Kozak, M., J. Mol. Biol. 196:947-950 (1987)), followed by a number of nucleotides of the sequence of the complete PSF-2 polypeptide shown in FIGS. 1A and 1B and in SEQ ID NO:1, beginning with the AUG initiation codon. The 3′ primer has the sequence 5′-GCA GCA TCT AGA TTA GTA GTA ATC GTC ATT CTC TTC ACT CTC AGC CTC-3′ (SEQ ID NO:26) containing the Xba I restriction site followed by a number of nucleotides complementary to the 3′ noncoding sequence in FIGS. 1A and 1B and in SEQ ID NO:1.

In a specific embodiment, to generate transgenic animals, the cDNA sequence encoding the full length PSF-2 polypeptide in the deposited clone is subcloned into the expression vector pTR. PCR amplification of the insert is accomplished using oligonucleotide primers corresponding to the 5′ and 3′ sequences of the gene. The 5′ primer has the sequence 5′-GCA GCA GGA TCC GCC ATC ATG CTG CCG CCG CCG CGG CCC GCA GCT GCC TTG-3′ (SEQ ID NO:25) containing the Bam HI restriction enzyme site, an efficient signal for initiation of translation in eukaryotic cells (Kozak, M., J. Mol. Biol. 196:947-950 (1987)), followed by a number of nucleotides of the sequence of the complete PSF-2 polypeptide shown in FIGS. 1A and 1B and in SEQ ID NO:1, beginning with the AUG initiation codon. The 3′ primer has the sequence 5′-GCA GCA TCT AGA TTA GTA GTA ATC GTC ATT CTC TTC ACT CTC AGC CTC-3′ (SEQ ID NO:26) containing the Xba I restriction site followed by a number of nucleotides complementary to the 3′ noncoding sequence in FIGS. 1A and 1B and in SEQ ID NO:1.

One of ordinary skill in the art would immediately realize that many other PSF-2 polynucleotides of the invention may also be inserted into this or a similar vector to create transgenic animals that exhibit ubiquitous expression of a PSF-2 of the invention. Alternatively, polynucleotides of the invention may be inserted in a vector which controls tissue specific expression of a PSF-2 of the invention by virtue of a tissue specific promoter. For example, a construct having a transferrin promoter would be expected to express a PSF-2 polypeptide of the invention primarily in the liver of a transgenic animal.

In addition to expressing the polypeptide of the present invention in a ubiquitous or tissue specific manner in transgenic animals, it would also be routine for one skilled in the art to generate constructs which regulate expression of the polypeptide by a variety of other means (for example, developmentally or chemically regulated expression).

Once transgenic animals have been generated, the expression of the recombinant gene may be assayed utilizing standard techniques. Initial screening may be accomplished by Southern blot analysis or PCR techniques to analyze animal tissues to verify that integration of the transgene has taken place. The level of mRNA expression of the transgene in the tissues of the transgenic animals may also be assessed using techniques which include, but are not limited to, Northern blot analysis of tissue samples obtained from the animal, in situ hybridization analysis, reverse transcriptase-PCR (rt-PCR), and “Taqman” PCR. Samples of transgenic gene-expressing tissue may also be evaluated immunocytochemically or immunohistochemically using antibodies specific for the transgene product.

Once the founder animals are produced, they may be bred, inbred, outbred, or crossbred to produce colonies of the particular animal. Examples of such breeding strategies include, but are not limited to: outbreeding of founder animals with more than one integration site in order to establish separate lines; inbreeding of separate lines in order to produce compound transgenics that express the transgene at higher levels because of the effects of additive expression of each transgene; crossing of heterozygous transgenic animals to produce animals homozygous for a given integration site in order to both augment expression and eliminate the need for screening of animals by DNA analysis; crossing of separate homozygous lines to produce compound heterozygous or homozygous lines; and breeding to place the transgene on a distinct background that is appropriate for an experimental model of interest.

Transgenic animals of the invention have uses which include, but are not limited to, animal model systems useful in elaborating the biological function of PSF-2 polypeptides, studying conditions and/or disorders associated with aberrant PSF-2 expression, and in screening for compounds effective in ameliorating such conditions and/or disorders.

### Example 31

PSF-2 Knock-Out Animals

Endogenous PSF-2 gene expression can also be reduced by inactivating or “knocking out” the PSF-2 gene and/or its promoter using targeted homologous recombination. (E.g., see Smithies et al., Nature 317:230-234 (1985); Thomas & Capecchi, Cell 51:503-512 (1987); Thompson et al., Cell 5:313-321 (1989); each of which is incorporated by reference herein in its entirety). For example, a mutant, non-functional polynucleotide of the invention (or a completely unrelated DNA sequence) flanked by DNA homologous to the endogenous polynucleotide sequence (either the coding regions or regulatory regions of the gene) can be used, with or without a selectable marker and/or a negative selectable marker, to transfect cells that express polypeptides of the invention in vivo. In another embodiment, techniques known in the art are used to generate knockouts in cells that contain, but do not express the gene of interest. Insertion of the DNA construct, via targeted homologous recombination, results in inactivation of the targeted gene. Such approaches are particularly suited in research and agricultural fields where modifications to embryonic stem cells can be used to generate animal offspring with an inactive targeted gene (e.g., see Thomas & Capecchi 1987 and Thompson 1989, supra). However this approach can be routinely adapted for use in humans provided the recombinant DNA constructs are directly administered or targeted to the required site in vivo using appropriate viral vectors that will be apparent to those of skill in the art.

In further embodiments of the invention, cells that are genetically engineered to express the polypeptides of the invention, or alternatively, that are genetically engineered not to express the polypeptides of the invention (e.g., knockouts) are administered to a patient in vivo. Such cells may be obtained from the patient (i.e., animal, including human) or an MHC compatible donor and can include, but are not limited to fibroblasts, bone marrow cells, blood cells (e.g., lymphocytes), adipocytes, muscle cells, endothelial cells etc. The cells are genetically engineered in vitro using recombinant DNA techniques to introduce the coding sequence of polypeptides of the invention into the cells, or alternatively, to disrupt the coding sequence and/or endogenous regulatory sequence associated with the polypeptides of the invention, e.g., by transduction (using viral vectors, and preferably vectors that integrate the transgene into the cell genome) or transfection procedures, including, but not limited to, the use of plasmids, cosmids, YACs, naked DNA, electroporation, liposomes, etc. The coding sequence of the polypeptides of the invention can be placed under the control of a strong constitutive or inducible promoter or promoter/enhancer to achieve expression, and preferably secretion, of the PSF-2 polypeptides. The engineered cells which express and preferably secrete the polypeptides of the invention can be introduced into the patient systemically, e.g., in the circulation, or intraperitoneally.

Alternatively, the cells can be incorporated into a matrix and implanted in the body, e.g., genetically engineered fibroblasts can be implanted as part of a skin graft; genetically engineered endothelial cells can be implanted as part of a lymphatic or vascular graft. (See, for example, Anderson et al. U.S. Pat. No. 5,399,349; and Mulligan & Wilson, U.S. Pat. No. 5,460,959 each of which is incorporated by reference herein in its entirety).

When the cells to be administered are non-autologous or non-MHC compatible cells, they can be administered using well known techniques which prevent the development of a host immune response against the introduced cells. For example, the cells may be introduced in an encapsulated form which, while allowing for an exchange of components with the immediate extracellular environment, does not allow the introduced cells to be recognized by the host immune system.

Knock-out animals of the invention have uses which include, but are not limited to, animal model systems useful in elaborating the biological function of PSF-2 polypeptides, studying conditions and/or disorders associated with aberrant PSF-2 expression, and in screening for compounds effective in ameliorating such conditions and/or disorders.

### Example 32

Assays Detecting Stimulation or Inhibition of B Cell Proliferation and Differentiation

Generation of functional humoral immune responses requires both soluble and cognate signaling between B-lineage cells and their microenvironment. Signals may impart a positive stimulus that allows a B-lineage cell to continue its programmed development, or a negative stimulus that instructs the cell to arrest its current developmental pathway. To date, numerous stimulatory and inhibitory signals have been found to influence B cell responsiveness including IL-2, IL-4, IL-5, IL-6, IL-7, IL10, IL-13, IL-14 and IL-15. Interestingly, these signals are by themselves weak effectors but can, in combination with various co-stimulatory proteins, induce activation, proliferation, differentiation, homing, tolerance and death among B cell populations.

One of the most well-studied classes of B-cell co-stimulatory proteins is the TNF-superfamily. Within this family CD40, CD27, and CD30 along with their respective ligands CD154, CD70, and CD153 have been found to regulate a variety of immune responses. Assays which allow for the detection and/or observation of the proliferation and differentiation of these B-cell populations and their precursors are valuable tools in determining the effects various proteins may have on these B-cell populations in terms of proliferation and differentiation. Listed below are two assays designed to allow for the detection of the differentiation, proliferation, or inhibition of B-cell populations and their precursors.

In Vitro Assay—Purified PSF-2 protein, or truncated forms thereof, is assessed for its ability to induce activation, proliferation, differentiation or inhibition and/or death in B-cell populations and their precursors. The activity of PSF-2 protein on purified human tonsillar B cells, measured qualitatively over the dose range from 0.1 to 10,000 ng/mL, is assessed in a standard B-lymphocyte co-stimulation assay in which purified tonsillar B cells are cultured in the presence of either formalin-fixed Staphylococcus aureus Cowan I (SAC) or immobilized anti-human IgM antibody as the priming agent. Second signals such as IL-2 and IL-15 synergize with SAC and IgM crosslinking to elicit B cell proliferation as measured by tritiated-thymidine incorporation. Novel synergizing agents can be readily identified using this assay. The assay involves isolating human tonsillar B cells by magnetic bead (MACS) depletion of CD3-positive cells. The resulting cell population is greater than 95% B cells as assessed by expression of CD45R(B220).

Various dilutions of each sample are placed into individual wells of a 96-well plate to which are added 10⁵ B-cells suspended in culture medium (RPMI 1640 containing 10% FBS, 5×10⁻⁵M 2-ME, 100 U/ml penicillin, 10 ug/ml streptomycin, and 10⁻⁵ dilution of SAC) in a total volume of 150 ul. Proliferation or inhibition is quantitated by a 20 h pulse (1 uCi/well) with ³H-thymidine (6.7 Ci/mM) beginning 72 h post factor addition. The positive and negative controls are IL2 and medium respectively.

In Vivo Assay—BALB/c mice are injected (i.p.) twice per day with buffer only, or 2 mg/Kg of PSF-2 protein, or truncated forms thereof. Mice receive this treatment for 4 consecutive days, at which time they are sacrificed and various tissues and serum collected for analyses. Comparison of H&E sections from normal and PSF-2 protein-treated spleens identify the results of the activity of PSF-2 protein on spleen cells, such as the diffusion of peri-arterial lymphatic sheaths, and/or significant increases in the nucleated cellularity of the red pulp regions, which may indicate the activation of the differentiation and proliferation of B-cell populations. Immunohistochemical studies using a B cell marker, anti-CD45R(B220), are used to determine whether any physiological changes to splenic cells, such as splenic disorganization, are due to increased B-cell representation within loosely defined B-cell zones that infiltrate established T-cell regions.

Flow cytometric analyses of the spleens from PSF-2 protein-treated mice is used to indicate whether PSF-2 protein specifically increases the proportion of ThB+, CD45R(B220)dull B cells over that which is observed in control mice.

Likewise, a predicted consequence of increased mature B-cell representation in vivo is a relative increase in serum Ig titers. Accordingly, serum IgM and IgA levels are compared between buffer and PSF-2 protein-treated mice.

The studies described in this example test activity in PSF-2 protein. However, one skilled in the art could easily modify the exemplified studies to test the activity of PSF-2 polynucleotides (e.g., gene therapy), agonists, and/or antagonists of PSF-2.

### Example 33

T Cell Proliferation Assay

A CD3-induced proliferation assay is performed on PBMCs and is measured by the uptake of ³H-thymidine. The assay is performed as follows. Ninety-six well plates are coated with 100 μl/well of mAb to CD3 (HIT3a, Pharmingen) or isotype-matched control mAb (B33.1) overnight at 4° C. (1 μg/ml in 0.05M bicarbonate buffer, pH 9.5), then washed three times with PBS. PBMC are isolated by F/H gradient centrifugation from human peripheral blood and added to quadruplicate wells (5×10⁴/well) of mAb coated plates in RPMI containing 10% FCS and P/S in the presence of varying concentrations of PSF-2 protein (total volume 200 μl). Relevant protein buffer and medium alone are controls. After 48 hr. culture at 37° C., plates are spun for 2 min. at 1000 rpm and 100 μl of supernatant is removed and stored −20° C. for measurement of IL-2 (or other cytokines) if effect on proliferation is observed. Wells are supplemented with 100 μl of medium containing 0.5 μCi of ³H-thymidine and cultured at 37° C. for 18-24 hr. Wells are harvested and incorporation of ³H-thymidine used as a measure of proliferation. Anti-CD3 alone is the positive control for proliferation. IL-2 (100 U/ml) is also used as a control which enhances proliferation. Control antibody which does not induce proliferation of T cells is used as the negative controls for the effects of PSF-2 proteins.

The studies described in this example tested activity in PSF-2 protein. However, one skilled in the art could easily modify the exemplified studies to test the activity of PSF-2 polynucleotides (e.g., gene therapy), agonists, and/or antagonists of PSF-2.

### Example 34

Effect of PSF-2 on the Expression of MHC Class II, Costimulatory and Adhesion Molecules and Cell Differentiation of Monocytes and Monocyte-Derived Human Dendritic Cells

Dendritic cells are generated by the expansion of proliferating precursors found in the peripheral blood: adherent PBMC or elutriated monocytic fractions are cultured for 7-10 days with GM-CSF (50 ng/ml) and IL-4 (20 ng/ml). These dendritic cells have the characteristic phenotype of immature cells (expression of CD1, CD80, CD86, CD40 and MHC class II antigens). Treatment with activating factors, such as TNF-α, causes a rapid change in surface phenotype (increased expression of MHC class I and II, costimulatory and adhesion molecules, downregulation of FCγRII, upregulation of CD83). These changes correlate with increased antigen-presenting capacity and with functional maturation of the dendritic cells.

FACS analysis of surface antigens is performed as follows. Cells are treated 1-3 days with increasing concentrations of PSF-2 or LPS (positive control), washed with PBS containing 1% BSA and 0.02 mM sodium azide, and then incubated with 1:20 dilution of appropriate FITC- or PE-labeled monoclonal antibodies for 30 minutes at 4° C. After an additional wash, the labeled cells are analyzed by flow cytometry on a FACScan (Becton Dickinson).

Effect on the Production of Cytokines.

Cytokines generated by dendritic cells, in particular IL-12, are important in the initiation of T-cell dependent immune responses. IL-12 strongly influences the development of Th1 helper T-cell immune response, and induces cytotoxic T and NK cell function. An ELISA is used to measure the IL-12 release as follows. Dendritic cells (10⁶/ml) are treated with increasing concentrations of PSF-2 for 24 hours. LPS (100 ng/ml) is added to the cell culture as positive control. Supernatants from the cell cultures are then collected and analyzed for IL-12 content using commercial ELISA kit (e.g, R & D Systems (Minneapolis, Minn.)). The standard protocols provided with the kits are used.

Effect on the Expression of MHC Class II, Costimulatory and Adhesion Molecules.

Three major families of cell surface antigens can be identified on monocytes: adhesion molecules, molecules involved in antigen presentation, and Fc receptor. Modulation of the expression of MHC class II antigens and other costimulatory molecules, such as B7 and ICAM-1, may result in changes in the antigen presenting capacity of monocytes and ability to induce T cell activation. Increase expression of Fc receptors may correlate with improved monocyte cytotoxic activity, cytokine release and phagocytosis.

FACS analysis is used to examine the surface antigens as follows. Monocytes are treated 1-5 days with increasing concentrations of PSF-2 or LPS (positive control), washed with PBS containing 1% BSA and 0.02 mM sodium azide, and then incubated with 1:20 dilution of appropriate FITC- or PE-labeled monoclonal antibodies for 30 minutes at 4° C. After an additional wash, the labeled cells are analyzed by flow cytometry on a FACScan (Becton Dickinson).

Monocyte Activation and/or Increased Survival.

Assays for molecules that activate (or alternatively, inactivate) monocytes and/or increase monocyte survival (or alternatively, decrease monocyte survival) are known in the art and may routinely be applied to determine whether a molecule of the invention functions as an inhibitor or activator of monocytes. PSF-2, agonists, or antagonists of PSF-2 can be screened using the three assays described below. For each of these assays, Peripheral blood mononuclear cells (PBMC) are purified from single donor leukopacks (American Red Cross, Baltimore, Md.) by centrifugation through a Histopaque gradient (Sigma). Monocytes are isolated from PBMC by counterflow centrifugal elutriation.

Monocvte Survival Assay.

Human peripheral blood monocytes progressively lose viability when cultured in absence of serum or other stimuli. Their death results from internally regulated process (apoptosis). Addition to the culture of activating factors, such as TNF-alpha dramatically improves cell survival and prevents DNA fragmentation. Propidium iodide (PI) staining is used to measure apoptosis as follows. Monocytes are cultured for 48 hours in polypropylene tubes in serum-free medium (positive control), in the presence of 100 ng/ml TNF-alpha (negative control), and in the presence of varying concentrations of the compound to be tested. Cells are suspended at a concentration of 2×10⁶/ml in PBS containing PI at a final concentration of 5 μg/ml, and then incubaed at room temperature for 5 minutes before FACScan analysis. PI uptake has been demonstrated to correlate with DNA fragmentation in this experimental paradigm.

Effect on Cytokine Release.

An important function of monocytes/macrophages is their regulatory activity on other cellular populations of the immune system through the release of cytokines after stimulation. An ELISA to measure cytokine release is performed as follows. Human monocytes are incubated at a density of 5×10⁵ cells/ml with increasing concentrations of PSF-2 and under the same conditions, but in the absence of PSF-2. For IL-12 production, the cells are primed overnight with IFN (100 U/ml) in presence of PSF-2. LPS (10 ng/ml) is then added. Conditioned media are collected after 24 h and kept frozen until use. Measurement of TNF-alpha, IL-10, MCP-1 and IL-8 is then performed using a commercially available ELISA kit (e..g, R & D Systems (Minneapolis, Minn.)) and applying the standard protocols provided with the kit.

Oxidative Burst.

Purified monocytes are plated in 96-w plate at 2-1×10⁵ cell/well. Increasing concentrations of PSF-2 are added to the wells in a total volume of 0.2 ml culture medium (RPMI 1640+10% FCS, glutamine and antibiotics). After 3 days incubation, the plates are centrifuged and the medium is removed from the wells. To the macrophage monolayers, 0.2 ml per well of phenol red solution (140 mM NaCl, 10 mM potassium phosphate buffer pH 7.0, 5.5 mM dextrose, 0.56 mM phenol red and 19 U/ml of HRPO) is added, together with the stimulant (200 nM PMA). The plates are incubated at 37° C. for 2 hours and the reaction is stopped by adding 20 μl 1N NaOH per well. The absorbance is read at 610 nm. To calculate the amount of H₂O₂ produced by the macrophages, a standard curve of a H₂O₂ solution of known molarity is performed for each experiment.

The studies described in this example tested activity in PSF-2 protein. However, one skilled in the art could easily modify the exemplified studies to test the activity of PSF-2 polynucleotides (e.g., gene therapy), agonists, and/or antagonists of PSF-2.

### Example 35

PSF-2 Biological Effects

Astrocyte and Neuronal Assays.

Recombinant PSF-2, expressed in E. coli and purified as described above, can be tested for activity in, promoting the survival, neurite outgrowth, or phenotypic differentiation of cortical neuronal cells and for inducing the proliferation of glial fibrillary acidic protein immunopositive cells, astrocytes. The selection of cortical cells for the bioassay is based on the prevalent expression of FGF-1 and FGF-2 in cortical structures and on the previously reported enhancement of cortical neuronal survival resulting from FGF-2 treatment. A thymidine incorporation assay, for example, can be used to elucidate PSF-2's activity on these cells.

Moreover, previous reports describing the biological effects of FGF-2 (basic FGF) on cortical or hippocampal neurons in vitro have demonstrated increases in both neuron survival and neurite outgrowth (Walicke, P. et al., “Fibroblast growth factor promotes survival of dissociated hippocampal neurons and enhances neurite extension.” Proc. Natl. Acad. Sci. USA 83:3012-3016. (1986), assay herein incorporated by reference in its entirety). However, reports from experiments done on PC-12 cells suggest that these two responses are not necessarily synonymous and may depend on not only which FGF is being tested but also on which receptor(s) are expressed on the target cells. Using the primary cortical neuronal culture paradigm, the ability of PSF-2 to induce neurite outgrowth can be compared to the response achieved with FGF-2 using, for example, a thymidine incorporation assay.

Fibroblast and Endothelial Cell Assays.

Human lung fibroblasts are obtained from Clonetics (San Diego, Calif.) and maintained in growth media from Clonetics. Dermal microvascular endothelial cells are obtained from Cell Applications (San Diego, Calif.). For proliferation assays, the human lung fibroblasts and dermal microvascular endothelial cells can be cultured at 5,000 cells/well in a 96-well plate for one day in growth medium. The cells are then incubated for one day in 0.1% BSA basal medium. After replacing the medium with fresh 0.1% BSA medium, the cells are incubated with the test proteins for 3 days. Alamar Blue (Alamar Biosciences, Sacramento, Calif.) is added to each well to a final concentration of 10%. The cells are incubated for 4 hr. Cell viability is measured by reading in a CytoFluor fluorescence reader. For the PGE₂ assays, the human lung fibroblasts are cultured at 5,000 cells/well in a 96-well plate for one day. After a medium change to 0.1% BSA basal medium, the cells are incubated with FGF-2 or PSF-2 with or without IL-1α for 24 hours. The supernatants are collected and assayed for PGE₂ by EIA kit (Cayman, Ann Arbor, Mich.). For the IL-6 assays, the human lung fibroblasts are cultured at 5,000 cells/well in a 96-well plate for one day. After a medium change to 0.1% BSA basal medium, the cells are incubated with FGF-2 or PSF-2 with or without IL-1α for 24 hours. The supernatants are collected and assayed for IL-6 by ELISA kit (Endogen, Cambridge, Mass.).

Human lung fibroblasts are cultured with FGF-2 or PSF-2 for 3 days in basal medium before the addition of Alamar Blue to assess effects on growth of the fibroblasts. FGF-2 should show a stimulation at 10-2500 ng/ml which can be used to compare stimulation with PSF-2.

Parkinson Models.

The loss of motor function in Parkinson's disease is attributed to a deficiency of striatal dopamine resulting from the degeneration of the nigrostriatal dopaminergic projection neurons. An animal model for Parkinson's that has been extensively characterized involves the systemic administration of 1-methyl-4 phenyl 1,2,3,6-tetrahydropyridine (MPTP). In the CNS, MPTP is taken-up by astrocytes and catabolized by monoamine oxidase B to 1-methyl-4-phenyl pyridine (MPP⁺) and released. Subsequently, MPP⁺ is actively accumulated in dopaminergic neurons by the high-affinity reuptake transporter for dopamine. MPP⁺ is then concentrated in mitochondria by the electrochemical gradient and selectively inhibits nicotidamide adenine disphosphate: ubiquinone oxidoreductionase (complex I), thereby interfering with electron transport and eventually generating oxygen radicals.

It has been demonstrated in tissue culture paradigms that FGF-2 (basic FGF) has trophic activity towards nigral dopaminergic neurons (Ferrari et al., Dev. Biol. 1989). Recently, Dr. Unsicker's group has demonstrated that administering FGF-2 in gel foam implants in the striatum results in the near complete protection of nigral dopaminergic neurons from the toxicity associated with MPTP exposure (Otto and Unsicker, J. Neuroscience, 1990).

Based on the data with FGF-2, PSF-2 can be evaluated to determine whether it has an action similar to that of FGF-2 in enhancing dopaminergic neuronal survival in vitro and it can also be tested in vivo for protection of dopaminergic neurons in the striatum from the damage associated with MPTP treatment. The potential effect of PSF-2 is first examined in vitro in a dopaminergic neuronal cell culture paradigm. The cultures are prepared by dissecting the midbrain floor plate from gestation day 14 Wistar rat embryos. The tissue is dissociated with trypsin and seeded at a density of 200,000 cells/cm² on polyorthinine-laminin coated glass coverslips. The cells are maintained in Dulbecco's Modified Eagle's medium and F12 medium containing hormonal supplements (N1). The cultures are fixed with paraformaldehyde after 8 days in vitro and are processed for tyrosine hydroxylase, a specific marker for dopminergic neurons, immunohistochemical staining. Dissociated cell cultures are prepared from embryonic rats. The culture medium is changed every third day and the factors are also added at that time.

Since the dopaminergic neurons are isolated from animals at gestation day 14, a developmental time which is past the stage when the dopaminergic precursor cells are proliferating, an increase in the number of tyrosine hydroxylase immunopositive neurons would represent an increase in the number of dopaminergic neurons surviving in vitro. Therefore, if PSF-2 acts to prolong the survival of dopaminergic neurons, it would suggest that PSF-2 may be involved in Parkinson's Disease.

The studies described in this example test activity in PSF-2 protein. However, one skilled in the art could easily modify the exemplified studies to test the activity of PSF-2 polynucleotides (e.g., gene therapy), agonists, and/or antagonists of PSF-2.

### Example 36

The Effect of PSF-2 on the Growth of Vascular Endothelial Cells

On day 1, human umbilical vein endothelial cells (HWEC) are seeded at 2-5×10⁴ cells/35 mm dish density in M199 medium containing 4% fetal bovine serum (FBS), 16 units/ml heparin, and 50 units/ml endothelial cell growth supplements (ECGS, Biotechnique, Inc.). On day 2, the medium is replaced with M199 containing 10% FBS, 8 units/ml heparin. PSF-2 protein of SEQ ID NO. 2, and positive controls, such as VEGF and basic FGF (bFGF) are added, at varying concentrations. On days 4 and 6, the medium is replaced. On day 8, cell number is determined with a Coulter Counter.

An increase in the number of HUVEC cells indicates that PSF-2 may proliferate vascular endothelial cells.

The studies described in this example test activity in PSF-2 protein. However, one skilled in the art could easily modify the exemplified studies to test the activity of PSF-2 polynucleotides (e.g., gene therapy), agonists, and/or antagonists of PSF-2.

### Example 37

Stimulatory Effect of PSF-2 on the Proliferation of Vascular Endothelial Cells

For evaluation of mitogenic activity of growth factors, the colorimetric MTS (3-(4,5-dimethylthiazol-2-yl)-5-(3-carboxymethoxyphenyl)-2-(4-sulfophenyl)2H-tetrazolium) assay with the electron coupling reagent PMS (phenazine methosulfate) was performed (CellTiter 96 AQ, Promega). Cells are seeded in a 96-well plate (5,000 cells/well) in 0.1 mL serum-supplemented medium and are allowed to attach overnight. After serum-starvation for 12 hours in 0.5% FBS, conditions (bFGF, VEGF₁₆₅ or PSF-2 in 0.5% FBS) with or without Heparin (8 U/ml) are added to wells for 48 hours. 20 mg of MTS/PMS mixture (1:0.05) are added per well and allowed to incubate for 1 hour at 37° C. before measuring the absorbance at 490 nm in an ELISA plate reader. Background absorbance from control wells (some media, no cells) is subtracted, and seven wells are performed in parallel for each condition. See, Leak et al. In Vitro Cell. Dev. Biol. 30A:512-518 (1994).

The studies described in this example test activity in PSF-2 protein. However, one skilled in the art could easily modify the exemplified studies to test the activity of PSF-2 polynucleotides (e.g., gene therapy), agonists, and/or antagonists of PSF-2.

### Example 38

Inhibition of PDGF-Induced Vascular Smooth Muscle Cell Proliferation Stimulatory Effect

HAoSMC proliferation can be measured, for example, by BrdUrd incorporation. Briefly, subconfluent, quiescent cells grown on the 4-chamber slides are transfected with CRP or FITC-labeled AT2-3LP. Then, the cells are pulsed with 10% calf serum and 6 mg/ml BrdUrd. After 24 h, immunocytochemistry is performed by using BrdUrd Staining Kit (Zymed Laboratories). In brief, the cells are incubated with the biotinylated mouse anti-BrdUrd antibody at 4° C. for 2 h after being exposed to denaturing solution and then incubated with the streptavidin-peroxidase and diaminobenzidine. After counterstaining with hematoxylin, the cells are mounted for microscopic examination, and the BrdUrd-positive cells are counted. The BrdUrd index is calculated as a percent of the BrdUrd-positive cells to the total cell number. In addition, the simultaneous detection of the BrdUrd staining (nucleus) and the FITC uptake (cytoplasm) is performed for individual cells by the concomitant use of bright field illumination and dark field-UV fluorescent illumination. See, Hayashida et al., J. Biol. Chem. 6:271(36):21985-21992 (1996).

The studies described in this example test activity in PSF-2 protein. However, one skilled in the art could easily modify the exemplified studies to test the activity of PSF-2 polynucleotides (e.g., gene therapy), agonists, and/or antagonists of PSF-2.

### Example 39

Stimulation of Endothelial Migration

This example will be used to explore the possibility that PSF-2 may stimulate lymphatic endothelial cell migration. Endothelial cell migration assays are performed using a 48 well microchemotaxis chamber (Neuroprobe Inc., Cabin John, MD; Falk, W., et al., J. Immunological Methods 1980;33:239-247). Polyvinylpyrrolidone-free polycarbonate filters with a pore size of 8 um (Nucleopore Corp. Cambridge, Mass.) are coated with 0.1% gelatin for at least 6 hours at room temperature and dried under sterile air. Test substances are diluted to appropriate concentrations in M199 supplemented with 0.25% bovine serum albumin (BSA), and 25 ul of the final dilution is placed in the lower chamber of the modified Boyden apparatus. Subconfluent, early passage (2-6) HUVEC or BMEC cultures are washed and trypsinized for the minimum time required to achieve cell detachment. After placing the filter between lower and upper chamber, 2.5×10⁵ cells suspended in 50 ul M199 containing 1% FBS are seeded in the upper compartment. The apparatus is then incubated for 5 hours at 37° C. in a humidified chamber with 5% CO2 to allow cell migration. After the incubation period, the filter is removed and the upper side of the filter with the non-migrated cells is scraped with a rubber policeman. The filters are fixed with methanol and stained with a Giemsa solution (Diff-Quick, Baxter, McGraw Park, Ill.). Migration is quantified by counting cells of three random high-power fields (40×) in each well, and all groups are performed in quadruplicate.

The studies described in this example test activity in PSF-2 protein. However, one skilled in the art could easily modify the exemplified studies to test the activity of PSF-2 polynucleotides (e.g., gene therapy), agonists, and/or antagonists of PSF-2.

### Example 40

Stimulation of Nitric Oxide Production by Endothelial Cells

Nitric oxide released by the vascular endothelium is believed to be a mediator of vascular endothelium relaxation. Thus, PSF-2 activity can be assayed by determining nitric oxide production by endothelial cells in response to PSF-2.

Nitric oxide is measured in 96-well plates of confluent microvascular endothelial cells after 24 hours starvation and a subsequent 4 hr exposure to various levels of a positive control (such as VEGF-1) and PSF-2. Nitric oxide in the medium is determined by use of the Griess reagent to measure total nitrite after reduction of nitric oxide-derived nitrate by nitrate reductase. The effect of PSF-2 on nitric oxide release is examined on HUVEC.

Briefly, NO release from cultured HUVEC monolayer is measured with a NO-specific polarographic electrode connected to a NO meter (Iso-NO, World Precision Instruments Inc.) (1049). Calibration of the NO elements is performed according to the following equation:

2KNO₂+2KI+2H₂SO₄62NO+I₂+2H₂O+2K₂SO₄

The standard calibration curve is obtained by adding graded concentrations of KNO₂ (0, 5, 10, 25, 50, 100, 250, and 500 nmol/L) into the calibration solution containing KI and H₂SO₄. The specificity of the Iso-NO electrode to NO is previously determined by measurement of NO from authentic NO gas (1050). The culture medium is removed and HUVECs are washed twice with Dulbecco's phosphate buffered saline. The cells are then bathed in 5 ml of filtered Krebs-Henseleit solution in 6-well plates, and the cell plates are kept on a slide warmer (Lab Line Instruments Inc.) to maintain the temperature at 37° C. The NO sensor probe is inserted vertically into the wells, keeping the tip of the electrode 2 mm under the surface of the solution, before addition of the different conditions. S-nitroso acetyl penicillamin (SNAP) is used as a positive control. The amount of released NO is expressed as picomoles per 1×10⁶ endothelial cells. All values reported are means of four to six measurements in each group (number of cell culture wells). See, Leak et al. Biochem. and Biophys. Res. Comm., 217:96-105 (1995).

The studies described in this example tested activity in PSF-2 protein. However, one skilled in the art could easily modify the exemplified studies to test the activity of PSF-2 polynucleotides (e.g., gene therapy), agonists, and/or antagonists of PSF-2.

### Example 41

Effect of PSF-2 on Cord Formation in Angiogenesis

Another step in angiogenesis is cord formation, marked by differentiation of endothelial cells. This bioassay measures the ability of microvascular endothelial cells to form capillary-like structures (hollow structures) when cultured in vitro.

CADMEC (microvascular endothelial cells) are purchased from Cell Applications, Inc. as proliferating (passage 2) cells and are cultured in Cell Applications' CADMEC Growth Medium and used at passage 5. For the in vitro angiogenesis assay, the wells of a 48-well cell culture plate are coated with Cell Applications' Attachment Factor Medium (200 ml/well) for 30 min. at 37° C. CADMEC are seeded onto the coated wells at 7,500 cells/well and cultured overnight in Growth Medium. The Growth Medium is then replaced with 300 mg Cell Applications' Chord Formation Medium containing control buffer or PSF-2 (0.1 to 100 ng/ml) and the cells are cultured for an additional 48 hr. The numbers and lengths of the capillary-like chords are quantitated through use of the Boeckeler VIA-170 video image analyzer. All assays are done in triplicate.

Commercial (R&D) VEGF (50 ng/ml) is used as a positive control. b-esteradiol (1 ng/ml) is used as a negative control. The appropriate buffer (without protein) is also utilized as a control.

The studies described in this example test activity in PSF-2 protein. However, one skilled in the art could easily modify the exemplified studies to test the activity of PSF-2 polynucleotides (e.g., gene therapy), agonists, and/or antagonists of PSF-2.

### Example 42

Angiogenic Effect on Chick Chorioallantoic Membrane

Chick chorioallantoic membrane (CAM) is a well-established system to examine angiogenesis. Blood vessel formation on CAM is easily visible and quantifiable. The ability of PSF-2 to stimulate angiogenesis in CAM can be examined.

Fertilized eggs of the White Leghorn chick (Gallus gallus) and the Japanese qual (Coturnix coturnix) are incubated at 37.8° C. and 80% humidity. Differentiated CAM of 16-day-old chick and 13-day-old qual embryos is studied with the following methods.

On Day 4 of development, a window is made into the egg shell of chick eggs. The embryos are checked for normal development and the eggs sealed with cellotape. They are further incubated until Day 13. Thermanox coverslips (Nunc, Naperville, Ill.) are cut into disks of about 5 mm in diameter. Sterile and salt-free growth factors are dissolved in distilled water and about 3.3 mg/5 ml are pipetted on the disks. After air-drying, the inverted disks are applied on CAM. After 3 days, the specimens are fixed in 3% glutaraldehyde and 2% formaldehyde and rinsed in 0.12 M sodium cacodylate buffer. They are photographed with a stereo microscope [Wild M8] and embedded for semi- and ultrathin sectioning as described above. Controls are performed with carrier disks alone.

The studies described in this example test activity in PSF-2 protein. However, one skilled in the art could easily modify the exemplified studies to test the activity of PSF-2 polynucleotides (e.g., gene therapy), agonists, and/or antagonists of PSF-2.

### Example 43

Angiogenesis Assay Using a Matrigel Implant in Mouse

In vivo angiogenesis assay of PSF-2 measures the ability of an existing capillary network to form new vessels in an implanted capsule of murine extracellular matrix material (Matrigel). The protein is mixed with the liquid Matrigel at 4 degree C. and the mixture is then injected subcutaneously in mice where it solidifies. After 7 days, the solid “plug” of Matrigel is removed and examined for the presence of new blood vessels. Matrigel is purchased from Becton Dickinson Labware/Collaborative Biomedical Products.

When thawed at 4 C the Matrigel material is a liquid. The Matrigel is mixed with PSF-2 at 150 ng/ml at 4 degree C. and drawn into cold 3 ml syringes. Female C57B1/6 mice approximately 8 weeks old are injected with the mixture of Matrigel and experimental protein at 2 sites at the midventral aspect of the abdomen (0.5 ml/site). After 7 days, the mice are sacrificed by cervical dislocation, the Matrigel plugs are removed and cleaned (i.e., all clinging membranes and fibrous tissue is removed). Replicate whole plugs are fixed in neutral buffered 10% formaldehyde, embedded in paraffin and used to produce sections for histological examination after staining with Masson's Trichrome. Cross sections from 3 different regions of each plug are processed. Selected sections are stained for the presence of vWF. The positive control for this assay is bovine basic FGF (150 ng/ml). Matrigel alone is used to determine basal levels of angiogenesis.

The studies described in this example test activity in PSF-2 protein. However, one skilled in the art could easily modify the exemplified studies to test the activity of PSF-2 polynucleotides (e.g., gene therapy), agonists, and/or antagonists of PSF-2.

### Example 44

Rescue of Ischemia in Rabbit Lower Limb Model

To study the in vivo effects of PSF-2 on ischemia, a rabbit hindlimb ischemia model is created by surgical removal of one femoral arteries as described previously (Takeshita, S. et al., Am J. Pathol 147:1649-1660 (1995)). The excision of the femoral artery results in retrograde propagation of thrombus and occlusion of the external iliac artery. Consequently, blood flow to the ischemic limb is dependent upon collateral vessels originating from the internal iliac artery (Takeshita, S. et al. Am J. Pathol 147:1649-1660 (1995)). An interval of 10 days is allowed for post-operative recovery of rabbits and development of endogenous collateral vessels. At 10 day post-operatively (day 0), after performing a baseline angiogram, the internal iliac artery of the ischemic limb is transfected with 500 mg naked PSF-2 expression plasmid by arterial gene transfer technology using a hydrogel-coated balloon catheter as described (Riessen, R. et al. Hum Gene Ther. 4:749-758 (1993); Leclerc, G. et al. J. Clin. Invest. 90: 936-944 (1992)). When PSF-2 is used in the treatment, a single bolus of 500 mg PSF-2 protein or control is delivered into the internal iliac artery of the ischemic limb over a period of 1 min. through an infusion catheter. On day 30, various parameters are measured in these rabbits: (a) BP ratio—The blood pressure ratio of systolic pressure of the ischemic limb to that of normal limb; (b) Blood Flow and Flow Reserve—Resting FL: the blood flow during undilated condition and Max FL: the blood flow during fully dilated condition (also an indirect measure of the blood vessel amount) and Flow Reserve is reflected by the ratio of max FL: resting FL; (c) Angiographic Score—This is measured by the angiogram of collateral vessels. A score is determined by the percentage of circles in an overlaying grid that with crossing opacifled arteries divided by the total number m the rabbit thigh; (d) Capillary density—The number of collateral capillaries determined in light microscopic sections taken from hindlimbs.

The studies described in this example test activity in PSF-2 protein. However, one skilled in the art could easily modify the exemplified studies to test the activity of PSF-2 polynucleotides (e.g., gene therapy), agonists, and/or antagonists of PSF-2.

### Example 45

Effect of PSF-2 on Vasodilation

Since dilation of vascular endothelium is important in reducing blood pressure, the ability of PSF-2 to affect the blood pressure in spontaneously hypertensive rats (SHR) is examined. Increasing doses (0, 10, 30, 100, 300, and 900 mg/kg) of the PSF-2 are administered to 13-14 week old spontaneously hypertensive rats (SHR). Data are expressed as the mean+/−SEM. Statistical analysis are performed with a paired t-test and statistical significance is defined as p<0.05 vs. the response to buffer alone.

The studies described in this example test activity in PSF-2 protein. However, one skilled in the art could easily modify the exemplified studies to test the activity of PSF-2 polynucleotides (e.g., gene therapy), agonists, and/or antagonists of PSF-2.

### Example 46

Rat Ischemic Skin Flap Model

The evaluation parameters include skin blood flow, skin temperature, and factor VIII immunohistochemistry or endothelial alkaline phosphatase reaction. PSF-2 expression, during the skin ischemia, is studied using in situ hybridization.

The study in this model is divided into three parts as follows:

a) Ischemic skin

b) Ischemic skin wounds

c) Normal wounds

The experimental protocol includes:

d) Raising a 3×4 cm, single pedicle full-thickness random skin flap (myocutaneous flap over the lower back of the animal).

e) An excisional wounding (4-6 mm in diameter) in the ischemic skin (skin-flap).

f) Topical treatment with PSF-2 of the excisional wounds (day 0, 1, 2, 3, 4 post-wounding) at the following various dosage ranges: 1 mg to 100 mg.

g) Harvesting the wound tissues at day 3, 5, 7, 10, 14 and 21 post-wounding for histological, immunohistochemical, and in situ studies.

The studies described in this example test activity in PSF-2 protein. However, one skilled in the art could easily modify the exemplified studies to test the activity of PSF-2 polynucleotides (e.g., gene therapy), agonists, and/or antagonists of PSF-2.

### Example 47

Peripheral Arterial Disease Model

Angiogenic therapy using PSF-2 is a novel therapeutic strategy to obtain restoration of blood flow around the ischemia in case of peripheral arterial diseases. The experimental protocol includes:

a) One side of the femoral artery is ligated to create ischemic muscle of the hindlimb, the other side of hindlimb serves as a control.

b) PSF-2 protein, in a dosage range of 20 mg-500 mg, is delivered intravenously and/or intramuscularly 3 times (perhaps more) per week for 2-3 weeks.

c) The ischemic muscle tissue is collected after ligation of the femoral artery at 1, 2, and 3 weeks for the analysis of PSF-2 expression and histology. Biopsy is also performed on the other side of normal muscle of the contralateral hindlimb.

The studies described in this example test activity in PSF-2 protein. However, one skilled in the art could easily modify the exemplified studies to test the activity of PSF-2 polynucleotides (e.g., gene therapy), agonists, and/or antagonists of PSF-2.

### Example 48

Ischemic Myocardial Disease Model

PSF-2 is evaluated as a potent mitogen capable of stimulating the development of collateral vessels, and restructuring new vessels after coronary artery occlusion. Alteration of PSF-2 expression is investigated in situ. The experimental protocol includes:

a) The heart is exposed through a left-side thoracotomy in the rat. Immediately, the left coronary artery is occluded with a thin suture (6-0) and the thorax is closed.

b) PSF-2 protein, in a dosage range of 20 mg-500 mg, is delivered intravenously and/or intramuscularly 3 times (perhaps more) per week for 2-4 weeks.

c) Thirty days after the surgery, the heart is removed and cross-sectioned for morphometric and in situ analyzes.

The studies described in this example test activity in PSF-2 protein. However, one skilled in the art could easily modify the exemplified studies to test the activity of PSF-2 polynucleotides (e.g., gene therapy), agonists, and/or antagonists of PSF-2.

### Example 49

Rat Corneal Wound Healing Model

This animal model shows the effect of PSF-2 on neovascularization. The experimental protocol includes:

a) Making a 1-1.5 mm long incision from the center of cornea into the stromal layer.

b) Inserting a spatula below the lip of the incision facing the outer corner of the eye.

c) Making a pocket (its base is 1-1.5 mm form the edge of the eye).

d) Positioning a pellet, containing 50 ng-5 ug of PSF-2, within the pocket.

e) PSF-2 treatment can also be applied topically to the corneal wounds in a dosage range of 20 mg-500 mg (daily treatment for five days).

The studies described in this example test activity in PSF-2 protein. However, one skilled in the art could easily modify the exemplified studies to test the activity of PSF-2 polynucleotides (e.g., gene therapy), agonists, and/or antagonists of PSF-2.

### Example 50

Diabetic Mouse and Glucocorticoid-Impaired Wound Healing Models

A. Diabetic db+/db+ Mouse Model.

To demonstrate that PSF-2 accelerates the healing process, the genetically diabetic mouse model of wound healing is used. The full thickness wound healing model in the db+/db+ mouse is a well characterized, clinically relevant and reproducible model of impaired wound healing. Healing of the diabetic wound is dependent on formation of granulation tissue and re-epithelialization rather than contraction (Gartner, M. H. et al., J. Surg. Res. 52:389 (1992); Greenhalgh, D. G. et al., Am. J. Pathol. 136:1235 (1990)).

The diabetic animals have many of the characteristic features observed in Type II diabetes mellitus. Homozygous (db+/db+) mice are obese in comparison to their normal heterozygous (db+/+m) littermates. Mutant diabetic (db+/db+) mice have a single autosomal recessive mutation on chromosome 4 (db+) (Coleman et al. Proc. Natl. Acad. Sci. USA 77:283-293 (1982)). Animals show polyphagia, polydipsia and polyuria. Mutant diabetic mice (db+/db+) have elevated blood glucose, increased or normal insulin levels, and suppressed cell-mediated immunity (Mandel et al., J. Immunol. 120:1375 (1978); Debray-Sachs, M. et al., Clin. Exp. Immunol. 51(1):1-7 (1983); Leiter et al., Am. J. of Pathol. 114:46-55 (1985)). Peripheral neuropathy, myocardial complications, and microvascular lesions, basement membrane thickening and glomerular filtration abnormalities have been described in these animals (Norido, F. et al., Exp. Neurol. 83(2):221-232 (1984); Robertson et al., Diabetes 29(1):60-67 (1980); Giacomelli et al., Lab Invest. 40(4):460-473 (1979); Coleman, D. L., Diabetes 31 (Suppl):1-6 (1982)). These homozygous diabetic mice develop hyperglycemia that is resistant to insulin analogous to human type II diabetes (Mandel et al., J. Immunol. 120:1375-1377 (1978)).

The characteristics observed in these animals suggests that healing in this model may be similar to the healing observed in human diabetes (Greenhalgh, et al., Am. J. of Pathol. 136:1235-1246 (1990)).

Genetically diabetic female C57BL/KsJ (db+/db+) mice and their non-diabetic (db+/+m) heterozygous littermates are used in this study (Jackson, Laboratories). The animals are purchased at 6 weeks of age and are 8 weeks old at the beginning of the study. Animals are individually housed and received food and water ad libitum. All manipulations are performed using aseptic techniques. The experiments are conducted according to the rules and guidelines of Human Genome Sciences, Inc. Institutional Animal Care and Use Committee and the Guidelines for the Care and Use of Laboratory Animals.

Wounding protocol is performed according to previously reported methods (Tsuboi, R. and Rifkin, D. B., J. Exp. Med. 172:245-251 (1990)). Briefly, on the day of wounding, animals are anesthetized with an intraperitoneal injection of Avertin (0.01 mg/mL), 2,2,2-tribromoethanol and 2-methyl-2-butanol dissolved in deionized water. The dorsal region of the animal is shaved and the skin washed with 70% ethanol solution and iodine. The surgical area is dried with sterile gauze prior to wounding. An 8 mm full-thickness wound is then created using a Keyes tissue punch. Immediately following wounding, the surrounding skin is gently stretched to eliminate wound expansion. The wounds are left open for the duration of the experiment. Application of the treatment is given topically for 5 consecutive days commencing on the day of wounding. Prior to treatment, wounds are gently cleansed with sterile saline and gauze sponges.

Wounds are visually examined and photographed at a fixed distance at the day of surgery and at two day intervals thereafter. Wound closure is determined by daily measurement on days 1-5 and on day 8. Wounds are measured horizontally and vertically using a calibrated Jameson caliper. Wounds are considered healed if granulation tissue is no longer visible and the wound is covered by a continuous epithelium.

PSF-2 is administered using at a range different doses of PSF-2, from 4 mg to 500 mg per wound per day for 8 days in vehicle. Vehicle control groups received 50 mL of vehicle solution.

Animals are euthanized on day 8 with an intraperitoneal injection of sodium pentobarbital (300 mg/kg). The wounds and surrounding skin are then harvested for histology and immunohistochemistry. Tissue specimens are placed in 10% neutral buffered formalin in tissue cassettes between biopsy sponges for further processing.

Three groups of 10 animals each (5 diabetic and 5 non-diabetic controls) are evaluated: 1) Vehicle placebo control, 2) untreated; and 3) treated group.

Wound closure is analyzed by measuring the area in the vertical and horizontal axis and obtaining the total square area of the wound. Contraction is then estimated by establishing the differences between the initial wound area (day 0) and that of post treatment (day 8). The wound area on day 1 is 64 mm², the corresponding size of the dermal punch. Calculations are made using the following formula:

[Open area on day 8]-[Open area on day 1]/[Open area on day 1]

Specimens are fixed in 10% buffered formalin and paraffin embedded blocks are sectioned perpendicular to the wound surface (5 mm) and cut using a Reichert-Jung microtome. Routine hematoxylin-eosin (H&E) staining is performed on cross-sections of bisected wounds. Histologic examination of the wounds are used to assess whether the healing process and the morphologic appearance of the repaired skin is altered by treatment with PSF-2. This assessment included verification of the presence of cell accumulation, inflammatory cells, capillaries, fibroblasts, re-epithelialization and epidermal maturity (Greenhalgh, D. G. et al., Am. J. Pathol. 136:1235 (1990)). A calibrated lens micrometer is used by a blinded observer.

Tissue sections are also stained immunohistochemically with a polyclonal rabbit anti-human keratin antibody using ABC Elite detection system. Human skin is used as a positive tissue control while non-immune IgG is used as a negative control. Keratinocyte growth is determined by evaluating the extent of reepithelialization of the wound using a calibrated lens micrometer.

Proliferating cell nuclear antigen/cyclin (PCNA) in skin specimens is demonstrated by using anti-PCNA antibody (1:50) with an ABC Elite detection system. Human colon cancer can serve as a positive tissue control and human brain tissue can be used as a negative tissue control. Each specimen includes a section with omission of the primary antibody and substitution with non-immune mouse IgG. Ranking of these sections is based on the extent of proliferation on a scale of 0-8, the lower side of the scale reflecting slight proliferation to the higher side reflecting intense proliferation.

Experimental data are analyzed using an unpaired t test. A p value of <0.05 is considered significant.

B. Steroid Impaired Rat Model

The inhibition of wound healing by steroids has been well documented in various in vitro and in vivo systems (Wahl, S. M. Glucocorticoids and Wound healing. In: Anti-Inflammatory Steroid Action: Basic and Clinical Aspects. 280-302 (1989); Wahl, S. M.et al., J. Immunol. 115: 476-481 (1975); Werb, Z. et al., J. Exp. Med. 147:1684-1694 (1978)). Glucocorticoids retard wound healing by inhibiting angiogenesis, decreasing vascular permeability (Ebert, R. H., et al., An. Intern. Med. 37:701-705 (1952)), fibroblast proliferation, and collagen synthesis (Beck, L. S. et al., Growth Factors. 5: 295-304 (1991); Haynes, B. F. et al., J. Clin. Invest. 61: 703-797 (1978)) and producing a transient reduction of circulating monocytes (Haynes, B. F., et al., J. Clin. Invest. 61: 703-797 (1978); Wahl, S. M., “Glucocorticoids and wound healing”, In: Antiinflammatory Steroid Action: Basic and Clinical Aspects, Academic Press, New York, pp. 280-302 (1989)). The systemic administration of steroids to impaired wound healing is a well establish phenomenon in rats (Beck, L. S. et al., Growth Factors. 5: 295-304 (1991); Haynes, B. F., et al., J. Clin. Invest. 61: 703-797 (1978); Wahl, S. M., “Glucocorticoids and wound healing”, In: Antiinflammatory Steroid Action: Basic and Clinical Aspects, Academic Press, New York, pp. 280-302 (1989); Pierce, G. F. et al., Proc. Natl. Acad. Sci. USA 86: 2229-2233 (1989)).

To demonstrate that PSF-2 can accelerate the healing process, the effects of multiple topical applications of PSF-2 on full thickness excisional skin wounds in rats in which healing has been impaired by the systemic administration of methylprednisolone is assessed.

Young adult male Sprague Dawley rats weighing 250-300 g (Charles River Laboratories) are used in this example. The animals are purchased at 8 weeks of age and are 9 weeks old at the beginning of the study. The healing response of rats is impaired by the systemic administration of methylprednisolone (17 mg/kg/rat intramuscularly) at the time of wounding. Animals are individually housed and received food and water ad libitum. All manipulations are performed using aseptic techniques. This study is conducted according to the rules and guidelines of Human Genome Sciences, Inc. Institutional Animal Care and Use Committee and the Guidelines for the Care and Use of Laboratory Animals.

The wounding protocol is followed according to section A, above. On the day of wounding, animals are anesthetized with an intramuscular injection of ketamine (50 mg/kg) and xylazine (5 mg/kg). The dorsal region of the animal is shaved and the skin washed with 70% ethanol and iodine solutions. The surgical area is dried with sterile gauze prior to wounding. An 8 mm full-thickness wound is created using a Keyes tissue punch. The wounds are left open for the duration of the experiment. Applications of the testing materials are given topically once a day for 7 consecutive days commencing on the day of wounding and subsequent to methylprednisolone administration. Prior to treatment, wounds are gently cleansed with sterile saline and gauze sponges.

Wounds are visually examined and photographed at a fixed distance at the day of wounding and at the end of treatment. Wound closure is determined by daily measurement on days 1-5 and on day 8. Wounds are measured horizontally and vertically using a calibrated Jameson caliper. Wounds are considered healed if granulation tissue is no longer visible and the wound is covered by a continuous epithelium.

PSF-2 is administered using at a range different doses of PSF-2, from 4 mg to 500 mg per wound per day for 8 days in vehicle. Vehicle control groups received 50 mL of vehicle solution.

Animals are euthanized on day 8 with an intraperitoneal injection of sodium pentobarbital (300 mg/kg). The wounds and surrounding skin are then harvested for histology. Tissue specimens are placed in 10% neutral buffered formalin in tissue cassettes between biopsy sponges for further processing.

Four groups of 10 animals each (5 with methylprednisolone and 5 without glucocorticoid) are evaluated: 1) Untreated group 2) Vehicle placebo control 3) PSF-2 treated groups.

Wound closure is analyzed by measuring the area in the vertical and horizontal axis and obtaining the total area of the wound. Closure is then estimated by establishing the differences between the initial wound area (day 0) and that of post treatment (day 8). The wound area on day 1 is 64 mm², the corresponding size of the dermal punch. Calculations are made using the following formula:

[Open area on day 8]-[Open area on day 1]/[Qpen area on day 1]

Specimens are fixed in 10% buffered formalin and paraffin embedded blocks are sectioned perpendicular to the wound surface (5 mm) and cut using an Olympus microtome. Routine hematoxylin-eosin (H&E) staining is performed on cross-sections of bisected wounds. Histologic examination of the wounds allows assessment of whether the healing process and the morphologic appearance of the repaired skin is improved by treatment with PSF-2. A calibrated lens micrometer is used by a blinded observer to determine the distance of the wound gap.

Experimental data are analyzed using an unpaired t test. A p value of <0.05 is considered significant.

The studies described in this example test activity in PSF-2 protein. However, one skilled in the art could easily modify the exemplified studies to test the activity of PSF-2 polynucleotides (e.g., gene therapy), agonists, and/or antagonists of PSF-2.

### Example 51

Lymphadema Animal Model

The purpose of this experimental approach is to create an appropriate and consistent lymphedema model for testing the therapeutic effects of PSF-2 in lymphangiogenesis and re-establishment of the lymphatic circulatory system in the rat hind limb. Effectiveness is measured by swelling volume of the affected limb, quantification of the amount of lymphatic vasculature, total blood plasma protein, and histopathology. Acute lymphedema is observed for 7-10 days. Perhaps more importantly, the chronic progress of the edema is followed for up to 3-4 weeks.

Prior to beginning surgery, blood sample is drawn for protein concentration analysis. Male rats weighing approximately ˜350 g are dosed with Pentobarbital. Subsequently, the right legs are shaved from knee to hip. The shaved area is swabbed with gauze soaked in 70% EtOH. Blood is drawn for serum total protein testing. Circumference and volumetric measurements are made prior to injecting dye into paws after marking 2 measurement levels (0.5 cm above heel, at mid-pt of dorsal paw). The intradermal dorsum of both right and left paws are injected with 0.05 ml of 1% Evan's Blue. Circumference and volumetric measurements are then made following injection of dye into paws.

Using the knee joint as a landmark, a mid-leg inguinal incision is made circumferentially allowing the femoral vessels to be located. Forceps and hemostats are used to dissect and separate the skin flaps. After locating the femoral vessels, the lymphatic vessel that runs along side and underneath the vessel(s) is located. The main lymphatic vessels in this area are then electrically coagulated or suture ligated.

Using a microscope, muscles in back of the leg (near the semitendinosis and adductors) are bluntly dissected. The popliteal lymph node is then located. The 2 proximal and 2 distal lymphatic vessels and distal blood supply of the popliteal node are then and ligated by suturing. The popliteal lymph node, and any accompanying adipose tissue, is then removed by cutting connective tissues.

Care is taken to control any mild bleeding resulting from this procedure. After lymphatics are occluded, the skin flaps are sealed by using liquid skin (Vetbond) (A J Buck). The separated skin edges are sealed to the underlying muscle tissue while leaving a gap of ˜0.5 cm around the leg. Skin also may be anchored by suturing to underlying muscle when necessary.

To avoid infection, animals are housed individually with mesh (no bedding). Recovering animals are checked daily through the optimal edematous peak, which typically occurred by day 5-7. The plateau edematous peak are then observed. To evaluate the intensity of the lymphedema, the circumference and volumes of 2 designated places on each paw before operation and daily for 7 days are measured. The effect plasma proteins on lymphedema is determined and whether protein analysis is a useful testing perimeter is also investigated. The weights of both control and edematous limbs are evaluated at 2 places. Analysis is performed in a blind manner.

Circumference Measurements: Under brief gas anesthetic to prevent limb movement, a cloth tape is used to measure limb circumference. Measurements are done at the ankle bone and dorsal paw by 2 different people then those 2 readings are averaged. Readings are taken from both control and edematous limbs.

Volumetric Measurements: On the day of surgery, animals are anesthetized with Pentobarbital and are tested prior to surgery. For daily volumetrics animals are under brief halothane anesthetic (rapid immobilization and quick recovery), both legs are shaved and equally marked using waterproof marker on legs. Legs are first dipped in water, then dipped into instrument to each marked level then measured by Buxco edema software(Chen/Victor). Data is recorded by one person, while the other is dipping the limb to marked area.

Blood-plasma protein measurements: Blood is drawn, spun, and serum separated prior to surgery and then at conclusion for total protein and Ca2+ comparison.

Limb Weight Comparison: After drawing blood, the animal is prepared for tissue collection. The limbs are amputated using a quillitine, then both experimental and control legs are cut at the ligature and weighed. A second weighing is done as the tibio-cacaneal joint is disarticulated and the foot is weighed.

Histological Preparations: The transverse muscle located behind the knee (popliteal) area is dissected and arranged in a metal mold, filled with freezeGel, dipped into cold methylbutane, placed into labeled sample bags at −80EC until sectioning. Upon sectioning, the muscle is observed under fluorescent microscopy for lymphatics.

The studies described in this example test activity in PSF-2 protein. However, one skilled in the art could easily modify the exemplified studies to test the activity of PSF-2 polynucleotides (e.g., gene therapy), agonists, and/or antagonists of PSF-2.

It will be clear that the invention may be practiced otherwise than as particularly described in the foregoing description and examples. Numerous modifications and variations of the present invention are possible in light of the above teachings and, therefore, are within the scope of the appended claims.

The entire disclosure of each document cited (including patents, patent applications, journal articles, abstracts, laboratory manuals, books, or other disclosures) in the Background of the Invention, Detailed Description, and Examples is hereby incorporated herein by reference. Moreover, the sequence listing is herein incorporated by reference.

Further, the Sequence Listing submitted herewith, and the Sequence Listing submitted in copending application Ser. No. 09/461,419, filed Dec. 16, 1999 and in Serial No. 60/113,009, filed Dec. 18, 1998, in both computer-readable and paper formats (in each case), are hereby incorporated by reference in their entireties.

What is claimed is:

1. An isolated polynucleotide comprising a nucleotide sequence at least 95% identical to a polynucleotide selected from the group consisting of:
(a) a polynucleotide fragment of SEQ ID NO:1 or of the cDNA sequence included in ATCC Deposit No:203521;
(b) a polynucleotide encoding a polypeptide fragment of SEQ ID NO:2 or of the cDNA sequence included in ATCC Deposit No:203521;
(c) a polynucleotide encoding a polypeptide domain of SEQ ID NO:2 or of the cDNA sequence included in ATCC Deposit No:203521;
(d) a polynucleotide encoding a polypeptide epitope of SEQ ID NO:2 or of the cDNA sequence included in ATCC Deposit No:203521;
(e) a polynucleotide encoding a polypeptide fragment of SEQ ID NO:2 or of the cDNA sequence included in ATCC Deposit No:203521 having biological activity;
(f) a polynucleotide encoding a polypeptide comprising the amino acid sequence m¹-n¹ of SEQ ID NO:2, wherein m¹ is an integer of 2 to 299, and wherein n¹ is an integer of 6 to 303;
(g) a polynucleotide which encodes a polypeptide variant of SEQ ID NO:2, resulting from conservative substitutions;
(h) a polynucleotide which is an allelic variant of SEQ ID NO:1;
(i) a polynucleotide which encodes a species homologue of the SEQ ID NO:2;
(j) the complement of (a), (b), (c), (d), (e), (f), (g), (h), or (i); and
(k) a polynucleotide capable of hybridizing under stringent conditions to the polynucleotide of (a), (b), (c), (d), (e), (f), (g), (h), (i), or (j), wherein said polynucleotide does not hybridize under stringent conditions to a polynucleotide having a nucleotide sequence of only A residues or of only T residues.

2. The isolated polynucleotide of claim 1, wherein the polynucleotide fragment encodes:
(a) a mature PSF-2;
(b) a secreted PSF-2;
(c) a proprotein PSF-2; or
(d) a preproprotein PSF-2.

3. The polynucleotide of claim 1 fused to a polynucleotide which encodes a heterologous polypeptide.

4. An isolated polynucleotide which hybridizes under stringent conditions to:
(a) the complement of SEQ ID NO:1;
(b) the cDNA contained in ATCC Deposit No.203521; and
(c) the complement of PSF-2.

5. The polynucleotide of claim 4, wherein the polynucleotide encodes a polypeptide which encodes:
(a) a biologically active fragment of PSF-2; or
(b) a polypeptide which binds an antibody for PSF-2.

6. A recombinant vector comprising the isolated polynucleotide of claim 1.

7. A genetically engineered host cell comprising the polynucleotide of claim 1.

8. A genetically engineered host cell comprising the polynucleotide of claim 1 operatively associated with a regulatory sequence that controls gene expression.

9. A method for producing a PSF-2 polypeptide, comprising:
(a) culturing the genetically engineered host cell of claim 8 under conditions suitable to produce the polypeptide; and
(b) recovering the polypeptide from the cell culture.

10. An isolated polypeptide comprising an amino acid sequence at least 95% identical to a polypeptide selected from the group consisting of:
(a) a polypeptide fragment of SEQ ID NO:2 or of the encoded sequence included in ATCC Deposit No:203521;
(b) a polypeptide fragment of SEQ ID NO:2 or of the encoded sequence included in ATCC Deposit No:203521 having biological activity;
(c) a polypeptide domain of SEQ ID NO:2 or of the encoded sequence included in ATCC Deposit No:203521;
(d) a polypeptide epitope of SEQ ID NO:2 or of the encoded sequence included in ATCC Deposit No:203521;
(e) a polypeptide comprising the amino acid sequence m¹-n¹ of SEQ ID NO:2, wherein m¹ is an integer of 2 to 299, and wherein n¹ is an integer of 6 to 303.
(f) a mature PSF-2;
(g) a secreted PSF-2;
(h) a variant of SEQ ID NO:2 resulting from conservative substitutions;
(j) an allelic variant of SEQ ID NO:2; and
(k) a species homologue of the SEQ ID NO:2.

11. The isolated polypeptide of claim 10 fused to a heterologous polypeptide.

12. An isolated antibody that binds specifically to the isolated polypeptide of claim 10.

13. A recombinant host cell that expresses the isolated polypeptide of claim 10.

14. A method for preventing, treating, or ameliorating a medical condition which comprises administering to a mammalian subject a therapeutically effective amount of the polynucleotide of claim 1.

15. A method for preventing, treating, or ameliorating a medical condition which comprises administering to a mammalian subject a therapeutically effective amount of the polypeptide of claim 10.

16. A method of diagnosing a pathological condition or a susceptibility to a pathological condition in a subject related to expression or activity of a protein comprising:
(a) determining the presence or absence of a mutation in the polynucleotide of claim 1; and
(b) diagnosing a pathological condition or a susceptibility to a pathological condition based on the presence or absence of said mutation.

17. A method of diagnosing a pathological condition or a susceptibility to a pathological condition in a subject related to expression or activity of a protein comprising:
(a) determining the presence or amount of expression of the polypeptide of claim 10 in a biological sample; and
(b) diagnosing a pathological condition or a susceptibility to a pathological condition based on the presence or amount of expression of the polypeptide.

18. A method for identifying binding partner to the polypeptide of claim 10 comprising:
(a) contacting the polypeptide of claim 10 with a binding partner; and
(b) determining whether the binding partner binds to the polypeptide.

19. The gene corresponding to the polynucleotide of claim 1.

20. A method of identifying an activity associated with the polypeptide of claim 10, wherein the method comprises:
(a) expressing polypeptide of claim 10 from a recombinant host cell;
(b) isolating the supernatant;
(c) detecting an activity in a biological assay; and
(d) identifying the protein in the supernatant having the activity.


SEQUENCE LISTING (30 sequences)

SEQ ID NO: 1 - DNA, 1813 bp, Homo sapiens
  CDS (154)..(1065)
  sig_peptide (154)..(243)
  mat_peptide (244)..(1065)
tggccggcgggcccggctgactgcgcctctgctttctttccataaccttttctttcggactcgaatcacggctgctgcgaagggtctagttccggacactagggtgcccgaacgcgctgatgccccgagtgctcgcagggcttcccgctaaccatgctgccgccgccgcggcccgcagctgccttggcgctgcctgtgctcctgctactgctggtggtgctgacgccgcccccgaccggcgcaaggccatccccaggcccagattacctgcggcgcggctggatgcggctgctagcggagggcgagggctgcgctccctgccggccagaagagtgcgccgcgccgcggggctgcctggcgggcagggtgcgcgacgcgtgcggctgctgctgggaatgcgccaacctcgagggccagctctgcgacctggaccccagtgctcacttctacgggcactgcggcgagcagcttgagtgccggctggacacaggcggcgacctgagccgcggagaggtgccggaacctctgtgtgcctgtcgttcgcagagtccgctctgcgggtccgacggtcacacctactcccagatctgccgcctgcaggaggcggcccgcgctcggcccgatgccaacctcactgtggcacacccggggccctgcgaatcggggccccagatcgtgtcacatccatatgacacttggaatgtgacagggcaggatgtgatctttggctgtgaagtgtttgcctaccccatggcctccatcgagtggaggaaggatggcttggacatccagctgccaggggatgacccccacatctctgtgcagtttaggggtggaccccagaggtttgaggtgactggctggctgcagatccaggctgtgcgtcccagtgatgagggcacttaccgctgccttgcccgcaatgccctgggtcaagtggaggcccctgctagcttgacagtgctcacacctgaccagctgaactctacaggcatcccccagctgcgatcactaaacctggttcctgaggaggaggctgagagtgaagagaatgacgattactactaggtccagagctctggcccatgggggtgggtgagcggctatagtgttcatccctgctcttgaaaagacctggaaaggggagcagggtcccttcatcgactgctttcatgctgtcagtagggatgatcatgggaggcctatttgactccaaggtagcagtgtggtaggatagagacaaaagctggaggagggtagggagagaagctgagaccaggaccggtggggtacaaaggggcccatgcaggagatgccctggccagtaggacctccaacaggttgtttcccaggctggggtgggggcctgagcagacacagaggtgcaggcaccaggattctccacttcttccagccctgctgggccacagttctaactgcccttcctcccaggccctggttcttgctatttcctggtccccaacgtttatctagcttgtttgccctttccccaaactcatcttccagaacttttccctctctcctaagccccagttgcacctactaactgcagtcccttttgctgtctgccgtcttttgtacaagagagagaacagcggagcatgacttagttcagtgcagagagataggtgaggccagctcgagatcttataccactctgtattggacaaaggctagcacagggctaggcaccaataaagatttctaatgatgcacagaaaaaaaaaaaaaaaaaaaaaaaaaaaaaaaaaaaaaaaaaaaaaaaaaaaaaaaaaaaaaaaaaaaaaaaaaa

SEQ ID NO: 2 - protein, 304 aa, Homo sapiens [sequence omitted]

SEQ ID NO: 3 - protein, 281 aa, Mus musculus
MERPPRALLLGAAGLLLLLLPLSSSSSSDACGPCVPASCPALPRLGCPLGETRDACGCCPVCARGEGEPCGGGAAGRGHCAPGMECVKSRKRRKGKAGAAAGGPATLAVCVCKSRYPVCGSNGITYPSGCQLRAASLRAESRGEKAITQVSKGTCEQGPSIVTPPKDIWNVTGAKVFLSCEVIGIPTPVLIWNKVKRDHSGVQRTELLPGDRENLAIQTRGGPEKHEVTGWVLVSPLSKEDAGEYECHASNSQGQASAAAKITVVDALHEIPLKKGEGAQL

SEQ ID NO: 4 - DNA, 733 bp, Homo sapiens
gggatccggagcccaaatcttctgacaaaactcacacatgcccaccgtgcccagcacctgaattcgagggtgcaccgtcagtcttcctcttccccccaaaacccaaggacaccctcatgatctcccggactcctgaggtcacatgcgtggtggtggacgtaagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccaacccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgcgacggccgcgactctagaggat

SEQ ID NO: 5 - protein, 282 aa, Homo sapiens
MERPSLRALLLGAAGLLLLLLPLSSSSSSDTCGPCEPASCPPLPPLGCLLGETRDACGCCPMCARGEGEPCGGGGAGRGYCAPGMECVKSRKRRKGKAGAAAGGPGVSGVCVCKSRYPVCGSDGTTYPSGCQLRAASQRAESRGEKAITQVSKGTCEQGPSIVTPPKDIWNVTGAQVYLSCEVIGIPTPVLIWNKVKRGHYGVQRTELLPGDRDNLAIQTRGGPEKHEVTGWVLVSPLSKEDAGEYECHASNSQGQASASAKITVVDALHEIPVKKGEGAEL

SEQ ID NO: 6 - DNA, 86 bp, Artificial sequence
  5′ primer
gcgcctcgagatttccccgaaatctagatttccccgaaatgatttccccgaaatgatttccccgaaatatctgccatctcaattag

SEQ ID NO: 7 - DNA, 27 bp, Artificial sequence
  3′ primer
gcggcaagctttttgcaaagcctaggc

SEQ ID NO: 8 - DNA, 271 bp, Artificial sequence
  GAS promoter element linked to the SV40 promoter
ctcgagatttccccgaaatctagatttccccgaaatgatttccccgaaatgatttccccgaaatatctgccatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctt

SEQ ID NO: 9 - DNA, 32 bp, Artificial sequence
  5′ primer
gcgctcgagggatgacagcgatagaaccccgg

SEQ ID NO: 10 - DNA, 31 bp, Artificial sequence
  3′ primer
gcgaagcttcgcgactccccggatccgcctc

SEQ ID NO: 11 - DNA, 12 bp, Artificial sequence
  NF-kappaB binding site
ggggactttccc

SEQ ID NO: 12 - DNA, 73 bp, Artificial sequence
  5′ primer
gcggcctcgaggggactttcccggggactttccggggactttccgggactttccatcctgccatctcaattag

SEQ ID NO: 13 - DNA, 256 bp, Artificial sequence
  NF-kB/SV40 fragment
ctcgaggggactttcccggggactttccggggactttccgggactttccatctgccatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctt

SEQ ID NO: 14 - DNA, 43 bp, Artificial sequence
  5′ primer
gcagcacatatgaggccatccccaggcccagattacctgcggc

SEQ ID NO: 15 - DNA, 45 bp, Artificial sequence
  3′ primer
gcagcaggtaccttagtagtaatcgtcattctcttcactctcagc

SEQ ID NO: 16 - DNA, 48 bp, Artificial sequence
  5′ primer
gcagcaggatccgccatcatgctgccgccgccgcggcccgcagctgcc

SEQ ID NO: 17 - DNA, 45 bp, Artificial sequence; identical to SEQ ID NO: 15
  3′ primer

SEQ ID NO: 18 - DNA, 458 bp, Homo sapiens
  misc_feature (93)..(93): n equal a, t, g, or c
ggcagaggtggaggaaggatggcttggacatccagctgccaggggatgacccccacatctctgtgcagtttaggggtggaccccagaggtttnaggtgactggctggctgcagatccaggctgtgcgtcccagtgaatgagggcacttaccgctgccttgcccgcaatgccctgggtcaagtggaggcccctgctnagcttgacagtgctcacacctgaaccagctgaactctacaggcatcccccagctgcgaatcactaaacctggttcctgaggaggaggctgagaagtgaagagaatgaacgatttactanttaggtnccagagctctggcccatggggggttgggtnagngggttatagttgtttcatccctggttcttggaaaaaaccttggaaaggggagcanggtnccttcatcgattgttttcaagttttcatngggttgnntcaggga

SEQ ID NO: 19 - DNA, 354 bp, Homo sapiens
  misc_feature (109)..(109): n equals a, t, g, or c
ggcagagctggacaagcccttttttcttctcacgtcccacctcgatgcactgccctgggagggggccccaagaccttagactggatgcctttgctgggccatgctattntnagacctccnngncttcacccccagggccccagatcgtgtcacatccatatgaacacttgggaatgtgacagggcaggatgtggatctttggctgtgaaagtgtttgcctaccccatggnctccatcgagtggaggnaaggatgggcttgggacatncagcttgccaggggnttgacccccacatctttgtgncagttttnggggtgggaccccagaggttttaaggtgaantggctggctgca

SEQ ID NO: 20 - DNA, 304 bp, Homo sapiens
  misc_feature (24)..(24): n equals a, t, g, or c
ggcagagactggacaagccctttnttcttctnacgtcccacctcgatgcactgccctgggtgggggccccaagaccttagactggatgcctttgctgggccatgctattctnagacctcccgccttcacccccagggccccaaatcgtgtcacatccatatgaacacttgggaatgtgaacagggcaggatgtgaatctttggctgtgaaagtgtttgcctaccccatggnctccatcgagtgggagggaaggatggcttggnacatccagctgccaagggatgacccncacatntttntggca

SEQ ID NO: 21 - DNA, 346 bp, Homo sapiens
  misc_feature (7)..(7): n equals a, t, g, or c
ggcaganctggacaagccctttcttntnctnacgtcccacctcgatgcactgccctgggaagggggccccaagaccttagactggatgcntttcctgggccatgctattctnaaacctcccgccttcacccccaggggccccaaatcgtgtcacatccatatgaacacttgggaatgtgaacagggncaggatgtgaatctttgggctgtgaaagtgtttgcctaccccatgggcctccatcgagtggaggtaaggtatgggcttgggacatccagctngccagggggatgaccccnnacatctttntgccanttttaggggtgggaccccaaaggttttgaggtg

SEQ ID NO: 22 - DNA, 458 bp, Homo sapiens
ttttgaagcatcttttttttattcagatttttccagtcaagtaaattgtctcaaggcccctggcccaggtctgagtgtcactaccagaaggatccaccagatggcactccttcgctacttcctgtactcccaacttgccagagatgtcgcctctttccgctaaggcacaaaacttgcagagccaggtgttccccaggcgcttccccgcacgccaccgcccgcgccatccccccagcccccaccagcgtggtgagggtgtcctcggtccccaaggtctgcagtcttggccctctctgagtgtcatatggatgtgacacgatctggggccccgattcgcagggccccgggtgtgccacagtgaggttggcatcgggccgagcgcgggccgcctcctgcaggcggcagatctgggagtaggtgtgaccgtcggacccgcagagcggactctgcgaacgaca

SEQ ID NO: 23 - DNA, 305 bp, Homo sapiens
aatgccctgggtcaagtggaggccctgctagcttgacagtgctcacacctgaccagctgaactctacaggcatccccagctgcgatcactaaacctggttcctgaggaggaggctgagagtgaagagaatgacgattactactaggtccagagctctggcccatggggtgggtgagcggctatagtgttcatccctgctcttgaaagacctggaaagggagcagggtccttcatcgactgctttcatgctgtcagtagggatgatcatgggaggctatttgactccaaggtagcagtgtggtagg

SEQ ID NO: 24 - DNA, 42 bp, Artificial sequence
  5′ primer
gcagcaggatccatgctgccgccgccgcggcccgcagctgcc

SEQ ID NO: 25 - DNA, 54 bp, Artificial sequence
  5′ primer
gcagcatctagagtagtaatcgtcattctcttcactctcagcctcctcctcagg

SEQ ID NO: 26 - DNA, 48 bp, Artificial sequence
  3′ primer
gcagcatctagattagtagtaatcgtcattctcttcactctcagcctc

SEQ ID NO: 27 - DNA, 41 bp, Artificial sequence
  5′ primer
gcagcagcatgccatccccaggcccagattacctgcggcgc

SEQ ID NO: 28 - DNA, 42 bp, Artificial sequence
  3′ primer
gcagcaggatccgtagtaatcgtcattctcttcactctcagc

SEQ ID NO: 29 - DNA, 43 bp, Artificial sequence
  5′ primer
gcagcaggatccaggccatccccaggcccagattacctgcggc

SEQ ID NO: 30 - DNA, 42 bp, Artificial sequence
  3′ primer
gcagcaaagcttctagtagtaatcgtcattctcttcactctc